(19) United States
(12) Patent Application Publication
(10) Pub. No.: US 2023/0265504 A1
(43) Pub. Date: Aug. 24, 2023

(54) HIGHLY MULTIPLEXED DETECTION OF GENE EXPRESSION WITH HYBRIDIZATION CHAIN REACTION

(71) Applicant: Kanvas Biosciences, Inc., Monmouth Junction, NJ (US)

(72) Inventors: Philip S. Burnham, Monmouth Junction, NJ (US); Gregory T. Booth, Monmouth Junction, NJ (US); Hannah Bronson, Monmouth Junction, NJ (US); Matthew P. Cheng, Monmouth Junction, NJ (US); Iwijn De Vlaminck, Ithaca, NY (US); Hao Shi, Monmouth Junction, NJ (US); Prateek Sehgal, Monmouth Junction, NJ (US)

(73) Assignee: Kanvas Biosciences, Inc., Monmouth Junction, NJ (US)

(21) Appl. No.: 18/171,850

(22) Filed: Feb. 21, 2023

Related U.S. Application Data
(60) Provisional application No. 63/312,253, filed on Feb. 21, 2022.
(60) Provisional application No. 63/317,180, filed on Mar. 7, 2022.
(60) Provisional application No. 63/339,291, filed on May 6, 2022.

(51) Int. Cl. C12Q 1/6874; C12Q 1/682; C12Q 1/04; C12Q 1/686

(52) U.S. Cl. CPC C12Q 1/6874; C12Q 1/682; C12Q 1/04; C12Q 1/686; C12Q 2600/178; C12Q 2600/16

(57) ABSTRACT

Described herein are methods for rapid, highly multiplexible detection of nucleotides in samples and constructs made to be used in said methods.

### CROSS-REFERENCE TO RELATED APPLICATIONS

This application claims the benefit of U.S. Provisional Application No. 63/312,253, filed on Feb. 21, 2022, U.S. Provisional Application No. 63/317,180, filed on Mar. 7, 2022 and U.S. Provisional Application No. 63/339,291, filed on May 6, 2022, each of which is incorporated by reference in their entireties.

### SEQUENCE LISTING

This application includes and incorporates by reference in its entirety a Sequence Listing XML in the required .xml format. The Sequence Listing XML file that has been electronically filed contains the information of the nucleotide and/or amino acid sequences disclosed in the patent application using the symbols and format in accordance with the requirements of 37 C.F.R. §§ 1.832 through 1.834.

The Sequence Listing XML filed herewith serves as the electronic copy required by § 1.834(b)(1).

The Sequence Listing XML is identified as follows: “KANVAS_002_SEQ_LIST.xml” (1,125 kilo bytes in size), which was created on Feb. 20, 2023.

### TECHNICAL FIELD

This invention relates to methods for highly-multiplexed, rapid detection of nucleotides in samples, and constructs to be used in said methods.

### BACKGROUND

Microbiota often form complex communities with each other and their environment, which can include eukaryotic cells. The spatial localization of these microbes can have effects on the ecosystem, though describing the functions of each bacterium is a challenge.

### SUMMARY

Spatial transcriptomics, a method to identify specific mRNA molecules in cells in their native biological context, can be a powerful tool but has thus far been largely developed for eukaryotic systems, leaving methods to profile the spatial properties of microbial communities untouched. To accurately and comprehensively profile the microbiome transcriptome, a method that has high target multiplexity, capable of labelling potentially millions of gene targets, can be required. The development of such a method could revolutionize our understanding of microbial community assembly and lead to new diagnostic and therapeutic applications.

In one aspect, a method for analyzing a sample, can include contacting at least one encoding probe with the sample to produce a first complex, adding at least two different DNA amplifiers to the first complex to produce a second complex, and adding emissive readout probes to the second complex. Each encoding probe can include a targeting sequence and an initiator sequence. Each DNA amplifier can include an initiator complimentary sequence and a readout sequence. Each emissive readout probe can include a label and a complimentary sequence to the readout sequence of a corresponding DNA amplifier.

In another aspect, a method for analyzing a sample can include generating a set of probes, wherein each probe includes:
(i) a targeting sequence;
(ii) at least one initiator sequence; and
(iii) at least two DNA amplifiers, wherein each DNA amplifier includes an initiator complimentary sequence and a readout sequence;
contacting the set of probes with the sample to permit hybridization of the probes to nucleotides present in the sample to produce a complex;
adding a set of emissive readout probes to the complex, wherein each emissive readout probe includes a label and a sequence complimentary to the readout sequence of a corresponding DNA amplifier;
detecting the emissive readout probes in the sample;
determining the spectra of “signal” (such as, e.g., puncta, blobs) and assigning them to a bacterium; and
decoding the spectra into a single, targeted transcript through means of signal deconvolution, error correction, comparison to reference standards.

In another aspect, a method for analyzing a cell can include:
contacting at least one encoding probe with the cell to produce a first complex, wherein each encoding probe includes an mRNA targeting sequence and an initiator sequence;
adding two different DNA amplifiers to the first complex to produce a second complex, wherein each DNA amplifier includes an initiator complimentary sequence and a readout sequence; and
adding two emissive readout probes to the second complex, wherein each emissive readout probe includes a fluorophore and a complimentary sequence to the readout sequence of a corresponding DNA amplifier.

In another aspect, a construct can include a targeting sequence that is complementary to a region of interest on a DNA/RNA sequence, a first initiator sequence, a second initiator sequence that is different from the first initiator sequence, a first amplifier sequence including a readout sequence on the 5′ end of the sequence, a second amplifier sequence including a readout sequence on the 3′ end of the sequence, wherein the second amplifier sequence is different from the first amplifier sequence, and an emissive readout sequence including a sequence complimentary to the readout sequence of the first and/or second amplifier sequences and a label on the 5′ and/or 3′ end of the complimentary sequence.

In another aspect, a construct can include a targeting sequence that is a region of interest on a nucleotide, a first initiator sequence, a second initiator sequence that is different from the first initiator sequence, a first amplifier sequence including a third initiator sequence, a second amplifier sequence including a fourth initiator sequence, a third amplifier sequence including a readout sequence on the 5′ end of the sequence, a fourth amplifier sequence including a readout sequence on the 3′ end of the sequence, wherein the first, second, third, and fourth amplifier sequences are different from each other, and an emissive readout sequence including a sequence complimentary to the readout sequence of the third and/or fourth amplifier sequences and a label on the 5′ and/or 3′ end of the complimentary sequence.

In another aspect, a library of constructs can include a plurality of barcoded probes, wherein each barcoded probe can include:
a targeting sequence that is a region of interest on a nucleotide;
at least one initiator sequence;
two DNA amplifiers, wherein each DNA amplifier includes a readout sequence; and
an emissive readout probe, wherein each emissive readout probe includes a label and a sequence complimentary to the readout sequence of a corresponding DNA amplifier.
wherein at least two barcoded probes of the plurality of barcoded probes include targeting sequences that is specific to different regions of interest.

In another aspect, a library of constructs can include a plurality of barcoded probes, wherein each barcoded probe can include:
a targeting sequence that is a region of interest on a nucleotide;
a first initiator sequence;
a first and a second DNA amplifier, wherein each first and second DNA amplifier includes a second initiator sequence
a third and a fourth DNA amplifier, wherein each third and fourth DNA amplifier includes a readout sequence; and
an emissive readout probe, wherein each emissive readout probe includes a label and a sequence complimentary to the readout sequence of a corresponding third and/or fourth DNA amplifier;
wherein at least two barcoded probes of the plurality of barcoded probes include targeting sequences that are specific to different regions of interest.

In another aspect, a method for analyzing a sample can include:
contacting at least one encoding probe with the sample to produce a first complex, wherein each encoding probe can include a targeting sequence and an initiator sequence;
adding at least two different DNA amplifiers to the first complex to produce a second complex, wherein each DNA amplifier can include an initiator complimentary sequence and a readout (landing pad) sequence;
adding a set of first emissive readout probes to the second complex, wherein each of the first emissive readout probes can include a label and a complimentary sequence to the readout (landing pad) sequence of a corresponding DNA amplifier;
acquiring one or more emission spectra from the set of first emissive readout probes;
adding a set of HiPR-Swap first exchange probes to the sample, wherein each of the first exchange probes include a 100% complementary sequence to the first emissive readout probe sequence,
hybridizing the first exchange probes to the first emissive readout probes to form a third complex;
removing the third complex from the sample,
adding a set of second emissive readout probes to the second complex, wherein each of the second emissive readout probes can include a label and a complimentary sequence to the readout (landing pad) sequence of a corresponding DNA amplifier;
acquiring one or more emission spectra from the second emissive readout probes;
repeating the aforementioned steps for at least one different encoding probe.

Other aspects, embodiments, and features as disclosed herein will be apparent from the following description, the drawings, and the claims.

### BRIEF DESCRIPTION OF THE DRAWINGS

FIGS. 1A-1B are schematic drawings showing the HiPR-Cycle assay. FIG. 1A is a schematic drawing showing HiPR-Cycle, where step 1 shows a target sequence; step 2 shows an encoding probe hybridized to the target sequence, where the encoding probe has an initiator sequence; step 3 shows two DNA amplifiers, each having a different readout probe; step 4 shows amplification of the DNA amplifiers when bound to the initiator sequence of the encoding probe; and step 5 shows hybridization of the emissive readout probes to the respective amplified amplifiers. FIG. 1B is a schematic drawing showing the amplifiers (e.g., two DNA hairpins) used in the HiPR-Cycle assay Amplifiers come in pairs and are maintained as hairpin structures until they are added to the sample. The oligonucleotide design is a read-complementary sequence (15-20 nt), followed by an optional spacer (0-3 nt), a toehold region (7-12 nt, complementary to its pair's loop region), stem region (10-15 nt), loop region (7-12 nt), and complement stem region (10-15 nt). The total length is 57-65 nt in length. The hairpin structures are triggered to unfold by initiator complexes (complements of the toehold and stem region) that are concatenated to encoding probes. The amplified product then allows readout probes to hybridize to the structure. Amplifier pairs can have the same readout-complement sequence or different readout complement sequences, allowing for 1-2 dyes to hybridize to a single encoding branch.

FIG. 2 shows various images showing intensity of various readout codes under laser excitation. GFP+ E. coli were fixed and HiPR-Cycle was performed to encode GFP transcripts with zero, one, or two bits (possible bits correspond to emission in 405 nm channel or 647 nm channel). Each row is a single emission channel, excited by a different laser (top: 488 nm, middle: 405 nm, bottom: 633 nm). Columns represent unique experimental conditions for the 4 possible GFP-encodings (00=no encoding probes, 01=405 nm corresponding encoding probes, 10=633 nm corresponding encoding probes, 11=both 405 nm and 647 nm encoding probes).

FIG. 3 shows various images showing intensity of various readout codes under laser excitation, where 11-barcoded GFP mRNA transcripts are targeted with and without ribosomal RNA-targets. At left, each column represents excitation from a different laser wavelength (488 nm, 405 nm, 633 nm, and 561 nm). The top row is a single field of view for GFP+ E. coli encoded with GFP transcript, 11-barcode (corresponding to 647 nm and 405 nm readout probes). The bottom row is a single field of view for GFP+ E. coli encoded with GFP transcript, 11-barcode and containing HiPR-FISH probes targeting E. coli 16S+23S rRNA with a corresponding readout bound to Alexa Fluor 546 dye. Each image is a different emission channel, specified by inset wavelength. At right, a comparison of targeting E. coli 16S+23S rRNA with HiPR-FISH (top) and HiPR-Cycle probes is shown. Both samples were excited with 561 nm and the image is from 570 nm emission channel.

FIG. 4 shows a comparison of Readout 9 (Alexa fluor 405) emission in 414 nm channel after excitation with 405 nm laser for GFP transcripts in GFP+ E. coli. Fields of view are shown for performing the readout hybridization after overnight amplification (left), during overnight amplification (middle), and during 3 hour amplification (right).

FIG. 5 shows an example of concomitant GFP protein and transcript detection using a single readout (R2). The top panels show fluorescence signal in each channel including GFP (488 NM). Bottom panel overlays all channels and depicts mostly overlapping GFP (Green) and R2 (magenta) signal with little background from other channels.

FIG. 6 shows various images of a single field of view containing a mixed population of cells in which GFP transcripts were labeled with one barcode type: R9, R2 or R7. The top panels show fluorescent signal in each channel including GFP (488). The bottom panel overlays all channels and reveals mutually exclusive fluorescence of each readout probe within cells (GFP protein signal (488) was omitted from the merged image in order to emphasize signal from barcoded transcripts).

FIG. 7 is a graph showing the count of barcodes identified for individual cells within the field of view displayed in FIG. 6. To identify each cell's barcode, we used a thresholding approach for each “bit” (color). If the amount of signal from each barcode “bit” within a cell was surpassed, the cell's barcode would have a 1 at the respective position (otherwise 0). In this sample, only 3 barcodes (highlighted with red boxes) were present. If a cell had signal below threshold in each measured channel, it was omitted from further analysis.

FIG. 8 shows various images of HiPR-Cycle-based detection of the LacZ gene transcript in E. coli cells cultured with increasing concentration of Isopropyl ß-D-1-thiogalactopyranoside (IPTG). From left to right are images of cells grown in the presence of no IPTG, 0.1 mM IPTG, and 1 mM IPTG. Bottom panels show a zoomed in portion of the top row images (within the gray squares). Consistent with IPTG mediated induction of LacZ expression, spots corresponding to expected LacZ fluorescence are absent in the condition without IPTG and appear at higher doses of IPTG.

FIGS. 9A-9C show expression of heat shock-related genes after a drastic shock (37° C. to 53° C.). Merged images showing the E. coli cellular boundaries from segmenting bacteria based on rRNA signal (not shown) and expression of the stress response gene panel using HiPR-Cycle for a sample kept at 37° C. (FIG. 9A) and a sample shocked for 15 minutes (FIG. 9B). Intensity of the signal (emission at 423 nm from a 405 nm excitation laser) is contrasted equally for both images (FIG. 9C). The scaled intensity of the 423 nm signal (for stress response gene expression) is measured per-bacterium for the two conditions.

FIG. 10 is a schematic of initiators in the encoding probe. Top shows encoding probes contain one or two initiators bound to the target probe. Bottom shows encoding probes made from two separate probes that have neighboring target regions. Together, these probes create a continuous initiator sequence.

FIG. 11 shows confocal imaging revealing detection of amplification from round 1 (Alexa Fluor 488; left) and round 2 (Alexa Fluor 532; right) for encoding probes detecting LacZ expression. (Blue is Eubacterium stain for general 16S rRNA.

FIGS. 12A-12B are schematic representations of double amplification. In FIG. 12A, each initiator on the encoding probes corresponds to two sets of amplifiers. First, encoding probes are bound to mRNA targets, second the first amplifier set is added to specimens and an initial round of HCR is triggered. The flanking regions of the amplified constructs contain initiators for a second set of probes. A second set of amplifiers are added to probes, triggering a second stage of HCR. Fluorescently bound readout probes are added to the specimen and bound to the secondary amplified structure. FIG. 12B is a schematic of two amplifiers Amplifier set 1 binds directly to primary, encoding probes and contains flanking initiator sequences to trigger the hybridization chain reaction of amplifier set 2 Amplifier set 2 binds to the flanking region of amplifier set 1 and contains flanking readout sequences.

FIG. 13 shows that confocal imaging reveals a difference in signal intensity between standard amplification (left) and branched amplification (right) from encoding probes detecting LacZ expression. Image dimensions=135 μm×135 μm. Images are contrast normalized.

FIG. 14 shows that E. coli cells can expand uniformly when embedded in a swellable gel matrix. Fixed, GFP expressing E. coli cells were embedded in either non-expanding (left), or swellable polyacrylamide gels and GFP signal was imaged using a 488 nm excitation laser. After protease digestion for both gels, expansion of gel on the right was performed by washing the sodium-acrylate-containing gel in low salt solutions (0.05×SSC). The gel on the left does not contain sodium acrylate, and is thus non-swelling.

FIGS. 15A-15B show that multiple rounds of HiPR-Cycle can be performed on gel embedded bacterial cells. FIG. 15A is a schematic representation of HiPR-Cycle amplification products being imaged, washed away and then re-amplified off of gel integrated encoding probes. FIG. 15B: Encoding probe hybridization targeting 16s rRNA was performed on fixed E. coli following the standard HiPR-Cycle protocol. Cells were then treated with LableX to chemically modify DNA and RNA for matrix integration during gel embedding. Labeled cells were then embedded within non-expandable polyacrylamide gel matrix and proteins were digested and cleared. The HiPR-Cycle amplification was then performed by incubating the gel with amplification reagents and imaged (left panel). We then “washed away” non-gel-integrated HiPR-Cycle amplification products by washing the gel twice with nuclease free water and once with 1×PBS before imaging again (center panel). Finally, we performed HiPR-Cycle amplification again on the washed gel and imaged the resulting signal (right panel). Intensity of the signal (emission at 647 nm from a 633 nm excitation laser) is contrasted equally for all three images.

FIGS. 16A-16D show multiple sample conditions can be separately encoded for pooled analysis in a single field of view. FIG. 16A shows a fluorescent signal taken from a single field of view using five excitation lasers (405 nm, 488 nm, 514 nm, 561 nm, and 633 nm). The 405 nm, 488 nm and 633 nm lasers were used to excite the fluorescence from sample specific rRNA (16s) (blue box). The 561 nm and 514 nm laser were used to excite signal from atpD and clpB transcripts respectively (red box). FIG. 16B is a merged image showing signal from 3 distinct rRNA encoding probes. FIG. 16C is a merged image showing all emission channels overlayed. Each color represents a specific target (indicated in the legend at the bottom). FIG. 16D is a magnified image of gray highlighted area of FIG. 16C. Yellow and magenta arrows point to fluorescent spots from clpB and atpD transcripts, respectively.

FIGS. 17A-17E show that HiPR-Cycle reveals specific and broad gene expression profiles across multiple taxa in a single specimen. FIG. 17A shows multiple bacteria labeled with HIPR-FISH probes for P. aeruginosa (red), K. pneumonia (blue), and E. coli (green), within the HiPR-Cycle assay. The image is created by merging the emission channels in 633 nm (excited with 633 nm), 603 nm (excited with 514 nm), and 467 nm (excited with 405 nm). Segmentations of specific bacteria are used to determine gene expression of: FIG. 17B—rho, FIG. 17C—rne, FIG. 17D—clpB, and FIG. 17E—bla(4). Transcripts are shown with gray-scale intensity within the masked bacteria. In FIG. 17E, only signal in K. pneumoniae is shown for the purposes of illustrating bla(4) expression, and removing bleed through from other 405 nm stimulated channels. Scale bar is equivalent to 20 microns, white box in FIG. 17A is the subset for all other figures.

FIGS. 18A-18B show HiPR-Cycle details host gene expression while simultaneously labeling bacterial taxa. FIG. 18A shows a single field-of-view from merged emission channels shows the separation of the intestinal microbiome (multi-colored cells) from the mouse colon tissue. Nuclei of mouse cells are shown in blue from DAPI (4′,6-diamidino-2-phenylindole)stain, while muc2 gene expression, which is most highly expressed in goblet cells, is shown in yellow. Scale bar is 20 microns. From the white box in FIG. 16A, an airy scan was performed to show fluorescence of muc2 and DAPI detected at sub diffraction limits as shown in FIG. 18B: FISH spots are shown to be bright and punctate.

FIG. 19 shows that HiPR-cycle can be used to detect multiple genes simultaneously. The image shows the detection of Gcg (pink, Alexa Fluor 532), Aqp4 (yellow, Alexa Fluor 546), and Gsn (green, Alexa Fluor 488, and Rhodamine Red). Nuclei were DAPI stained (blue). The scale bar is 50 microns.

FIG. 20 shows that HiPR-cycle can detect gene expression with response/interaction to the microbiome. The image shows the detection of Lypd8 (green, Alexa Fluor 488) and Ubc (red, Alexa Fluor 647). In relation to bacterial genera, each is labeled with a unique dye. Image captured using a 20× objective on a Zeiss widefield epifluorescence microscope. Nuclei were DAPI stained (blue). The scale bar is 50 microns.

FIG. 21 shows that HiPR-Cycle can detect host cell types and microbial taxa. (Left) False colored image showing the identity of each nucleus after data processing (color correspondence shown to right; for example goblet cells are blue) (Left: inset; merged image of bacteria at the host-microbe interface). (Middle) Distances between host cells (rows; categorized by type) and bacterial genera (columns). Distance is shown according to heatmap. (Right) For each class of cell type and bacterial general, the distribution of centroid-centroid distance is shown (green=Duncaniella, purple=Anaeroplasma).

FIG. 22 shows that HiPR-Cycle can detect gene expression across biological kingdoms. Gene expression for kingdom-specific genes (e.g. clpB in E. coli) is shown for several genes in a mixed, fixed community of cells. DAPI is shown in pink. Outline in white are manual annotations.

FIG. 23 shows HiPR-Cycle enables the exchange of fluorescent readout probes in different rounds of imaging. In Round 1, amplified structures are probed with readout probe 11 (Alexa Fluor 488; green). In Round 2, readout probe 11 is stripped from amplified structures and readout probe 12 (Alexa Fluor 647; green) is bound. In Round 3, readout probe 12 is stripped from amplified structures and readout probe 11 is re-bound. Large images have dimensions 135 μm×135 μm.

FIG. 24 is an airy scan showing the detection of GFP proteins (left), HiPR-Cycle-based GFP proteins (middle) and GFP transcripts (right). Bottom row shown the highlighted region. (Blue is 16S rRNA).

FIG. 25 shows that HiPR-Cycle can be used with oligo-conjugated proteins to enable the detection of protein targets. Cultured NIH 3T3 fibroblasts were cultured, fixed, and detection of TubIII was attempted using an oligo-conjugated, secondary protein hybridization approach. (Left) When primary proteins are absent from the primary protein hybridization step, no signal is detected. (Right) When secondary proteins are included, a bright and punctate signal indicates the presence and position of tubulin. Nuclei were dyed with DAPI (magenta coloring). Scale bar=10 microns.

### DETAILED DESCRIPTION

It is to be appreciated that certain aspects, modes, embodiments, variations, and features of the present methods and compositions are described below in various levels of detail in order to provide a substantial understanding of the present disclosure.

### Definitions

Where values are described as ranges, endpoints are included. Furthermore, it will be understood that such disclosure includes the disclosure of all possible sub-ranges within such ranges, as well as specific numerical values that fall within such ranges irrespective of whether a specific numerical value or specific sub-range is expressly stated.

“5′-end” and “3′-end” refers to the directionality, e.g., the end-to-end orientation of a nucleotide polymer (e.g., DNA). The 5′-end of a polynucleotide is the end of the polynucleotide that has the fifth carbon.

The term “about,” as used herein, refers to +/−10% of a recited value.

“Complementary” refers to the topological compatibility or matching together of interacting surfaces of two nucleotides as understood by those of skill in the art. Thus, two sequences are “complementary” to one another if they are capable of hybridizing to one another to form a stable anti-parallel, double-stranded nucleic acid structure. A first nucleotide is complementary to a second nucleotide if the nucleotide sequence of the first nucleotide is substantially identical to the nucleotide sequence of the nucleotide binding partner of the second nucleotide, or if the first nucleotide can hybridize to the second nucleotide under stringent hybridization conditions. Thus, the nucleotide whose sequence is 5′-TATAC-3′ is complementary to a nucleotide whose sequence is 5′-GTATA-3′.

“Nucleotides,” “Nucleic acids,” “polynucleotide” or “oligonucleotide” refer to a polymeric-form of DNA and/or RNA (e.g., ribonucleotides, deoxyribonucleotides, or analogs thereof) of any length; e.g., a sequence of two or more ribonucleotides or deoxyribonucleotides. As used herein, the term “nucleotides” includes double- and single-stranded DNA, as well as double- and single-stranded RNA; it also includes modified and unmodified forms of a nucleotide (modifications to and of a nucleotide, for example, can include methylation, phosphorylation, and/or capping). In some embodiments, a nucleotide can be one of the following: a gene or gene fragment; genomic DNA; genomic DNA fragment; exon; intron; messenger RNA (mRNA); transfer RNA (tRNA); ribosomal RNA (rRNA); ribozyme; cDNA; recombinant nucleotide; branched nucleotide; plasmid; vector; isolated DNA of any sequence; isolated RNA of any sequence; any DNA described herein, any RNA described herein, primer or amplified copy of any of the foregoing.

In some embodiments, nucleotides can have any three-dimensional structure and may perform any function, known or unknown. The structure of nucleotides can also be referenced to by their 5′- or 3′-end or terminus, which indicates the directionality of the nucleotide sequence. Adjacent nucleotides in a single-strand of nucleotides are typically joined by a phosphodiester bond between their 3′ and 5′ carbons. However, different internucleotide linkages could also be used, such as linkages that include a methylene, phosphoramidate linkages, etc. This means that the respective 5′ and 3′ carbons can be exposed at either end of the nucleotide sequence, which may be called the 5′ and 3′ ends or termini. The 5′ and 3′ ends can also be called the phosphoryl (PO₄) and hydroxyl (OH) ends, respectively, because of the chemical groups attached to those ends. The term “nucleotides” also refers to both double- and single-stranded molecules.

In some embodiments, nucleotides can include modified nucleotides, such as methylated nucleotides and nucleotide analogs (including nucleotides with non-natural bases, nucleotides with modified natural bases such as aza- or deaza-purines, etc.). If present, modifications to the nucleotide structure can be imparted before or after assembly of the nucleotide sequence.

In some embodiments, the sequence of nucleotides can be interrupted by non-nucleotide components. One or more ends of the nucleotides can be protected or otherwise modified to prevent that end from interacting in a particular way (e.g. forming a covalent bond) with other nucleotides.

In some embodiments, nucleotides can be composed of a specific sequence of four nucleotide bases: adenine (A); cytosine (C); guanine (G); and thymine (T). Uracil (U) can also be present, for example, as a natural replacement for thymine when the nucleotide is RNA. Uracil can also be used in DNA. Thus, the term “sequence” refers to the alphabetical representation of nucleotides or any nucleic acid molecule, including natural and non-natural bases.

When used in terms of length, for example 20 nt, “nt” refers to nucleotide(s).

As used herein a “taxon” refers to a group of one or more populations of an organism or organisms. In some embodiments, a “taxon” refers to a phylum, a class, an order, a family, a genus, a species, or a train. In some embodiments, the disclosure includes providing a list of taxa of microorganisms. In some embodiments, the list of taxa of microorganisms is selected from a list of phyla, a list of classes, a list of orders, a list of families, a list of genera, or a list of species, of microorganisms.

In analysis of a sample, a species can be a target of interest. For example, a species can include a taxonomic species.

In the event of any term having an inconsistent definition between this application and a referenced document, the term is to be interpreted as defined herein.

### FISH

Bacteria can form biofilms, aggregations of microbial consortia, that are encased in a complex, self-produced polymeric matrix and that adhere to biological and non-biological surfaces. Environmental microbes frequently reside in biofilm microbial communities with rich taxonomic diversity and exquisite spatial organization. Biofilms have been observed on the intestinal mucosa of colorectal cancer patients, even on tumor-free mucosa far distant from the tumors. Patients with familial polyposis also harbor colonic biofilms that include tumorigenic bacteria.

The local environment of individual microbes can have strong influences on their physiology, which in turn shapes the ecology of the community. The oral plaque microbiome has been shown to exhibit intricate spatial structure, which is thought to contribute to the metabolic interactions within the microbial community, and between the community and the surrounding environment. In other instances, biofilm formation has been shown to lead to decreased antimicrobial resistance and virulence.

Sequencing strategies have revealed extensive genomic information of microbial communities from a wide range of environments, ranging from human body sites to the global ocean, but at the expense of the spatial structure of these communities.

Imaging methods based on fluorescence in-situ hybridization (FISH) have enabled studies of the spatial organization of biofilms but suffer significant multiplexity limitations. Existing FISH strategies distinguish different taxa by conjugating each taxon-specific oligonucleotide probe with a unique fluorophore or a combination of fluorophores. The spectral overlap between commercially available fluorophores and the limited range of wavelength typically used in fluorescence imaging significantly limit the number of taxa that can be probed in a single experiment using current FISH-based strategies. The state-of-the-art method allows distinction of 15 taxa, which falls short of the diversity typically observed in natural biofilm communities.

Quantitative measurements of spatial organization in microbial communities are limited by existing image segmentation algorithms. Single cell segmentation will allow physical measurements of cell size, cell shape, cell-to-cell distance, and cellular adjacency network. Previous reports have used various coarse grained metrics to quantitatively dissect spatial organization of environmental microbial communities. However, microbes in environmental biofilms are typically densely packed, which reduces the contrast between intracellular space and cells. Furthermore, cells from different taxa typically contain different amounts of ribosome, leading to a high dynamic range of biofilm images. Both factors make single-cell segmentation challenging in images of environmental biofilms.

The FISH probes typically used in existing methods are limited in their taxonomic coverage. Due to the aforementioned multiplexity limit, most existing methods either (a) use probes for a limited number of taxa at low taxonomic levels (e.g., genus or species) or (b) use probes designed at high taxonomic levels (e.g., phylum or class). Using probes for a limited number of low level taxa risks missing many low-abundance taxa. On the other hand, high taxonomic level probes do not provide high phylogenetic resolution, and can suffer from incomplete coverage of species within the target taxon.

Accordingly, methods for detection without multiplexity limit and other constraints from the art are needed.

### HiPR-Cycle

Deciphering what each cell within such communities is doing through gene expression and metabolic signatures represents the next frontier in understanding and interpreting microbial systems, with wide ranging applicability from clinical to agricultural domains (e.g., agricultural, clinical, pharmaceutical, biotechnological, medical, scientific, biotherapeutic, wastewater management domains). Here, we describe a novel technology for spatially-resolved multiplexed detection of gene expression within cells, which we refer to as Hybridization Chain Reaction (HCR) based High Phylogenetic Resolution (HiPR-Cycle). HiPR-Cycle uses HCR to extend DNA polymers upon encountering ‘initiator’ sequences attached to DNA probes which recognize target genes of interest within cells in situ. The HCR products that form at the site of detected transcripts bear High Phylogenetic Resolution microbiome mapping by Fluorescence in situ Hybridization (HiPR-FISH) readout probe binding sites, in numbers proportional to the size of the HCR product. Barcoding these products with, for example, 10 unique readout probes can be used to distinguish over 1000 distinct targets. Moreover, by physically amplifying the fluorescent signal of encoding probe binding events through HCR, the methods described herein are able to detect lowly expressed genes otherwise overlooked.

Hybridization Chain Reaction (HCR) is a method for the triggered hybridization of nucleic acid molecules starting from metastable hairpin monomers or other metastable nucleic acid structures. See, for example, Dirks, R. and Pierce, N. Proc. Natl. Acad. Sci. USA 101(43): 15275-15278 (2004), and U.S. Pat. Nos. 7,632,641; 8,105,778, 8,507,204, 10,450,599, and PCT Patent Publication WO 2021/221789, filed Mar. 4, 2021. The contents of the aforementioned disclosures are each incorporated herein by reference in their entireties.

High Phylogenetic Resolution microbiome mapping by Fluorescence in situ Hybridization (HiPR-FISH), is a versatile technology that uses binary encoding, spectral imaging, and machine learning based decoding to create micron-scale maps of the locations and identities of hundreds of microbial species in complex communities. See, for example, Shi, H. et al. “Highly multiplexed spatial mapping of microbial communities.” Nature vol. 588, 7839 (2020): 676-681, PCT Patent Publication WO 2019/173555, filed Mar. 7, 2019; PCT Patent Application No. PCT/US2022/080355, filed on Nov. 24, 2022, and U.S. application Ser. No. 18/058,171, filed on Nov. 24, 2022. The contents of the aforementioned disclosures are each incorporated herein by reference in their entireties.

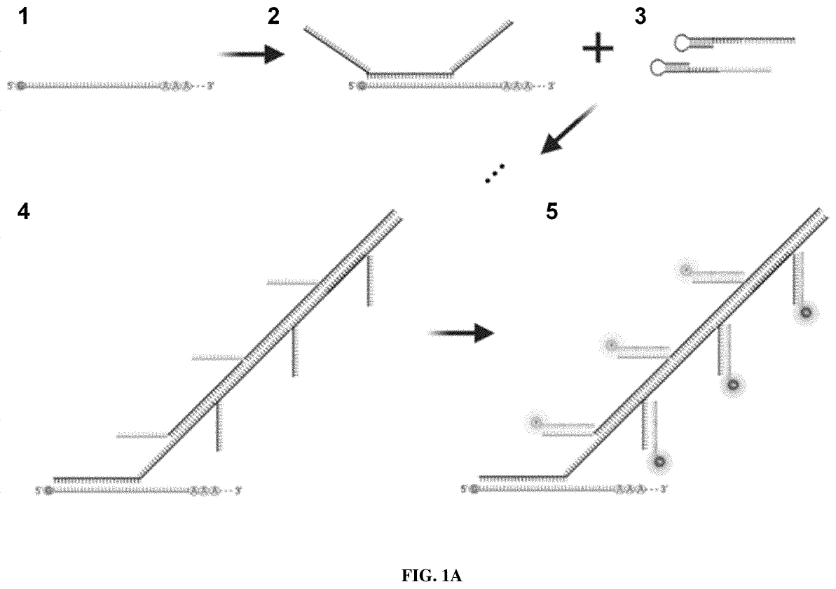

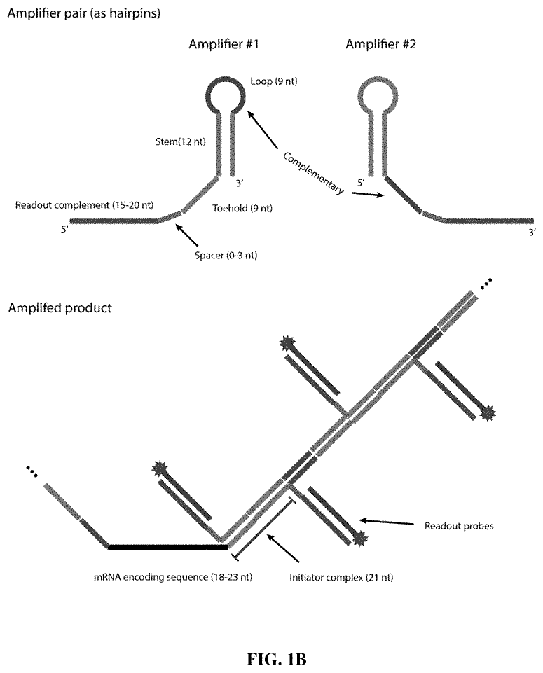

HiPR-Cycle has three primary steps: 1) encoding probe hybridization, 2) hybridization chain reaction (HCR)-based amplification, and 3) readout probe hybridization. FIG. 1A is a schematic of HiPR-Cycle assay design. FIG. 1B is a depiction of the amplifier sequence.

HiPR-Cycle can be described as follows, for example, when used to identified microbial samples:

Fixed microbial cultures are pipetted onto a glass microscope slide and allowed to dry. The cell walls of microbes can then be digested by adding lysozyme to the plated sample. To prepare the cells for encoding probe hybridization, encoding buffer (with no DNA probes) can be added to the plated sample. The pre-encoding buffer can be aspirated and new encoding buffer containing HiPR-Cycle encoding probes specific to target transcripts can be added. These encoding probes possess specific initiator sequence(s). Samples are incubated with the encoding probes. Residual encoding probes or those binding to off-target sites can be removed with 37° C. washes. At this point, samples are ready for amplification. Each reaction requires the presence of two distinct DNA amplifier species, which will cross react to form long chains in the presence of an initiator sequence. Amplification reactions can be conducted for 2 to 24 hours at room temperature. After amplification, samples are washed and a readout hybridization can be conducted by adding emissive readout probes. Once done, the sample can be dried and, once dried, mountant is applied to the sample. At this stage the sample can be imaged via microscopy.

As disclosed herein, a variety of nucleotide probes may be used to analyze a sample (e.g., by determining one or more nucleotides present in the sample).

Accordingly, a method for analyzing a sample can include:
contacting at least one encoding probe with the sample to produce a first complex, wherein each encoding probe can include a targeting sequence and an initiator sequence;
adding at least two different DNA amplifiers to the first complex to produce a second complex, wherein each DNA amplifier can include an initiator complimentary sequence and a readout sequence; and
adding emissive readout probes to the second complex, wherein each emissive readout probe can include a label and a complimentary sequence to the readout sequence of a corresponding DNA amplifier.

In some embodiments, more than one type of probe set (e.g., encoding probe, DNA amplifiers, and emissive readout probes) may be introduced to a sample. For example, there may be at least 2, at least 5, at least 10, at least 25, at least 50, at least 75, at least 100, at least 300, at least 1,000, at least 3,000, at least 10,000, or at least 30,000 distinguishable probe sets that are introduced to a sample. In some embodiments, the distinct probes are introduced simultaneously. In some embodiments, the distinct probes are introduced sequentially.

Encoding Probe Hybridization

In the methods described herein for analyzing a sample, the method can include contacting at least one encoding probe with the sample to produce a first complex, wherein each encoding probe includes a targeting sequence and an initiator sequence. This step may also be referred to as the “encoding probe hybridization” step. In here, at least one encoding probe is contacted with the sample to produce a first complex. The first complex can include the targeting sequence of the encoding probe hybridized to the nucleic acid target sequence (see, for example, step 2 of FIG. 1A).

In some embodiments, contacting the encoding probes with the sample is contacting the encoding probes with at least one nucleotide sequence of the sample. In some embodiments, contacting the encoding probes with the sample is hybridizing the encoding probe (e.g., via the targeting sequence present in the encoding probe) with a target sequence present in the sample.

In some embodiments, in order to contact the encoding probes with the sample, the sample can be digested or lysed so as to allow the encoding probes (and other probes described herein) to contact with the target sequence.

In some embodiments, to contact the at least one encoding probe with the sample to produce a first complex, encoding buffer is added to the sample. In some embodiments, the encoding buffer can include a denaturing/deionizing agent, a salt buffer, a detergent, a polyanionic polymer, a blocking agent, acids, or combinations thereof. In some embodiments, the encoding buffer can include more than one type of agent, for example, the encoding buffer can include two or more polyanionic polymers and/or two or more blocking agents. In some embodiments, the encoding buffer can include a denaturing/deionizing agent, a salt buffer, a detergent, two polyanionic polymers, two blocking agents, and an acid.

In some embodiments, the encoding buffer can include a denaturing/deionizing agent. In some embodiments, the denaturing/deionizing agent can be formamide, ethylene carbonate, or urea. In some embodiments, the encoding buffer can include about 10% (v/v) to about 50% (v/v), about 15% (v/v) to about 45% (v/v), about 20% (v/v) to about 40% (v/v), about 25% (v/v) to about 35% (v/v), about 10% (v/v), 15% (v/v), 20% (v/v), 25% (v/v), or 30% (v/v) of a denaturing/deionizing agent (e.g., formamide).

In some embodiments, the encoding buffer can include a salt buffer. In some embodiments, the salt buffer is saline sodium citrate (SSC), NaCl, or MgCl₂. In some embodiments, the encoding buffer can include about 2× to about 20×, about 5× to about 10×, or about 5× of a salt buffer (e.g., saline sodium citrate (SSC)).

In some embodiments, the encoding buffer can include at least one polyanionic polymer. In some embodiments, the encoding buffer can include one polyanionic polymer. In some embodiments, the encoding buffer can include two polyanionic polymers. In some embodiments, the polyanionic polymer can be dextran sulfate, heparin, or polyglutamic acid. In some embodiments, the encoding buffer can include about 2.5% (v/v) to about 25% (v/v), about 5% (v/v) to about 15% (v/v), about 7.5% (v/v) to about 12.5% (v/v), about 5% (v/v), or about 10% (v/v) of a polyanionic polymer (e.g., dextran sulfate). In some embodiments, the encoding buffer can include about 20 μg/mL to about 80 μg/mL, about 30 μg/mL to about 70 μg/mL, about 40 μg/mL to about 60 μg/mL, or about 50 μg/mL of a polyanionic polymer (e.g., heparin).

In some embodiments, the encoding buffer can include a detergent. In some embodiments, the detergent can be Tween 20, Tween 80, sodium dodecyl sulfate (SDS), Triton X-100, Triton X-114, NP-40, Brij-35, Brij-58. N-Dodecyl-beta-maltoside, Octyl-beta-glucoside, octylthioglucoside (OTG). In some embodiments, the encoding buffer can include about 0.01% (v/v) to about 1.0% (v/v), about 0.05% (v/v) to about 0.5% (v/v), or about 0.1% (v/v), or about 0.05% (v/v) of detergent (e.g., Tween 20).

In some embodiments, the encoding buffer can include an acid. In these embodiments, the acid lowers the pH of the buffer. In some embodiments, the acid can be citric acid. In some embodiments, the encoding buffer can include about 1 mM to about 30 mM, about 5 mM to about 15 mM, about 5 mM to about 10 mM, about 7 mM to about 10 mM, or about 9 mM of an acid (e.g., citric acid).

In some embodiments, the encoding buffer can include at least one blocking agent. In some embodiments, the encoding buffer can include one blocking agent. In some embodiments, the blocking agents can be Denhardt's solution, bovine serum albumin (BSA), salmon sperm DNA, Ficoll, polyvinyl pyrrolidone (PVP), E. coli tRNA, casein solution, or random hexamers. In some embodiments, the encoding buffer can include about 0.1× to about 10×, about 0.5× to about 5×, about 1× to about 2×, or about 1× of a blocking agent (e.g., Denhardt's solution).

In some embodiments, the encoding buffer can include formamide, SSC, dextran sulfate, Tween 20, citric acid (pH 6), heparin, and Denhardt's solution. In some embodiments citric acid and/or heparin can be omitted from the encoding buffer composition. In some embodiments, the encoding buffer can include 30% formamide, 5×SSC, 10% dextran sulfate, 0.1% Tween 20, 9 mM citric acid (pH 6), 50 μg/mL heparin and 1×Denhardt's solution.

In some embodiments, the encoding buffer can include SSC, dextran sulfate, ethylene carbonate, SDS, and Denhardt's solution. In some embodiments, the encoding buffer can include 2×SSC, 10% dextran sulfate, 10% ethylene carbonate, 0.01% SDS, and 5×Denhardt's solution.

Amplification

Following the hybridization of the encoding probe with the target sequence to form a first complex, at least two different DNA amplifiers are added to the first complex to produce a second complex, wherein each DNA amplifier can include an initiator complimentary sequence and a readout sequence. In some embodiments, this step may be referred to as the “amplification” step. In here, each amplification step/reaction requires the presence of two different DNA amplifiers, which cross react to form long nucleotide chains in the presence of an initiator sequence (see, for example, steps 3 and 4 in FIG. 1A).

In some embodiments, prior to adding the two DNA amplifiers to the first complex, each DNA amplifier is briefly heated (e.g., at 95° C. for 2 minutes) to denature any unwanted structure, followed by a cooling period (e.g., to room temperature) where the DNA amplifier (e.g., hairpin structure) reforms.

In order to form the second complex (e.g., perform amplification step), amplification buffer is added to the sample. In some embodiments, the amplification buffer can include a salt buffer, a detergent a polyanionic polymer, a denaturing/deionizing reagent, or combinations thereof. In some embodiments, the amplification buffer can include a salt buffer, a detergent a polyanionic polymer, and a denaturing/deionizing reagent.

In some embodiments, the amplification buffer can include a salt buffer. In some embodiments, the salt buffer is saline sodium citrate (SSC), NaCl, or MgCl₂. In some embodiments, the amplification buffer can include about 2× to about 20×, about 5× to about 10×, or about 5× of a salt buffer (e.g., saline sodium citrate (SSC)).

In some embodiments, the amplification buffer can include a detergent. In some embodiments, the detergent can be Tween 20, Tween 80, sodium dodecyl sulfate (SDS), Triton X-100, Triton X-114, NP-40, Brij-35, Brij-58. N-Dodecyl-beta-maltoside, Octyl-beta-glucoside, octylthioglucoside (OTG). In some embodiments, the amplification buffer can include about 0.01% (v/v) to about 1.0% (v/v), about 0.05% (v/v) to about 0.5% (v/v), or about 0.1% (v/v), or about 0.05% (v/v) of detergent (e.g., Tween 20).

In some embodiments, the amplification buffer can include at least one polyanionic polymer. In some embodiments, the amplification buffer can include one polyanionic polymer. In some embodiments, the amplification buffer can include two polyanionic polymers. In some embodiments, the polyanionic polymer can be dextran sulfate, heparin, or polyglutamic acid. In some embodiments, the amplification buffer can include about 2.5% (v/v) to about 25% (v/v), about 5% (v/v) to about 15% (v/v), about 7.5% (v/v) to about 12.5% (v/v), about 5% (v/v), or about 10% (v/v) of a polyanionic polymer (e.g., dextran sulfate). In some embodiments, the amplification buffer can include about 20 μg/mL to about 80 μg/mL, about 30 μg/mL to about 70 μg/mL, about 40 μg/mL to about 60 μg/mL, or about 50 μg/mL of a polyanionic polymer (e.g., heparin).

In some embodiments, the amplification buffer can include a denaturing/deionizing agent. In some embodiments, the denaturing/deionizing agent can be formamide, ethylene carbonate, or urea. In some embodiments, the amplification buffer can include about 10% (v/v) to about 50% (v/v), about 15% (v/v) to about 45% (v/v), about 20% (v/v) to about 40% (v/v), about 25% (v/v) to about 35% (v/v), about 10% (v/v), 15% (v/v), 20% (v/v), 25% (v/v), or 30% (v/v) of a denaturing/deionizing agent (e.g., formamide).

In some embodiments, the amplification buffer can include SSC, dextran sulfate, and Tween 20. In some embodiments, the amplification buffer can include 5×SSC, 10% dextran sulfate, and 0.1% Tween 20.

In some embodiments, the amplification reaction can be conducted at about 4° C. to about 37° C., or at room temperature, at 4° C. or at 37° C., depending on the properties of the amplifier probes. In some embodiments, the amplification reaction can be conducted for about 30 minutes to about 24 hours, or about 2 hours to about 12 hours, or about 2 hours to about 5 hours, or about 2 hours to about 4 hours, about 2 hours to about 3 hours, or about 3 hours, or about 2 hours.

In some embodiments, after the amplification reaction is completed, a washing step can be performed. In some embodiments, the washing step can be with a washing buffer comprising about 2×, 3×, 4×, or 5×SSCT (2×SSC+0.1% Tween 20). In some embodiments, the washing step can be conducted at about room temperature to about 48° C.

In some embodiments, contacting at least one encoding probe with the sample to produce a first complex and adding at least two different DNA amplifiers to the first complex to produce a second complex are performed at the same time. In some embodiments, the amplification step can be performed simultaneously with the readout probe hybridization step.

Readout Probe Hybridization

After the amplification step is complete, emissive readout probes are added to the second complex, wherein each emissive readout probe can include a label and a complimentary sequence to the readout sequence of a corresponding DNA amplifier. In some embodiments, this step may be referred to as the “readout probe hybridization” step. In here, the emissive readout probes hybridize to their complementary sequences present in the second complex (see, for example, step 5 of FIG. 1A).

In some embodiments, the readout probes are added so they achieve a final concentration of about 10 nM to about 20 μM, or about 10 nM to about 10 μM, or about 100 nM to about 1 μM, about 200 nM to about 500 nM, or about 200 nM, about 300 nM, about 400 nM, or about 500 nM for each readout probe. In some embodiments, the readout probes are added so they achieve a final concentration of about 400 nM.

In some embodiments, to hybridize the readout probes to the second complex, readout buffer is added to the sample. In some embodiments, the readout buffer can include a denaturing/deionizing agent, a salt buffer, a detergent, a polyanionic polymer, a blocking agent, or combinations thereof. In some embodiments, the readout buffer includes more than one type of agent, for example, the readout buffer can include two or more polyanionic polymers and/or two or more blocking agents.

In some embodiments, the readout buffer can include a denaturing/deionizing agent. In some embodiments, the denaturing/deionizing agent can be formamide, ethylene carbonate, or urea. In some embodiments, the readout buffer can include about 10% (v/v) to about 50% (v/v), about 15% (v/v) to about 45% (v/v), about 20% (v/v) to about 40% (v/v), about 25% (v/v) to about 35% (v/v), about 10% (v/v), 15% (v/v), 20% (v/v), 25% (v/v), or 30% (v/v) of a denaturing/deionizing agent (e.g., formamide or ethylene carbonate).

In some embodiments, the readout buffer can include a salt buffer. In some embodiments, the salt buffer is saline sodium citrate (SSC), NaCl, or MgCl₂. In some embodiments, the readout buffer can include about 2× to about 20×, about 5× to about 10×, about 5×, or about 2× of a salt buffer (e.g., saline sodium citrate (SSC)).

In some embodiments, the readout buffer can include at least one polyanionic polymer. In some embodiments, the readout buffer can include one polyanionic polymer. In some embodiments, the polyanionic polymer can be dextran sulfate, heparin, or polyglutamic acid. In some embodiments, the readout buffer can include about 2.5% (v/v) to about 25% (v/v), about 5% (v/v) to about 15% (v/v), about 7.5% (v/v) to about 12.5% (v/v), about 5% (v/v), or about 10% (v/v) of a poly anionic polymer (e.g., dextran sulfate).

In some embodiments, the readout buffer can include a detergent. In some embodiments, the detergent can be Tween 20, Tween 80, sodium dodecyl sulfate (SDS), Triton X-100, Triton X-114, NP-40, Brij-35, Brij-58. N-Dodecyl-beta-maltoside, Octyl-beta-glucoside, octylthioglucoside (OTG). In some embodiments, the encoding buffer can include about 0.005 (v/v) to about 1.0% (v/v), about 0.01% (v/v) to about 0.05% (v/v), about 0.05% (v/v) to about 0.5% (v/v), or about 0.1% (v/v), about 0.01% (v/v), or about 0.05% (v/v) of detergent (e.g., SDS).

In some embodiments, the readout buffer can include at least one blocking agent. In some embodiments, the readout buffer can include one blocking agent. In some embodiments, the blocking agents can be Denhardt's solution, bovine serum albumin (BSA), salmon sperm DNA, Ficoll, polyvinyl pyrrolidone (PVP), E. coli tRNA, casein solution, or random hexamers. In some embodiments, the readout buffer can include about 0.1× to about 10×, about 0.5× to about 5×, about 1× to about 2×, or about 1× of a blocking agent (e.g., Denhardt's solution).

In some embodiments, the readout buffer can include SSC, Denhardt's solution, dextran sulfate, ethylene carbonate, and SDS. In some embodiments, the readout buffer can include 2×SSC, 5×Denhardt's solution, 10% (v/v) dextran sulfate, 10% (v/v) ethylene carbonate, and 0.01% (v/v) SDS.

In some embodiments, the readout buffer can include SSC, Denhardt's solution, dextran sulfate, formamide, and SDS. In some embodiments, the readout buffer can include 2×SSC, 5×Denhardt's solution, 10% (v/v) dextran sulfate, 10% (v/v) formamide, and 0.01% (v/v) SDS.

In some embodiments, the readout hybridization reaction can be conducted at about 4° C. to about 37° C., or at room temperature, at 4° C. or at 37° C., depending on the properties of the amplifier probes In some embodiments, the readout hybridization reaction can be conducted for about 2 hours to about 24 hours, or about 2 hours to about 12 hours, or about 2 hours to about 5 hours, or about 2 hours to about 4 hours, about 2 hours to about 3 hours, or about 3 hours, or about 2 hours.

In some embodiments, after each reaction and before proceeding to the next one, the samples or probes are washed with a “wash buffer.”

In some embodiments, the wash buffer can include a denaturing/deionizing agent, a salt buffer, a detergent, a polyanionic polymer, acids, a pH stabilizer, a chelating agent, or combinations thereof. In some embodiments, the wash buffer can include more than one type of agent, for example, the wash buffer can include two or more detergents. In some embodiments, the wash buffer can include a denaturing/deionizing agent, a salt buffer, a detergent, a polyanionic polymer, and an acid. In some embodiments, the wash buffer can include a salt buffer and a detergent. In some embodiments, the wash buffer can include a salt buffer, a pH stabilizer, and a chelating agent.

In some embodiments, the wash buffer can include a denaturing/deionizing agent. In some embodiments, the denaturing/deionizing agent can be formamide, ethylene carbonate, or urea. In some embodiments, the wash buffer can include about 10% (v/v) to about 50% (v/v), about 15% (v/v) to about 45% (v/v), about 20% (v/v) to about 40% (v/v), about 25% (v/v) to about 35% (v/v), about 10% (v/v), 15% (v/v), 20% (v/v), 25% (v/v), or 30% (v/v) of a denaturing/deionizing agent (e.g., formamide).

In some embodiments, the wash buffer can include a salt buffer. In some embodiments, the salt buffer is saline sodium citrate (SSC), NaCl, or MgCl₂. In some embodiments, the wash buffer can include about 2× to about 20×, about 5× to about 10×, or about 5× of a salt buffer (e.g., saline sodium citrate (SSC)).

In some embodiments, the wash buffer can include a polyanionic polymer. In some embodiments, the polyanionic polymer can be dextran sulfate, heparin, or polyglutamic acid. In some embodiments, the wash buffer can include about 2.5% (v/v) to about 25% (v/v), about 5% (v/v) to about 15% (v/v), about 7.5% (v/v) to about 12.5% (v/v), about 5% (v/v), or about 10% (v/v) of a polyanionic polymer (e.g., dextran sulfate). In some embodiments, the wash buffer can include about 20 μg/mL to about 80 μg/mL, about 30 μg/mL to about 70 μg/mL, about 40 μg/mL to about 60 μg/mL, or about 50 μg/mL of a polyanionic polymer (e.g., heparin).

In some embodiments, the wash buffer can include a detergent. In some embodiments, the detergent can be Tween 20, Tween 80, sodium dodecyl sulfate (SDS), Triton X-100, Triton X-114, NP-40, Brij-35, Brij-58. N-Dodecyl-beta-maltoside, Octyl-beta-glucoside, octylthioglucoside (OTG). In some embodiments, the wash buffer can include about 0.01% (v/v) to about 1.0% (v/v), about 0.05% (v/v) to about 0.5% (v/v), or about 0.1% (v/v), or about 0.05% (v/v) of detergent (e.g., Tween 20).

In some embodiments, the wash buffer can include an acid. In these embodiments, the acid lowers the pH of the buffer. In some embodiments, the acid can be citric acid. In some embodiments, the wash buffer can include about 1 mM to about 30 mM, about 5 mM to about 15 mM, about 5 mM to about 10 mM, about 7 mM to about 10 mM, or about 9 mM of an acid (e.g., citric acid).

In some embodiments, the wash buffer can include a pH stabilizer. In some embodiments, the pH stabilizer can be at least one of tris-HCl, citric acid, SSC, 4-(2-hydroxyethyl)-1-piperazineethanesulfonic acid (HEPES), sucrose/EDTA/Tris-HCl (SET), potassium phosphate, tris(hydroxymethyl)methylamino]propanesulfonic acid (TAPS), NaOH, 3-(N-morpholino)propanesulfonic acid (MOPS), Tricine, Bicine, sodium pyrophosphate, piperazine-N,N′-bis(2-ethanesulfonic acid) (PIPES), SSPE. In some embodiments, the pH stabilizer can be tris-HCl. In some embodiments, the wash buffer can include about 5 mM to about 30 mM, about 10 mM to about 20 mM, about 10 mM, or about 20 mM of a pH stabilizer (e.g., tris-HCl).

In some embodiments, the wash buffer can include a chelating agent. In some embodiments, the chelating agent is at least one of EDTA, Ethylene glycol tetraacetic acid (EGTA), Salicylic acid, Triethanolamine (TEA), or Dimercaptopropanol. In some embodiments, the chelating agent is EDTA. In some embodiments, the was buffer can include about 1 mM to about 10 mM, about 2 mM to about 5 mM, or about 5 mM of a chelating agent (e.g., EDTA).

In some embodiments, the wash buffer can include formamide, SSC, Tween 20, citric acid (pH 6), and heparin. In some embodiments citric acid and/or heparin can be omitted from the wash buffer composition. In some embodiments, the wash buffer can include 30% formamide, 5×SSC, 0.1% Tween 20, optional 9 mM citric acid (pH 6), and optional 50 μg/mL heparin.

In some embodiments, the wash buffer can include SSC and Tween 20. In some embodiments, the wash buffer can include 5×SSC and 0.1% Tween 20. In some embodiments, the wash buffer can include NaCl, tris-HCl, and EDTA. In some embodiments, the wash buffer can include 215 mM NaCl, 20 mM tris-EDTA, and 5 mM EDTA.

In another aspect, a method for analyzing a sample can include:
generating a set of probes, wherein each probe can include:
   a targeting sequence;
   at least one initiator sequence; and
   at least two DNA amplifiers, wherein each DNA amplifier can include an initiator complimentary sequence and a readout sequence;
contacting the set of probes with the sample to permit hybridization of the probes to nucleotides present in the sample to produce a complex;
adding a set of emissive readout probes to the complex, wherein each emissive readout probe can include a label and a sequence complimentary to the readout sequence of a corresponding DNA amplifier; and
detecting the emissive readout probes in the sample.

Sample

In some embodiments, the sample is at least one of a cell, a cell suspension, a tissue biopsy, a tissue specimen, urine, stool, blood, serum, plasma, bone biopsies, bone marrow, respiratory specimens, sputum, induced sputum, tracheal aspirates, bronchoalveolar lavage fluid, sweat, saliva, tears, ocular fluid, cerebral spinal fluid, pericardial fluid, pleural fluid, peritoneal fluid, placenta, amnion, pus, nasal swabs, nasopharyngeal swabs, oropharyngeal swabs, ocular swabs, skin swabs, wound swabs, mucosal swabs, buccal swabs, vaginal swabs, vulvar swabs, nails, nail scrapings, hair follicles, corneal scrapings, gavage fluids, gargle fluids, abscess fluids, wastewater, or plant biopsies.

In some embodiments, the sample is a cell. In some embodiments, the cell is a bacterial cell. In some embodiments, the cell is a eukaryotic cell. In some embodiments the eukaryotic cell is a unicellular organism including protozoa, chromista, algae, or fungi. In some embodiments the eukaryotic cell is part of a multicellular organism from chromista, plantae, fungi, or animalia. In some embodiments the sample is a tissue composed of cells. In some embodiments the cell contains foreign DNA/RNA from viruses, plasmids, and bacteria.

In some embodiments, the sample can include a plurality of cells. In some embodiments, each cell in the plurality of cells can include a specific targeting sequence, which may or may not be the same from the other targeting sequences.

In some embodiments, the sample is a human oral microbiome sample. In some embodiments, the sample is a whole organism.

In some embodiments, the sample is obtained from a patient diagnosed with, or suspected to be suffering from an infection, disease, or disorder. In some embodiments, the patient has been diagnosed with, or is suspected to be suffering from a bacterial, viral, fungal, or parasitic infection. In some embodiments, the infection includes, but is not limited to, Acute Flaccid Myelitis, Anaplasmosis, Anthrax, Babesiosis, Botulism, Brucellosis, Campylobacteriosis, Carbapenem-resistant Infection (CRE/CRPA), Chancroid, Chickenpox, Chikungunya Virus Infection (Chikungunya), Chlamydia, Ciguatera (Harmful Algae Blooms (HABs)), Clostridium difficile Infection, Clostridium perfringens (Epsilon Toxin), Coccidioidomycosis fungal infection (Valley fever), COVID-19 (Coronavirus Disease 2019), Creutzfeldt-Jacob Disease, transmissible spongiform encephalopathy (CJD), Cryptosporidiosis (Crypto), Cyclosporiasis, Dengue, 1,2,3,4 (Dengue Fever), Diphtheria, E. coli infection, Shiga toxin-producing (STEC), Eastern Equine Encephalitis (EEE), Ebola Hemorrhagic Fever (Ebola), Ehrlichiosis, Encephalitis, Arboviral or parainfectious, Enterovirus Infection, D68 (EV-D68), Enterovirus Infection, Non-Polio (Non-Polio Enterovirus), Giardiasis (Giardia), Glanders, Gonococcal Infection (Gonorrhea), Granuloma inguinale, Haemophilus Influenza disease, Type B (Hib or H-flu), Hantavirus Pulmonary Syndrome (HPS), Hemolytic Uremic Syndrome (HUS), Hepatitis (A, B, C, D, and/or E), Herpes Herpes Zoster, zoster VZV (Shingles), Histoplasmosis infection (Histoplasmosis), Human Immunodeficiency Virus/AIDS (HIV/AIDS), Human Papillomavirus (HPV), Influenza (Flu), Lead Poisoning, Legionellosis (Legionnaires Disease), Leishmaniasis, Leprosy (Hansens Disease), Leptospirosis, Listeriosis (Listeria), Lyme Disease, Lymphogranuloma venereum infection (LGV), Malaria, Measles, Melioidosis, Meningitis, Viral (Meningitis, viral), Meningococcal Disease, Bacterial (Meningitis, bacterial), Middle East Respiratory Syndrome Coronavirus (MERS-CoV), Mononucleosis, Multisystem Inflammatory Syndrome in Children (MIS-C), Mumps, Norovirus, Paralytic Shellfish Poisoning (Paralytic Shellfish Poisoning, Ciguatera), Pediculosis (Lice, Head and Body Lice), Pelvic Inflammatory Disease (PID), Pertussis (Whooping Cough), Plague; Bubonic, Septicemic, Pneumonic (Plague), Pneumococcal Disease (Pneumonia), Poliomyelitis (Polio), Powassan, Psittacosis (Parrot Fever), Phthiriasis (Crabs; Pubic Lice Infestation), Pustular Rash diseases (Small pox, monkeypox, cowpox), Q-Fever, Rabies, Ricin Poisoning, Rickettsiosis (Rocky Mountain Spotted Fever), Rubella, Salmonellosis gastroenteritis (Salmonella), Scabies Infestation (Scabies), Scombroid, Septic Shock (Sepsis), Severe Acute Respiratory Syndrome (SARS), Shigellosis gastroenteritis (Shigella), Smallpox, Staphylococcal Infection, Methicillin-resistant (MRSA), Staphylococcal Food Poisoning, Enterotoxin-B Poisoning (Staph Food Poisoning), Staphylococcal Infection, Vancomycin Intermediate (VISA), Staphylococcal Infection, Vancomycin Resistant (VRSA), Streptococcal Disease, Group A (invasive) (Strep A (invasive)), Streptococcal Disease, Group B (Strep-B), Streptococcal Toxic-Shock Syndrome, STSS, Toxic Shock (STSS, TSS), Syphilis, primary, secondary, early latent, late latent, congenital, Tetanus, Toxoplasmosis, Trichomoniasis (Trichomonas infection), Trichinosis Infection (Trichinosis), Tuberculosis (Latent) (LTBI), Tuberculosis (TB), Tularemia (Rabbit fever), Typhus, Typhoid Fever, Group D, Vaginosis, bacterial (Yeast Infection), Vaping-Associated Lung Injury (e-Cigarette Associated Lung Injury), Varicella (Chickenpox), Vibrio cholerae (Cholera), Vibriosis (Vibrio), Viral Hemorrhagic Fever (Ebola, Lassa, Marburg), West Nile Virus, Yellow Fever, Yersenia (Yersinia), or Zika Virus Infection (Zika).

In some embodiments, when the sample is obtained from a patient, the patient has been diagnosed with, or is suspected to be suffering from an infection caused by a bacterium selected from the group consisting of: Acinetobacter, Actinomyces, Aerococcus, Bacteroides, Bartonella, Brucella, Bordetella, Burkholderia, Campylobacter, Chlamydia, Citrobacter, Clostridium, Corynebacterium, Edwardsiella, Elizabethkingia, Enterobacter, Enterococcus, Escherichia, Fusobacterium, Haemophilus, Helicobacter, Klebsiella, Legionella, Leptospira, Listeria, Morganella, Mycobacterium, Mycoplasma, Neisseria, Pantoea, Prevotella, Proteus, Providencia, Pseudomonas, Raoultella, Salmonella, Serratia, Shigella, Staphylococcus, Stenotrophomonas, Streptococcus, Ureaplasma, and Vibrio.

In some embodiments, when the sample is obtained from a patient, the patient has been diagnosed with, or is suspected to be suffering from an infection caused by a virus selected from the group consisting of: bacteriophage, RNA bacteriophage (e.g., MS2, AP205, PP7 and Qβ), Infectious Haematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, and Poliovirus, Norovirus, Zika virus, Denge Virus, Rabies Virus, Newcastle Disease Virus, and White Spot Syndrome Virus.

In some embodiments, when the sample is obtained from a patient, the patient has been diagnosed with, or is suspected to be suffering from an infection caused by a parasite selected from the group consisting of: Plasmodium, Trypanosoma, Toxoplasma, Giardia, Leishmania, Cryptosporidium, helminthic parasites: Trichuris spp., Enterobius spp., Ascaris spp., Ancylostoma spp. and Necatro spp., Strongyloides spp., Dracunculus spp., Onchocerca spp. and Wuchereria spp., Taenia spp., Echinococcus spp., and Diphyllobothrium spp., Fasciola spp., and Schistosoma spp.

Encoding Probes

Encoding probes are probes that bind directly to a target or targeting sequence and contain either 1 or 2 branches extending away from the hybridization site. The branches can either correspond to the readout sequences, initiator sequences, and/or sequences that comprise at least one site for secondary hybridization events. Encoding probes, for example, are designed to target bacterial ribosomal RNA (rRNA) and messenger RNA (mRNA) targets.

For example, rRNA-probes can contain (5′ to 3′):
a. Primer sequences to enrich probe pool.
b. A readout-complementary sequence.
c. rRNA target complementary sequence.
d. A readout-complementary sequence (can be same or different than b).
e. Primer sequences to enrich probe pool.

mRNA-probes contain (5′ to 3′):
a. Primer sequences to enrich probe pool.
b. An initiator sequence.
c. mRNA target complementary sequence.
d. An initiator sequence (can be same or different than b).
e. Primer sequences to enrich probe pool.

In some embodiments, each encoding probe can include a targeting sequence and at least one sequence that comprise at least one site for secondary hybridization events. In some embodiments, each encoding probe can include a targeting sequence and at least one sequence that comprises multiple (e.g., two or more) sites for secondary hybridization events. In some embodiments, each encoding probe can include a targeting sequence and at least one initiator sequence. In some embodiments, each encoding probe can include a targeting sequence and an initiator sequence.

Primer Sequences

In some embodiments, the primer sequence can include about 10 to about 30, about 15 to about 25, about 18 to about 23, about 15, 16, 17, 18, 19, 20, 21, 22, or 23 nucleotides long.

Targeting Sequence

In some embodiments, the targeting sequence targets at least one of messenger RNA (mRNA), micro RNA (miRNA), long non coding RNA (lncRNA), ribosomal RNA (rRNA), small interfering RNA (siRNA), transfer RNA (tRNA), Crispr RNA (crRNA), trans-activating cirspr RNA (tracrRNA), mitochondria RNA, Intronic RNA, viral mRNA, viral genomic RNA, environmental RNA, double-stranded RNA (dsRNA), small nuclear RNA (snRNA), small nucleolar (snoRNA), piwi-interacting RNA (piRNA), genomic DNA, synthetic DNA, DNA, plasmid DNA, a plasmid, viral DNA, retroviral DNA, environmental DNA, extracellular DNA, a protein, a small molecule, or an antigenic target. In some embodiments, the target is mRNA. In some embodiments, the target is rRNA. In some embodiments, the target is mRNA and rRNA.

In some embodiments, the targeting sequence of the encoding probe is substantially complementary to a specific target sequence. By “substantially complementary” it is meant that the nucleic acid fragment is capable of hybridizing to at least one nucleic acid strand or duplex even if less than all nucleobases do not base pair with a counterpart nucleobase. In some embodiments, a “substantially complementary” nucleic add contains at least one sequence in which about 70%, about 71%, about 72%, about 73%, about 74%, about 75%, about 76%, about 77%, about 77%, 8%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of basepairing with at least one single or double stranded nucleic acid molecule during hybridization.

In some embodiments, the targeting sequence is designed to have a predicted melting temperature of between about 55° C. and about 65° C. In some embodiments, the predicted melting temperature of the targeting sequence is 55° C., 56° C., 57° C., 58° C., 59° C., 60° C., 61° C., 62° C., 63° C., 64° C. or 65° C. In some embodiments, the targeting sequence can have a GC content of about 55%, 60%, 65% or 70%.

In some embodiments, the targeting sequence can include about 10 to about 35, about 15 to about 30, about 18 to about 30, about 15, 16, 17, 18, 19, 20, 21, 22, 23, 24, or 25 nucleotides long.

In some embodiments, the targeting sequence of an encoding probe is designed using publicly-available sequence data. In some embodiments, the targeting sequence of an encoding probe design is designed using custom catalogues of the target/sample. In some embodiments, the targeting sequence of an encoding probe is designed using a database that is relevant for a system. In a specific embodiment, the system is the gut microbiome. In some embodiments, the targeting sequence of an encoding probe is designed using a database that is relevant for a disease or infection.

Initiator Sequence

In some embodiments, the encoding probe can include the initiator sequence on the 5′ end and/or the 3′ end. In some embodiments, the encoding probe can include an initiator sequence on the 5′ end. In some embodiments, the encoding probe can include an initiator sequence on the 3′ end. In some embodiments, the encoding probe can include an initiator sequence on the 5′ end and an initiator sequence on the 3′ end. In some embodiments, the two initiator sequences have different sequences. In some embodiments, the two initiator sequences have the same sequence. In some embodiments, the encoding probe can include the at least one sequence that comprise at least one site for secondary hybridization events on the 5′ end and/or the 3′ end.

In some embodiments, the initiator sequence is about 10 to about 30, about 15 to about 25, about 18 to about 23, about 15, 16, 17, 18, 19, 20, 21, 22, or 23 nucleotides long. In some embodiments, the initiator sequence is substantially complementary to the toehold sequence of the DNA amplifier.

In some embodiments, an encoding probe can include two fractional encoding probes that have neighboring target regions. The two fractional encoding probes bind to the target to colocalize a full initiator. The colocalized full initiator is required to initiate the hybridization chain reaction by a corresponding amplifier. In some embodiments, there is an energetically unfavorable junction between the two duplexes. In some embodiments, by configuring the fractional initiators to bind to overlapping regions of the amplifier, the duplex can relax into an energetically more favorable conformation, increasing the affinity between the colocalized full initiator and the amplifier. In some embodiments the affinity between the two encoding probes and the target can be increased by configuring the target-binding regions of the two encoding probes to bind to overlapping regions of the target so as to permit the junction between the molecules to relax to an energetically favorable conformation. In some embodiments, the two fractional encoding probes have about the same nucleotide length. In some embodiments, the two fractional encoding probes have different nucleotide lengths, for example one fractional encoding probe may have about 25% nucleotide length and the other the 75% nucleotide length.

The encoding probes, and other probes described herein, may be introduced into the sample (e.g., cell) using any suitable method. In some cases, the sample may be sufficiently permeabilized such that the probes may be introduced into the sample by flowing a fluid containing the probes around the sample (e.g., cells). In some cases, the samples (e.g., cells) may be sufficiently permeabilized as part of a fixation process. In some embodiments, samples (e.g., cells) may be permeabilized by exposure to certain chemicals such as ethanol, methanol, Triton, or the like. In some embodiments, techniques such as electroporation or microinjection may be used to introduce the probes into a sample (e.g., cell).

DNA Amplifier Sequences

“DNA amplifiers,” “amplifiers,” and “amplifier sequences” are used interchangeably when referring to the HiPR-Cycle method described herein.

Amplifier sequences are metastable hairpin sequences that come in pairs (###_H1 and ###_H2), the design is based on HCR amplifier probes and contains a readout-complementary sequencing at the 5′-end (in the case of ###_H1) or 3′-end (###_H2), adjacent to the initiator sequence. Amplifier sequences are stored in a high salt buffer (e.g., 120 mM NaCl), and are heated (e.g., 95° C. for 1.5 min) and annealed (e.g., room temperature for 30 min) prior to addition to the sample.

In some embodiments the amplifiers are stored in high salt buffer, such as, 100 mM, 120 mM, 200 mM, 250 mM, 500 mM, 750 mM, or 1 M NaCl. In some embodiments, the amplifier sequences are heated at high temperatures (e.g., about 95° C. to 100° C.) for a short period of time (e.g., 1, 2, 3, 4 or 5 minutes) followed by a cooling period of about 15 min to 1 hour, e.g., 30 min, to room temperature.

In some embodiments, at least two amplifier probes (one pair) are used for at least one readout probe. In some embodiments, at least two amplifier probes (one pair) are used for multiple (e.g., two or more) readout probes. In some embodiments, at least two amplifier probes (one pair) are used for each readout probe. For example, each amplifier probe can have:
a. A readout complementary sequence (15-20 nt).
b. An optional first spacer sequence (0-5 nt).
c. A toehold sequence. (9 nt)
d. A stem sequence.
e. A loop sequence (9 nt) complementary to the initiator on the paired amplifier).
f. A stem-complementary sequence.

In other examples, each amplifier probe can have:
a. A readout complementary sequence (15-20 nt).
b. An optional first spacer sequence (0-5 nt).
c. A toehold sequence. (9 nt)
d. A stem sequence.
e. An optional second spacer sequence (0-5 nt).
f. A loop sequence (9 nt) complementary to the initiator on the paired amplifier).
g. A stem-complementary sequence.

In other examples, each amplifier probe can have:
a. A stem-complementary sequence.
b. A loop sequence (9 nt) complementary to the initiator on the paired amplifier).
c. An optional second spacer sequence (0-5 nt).
d. A stem sequence.
e. A toehold sequence (9 nt)
f. An optional first spacer sequence (0-5 nt).
g. A readout complementary sequence (15-20 nt).

The readout complementary sequence of the amplifier probe/DNA amplifier is a nucleotide sequence that is about 10 to about 25, about 15 to about 20, about 15, 16, 17, 18, 19, or 20 nucleotides long and has a nucleotide sequence that is the complement of the emissive readout probe sequence. In some embodiments, the readout sequence present in the amplifier probe is also known as a “landing pad sequence.” In some embodiments, the readout complementary sequence present in the amplifier probe is also known as a “landing pad sequence.”

Each of the optional first and second spacer sequences of the amplifier probe/DNA amplifier is about 1 to 5, about 1, 2, 3, 4, or 5 nucleotides long.

The toehold sequence of the amplifier probe/DNA amplifier is a nucleotide sequence that is about 10 to about 30, about 15 to about 25, about 18 to about 23, about 15, 16, 17, 18, 19, 20, 21, 22, or 23 nucleotides long and has a nucleotide sequence that is the complement to the initiator sequence of the encoding probe.

The stem sequence of the amplifier probe/DNA amplifier is a nucleotide sequence that is about 5 to about 15, about 7 to about 10, about 5, 6, 7, 8, 9, or 10 nucleotides long and has a nucleotide sequence that is a complement to its other stem.

The loop sequence of the amplifier probe/DNA amplifier is a nucleotide sequence that is about 5 to about 15, about 7 to about 10, about 5, 6, 7, 8, 9, or 10 nucleotides long and has a nucleotide sequence that is a complement to the toehold sequence of its pair DNA amplifier.

The stem-complimentary sequence of the amplifier probe/DNA amplifier is a nucleotide sequence that is about 5 to about 15, about 7 to about 10, about 5, 6, 7, 8, 9, or 10 nucleotides long and has a nucleotide sequence that is a complement to its other stem.

In some embodiments, one of the two DNA amplifiers can include, from 5′ to 3′, a readout sequence (R.1), a toehold sequence (T.1), a stem sequence (S.1), a loop sequence (L.1), and a complement stem sequence (cS.1). In some embodiments, one of the two DNA amplifiers can include, from 5′ to 3′, a readout sequence (R.1), a first spacer sequence (Sp.1-1), a toehold sequence (T.1), a stem sequence (S.1), a loop sequence (L.1), and a complement stem sequence (cS.1). In some embodiments, one of the two DNA amplifiers can include, from 5′ to 3′, a readout sequence (R.1), a first spacer sequence (Sp.1-1), a toehold sequence (T.1), a stem sequence (S.1), a second spacer sequence (Sp.1-2), a loop sequence (L.1), and a complement stem sequence (cS.1).

In some embodiments, one of the two DNA amplifiers can include, from 5′ to 3′, a stem sequence (S.2), a loop sequence (L.2), a complement stem sequence (cS.2), a toehold sequence (T.2), and a readout sequence (R.2).

In some embodiments, one of the two DNA amplifiers can include, from 5′ to 3′, a stem sequence (S.2), a loop sequence (L.2), a complement stem sequence (cS.2), a toehold sequence (T.2), a first spacer sequence (Sp.2-1), and a readout sequence (R.2). In some embodiments, one of the two DNA amplifiers can include, from 5′ to 3′, a stem sequence (S.2), a loop sequence (L.2), a second spacer sequence (Sp. 2-2), a complement stem sequence (cS.2), a toehold sequence (T.2), a first spacer sequence (Sp.2-1), and a readout sequence (R.2).

In some embodiments, one of the two DNA amplifiers can include, from 5′ to 3′, a toehold sequence (T.1), a stem sequence (S.1), a loop sequence (L.1), a complement stem sequence (cS.1), and a readout sequence (R.1). In some embodiments, one of the two DNA amplifiers can include, from 5′ to 3′, a readout sequence (R.2), a stem sequence (S.2), a loop sequence (L.2), a complement stem sequence (cS.2), and a toehold sequence (T.2).

In some embodiments, the DNA amplifiers can further include a first spacer sequence and/or a second spacer sequence. In some embodiments, the DNA amplifiers further can include a first spacer sequence. In some embodiments, the DNA amplifiers further can include a second spacer sequence. In some embodiments, the DNA amplifiers further can include a first spacer sequence and a second spacer sequence. In some embodiments, the first spacer sequence is on the 3′ end of the readout sequence and to the 5′ end of the toehold sequence of the DNA amplifier. In some embodiments, the first spacer sequence is on the 3′ end of the toehold sequence and to the 5′ end of the readout sequence of the DNA amplifier. In some embodiments, the first spacer sequence is 1, 2, 3, 4, or 5 nucleotides long. In some embodiments, the first spacer sequence is a random string of three nucleotides. In some embodiments, the second spacer sequence is on the 3′ end of the stem sequence and to the 5′ end of the loop sequence complementary to the initiator of the DNA amplifier. In some embodiments, the second spacer sequence is on the 3′ end of the loop sequence complementary to the initiator and to the 5′ end of the stem sequence of the DNA amplifier. In some embodiments, the second spacer sequence is 1, 2, 3, 4, or 5 nucleotides long. In some embodiments, the second spacer sequence is a random string of three nucleotides.

In some embodiments, the readout sequence of the DNA amplifier can include 15 to 30 nucleotides, or 15, 16, 17, 18, 19, 20, 21, 22, 23, 24, 25, 26, 27, 28, 29, or 30 nucleotides. In some embodiments, the readout sequence of each DNA amplifier is the same sequence. In some embodiments, the readout sequence of each DNA amplifier is the different. In some embodiments, the readout sequence of DNA amplifier has a 50% or less sequence identity to the other the readout sequence of DNA amplifier.

In some embodiments, the toehold sequence (T.1) is a sequence complementary to the loop sequence (L.2) of the other DNA amplifier. In some embodiments, the loop sequence (L.1) is a sequence complementary to the toehold sequence (T.2) of the other DNA amplifier. In some embodiments, the toehold sequence is 7, 8, 9, 10, 11, 12, 13, 14, 15, 16, or 17 nucleotides long. In some embodiments, the loop sequence is 7, 8, 9, 10, 11, 12, 13, 14, 15, 16, or 17 nucleotides long. In some embodiments, the stem region and its complementary sequence are each 7, 8, 9, 10, 11, 12, 13, 14, 15, 16, 17, 18, 19, 20, or 21 nucleotides long.

In some embodiments, the method can include adding four DNA amplifiers.

In some embodiments, one of the four DNA amplifiers can include, from 5′ to 3′ a amplifier initiator sequence (HI.1), a toehold sequence (T.1), a stem sequence (S.1), a loop sequence (L.1), and a complement stem sequence (cS.1). In some embodiments, one of the four DNA amplifiers can include, from 5′ to 3′ a stem sequence (S.2), a loop sequence (L.2), complement stem sequence (cS.2), a toehold sequence (T.2), and an amplifier initiator sequence (HI.2). In some embodiments, one of the four DNA amplifiers can include, from 5′ to 3′, a readout sequence (R.1-2), a toehold sequence (T.1-2), a stem sequence (S.1-2), a loop sequence (L.1-2), and a complement stem sequence (cS.1-2). In some embodiments, one of the four DNA amplifiers can include, from 5′ to 3′, a stem sequence (S.2-1), a loop sequence (L.2-1), a complement stem sequence (cS.2-1), a toehold sequence (T.2-1), and a readout sequence (R.2-1).

In some embodiments, the four DNA amplifiers can further include a first and/or second spacer sequence, wherein the first and/or second spacer sequence is about 1 to 5, about 1, 2, 3, 4, or 5 nucleotides long.

In some embodiments, the amplifier initiator sequence (HI.1) is a sequence complementary to the loop sequence (L.1-2 or L.2-1) of one of the other DNA amplifiers can include the readout sequence. In some embodiments, the toehold sequence (T.1) is a sequence complementary to the loop sequence (L.2) of the other DNA amplifier can include the amplifier initiator sequence. In some embodiments, the loop sequence (L.1) is a sequence complementary to the toehold sequence (T.2) of the other DNA amplifier can include the amplifier initiator sequence. In some embodiments, the amplifier initiator sequence is unique so that its sequence is not complementary to any other sequence. In this instance, the initiator sequence is different from the rest of the sequences so that it does not prematurely trigger the amplification reaction.

Emissive Readout Probes

Emissive readouts probes are 15-20 nucleotide-long oligonucleotides bound with one of ten fluorescent dyes at the 5′- and/or 3′-end. In HiPR-Cycle, these sequences bind to the amplifier complexes that form. They can be added during or after the amplification step.

Readout probes (15-20 nt) can be designed as follows:
a. Are coupled to 1, 2, or more fluorescent dyes.
b. Are orthogonal to all biological sequences.
c. Are orthogonal to each other/each other's complementary sequences.

In some embodiments, the emissive readout sequence is about 10 to about 50, about 15 to about 50, about 15 to about 45, about 15 to about 35, about 15 to about 30, about 18 to about 24, about 10, 11, 12, 13, 14, 15, 16, 17, 18, 19, 20, 21, 22, 23, or 24 nucleotides long.

In some embodiments, the emissive readout probe can include a label on the 5′ or 3′ end. In some embodiments, the emissive readout probe can include a label on the 5′ end and a label on the 3′ end. In some embodiments, the labels are the same. In some embodiments, the labels are different.

In some embodiments, the label is a fluorescent entity (fluorophore) or phosphorescent entity. In some embodiments, the label is a cyanine dye (e.g., Cy2, Cy3, Cy3B, Cy5, Cy5.5, Cy7, etc.), Alexa Fluor dye, Atto dye, photo switchable dye, photoactivatable dye, fluorescent dye, metal nanoparticle, semiconductor nanoparticle or “quantum dots”, fluorescent protein such as GFP (Green Fluorescent Protein), or photoactivatable fluorescent protein, such as PAGFP, PSCFP, PSCFP2, Dendra, Dendra2, EosFP, tdEos, mEos2, mEos3, PAmCherry, PAtagRFP, mMaple, mMaple2, and mMaple3.

In some embodiments, the label is Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, Rox, Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740.

In some embodiments, the label is imaged using widefield microscopy, point scanning confocal microscopy, spinning disk confocal microscopy, lattice lightsheet microscopy, or light field microscopy.

In some embodiments, the detection strategy used is channel, spectral, channel and fluorescence lifetime, or spectral and fluorescence lifetime.

In some embodiments, the labels used in the present methods are imaged using a microscope. In some embodiments, the microscope is a confocal microscope. In some embodiments, the microscope is a fluorescence microscope. In some embodiments, the microscope is a light-sheet microscope. In some embodiments, the microscope is a super-resolution microscope.

In some embodiments, the sample is on an analyzing platform, wherein the analyzing platform is a microscope slide, at least one chamber, at least one microfluidic device, at least one well, at least one plate, or at least one filter membrane.

Increasing Sample Detection

In some embodiments, when it is necessary to increase the multiplexity of the method, the HiPR-Cycle method described herein can be performed multiple times/rounds, by physically expanding the sample, and/or by using branched amplification.

HiPR-Cycle has the ability to perform measurements at high multiplexity with barcoding and spectral readouts. This allows to theoretically detect 2^{d}−1 targets where d is the number of dyes used in an assay. The targets can be given barcodes (based on the encoding sequences) of d bits. The addition of more rounds allows the barcode to be extended. For R rounds, the maximum target multiplexity becomes 2^{d*R}−1 and allows for dR-bit barcodes. For example, for a 10-bit system (using 10 dyes), one such code may be 0010011101. In the 10-bit system with two rounds of HiPR-Cycle, 20-bit barcodes can be used, and achieve 1,048,575 targets. For example, a single gene could be labeled as 0010011101 in round one, and 0100010101 in round two, making its complete barcode 00100111010100010101. This is just a single code of the >1M available codes, which comes from concatenating the barcodes determined in the first and second round of imaging. Since HiPR-Cycle's multiplexing capabilities increase exponentially with both the number of distinguishable fluorescent dyes and the number of rounds, billions of potential targets are available (e.g. 3 rounds using 10 bits per round leads to 1.07 billion targets, as does 2 rounds using 15 bits per round).

In some embodiments, when there are two colors and two rounds, one can encode a gene as 00+01 and another as 01+01, one may have a 0101 gene, but there may be an issue preventing binding in the first case and it will be incorrectly read as 0001. Therefore, a practical maximum would then be (2^{(D)-1})^{R}.

In some embodiments, multiple rounds can be performed by (1) repeating HiPR-Cycle, in its entirety, twice (or more) using two (or more) different sets of n-bit (e.g., 10-bit) encoding probes; (2) repeating HiPR-Cycle amplification/readout twice (or more) using two different sets of n-bit (e.g., 10-bit) encoding probes; (3) bleaching readout probes; (4) chemical or restriction enzyme or CRISPR cleaving of readout probes; or (5) DNAse cleaving of probes.

In some embodiments, multiple rounds can be performed by repeating HiPR-Cycle, in its entirety, twice (or more) using two (or more) different sets of n-bit (e.g., 10-bit) encoding probes. In these embodiments, the encoding probes are encoded with between 1 and 10 initiators from a selection of 10 initiators. Each initiator corresponds to a unique set of amplifiers which all together hybridize a set of up 10 emissive readout probes. HiPR-Cycle is then performed (including imaging). Encoding probes and readout probes are physically removed from the sample using a stripping buffer, then another round of HiPR-Cycle is performed in its entirety. In some embodiments, the stripping buffer can include formamide and SSC. In some embodiments, the stripping buffer can include about 40% to about 70%, about 40%, 50%, 60%, or 70% formamide. In some embodiments, the stripping buffer can include about 2× to about 10×, about 2×, 5×, or 10×SSC. In some embodiments, the stripping buffer can include 60% formamide and 2×SSC.

In some embodiments, multiple rounds can be performed by repeating HiPR-Cycle amplification/readout twice (or more) using two different sets of n-bit (e.g., 10-bit) encoding probes. In these embodiments, the encoding probes are encoded with between 1 and 20 initiators from a selection of 20 initiators. Each initiator corresponds to a unique set of amplifiers which all together hybridize a set of up 10 emissive readout probes. In this particular embodiment, only amplifiers/readout probes corresponding to a unique color are used in each round. For example, in the case where there are only 3 readout probes (red, blue, green), but want to use 2 rounds of imaging. There could be a code such as 110010 where the first three digits are each a single color and the last three digits are each a single color. Here only the first three digits can be read in a single round, and the last three digits can be read in a single round. Then the amplification/readout steps are performed. The probes are then stripped using stripping buffer. The stripping buffer removes the amplifier and readout probes but does not remove the encoding probes. A second amplifier/readout is then performed with a unique set of amplifier probes.

In some embodiments, multiple rounds can be performed by bleaching readout probes. In these embodiments, the encoding probes can contain many initiators, and many corresponding amplifier and readout probes. Further, two readout probes may have the same fluorophore but different sequences. In this embodiment, HiPR-Cycle is performed and all of the targets are amplified. Readout probes are collected into sets, each set has unique fluorescent dyes and sequences. Readout probes are then added and imaging is done according to the methods described herein. A bleaching buffer can then be placed on the sample and high intensity/exposure laser (e.g., 647 nm at 100% intensity for 1 sec) can be used to bleach probes. The bleaching buffer can then be removed, the sample washed, and the next set of readout probes is added to the bleaching buffer. In this embodiment, the bleaching buffer can include SSC and VRC. In some embodiments, the bleaching buffer can include about 0.1× to about 5×, about 0.5× to about 2.5×, about 1× to about 2×, or about 2×. In some embodiments, the bleaching buffer can include about 0.5 mM to about 5 mM, about 1 mM to about 3 mM, or about 2 mM Vanadyl ribonucleoside complex (VRC). In some embodiments, the bleaching buffer can include 2×SSC and 2 mM VRC. Vanadyl ribonucleoside complex (VRC) is a potent inhibitor of various ribonucleases. This complex is compatible with cell fractionation methods as well as sucrose-gradient centrifugations. The 200 mM stock solution is reconstituted to a green-black clear solution by incubating the sealed vial at 65° C. Once open, the entire sample should be aliquoted into smaller samples and frozen. The vanadyl ribonucleoside complex should be added to all buffers to a final concentration of 10 mM. The buffers should not contain EDTA since one equivalent will totally dissociate the complex. Use of the vanadyl complex is not recommended in cell-free translation systems and with reverse transcriptase. The vanadyl complex can be used in the selective degradation of DNA while preserving RNA since pancreatic deoxyribonuclease I is not inhibited. Removal of the vanadyl ribonucleoside complex from the RNA can be accomplished by adding 10 equivalents of EDTA before ethanol precipitation.

In some embodiments, multiple rounds can be performed by chemical or restriction enzyme or CRISPR cleaving of readouts, which is similar to the bleaching probe method. In here, after a round of imaging the readout sequences are “cut” (with bound readout probes) off of the amplifiers and washed away.

In some embodiments, multiple rounds can be performed by a DNAse method, where after imaging, DNAse is added to the sample to remove all encoding, amplifier, and readout probes. Another round of HiPR-Cycle is then performed in its entirety.

In some embodiments, when it is necessary to increase the ability to identify and quantify targets in a sample, physically expanding the sample can be used in conjunction with the HiPR-Cycle methods described herein. In here, combining the imaging methods described herein with physical expansion allows for samples/cells/molecules to increase the physical distance between them. Further, covalently embedding targets of a sample within a gel matrix makes it possible to “clear” (e.g., digest/remove) unwanted biomolecules (proteins, lipids, etc.) that could contribute to light scattering and background autofluorescence. In these embodiments, a sample is embedded in a hydrogel (e.g., polyacrylamide or polyacrylamide-based gels) and HiPR-Cycle is then performed on the sample-embedded gel.

In some embodiments, when it is necessary to increase the multiplexity of the method, the HiPR-Cycle method described herein can be performed using branched amplification. Intermediates located between the encoding probes and amplifiers can result in an increase (e.g., 4-100 more sites) of initiator sites available per encoding probe compared to other HiPR-Cycle methods described herein. The intermediates contain sequences that can be hybridized to the encoding probes and to the amplifiers. To produce the intermediates, a first intermediate probe is hybridized to the encoding probe, then, a second intermediate probe is hybridized to the first intermediate probe. The second intermediate probe contains multiple binding/complementary sequences for initiator probes to hybridize to. The initiator probes contain (initiator) sequences upon which the amplifiers can bind to. The rest of the HiPR-Cycle method can then be performed as described herein.

Accordingly, in some embodiments, the HiPR-Cycle method can be utilized with sets containing first intermediate probes, second intermediate probes, and/or initiator probes. In some embodiments, the first intermediate probe includes a sequence complementary to a sequence present in an encoding probe and at least one handle sequence (e.g., 1-5 handle sequences). In some embodiments, the second intermediate probe includes a sequence complementary to the at least one handle sequence of the first intermediate probe and at least one initiator landing (or presenting) sequence. In some embodiments, the initiator probes include a sequence complementary to the at least one initiator landing (or presenting) sequence and one initiator sequence complementary to an initiator sequence present in an amplifier. In some embodiments, when branched amplification is utilized, the number of initiators sites/sequences available per encoding probe is from about 4 to about 100, about 6 to about 75, about 10 to about 50, about 15 to about 35, or about 9 to about 18.

HiPR-Swap

Another aspect of the disclosure is directed to a method of analyzing a sample by performing HiPR-Cycle with multiple imaging rounds exchanging emissive readout probes which are referred to herein as HiPR-Swap.

HiPR-Swap uses DNA exchange as a method to quickly, specifically, carefully replace readout probes without disturbing encoding and/or amplifier probes. See, for example, PCT Patent Application PCT/US2022/080355 and U.S. application Ser. No. 18/058,171, filed on Nov. 24, 2022. The contents of the aforementioned disclosures are each incorporated herein by reference in their entireties.

Accordingly, a method for analyzing a sample can include:
contacting at least one encoding probe with the sample to produce a first complex, wherein each encoding probe can include a targeting sequence and an initiator sequence;
adding at least two different DNA amplifiers to the first complex to produce a second complex, wherein each DNA amplifier can include an initiator complimentary sequence and a readout sequence;
adding a set of first emissive readout probes to the second complex, wherein each of the first emissive readout probes can include a label and a complimentary sequence to the readout sequence of a corresponding DNA amplifier;
acquiring one or more emission spectra from the set of first emissive readout probes;
adding a set of HiPR-Swap first exchange probes to the sample, wherein each of the first exchange probes include a 100% complementary sequence to the first emissive readout probe sequence,
hybridizing the first exchange probes to the first emissive readout probes to form a third complex;
removing the third complex from the sample,
adding a set of second emissive readout probes to the second complex, wherein each of the second emissive readout probes can include a label and a complimentary sequence to the readout sequence of a corresponding DNA amplifier;
acquiring one or more emission spectra from the second emissive readout probes;
repeating the aforementioned steps for at least one different encoding probe.

Landing Pad Sequences

In the HiPR-Swap method, readout and amplifier probes are designed such that the “landing pad” is shorter than or equal to in length to the readout probe. The landing pad being shorter than the readout probe creates a single-stranded overhang of the readout probe, as it extends past the end of the landing pad. After a readout probe is bound, an exchange probe can be added to the sample. The exchange probe can be constructed to be of equal length and a perfect reverse complement to the readout probe. In some embodiments, the exchange probe may contain locked nucleic acids to increase the stability of the exchange-readout pair. When added, the exchange probe seeds a hybridization to the exposed area of the readout probe. Over a short period of time the exchange probe completely hybridizes to the readout probe, thereby removing it from its complementary sequence where it can be washed away. Importantly, orthogonal readout and exchange probes can be added simultaneously to reduce assay time.

In some embodiments, the readout sequence present in the amplifier probe is referred to as a “landing pad.” In some embodiments, the landing pad includes a sequence that is complementary to the emissive readout sequence. In some embodiments, when HiPR-Swap is being utilized, the landing pad includes a sequence that is complementary to the emissive readout sequence.

In some embodiments, each landing pad sequence is about 10 to about 50, about 15 to about 50, about 15 to about 40, about 10 to about 30, about 15 to about 25, about 18 to about 23, about 15, 16, 17, 18, 19, 20, 21, 22, or 23 nucleotides long. In some embodiments, each landing pad sequence is substantially complementary to the first and/or second emissive readout sequences.

Exchange Probes

Exchange probes are each about 10-50 or 15-50 nucleotide-long oligonucleotides. In some embodiments, each exchange probe includes a 100% complementary sequence to a respective emissive readout probe sequence. In some embodiments, the emissive readout probe sequence is an emissive readout probe as described herein.

In some embodiments, the exchange sequence is about 10 to about 50, about 15 to about 50, about 15 to about 45, about 15 to about 35, about 15 to about 30, about 18 to about 24, about 10, 11, 12, 13, 14, 15, 16, 17, 18, 19, 20, 21, 22, 23, or 24 nucleotides long.

In some embodiments, the encoding and/or amplifier probes contain locked nucleic acids to stabilize the exchange reaction.

In some embodiments, adding an exchange probe to a sample, hybridizing the exchange probe to a first emissive readout probe, and removing a third complex from the sample are performed in the same step. In some embodiments, adding an exchange probe to the sample, hybridizing the exchange probe to the first emissive readout probe, and removing the third complex from the sample are performed sequentially. In some embodiments, adding an exchange probe to the sample, hybridizing the exchange probe to the first emissive readout probe, removing the third complex from the sample, and adding the second emissive readout probe are performed in the same step. In some embodiments, adding an exchange probe to the sample, hybridizing the exchange probe to the first emissive readout probe, removing the third complex from the sample, and adding the second emissive readout probe are performed sequentially.

In some embodiments, hybridizing the exchange probe to the first or second emissive readout probes results in de-hybridization of the first or second emissive readout probe from the readout (landing pad) sequence. In some embodiments, the step is achieved from about 30 seconds to about 1 hour. In some embodiments, the step is achieved within 30 seconds, 1 minute, 5 minutes, 10 minutes, 12 minutes, 15 minutes, 30 minutes, 45 minutes, or 1 hour. In some embodiments, the step is achieved within 1 hour. In some embodiments, the step is achieved overnight.

### Constructs and Libraries

Another aspect, a construct can include:
a targeting sequence that is a region of interest on a nucleotide;
a first initiator sequence;
a second initiator sequence that is different from the first initiator sequence;
a first amplifier sequence can include a readout sequence on the 5′ end of the sequence;
a second amplifier sequence can include a readout sequence on the 3′ end of the sequence, wherein the second amplifier sequence is different from the first amplifier sequence; and
an emissive readout sequence can include a sequence complimentary to the readout sequence of the first and/or second amplifier sequences and a label on the 5′ and/or 3′ end of the complimentary sequence.

In some embodiments, the region of interest on a nucleotide is at least one of messenger RNA (mRNA), micro RNA (miRNA), long non coding RNA (lncRNA), ribosomal RNA (rRNA), small interfering RNA (siRNA), transfer RNA (tRNA), Crispr RNA (crRNA), trans-activating cirspr RNA (tracrRNA), mitochondria RNA, Intronic RNA, viral mRNA, viral genomic RNA, environmental RNA, double-stranded RNA (dsRNA), small nuclear RNA (snRNA), small nucleolar (snoRNA), piwi-interacting RNA (piRNA), genomic DNA, synthetic DNA, DNA, plasmid DNA, a plasmid, viral DNA, retroviral DNA, environmental DNA, extracellular DNA, a protein, a small molecule, or an antigen.

In some embodiments, the region of interest on a nucleotide is mRNA. In some embodiments, the region of interest on a nucleotide is rRNA. In some embodiments, the region of interest on a nucleotide is mRNA and rRNA.

In some embodiments, the first initiator sequence is to the 5′ end of the targeting sequence. In some embodiments, the second initiator sequence is to the 3′ end of the targeting sequence.

In some embodiments, the first amplifier can include, from 5′ to 3′, a readout sequence (R.1), a toehold sequence (T.1), a stem sequence (S.1), a loop sequence (L.1), and a complement stem sequence (cS.1). In some embodiments, the first amplifier can include, from 5′ to 3′, a readout sequence (R.1), a first spacer sequence (Sp.1-1), a toehold sequence (T.1), a stem sequence (S.1), a loop sequence (L.1), and a complement stem sequence (cS.1). In some embodiments, the first amplifier can include, from 5′ to 3′, a readout sequence (R.1), a first spacer sequence (Sp.1-1), a toehold sequence (T.1), a stem sequence (S.1), a second spacer sequence (Sp.1-2), a loop sequence (L.1), and a complement stem sequence (cS.1).

In some embodiments, the second amplifier can include, from 5′ to 3′, a stem sequence (S.2), a loop sequence (L.2), a complement stem sequence (cS.2), a toehold sequence (T.2), and a readout sequence (R.2). In some embodiments, the second amplifier can include, from 5′ to 3′, a stem sequence (S.2), a loop sequence (L.2), a complement stem sequence (cS.2), a toehold sequence (T.2), a first spacer sequence (Sp.2-1), and a readout sequence (R.2). In some embodiments, the second amplifier can include, from 5′ to 3′, a stem sequence (S.2), a loop sequence (L.2), a second spacer sequence (Sp. 2-2), a complement stem sequence (cS.2), a toehold sequence (T.2), a first spacer sequence (Sp.2-1), and a readout sequence (R.2).

In some embodiments, each amplifier can further include a first and/or second spacer sequence, wherein the first and/or second spacer sequence is about 1 to 5 nucleotides long or about 1, 2, 3, 4, or 5 nucleotides long.

In some embodiments, the toehold sequence (T.1) of the first amplifier is a sequence complementary to the loop sequence (L.2) of the second amplifier.

In some embodiments, the loop sequence (L.1) of the first amplifier is a sequence complementary to the toehold sequence (T.2) of the second amplifier.

In some embodiments, the first and second amplifier have the same readout sequence. In some embodiments, the first and second amplifier have different readout sequences. In some embodiments, the readout sequence present in the amplifier probe is referred to as a “landing pad.” In some embodiments, the landing pad includes a sequence that is complementary to the emissive readout sequence.

In some embodiments, the emissive readout sequence can include a sequence complimentary to the readout sequence of the first amplifier sequence. In some embodiments, the emissive readout sequence can include a sequence complimentary to the readout sequence of the second amplifier sequence. In some embodiments, the emissive readout sequence can include a label on the 5′ end of the complimentary sequence. In some embodiments, the emissive readout sequence can include a label on the 3′ end of the complimentary sequence. In some embodiments, the emissive readout sequence can include a label on the 5′ end and 3′ end of the complimentary sequence.

In some embodiments, the label is a fluorescent entity (fluorophore) or phosphorescent entity. In some embodiments, the label is a cyanine dye (e.g., Cy2, Cy3, Cy3B, Cy5, Cy5.5, Cy7, etc.), Alexa Fluor dye, Atto dye, photo switchable dye, photoactivatable dye, fluorescent dye, metal nanoparticle, semiconductor nanoparticle or “quantum dots”, fluorescent protein such as GFP (Green Fluorescent Protein), or photoactivatable fluorescent protein, such as PAGFP, PSCFP, PSCFP2, Dendra, Dendra2, EosFP, tdEos, mEos2, mEos3, PAmCherry, PAtagRFP, mMaple, mMaple2, and mMaple3.

In some embodiments, the label is Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, Rox, Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740.

In some embodiments, a construct can include:
a targeting sequence that is a region of interest on a nucleotide;
a first initiator sequence;
a second initiator sequence that is different from the first initiator sequence;
a first amplifier sequence can include a third initiator sequence;
a second amplifier sequence can include a fourth initiator sequence;
a third amplifier sequence can include a readout sequence on the 5′ end of the sequence;
a fourth amplifier sequence can include a readout sequence on the 3′ end of the sequence, wherein the first, second, third, and fourth amplifier sequences are different from each other; and
an emissive readout sequence can include a sequence complimentary to the readout sequence of the third and/or fourth amplifier sequences and a label on the 5′ and/or 3′ end of the complimentary sequence.

In some embodiments, a construct described herein further includes at least one exchange probe and at least a second emissive readout probe, as described herein.

In some embodiments, a construct described herein further includes at least one first intermediate probe, at least one second intermediate probe, and at least one initiator (landing) probe, as described herein.

In another aspect, a library of constructs can include a plurality of barcoded probes, wherein each barcoded probe can include:
a targeting sequence that is a region of interest on a nucleotide;
at least one initiator sequence;
two DNA amplifiers, wherein each DNA amplifier can include a readout sequence; and
an emissive readout probe, wherein each emissive readout probe can include a label and a sequence complimentary to the readout sequence of a corresponding DNA amplifier.
wherein at least two barcoded probes of the plurality of barcoded probes include targeting sequences that is specific to different regions of interest.

In another aspect, a library of constructs can include a plurality of barcoded probes, wherein each barcoded probe can include:
a targeting sequence that is a region of interest on a nucleotide;
a first initiator sequence;
a first and a second DNA amplifier, wherein each first and second DNA amplifier can include a second initiator sequence
a third and a fourth DNA amplifier, wherein each third and fourth DNA amplifier can include a readout sequence; and
an emissive readout probe, wherein each emissive readout probe can include a label and a sequence complimentary to the readout sequence of a corresponding third and/or fourth DNA amplifier;
wherein at least two barcoded probes of the plurality of barcoded probes include targeting sequences that are specific to different regions of interest.

In some embodiments, a library of constructs can further include at least one exchange probe and at least a second emissive readout probe, as described herein.

In some embodiments, a library of constructs can further include at least one first intermediate probe, at least one second intermediate probe, and at least one initiator (landing) probe, as described herein.

### Barcoded Probes

The encoding probes used in the methods described herein, constructs and libraries described herein use barcoded probes. The barcoded probes represent a probe/sequence that is specific to a sample or target sequence in the sample with a unique code.

In some embodiments, the barcoded probes include the encoding probes, DNA amplifiers, and readout sequences described herein.

In some embodiments, each sample or target in the sample to be identified is assigned a unique n-bit binary code selected from a plurality of unique n-bit binary codes, where n is an integer greater than 1.

A “binary code” refers to a representation of target sequence in a sample using a string made up of a plurality of “0” and “1” from the binary number system. The binary code is made up of a pattern of n binary digits (n-bits), where n is an integer representing the number of labels used. The bigger the number n, the greater number of targets can be represented using the binary code. For example, a binary code of eight bits (an 8-bit binary code, using 8 different labels) can represent up to 255 (28-1) possible targets. (One is subtracted from the total possible number of codes because no target sequence is assigned a code of all zeros “00000000.” A code of all zeros would mean no decoding sequence, and thus no label, is attached. In other words, there are no non-labeled target sequences.) Similarly, a binary code of ten bits (a 10-bit binary code) can represent up to 1023 (210-1) possible target sequences. In some embodiments a binary code may be translated into and represented by a decimal number. For example, the 10-bit binary code “0001100001” can also be represented as the decimal number “97.”

Each digit in a unique binary code represents whether a readout probe and the fluorophore corresponding to that readout probe are present for the selected species. In some embodiments, each digit in the binary code corresponds to a Readout probe (from Readout probe 1 (R1) through Readout probe n (Rn) in an n-bit coding scheme). In a specific embodiment, the n is 10 and the digits of an n-bit code correspond to R1 through R10. In some embodiments, the fluorophores that correspond to R1 through Rn are determined arbitrarily. For example, n is 10, and R1 corresponds to an Alexa 488 fluorophore, R2 corresponds to an Alexa 546 fluorophore, R3 corresponds to a 6-ROX (6-Carboxy-X-Rhodamine, or Rhodamine Red X) fluorophore, R4 corresponds to a PacificGreen fluorophore, R5 corresponds to a PacificBlue fluorophore, R6 corresponds to an Alexa 610 fluorophore, R7 corresponds to an Alexa 647 fluorophore, R8 corresponds to a DyLight-510-LS fluorophore, R9 corresponds to an Alexa 405 fluorophore, and R10 corresponds to an Alex532 fluorophore. In some embodiments, other labels/fluorophores are used in the n-bit encoding system.

In some embodiments, the n-bit binary code is selected from the group consisting of 2-bit binary code, 3-bit binary code, 4-bit binary code, 5-bit binary code, 6-bit binary code, 7-bit binary code, 8-bit binary code, 9-bit binary code, 10-bit binary code, 11-bit binary code, 12-bit binary code, 13-bit binary code, 14-bit binary code, 15-bit binary code, 16-bit binary code, 17-bit binary code, 18-bit binary code, 19-bit binary code, 20-bit binary code, 21-bit binary code, 22-bit binary code, 23-bit binary code, 24-bit binary code, 25-bit binary code, 26-bit binary code, 27-bit binary code, 28 bit binary code, 29-bit binary code, and 30-bit binary code.

The methods and constructs described herein have significant advantages of those currently available in the art.

For example, HiPR-Cycle has extremely high target multiplexity (N):

N=2ⁿ−1,
where n is the number of bits/dyes used. This is per round. Other high multiplexity methods can typically identify 3-15 targets per round. For example, compared to the presently claimed method/bit system, other methods would need to use 60 rounds to get a similar multiplexity. Similarly, the HCR method by itself would need 200 rounds to achieve the same multiplexity.

HiPR-Cycle can also be performed in imaging rounds. This significant increases the potential for target multiplexity to:

N=2^{n*r}−1,
where r is the number of imaging rounds. So in two rounds, over a million targets can theoretically be identified, far surpassing other methods. The removal of probes can be performed using stripping buffers (high formamide and high temperatures) or photobleaching probes in existing samples.

The examples given above are essential for looking at gene expression in environmental microbiome samples. Even though a single bacteria strain has hundreds of genes on average, a collection of bacteria with S different strains will have potentially tens of thousands of different genes.

Because of the aforementioned, HiPR-Cycle is much cheaper than other methods targeting the same number of mRNAs (or other molecules) because it uses less reagent volume and reduces the number of fluorescently conjugated probes (the main driver of cost outside of encoding probes). For example, using two rounds of HiPR-Cycle and seven readout probes once could achieve 8000 targets, roughly the same number as 80 cycles of other methods that use 80-240 readout probes.

HiPR-Cycle is faster than other methods known in the art. HiPR-Cycle can be performed in 6-24 hours for a single round, and additional rounds could take an additional 1-12 hours. Currently, other methods can take days to a week for complete imaging or 24 hours per round. Because of this, HiPR-Cycle can be used to optically section tissue and resolve gene expression in 3D structures with higher z-ranges than with a wide field epifluorescence microscope. Thus, HiPR-Cycle could be used to look at gene expression in tissues in three dimensions.

HiPR-Cycle amplification can amplify signals of mRNA molecules. This is critical when rRNA and mRNA are to be measured simultaneously, as the rRNA signal is very high across cells. Without wishing to be bound to theory, the HiPR-Cycle amplification strategy, decreases amplification times by including emissive readout probes as described herein.

### Barcode Decoding

In some embodiments, a support vector machine is trained on reference data to predict the barcode of single cells in the synthetic communities and environmental samples. In a specific embodiment, the support vector machine is Support Vector Regression (SVR) from Python package. As used herein, the term “support-vector machine” (SVM) refers to a supervised learning model with associated learning algorithms that analyze data used for classification and regression analysis. Given a set of training examples, each marked as belonging to one or the other of two categories, an SVM training algorithm builds a model that assigns new examples to one category or the other, making it a non-probabilistic binary linear classifier. An SVM model is a representation of the examples as points in space, mapped so that the examples of the separate categories are divided by a clear gap that is as wide as possible. New examples are then mapped into that same space and predicted to belong to a category based on which side of the gap they fall.

In some embodiments, the reference spectra are obtained through a brute force approach involving the measurement of the spectra of all possible barcodes using barcoded test E. coli cells. In some embodiments, the n-bit binary encoding is a 10-bit binary encoding and tire reference spectra are obtained through measuring 1023 reference spectra.

In some embodiments the reference spectra are obtained by simulation of all possible spectra. In some embodiments, the simulated spectral data can be used as reference examples for the support vector machine. In some embodiments, the spectra corresponding to individual n-bit binary codes are simulated by adding together the measured spectra of each individual fluorophore (e.g., the reference spectrum for 0000010011 is generated by adding the spectra of R1, R2, and R5; or the reference spectrum for 1010010100 is generated by adding the spectra of R3, R5, R8 and R10). In some embodiments, the spectra corresponding to individual n-bit binary codes are simulated by adding the measured spectra of each individual fluorophore weighted by the relative contribution to the emission signal of each fluorophore. In some embodiments, the relative contribution of each fluorophore is calculated using a Forster Resonant Energy Transfer (FRET) model.

In some embodiments, specimens can be imaged using any one of the listed microscopy techniques and can include superresolution methods (e.g., Airy scan) to detect signals. A single or multiple field(s) of view can be acquired for each specimen. With multiple channels or excitations being performed with samples remaining in the same position for image acquisition. Image files (including metadata) can be saved (e.g., .czi or .nd2 filetypes). Then, data can be imported into a custom script. An optional noise reduction technique can be used to increase the signal-to-noise ratio in images. A generic or whole-cell stain (e.g., 16S rRNA, Eub, DAPI, etc.) can used to determine the boundaries of each bacterium. The channels across which the whole-cell stain is to be used can be integrated into a single image. A segmentation algorithm (e.g., trained U-net, HiPR-FISH-based segmentation, watershed algorithm, etc.) can be used to determine pixels belonging to bacteria and those belonging to the background. The algorithm can then be extended to determine the boundaries of adjacent bacteria. Individual masks can be generated for each bacterium, which receive a unique identifying label and a physical location identifier (e.g., X,Y,Z cartesian coordinates in the volumetric field of view). The channels corresponding to 16S rRNA imaging can be used to generate spectra and taxonomic barcodes using the HiPR-FISH analysis pipeline. Each bacterium identified can then be associated with a taxonomic ID. The channels corresponding to mRNA targeting probes can be integrated into a single image and a spot-detection algorithm can be used to generate a list of potential transcripts, providing each with a unique identifier and a physical location ID. Each ambiguous transcript can be assigned to a specific bacterium using the limits of the segmented mask, generated above. For each spot, a spectrum can be generated across all channels relevant to mRNA detection. The spectra can be compared to a library of spectra generated for combinations of fluorophores. A machine learning method (e.g. UMAP), can be used to generate the barcode of the spot from the trained data of the spectral library. Error correction can be performed to address potential issues; for example, colocalized transcripts could generate a signal that can be deconvoluted. Each bacterium can be assigned a list of identified transcripts. A downstream analysis can then be performed. For this downstream analysis, data structures including a matrix for each bacterial taxa identified can list, for example, each bacterium (columns) and each gene (rows), with the entries representing the number of transcripts for each gene in the cell. Then, physical interaction networks can show the proximity of each taxonomic group and, within each group, each cell state.

In another aspect, a method for analyzing a cell can include:
contacting at least one encoding probe with the cell to produce a first complex, wherein each encoding probe can include an mRNA targeting sequence and an initiator sequence;
adding two different DNA amplifiers to the first complex to produce a second complex, wherein each DNA amplifier can include an initiator complimentary sequence and a readout sequence; and
adding two emissive readout probes to the second complex, wherein each emissive readout probe can include a fluorophore and a complimentary sequence to the readout sequence of a corresponding DNA amplifier.

### EXAMPLES

### Example 1. HiPR-Cycle: Methods for Signal-Amplified Transcript Detection Compatible with Spectral Barcoding

Deciphering what each cell within taxonomically distinct microbes in a wide variety of samples and specimens is doing through gene expression and metabolic signatures represents the next frontier in understanding and interpreting microbial systems, with wide ranging applicability from clinical to agricultural domains. Here, we describe a novel technology for spatially-resolved multiplexed detection of gene expression within cells, called HiPR-Cycle. HiPR-Cycle uses hybridization chain reactions (HCR) to extend DNA polymers upon encountering ‘initiator’ sequences attached to DNA probes that recognize target genes of interest within cells in situ. The HCR products that form at the site of detected transcripts bear emissive readout probe binding sites, in numbers proportional to the size of the HCR product. Barcoding these HCR products with, for example, 10 readout probes can be used to distinguish >1000 distinct targets. Moreover, by physically amplifying the fluorescent signal of encoding probe binding events through HCR, we are able to detect lowly expressed genes, otherwise overlooked.

Materials

30% probe hybridization buffer: 30% formamide, 5× sodium chloride sodium citrate (SCC), 10% dextran sulfate, 0.1% Tween 20, optional 9 mM citric acid (pH 6.0), optional 50 μg/mL heparin, and optional 1×Denhardt's solution.

30% probe wash buffer: 30% formamide, 5× sodium chloride sodium citrate (SCC), 0.1% Tween 20, optional 9 mM citric acid (pH 6.0), and optional 50 μg/mL heparin.

Amplification buffer: 5× sodium chloride sodium citrate (SCC), 10% dextran sulfate, and 0.1% Tween 20.

Method

We placed samples on a slide and allowed to thoroughly dry. Then, added lysozyme to digest cell walls for 15 min at 37° C. and washed with 1×PBS for 10 min at room temperature. Then, added 30% formamide encoding buffer without probes to samples for 30 min at 37° C. followed by addition of 30% formamide encoding buffer with probes (400 nM) to samples for 3 hours at 37° C. and washed with wash buffer for 5 min at 37° C. We repeated this step two more times then washed with 5×SSC for 5 min at room temperature. The amplifier snap-cool procedure was started as follows: placed each amplifier in its own tube of strip tube and heated to 95° C. for 2 minutes in PCR block, removed and cooled at room temperature for 30 mins. After the washing, the amplification buffer (without probes) was added to the sample for 30 mins at room temperature. The snap-cool amplifiers were added to amplification buffer and placed onto samples. The sample was placed in a covered box to allow for amplification (overnight at room temperature). Then, we removed amplification buffer, washed with 5×SSC for 5 min at room temperature and repeated the step three more times. We then added HiPR readout buffer (2×SSC, 5×Denhardt's solution, 10% dextran sulfate, 10% ethylene carbonate, 0.01% SDS, 400 nM readout probes) for 1 hour at room temperature, washed with HiPR wash buffer (215 mM NaCl, 20 mM Tris-HCl pH 8.0, 10 mM EDTA) for 5 min at room temperature, and repeated this step two more times. We then rinsed with 5×SSCT and allowed to dry. We then added mountant and placed a coverslip over samples then proceeded to imaging.

Imaging was done with a Zeiss i880 confocal microscope with Zen Black (Zeiss) to take the images. For each laser excitation, photons were collected from the excitation wavelength up to about 690 nm in wavelength bins that were 8.9 nm wide. For instance, for 633 nm excitation photons were collected into 6 bins (633-642, 642-651, 651-660, 660-669, 669-678, 678-687 nm). For each image, 5 separate excitations were performed and about 90 channels are collected. Channels were selected or merged as needed to illustrate the success/failure of the assay. The laser settings were in accordance with Table 1.

TABLE 1
Laser Settings¹.

| Laser | | Pinhole | Laser | Pixel Size | Pixel Dwell | Bit | Scanning |
|---|---|---|---|---|---|---|---|
| Excitation (nm) | Laser | Size (nm) | Power (%) | (nm) | Time (μsec) | Depth | Direction |
| 405 | Diode | 51.7 | 4 | 70 | 8.4 | 16-bit | Bidirectional |
| | 405-30 | | | | | | |
| 488 | Argon | 55.8 | 4 | 70 | 8.4 | 16-bit | Bidirectional |
| 514 | Argon | 58.0 | 6 | 70 | 8.4 | 16-bit | Bidirectional |
| 561 | DPSS | 59.8 | 5 | 70 | 8.4 | 16-bit | Bidirectional |
| | 561-10 | | | | | | |
| 633 | HeNe633 | 63.8 | 6 | 70 | 8.4 | 16-bit | Bidirectional |

¹All used a scanning repeat of 1 s, Master Gain of 800, Digital Offset of 0, and Digital Gain of 1.

A 2000×2000 pixel image was typically taken. Other settings were also used, for example zoom in 2× and take a 1000×1000 image (so the resolution is the same), in this case, the pixel dwell time is doubled to 16.8 μsec.

The amplifier probes used in the following examples are shown in Table 2.

TABLE 2
Amplifier Probes

| SEQ ID | | |
|---|---|---|
| NO: | Probe Name | Sequence |
| 21 | Amplifier | AGGGTGTGTTTGTAAAGGGTTTGTTGCAAAGGAACGTCGAGCTG |
| | Probe 1 | TAATGGTGCTCGACGTTCC |
| 22 | Amplifier | GCTCGACGTTCCTTTGCAACAGGAACGTCGAGCACCATTACATA |
| | Probe 2 | GGGTGTGTTTGTAAAGGGT |
| 23 | Amplifier | TAGAGTTGATAGAGGGAGAATAGTACATGTCGTGGTGGTAGCTT |
| | Probe 3 | GTATGAAGCTACCACCACG |
| 24 | Amplifier | GCTACCACCACGACATGTACTCGTGGTGGTAGCTTCATACAATT |
| | Probe 4 | AGAGTTGATAGAGGGAGAA |
| 85 | Amplifier | ATAGGAAATGGTGGTAGTGTTATGTAAGATGCTCACCTGACGTT |
| | Probe 5 | CATGTAACGTCAGGTGAGC |
| 86 | Amplifier | CGTCAGGTGAGCATCTTACATGCTCACCTGACGTTACATGAATA |
| | Probe 6 | TAGGAAATGGTGGTAGTGT |
| 129 | Amplifier | AGGGTGTGTTTGTAAAGGGTTATGTAAGATGCTCACCTGACGTT |
| | Probe 7 | CATGTAACGTCAGGTGAGC |
| 130 | Amplifier | CGTCAGGTGAGCATCTTACATGCTCACCTGACGTTACATGAATA |
| | Probe 8 | GGGTGTGTTTGTAAAGGGT |
| 131 | Amplifier | TGTGGAGGGATTGAAGGATATTGTTGCAAAGGAACGTCGAGCTG |
| | Probe 9 | TAATGGTGCTCGACGTTCC |
| 132 | Amplifier | GCTCGACGTTCCTTTGCAACAGGAACGTCGAGCACCATTACATT |
| | Probe 10 | GTGGAGGGATTGAAGGATA |
| 242 | Amplifier | TAGAGTTGATAGAGGGAGAATTGTTGCAAAGGAACGTCGAGCT |
| | Probe 11 | GTAATGGTGCTCGACGTTCC |
| 243 | Amplifier | GCTCGACGTTCCTTTGCAACAGGAACGTCGAGCACCATTACATT |
| | Probe 12 | AGAGTTGATAGAGGGAGAA |
| 244 | Amplifier | AGGGTGTGTTTGTAAAGGGTTATACGACTTCGACGACCACCCAA |
| | Probe 13 | CTTGAATGGGTGGTCGTCG |
| 245 | Amplifier | GGGTGGTCGTCGAAGTCGTATCGACGACCACCCATTCAAGTTTA |
| | Probe 14 | GGGTGTGTTTGTAAAGGGT |
| 279 | Amplifier | TGTGGAGGGATTGAAGGATATTAGACTGAACCCACTCCGACGAT |
| | Probe 15 | CTGTCTTCGTCGGAGTGGG |
| 280 | Amplifier | CGTCGGAGTGGGTTCAGTCTACCCACTCCGACGAAGACAGATTT |
| | Probe 16 | GTGGAGGGATTGAAGGATA |
| 281 | Amplifier | GATGATGTAGTAGTAAGGGTTTAGACTGAACCCACTCCGACGAT |
| | Probe 17 | CTGTCTTCGTCGGAGTGGG |
| 282 | Amplifier | CGTCGGAGTGGGTTCAGTCTACCCACTCCGACGAAGACAGATTG |
| | Probe 18 | ATGATGTAGTAGTAAGGGT |
| 283 | Amplifier | TGAACTCGGCGGGTTAGGAATTTAGACTGAACCCACTCCGACGA |
| | Probe 19 | TCTGTCTTCGTCGGAGTGGG |
| 284 | Amplifier | CGTCGGAGTGGGTTCAGTCTACCCACTCCGACGAAGACAGATTC |
| | Probe 20 | TAAGGTTTTGAACTCGGCGG |
| 285 | Amplifier | GATGATGTAGTAGTAAGGGTTTATTCCTAACCCGCCGAGTTCAC |
| | Probe 21 | TAAGGTTTTGAACTCGGCGG |
| 286 | Amplifier | TGAACTCGGCGGGTTAGGAATCCGCCGAGTTCAAAACCTTAGTT |
| | Probe 22 | GATGATGTAGTAGTAAGGGT |
| 381 | Amplifier | TGTGGAGGGATTGAAGGATATATGGAAGATGCTCACCGACCGTT |
| | Probe 23 | CATGCAACGGTCGGTGAGC |
| 382 | Amplifier | CGGTCGGTGAGCATCTTCCATGCTCACCGACCGTTGCATGAATT |
| | Probe 24 | GTGGAGGGATTGAAGGATA |
| 383 | Amplifier | TGAAAGGAATGGGTTGTGGTTTAGCATGTACTGACGCTCCACTT |
| | Probe 25 | CAACCAAGTGGAGCGTCAG |
| 384 | Amplifier | GTGGAGCGTCAGTACATGCTACTGACGCTCCACTTGGTTGAATT |
| | Probe 26 | GAAAGGAATGGGTTGTGGT |
| 385 | Amplifier | ATAGGAAATGGTGGTAGTGTTAAATCCAATCCACCGACCAGCAA |
| | Probe 27 | TTGAGATGCTGGTCGGTGG |
| 386 | Amplifier | GCTGGTCGGTGGATTGGATTTCCACCGACCAGCATCTCAATTTAT |
| | Probe 28 | AGGAAATGGTGGTAGTGT |
| 795 | amplifier | AGAGTGAGTAGTAGTGGAGTTTATTCTACACGGAGCATGTGCAT |
| | probe 29 | ATCAACCGCACATGCTCCG |
| 796 | Amplifier | GCACATGCTCCGTGTAGAATACGGAGCATGTGCGGTTGATATTA |
| | Probe 30 | GAGTGAGTAGTAGTGGAGT |
| 797 | Amplifier | CTCTAACTTCCATCACATTAGTCCATAGGCCGGTATGCGAGCTTT |
| | Probe 31 | AAACGCATACCGGCC |
| 798 | Amplifier | CGCATACCGGCCTATGGACTAGGCCGGTATGCGTTTAAAGCTTA |
| | Probe 32 | CCCTCTAACTTCCATC |
| 799 | Amplifier | ACAACCCATTCCTTTCATGACTTAAACGCGTAACGGAGCGATCT |
| | Probe 33 | TAGAGCTCCGTTACGC |
| 800 | Amplifier | GCTCCGTTACGCGTTTAAGTCGCGTAACGGAGCTCTAAGATCTA |
| | Probe 34 | CCACAACCCATTCCTT |
| 801 | Amplifier | ACTCCCTACACCTCCAATTCATACGAATGCTCGGGTTGCTTACTA |
| | Probe 35 | ATCGCAACCCGAGCA |
| 802 | Amplifier | GCAACCCGAGCATTCGTATGATGCTCGGGTTGCGATTAGTAATT |
| | Probe 36 | TTACTCCCTACACCTC |
| 803 | amplifier | TTAGGTTGAGAATAGGATAGGAATACTCAGACGGAGGCAAGAA |
| | probe 37 | GCTTTTGCCTCCGTCTG |
| 804 | Amplifier | TGCCTCCGTCTGAGTATTCCTCAGACGGAGGCAAAAGCTTCTTA |
| | Probe 38 | GGTTAGGTTGAGAATA |
| 805 | Amplifier | AGTTGATAGAGGGAGAATATTGATCATCCTGGCACACACAGTAA |
| | Probe 39 | CATTGTGTGTGCCAGG |
| 806 | Amplifier | GTGTGTGCCAGGATGATCAATCCTGGCACACACAATGTTACTTT |
| | Probe 40 | AGAGTTGATAGAGGGA |
| 979 | Amplifier | ATAGGAAATGGTGGTAGTGTTTAGACTGAACCCACTCCGACGAT |
| | Probe 41 | CTGTCTTCGTCGGAGTGGG |
| 980 | Amplifier | CGTCGGAGTGGGTTCAGTCTACCCACTCCGACGAAGACAGATTA |
| | Probe 42 | TAGGAAATGGTGGTAGTGT |
| 981 | Amplifier | GATGTAGTAGTAAGGGTTATTCCTAACCCGCCGAGTTCACTAAG |
| | Probe 43 | GTTTTGAACTCGGCGG |
| 982 | Amplifier | TGAACTCGGCGGGTTAGGAATCCGCCGAGTTCAAAACCTTAGTG |
| | Probe 44 | ATGATGTAGTAGTAAG |
| 1185 | Amplifier | TTGGAGGTGTAGGGAGTAAATTGTTGCAAAGGAACGTCGAGCTG |
| | Probe 45 | TAATGGTGCTCGACGTTCC |
| 1186 | Amplifier | GCTCGACGTTCCTTTGCAACAGGAACGTCGAGCACCATTACATT |
| | Probe 46 | TGGAGGTGTAGGGAGTAAA |
| 1240 | Amplifier | GTAATTGAGTAGAAGGGTAATACGGTTTAGCGGTGCCAGTTTAA |
| | Probe 47 | TGCACTGGCACCGCTA |
| 1241 | Amplifier | CTGGCACCGCTAAACCGTATTTAGCGGTGCCAGTGCATTAAATG |
| | Probe 48 | GGATGAGGTAATTGAG |
| 1242 | Amplifier | TAATAGATATGAGGGTGTAGTCAACTACGGGCATCGTTGTTAAG |
| | Probe 49 | GCTTCAACGATGCCCG |
| 1243 | Amplifier | CAACGATGCCCGTAGTTGACTCGGGCATCGTTGAAGCCTTAATT |
| | Probe 50 | GGGAGGGTAATAGATA |

The readout probes used in the following examples are shown in Table 3.

TABLE 3
Readout Probes

| SEQ ID | | |
|---|---|---|
| NO: | Probe Name | Sequence |
| 25 | Readout Probe 1 | /5Alex488N/TATCCTTCAATCCCTCCACA |
| 26 | Readout Probe 2 | /5Alex546N/ACACTACCACCATTTCCTAT |
| 27 | Readout Probe 3 | /56-ROXN/ACTCCACTACTACTCACTCT/3Rox_N/ |
| 28 | Readout Probe 4 | /5PacificGreenN/ACCCTCTAACTTCCATCACA |
| 29 | Readout Probe 5 | /5PacificBlueN/ACCACAACCCATTCCTTTCA |
| 30 | Readout Probe 6 | /5Atto610N/TTTACTCCCTACACCTCCAA |
| 31 | Readout Probe 7 | /5Alex647N/ACCCTTTACAAACACACCCT |
| 32 | Readout Probe 8 | /5DyLight-510-LS/TCCTATTCTCAACCTAACCT/3DyLight-510- |
| | | LS/ |
| 33 | Readout Probe 9 | /5Alex405N/TTCTCCCTCTATCAACTCTA |
| 34 | Readout Probe 10 | /5Alex532N/ACCCTTACTACTACATCATC/3Alexa532N/ |

### Example 2. HiPR-Cycle Two-Bit System

As a proof of concept, we performed validation experiments with E. coli with GFP/ampR plasmid. The validation experiments included: Target fixed GFP+/− E. coli with mRNA encoding probes and a two bit encoding scheme leading to excitation of either Alexa 405 or Alexa 647.

As shown in FIG. 2, in situ gene targeting validated HiPR-Cycle in a two-bit system. The total assay time was 24 hours. Encoding with either or both bit(s) was possible and it produced a low background/high signal.

These experiments showed that HiPR-Cycle probe intensity correlated to protein expression and that genes can be specifically barcoded. There was also transcript abundance scales in channels with multiple barcodes. In contrast, the GFP⁻ cells have low barcode intensity. Further, confocal imaging in HiPR-Cycle revealed distinct spectra for different barcodes.

The encoding probes used in this example are shown in Table 4 Amplifier probes 1-4 (SEQ ID NO: 21-24), as shown in Table 2, were used in this example. Readout probes 1-10 (SEQ ID NO: 25-34), as shown in Table 3, were used in this example.

TABLE 4
Encoding and amplifier probes used in Example 2.

| SEQ ID | | |
|---|---|---|
| NO: | Probe Name | Sequence |
| 1 | Encoding Probe 1 | TGCCATGTGTAATCCCAGCAGCACAGCTCGACGTTCCTTTG |
| | | CAACA |
| 2 | Encoding Probe 2 | TGTGGTCTCTCTTTTCGTTGGGAAGAGCTCGACGTTCCTTTG |
| | | CAACA |
| 3 | Encoding Probe 3 | AGGGCAGATTGTGTGGACAGGTTAGCTCGACGTTCCTTTGC |
| | | AACA |
| 4 | Encoding Probe 4 | GGTAAAAGGACAGGGCCATCGCGTTGCTCGACGTTCCTTTG |
| | | CAACA |
| 5 | Encoding Probe 5 | GGTCTGCTAGTTGAACGCTTCCAAGAGCTCGACGTTCCTTT |
| | | GCAACA |
| 6 | Encoding Probe 6 | TCAAACTTGACTTCAGCACGTGTGAAGCTCGACGTTCCTTT |
| | | GCAACA |
| 7 | Encoding Probe 7 | ACCTTCGGGCATGGCACTCTACTGCTCGACGTTCCTTTGCA |
| | | ACA |
| 8 | Encoding Probe 8 | TCTCGCAAAGCATTGAACACCAATTGCTCGACGTTCCTTTG |
| | | CAACA |
| 9 | Encoding Probe 9 | AGTGACAAGTGTTGGCCATGGATGTGCTCGACGTTCCTTTG |
| | | CAACA |
| 10 | Encoding Probe 10 | TGTTGCATCACCTTCACCCTCTGGTGCTCGACGTTCCTTTGC |
| | | AACA |
| 11 | Encoding Probe 11 | TGCCATGTGTAATCCCAGCAGCACAGCTACCACCACGACAT |
| | | GTACT |
| 12 | Encoding Probe 12 | TGTGGTCTCTCTTTTCGTTGGGAAGAGCTACCACCACGACA |
| | | TGTACT |
| 13 | Encoding Probe 13 | AGGGCAGATTGTGTGGACAGGTTAGCTACCACCACGACAT |
| | | GTACT |
| 14 | Encoding Probe 14 | GGTAAAAGGACAGGGCCATCGCGTTGCTACCACCACGACA |
| | | TGTACT |
| 15 | Encoding Probe 15 | GGTCTGCTAGTTGAACGCTTCCAAGAGCTACCACCACGACA |
| | | TGTACT |
| 16 | Encoding Probe 16 | TCAAACTTGACTTCAGCACGTGTGAAGCTACCACCACGACA |
| | | TGTACT |
| 17 | Encoding Probe 17 | ACCTTCGGGCATGGCACTCTACTGCTACCACCACGACATGT |
| | | ACT |
| 18 | Encoding Probe 18 | TCTCGCAAAGCATTGAACACCAATTGCTACCACCACGACAT |
| | | GTACT |
| 19 | Encoding Probe 19 | AGTGACAAGTGTTGGCCATGGATGTGCTACCACCACGACA |
| | | TGTACT |
| 20 | Encoding Probe 20 | TGTTGCATCACCTTCACCCTCTGGTGCTACCACCACGACAT |
| | | GTACT |

### Example 3. HiPR-Cycle Two-Bit System is Compatible with HiPR-FISH

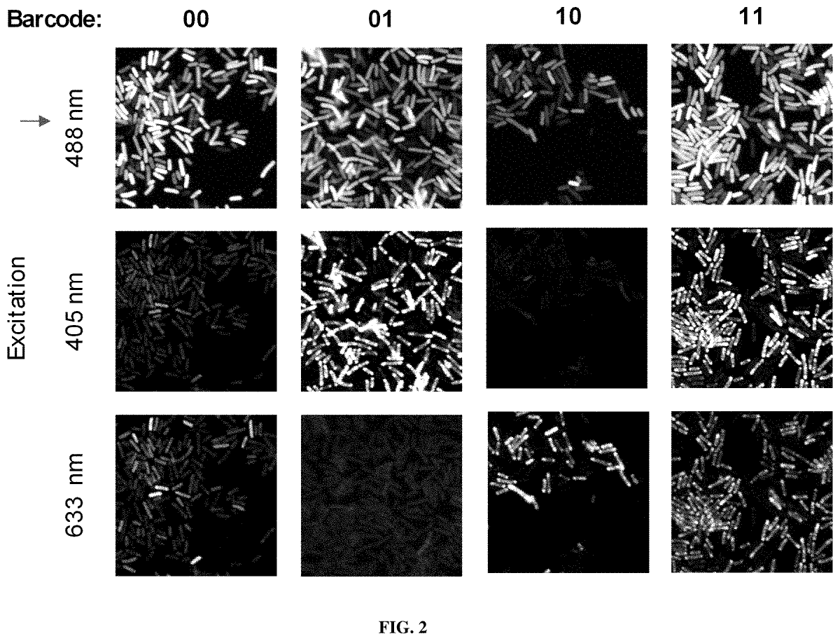

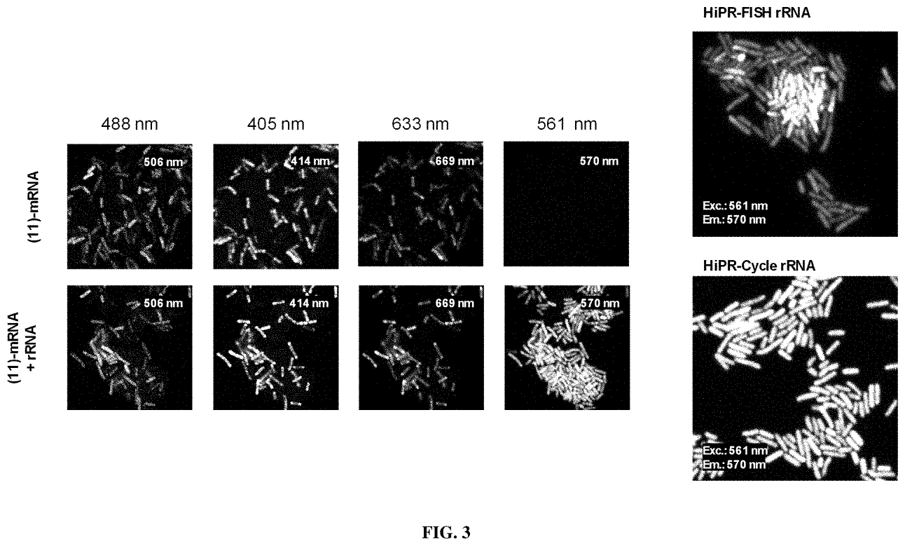

As shown in FIG. 3, we measured the use of direct HiPR-FISH encoding in the HiPR-Cycle assay.

Here, HiPR-FISH encoding probes designed to target the E. coli 16S and 23S rRNA segments (with direct R2 readout probes) were added in the encoding probe mixture with HiPR-Cycle encoding probes for GFP transcripts (with indirect R7 and R9 probes through hybridization chain reaction). The encoding was performed for 3 hours at 37° C., while the amplification was performed overnight using two sets of amplifier probes. A readout hybridization was performed with all ten readout probes for one hour at room temperature after amplification.

These experiments showed that there was no change in mRNA encoding with or without the inclusion of rRNA HiPR-FISH probes. Importantly, a high intensity signal was detected in the appropriate emission channel (570 nm after excitation with 561 nm laser), whereas it was undetectable when rRNA probes were not used.

As a further extension of the use of rRNA probes, we compared HiPR-FISH and HiPR-Cycle probes to detect rRNA. Here, we designed HiPR-Cycle probes that were identical to the previously used HiPR-FISH rRNA probes for E. coli, except we replaced the flanking readout regions of these probes with flanking initiators to trigger the amplifiers. FIG. 3 (Right) shows that rRNA-targeting HiPR-Cycle probes perform equal to or better than HiPR-FISH.

The encoding probes used in this example are shown in Table 5 Amplifier probes 1-2 and 5-6 (SEQ ID NO: 21-22 and 85-86), as shown in Table 2, were used in this example. Readout probes 1-10 (SEQ ID NO: 25-34), as shown in Table 3, were used in this example.

TABLE 5
Encoding probes used in Example 3.

| SEQ ID | | |
|---|---|---|
| NO: | Probe Name | Sequence |
| 35 | Encoding Probe | AGGGTGTGTTTGTAAAGGGTACCTCAGTTAATGATAGTGTG |
| | 21 | TCGTTT |
| 36 | Encoding Probe | AGGGTGTGTTTGTAAAGGGTAGGAAGGCACATTCTCATCTC |
| | 22 | ACT |
| 37 | Encoding Probe | AGGGTGTGTTTGTAAAGGGTCCACGTCAATGAGCAAAGGTA |
| | 23 | AAT |
| 38 | Encoding Probe | AGGGTGTGTTTGTAAAGGGTCCTCAGTTAATGATAGTGTGT |
| | 24 | CGATTG |
| 39 | Encoding Probe | AGGGTGTGTTTGTAAAGGGTCCTCAGTTAATGATAGTGTGT |
| | 25 | CGTTT |
| 40 | Encoding Probe | AGGGTGTGTTTGTAAAGGGTCTAAGTTAATGATAGTGTGTC |
| | 26 | GATTG |
| 41 | Encoding Probe | AGGGTGTGTTTGTAAAGGGTGATACACACACTGATTCAGGC |
| | 27 | AGA |
| 42 | Encoding Probe | AGGGTGTGTTTGTAAAGGGTGCGTCACCCCATTAAGAGGCT |
| | 28 | AGG |
| 43 | Encoding Probe | AGGGTGTGTTTGTAAAGGGTGTAAGCTCACAATATGTGCAT |
| | 29 | TAAA |
| 44 | Encoding Probe | AGGGTGTGTTTGTAAAGGGTGTATCATCTCTGAAAACTTCC |
| | 30 | GACC |
| 45 | Encoding Probe | AGGGTGTGTTTGTAAAGGGTGTGTCTCATCTCTGAAAACTT |
| | 31 | CCCAC |
| 46 | Encoding Probe | AGGGTGTGTTTGTAAAGGGTTAGGCTCACAATATGTGCATT |
| | 32 | AAA |
| 47 | Encoding Probe | AGGGTGTGTTTGTAAAGGGTTGCGTCACCCCATTAAGAGGC |
| | 33 | AGG |
| 48 | Encoding Probe | ATAGGAAATGGTGGTAGTGTAGTCTTGGTTTTCCGGATTTG |
| | 34 | GGA |
| 49 | Encoding Probe | ATAGGAAATGGTGGTAGTGTCATGTCAATGAGCAAAGGTAT |
| | 35 | TAAGAA |
| 50 | Encoding Probe | ATAGGAAATGGTGGTAGTGTCATGTCAATGAGCAAAGGTAT |
| | 36 | TATGA |
| 51 | Encoding Probe | ATAGGAAATGGTGGTAGTGTCGTCACCCCATTAAGAGGCTC |
| | 37 | GGT |
| 52 | Encoding Probe | ATAGGAAATGGTGGTAGTGTGAAACTAACACACACACTGAT |
| | 38 | TGTC |
| 53 | Encoding Probe | ATAGGAAATGGTGGTAGTGTGAGCCTTGGTTTTCCGGATTT |
| | 39 | CGG |
| 54 | Encoding Probe | ATAGGAAATGGTGGTAGTGTGGAGCCTTGGTTTTCCGGATT |
| | 40 | ACG |
| 55 | Encoding Probe | ATAGGAAATGGTGGTAGTGTGTAAGCTCACAATATGTGCAT |
| | 41 | AAA |
| 56 | Encoding Probe | ATAGGAAATGGTGGTAGTGTGTCACCCCATTAAGAGGCTCC |
| | 42 | GTG |
| 57 | Encoding Probe | ATAGGAAATGGTGGTAGTGTGTGCTCAGCCTTGGTTTTCCG |
| | 43 | CTA |
| 58 | Encoding Probe | ATAGGAAATGGTGGTAGTGTGTGTCTCATCTCTGAAAACTT |
| | 44 | CCGACC |
| 59 | Encoding Probe | ATAGGAAATGGTGGTAGTGTTGACACACACACTGATTCAGG |
| | 45 | GAG |
| 60 | Encoding Probe | CGTCAGGTGAGCATCTTACATAGTCTTGGTTTTCCGGATTTG |
| | 46 | GGA |
| 61 | Encoding Probe | CGTCAGGTGAGCATCTTACATCATGTCAATGAGCAAAGGTA |
| | 47 | TTAAGAA |
| 62 | Encoding Probe | CGTCAGGTGAGCATCTTACATCATGTCAATGAGCAAAGGTA |
| | 48 | TTATGA |
| 63 | Encoding Probe | CGTCAGGTGAGCATCTTACATCGTCACCCCATTAAGAGGCT |
| | 49 | CGGT |
| 64 | Encoding Probe | CGTCAGGTGAGCATCTTACATGAAACTAACACACACACTGA |
| | 50 | TTGTC |
| 65 | Encoding Probe | CGTCAGGTGAGCATCTTACATGAGCCTTGGTTTTCCGGATTT |
| | 51 | CGG |
| 66 | Encoding Probe | CGTCAGGTGAGCATCTTACATGGAGCCTTGGTTTTCCGGATT |
| | 52 | ACG |
| 67 | Encoding Probe | CGTCAGGTGAGCATCTTACATGTAAGCTCACAATATGTGCA |
| | 53 | TAAA |
| 68 | Encoding Probe | CGTCAGGTGAGCATCTTACATGTCACCCCATTAAGAGGCTC |
| | 54 | CGTG |
| 69 | Encoding Probe | CGTCAGGTGAGCATCTTACATGTGCTCAGCCTTGGTTTTCCG |
| | is | CTA |
| 70 | Encoding Probe | CGTCAGGTGAGCATCTTACATGTGTCTCATCTCTGAAAACTT |
| | 56 | CCGACC |
| 71 | Encoding Probe | CGTCAGGTGAGCATCTTACATTGACACACACACTGATTCAG |
| | 57 | GGAG |
| 72 | Encoding Probe | GCTCGACGTTCCTTTGCAACAACCTCAGTTAATGATAGTGT |
| | 58 | GTCGTTT |
| 73 | Encoding Probe | GCTCGACGTTCCTTTGCAACAAGGAAGGCACATTCTCATCT |
| | 59 | CACT |
| 74 | Encoding Probe | GCTCGACGTTCCTTTGCAACACCACGTCAATGAGCAAAGGT |
| | | AAAT |
| 75 | Encoding Probe | GCTCGACGTTCCTTTGCAACACCTCAGTTAATGATAGTGTGT |
| | 61 | CGATTG |
| 76 | Encoding Probe | GCTCGACGTTCCTTTGCAACACCTCAGTTAATGATAGTGTGT |
| | 62 | CGTTT |
| 77 | Encoding Probe | GCTCGACGTTCCTTTGCAACACTAAGTTAATGATAGTGTGTC |
| | 63 | GATTG |
| 78 | Encoding Probe | GCTCGACGTTCCTTTGCAACAGATACACACACTGATTCAGG |
| | 64 | CAGA |
| 79 | Encoding Probe | GCTCGACGTTCCTTTGCAACAGCGTCACCCCATTAAGAGGC |
| | 65 | TAGG |
| 80 | Encoding Probe | GCTCGACGTTCCTTTGCAACAGTAAGCTCACAATATGTGCA |
| | 66 | TTAAA |
| 81 | Encoding Probe | GCTCGACGTTCCTTTGCAACAGTATCATCTCTGAAAACTTCC |
| | 67 | GACC |
| 82 | Encoding Probe | GCTCGACGTTCCTTTGCAACAGTGTCTCATCTCTGAAAACTT |
| | 68 | CCCAC |
| 83 | Encoding Probe | GCTCGACGTTCCTTTGCAACATAGGCTCACAATATGTGCATT |
| | 69 | AAA |
| 84 | Encoding Probe | GCTCGACGTTCCTTTGCAACATGCGTCACCCCATTAAGAGG |
| | 70 | CAGG |

### Example 4. Optimization of HiPR-Cycle Two-Bit System

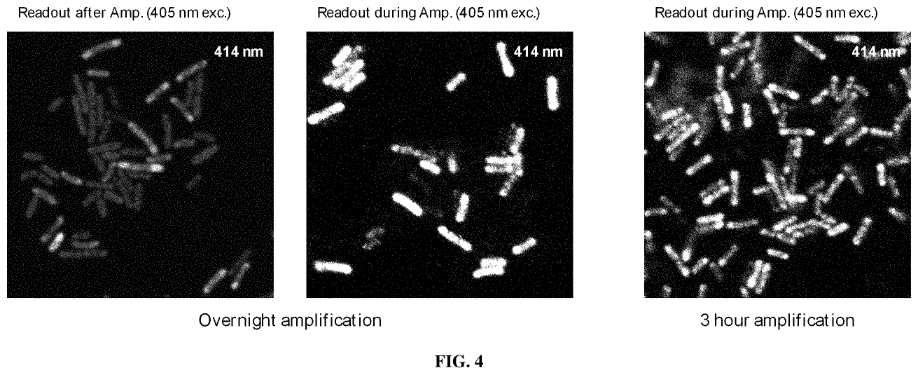

As shown in FIG. 4, combining the amplification and readout steps can reduce the assay time from overnight (12+ hours) to about 3 hours. In here, we performed three assays simultaneously: 1) one assay where the addition of readout probes was performed after an overnight (12+ hours) amplification, 2) another assay where readout probes were added during the overnight amplification step, and 3) another assay where readout probes were added during a 3-hour amplification step. In general, GFP+ E. coli (ATCC GFP25922) were fixed and adhered to charged, ultrastick glass slides. Cell walls were digested using a 15 minute lysozyme digestion at 37° C. followed by room temperature wash of 1×PBS. A pre-encoding incubation was performed at 37° C. for 30 minutes. Encoding probes for GFP transcripts that included initiators corresponding to Readout Probe 7 (R7; Alexa 645) and Readout Probe 9 (R9; Alexa 405) were hybridized during a 3-hour, 37° C. encoding hybridization (30% formamide buffer). Probes were washed away using encoding wash buffer (30% formamide, 5 minute washes at 37° C., multiple washes). Amplification was performed by adding amplifier probes (50 nM) corresponding to readout probes R7 and R9 (annealing: 95° C. for 2 minutes, room temperature for 30 minutes) to the sample after a 30 minute, room temperature incubation in pre-amplification buffer. Amplification was performed for either 3 hours or 12 hours. If readout probes (40 nM) were added at the same time as amplifier probes, samples were washed with 5×SSCT before sample mounting. If readout probes were added after amplification a readout probe hybridization was performed at room temperature for 30 minutes with all ten readout probes (readout probes 1-10; 40 nM). Samples were mounted in an imaging medium, a coverslip was placed over them, and they were imaged on a Zeiss i880 confocal microscope.

Imaging was performed using the 405 nm, 488 nm, and 633 nm lasers. Captured images from specific fields of view were contrasted equivalently. In the image, a single emission channel (414 nm) is shown for a field of view from each condition after stimulation with a 405 nm laser.

Amplifier probes 1-4 (SEQ ID NO: 21-24), as shown in Table 2, were used in this example. Readout probes 1-10 (SEQ ID NO: 25-34), as shown in Table 3, were used in this example. Encoding probes 1-20 (SEQ ID NO: 1-20), as shown in Table 4, were used in this example.

### Example 5. HiPR-Cycle Three-Bit System

We also performed experiments with E. coli with GFP/ampR plasmid. The experiments included: Target fixed GFP+/− E. coli with mRNA encoding probes and a three bit encoding scheme leading to excitation of Alexa 405, Alexa 561, or Alexa 633.

All E. coli were barcoded with a single set of HiPR-Cycle probes targeting GFP transcripts. In here, only one readout probe, Readout Probe 2, (R2) should have bound to the amplifiers used in this sample. As shown in FIG. 5, top panels show fluorescence signal in each channel including GFP (488 nm). Bottom panel overlays all channels and depicts mostly overlapping GFP (green) and R2 (magenta) signal with little background from other channels.

As expected GFP and R2 were highly detected across cells, while Readout Probe 7 (R7) and Readout Probe 9 (R9) were less abundant. Consistent with the design, only R2 showed heightened signal across cells. In this pure sample (R2 only) the majority of cells were correctly classified, suggesting limited background signal from other probes.

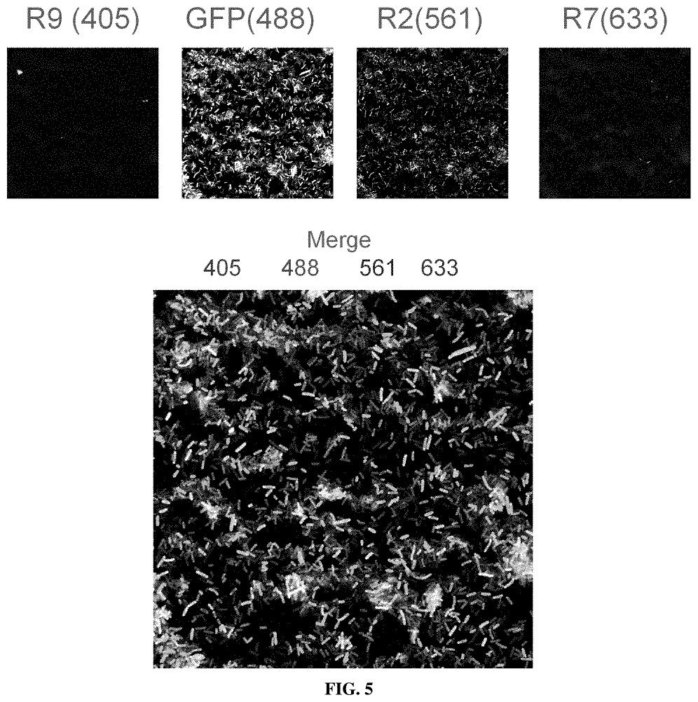

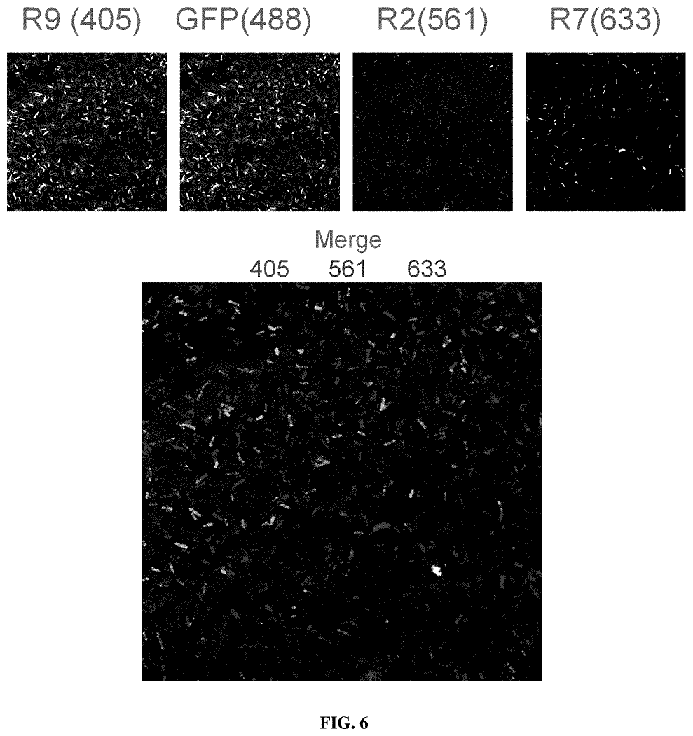

We also developed a synthetic mixture of uniquely barcoded cells. Sample 1 (Red): all cells barcoded with R7 readout probes=0001000001. Sample 2 (Blue): all cells barcoded with R9 readout probes=0100000001. Sample 3 (magenta): all cells barcoded with R2 readout probes=0000000011. We then mixed these samples 1:1:1. As shown in FIG. 6, the top panels show fluorescence signal in each channel including GFP (488 nm). The bottom panel overlays all channels and reveals mutually exclusive fluorescence of each readout probe within cells.

Signal thresholding identifies uniquely barcoded cells in mixtures. As expected only a subset of cells were positive for each readout fluorophore. Consistent with our mixture, only a subset of cells exhibit heightened signal for each readout fluorophore.

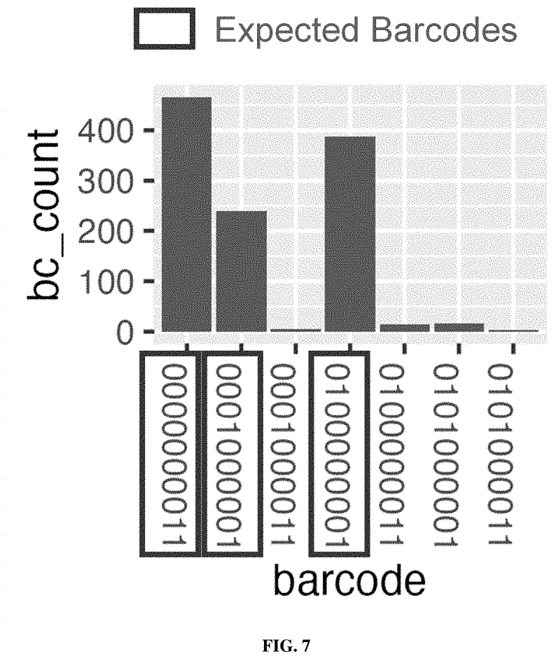

HiPR-Cycle barcoded cells can be resolved in mixtures. As shown in FIG. 7, consistent with the mixture composition (1:1:1), most cells were identified as having just one of the three probes suggesting limited cross talk between HiPR-Cycle probes.

Encoding probes 1-20 (SEQ ID NO: 1-20), as shown in Table 4, and encoding probes 71-80 as shown in Table 6 below were used in this example. Amplifier probes 1-6 (SEQ ID NO: 21-24 and 85-86), as shown in Table 2, were used in this example. Readout probes 1-10 (SEQ ID NO: 25-34), as shown in Table 3, were used in this example.

TABLE 6
Encoding probes used in Example 5.

| SEQ ID | | |
|---|---|---|
| NO: | Probe Name | Sequence |
| 87 | Encoding Probe | TGCCATGTGTAATCCCAGCAGCACACGTCAGGTGAGCATCT |
| | 71 | TACAT |
| 88 | Encoding Probe | TGTGGTCTCTCTTTTCGTTGGGAAGACGTCAGGTGAGCATC |
| | 72 | TTACAT |
| 89 | Encoding Probe | AGGGCAGATTGTGTGGACAGGTTACGTCAGGTGAGCATCT |
| | 73 | TACAT |
| 90 | Encoding Probe | GGTAAAAGGACAGGGCCATCGCGTTCGTCAGGTGAGCATC |
| | 74 | TTACAT |
| 91 | Encoding Probe | GGTCTGCTAGTTGAACGCTTCCAAGACGTCAGGTGAGCATC |
| | 75 | TTACAT |
| 92 | Encoding Probe | TCAAACTTGACTTCAGCACGTGTGAACGTCAGGTGAGCATC |
| | 76 | TTACAT |
| 93 | Encoding Probe | ACCTTCGGGCATGGCACTCTACTCGTCAGGTGAGCATCTTA |
| | 77 | CAT |
| 94 | Encoding Probe | TCTCGCAAAGCATTGAACACCAATTCGTCAGGTGAGCATCT |
| | 78 | TACAT |
| 95 | Encoding Probe | AGTGACAAGTGTTGGCCATGGATGTCGTCAGGTGAGCATCT |
| | 79 | TACAT |
| 96 | Encoding Probe | TGTTGCATCACCTTCACCCTCTGGTCGTCAGGTGAGCATCT |
| | 80 | TACAT |

### Example 6. Detecting Endogenous Genes in Bacterial Cells

One of the primary goals of the HiPR-Cycle technology is to detect endogenous bacterial gene expression. Because of the small cell size and sparsity of transcripts of most endogenous genes in bacteria, detecting measuring gene expression with fluorescence-based imaging poses a significant challenge. We therefore sought to use HiPR-Cycle to detect gene expression from an inducible gene endogenous to E. coli.

LacZ is a well-studied Beta-D-Galactosidase gene whose expression can be induced by the presence of galactose or a galactose mimic, Isopropyl ß-D-1-thiogalactopyranoside (IPTG), within the bacterial culture media. To examine the ability to detect LacZ expression with HiPR-Cycle, we grew E. coli cultures in the presence of IPTG at varying concentrations. Specifically E. coli were grown in media containing 0 mM, 0.1 mM, or 1 mM IPTG for 90 minutes before being collected for HiPR-Cycle.

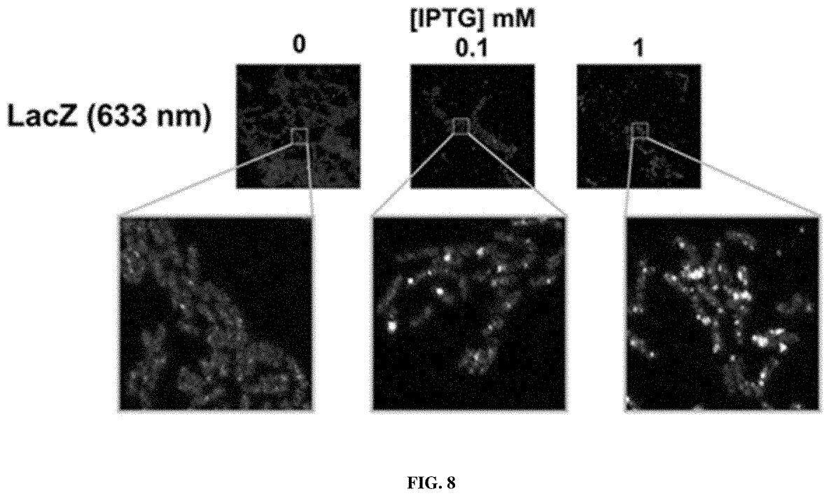

To detect LacZ transcripts, we designed 32 HiPR-Cycle encoding probes targeting the mRNA sequence of LacZ and performed HiPR-Cycle on bacteria from each of the three culture conditions. As can be seen in FIG. 8, in the absence of IPTG, bacteria showed very little fluorescent signal above background. However, with increasing dosage of IPTG present in the culture media, bright spots consistent with signal amplification from HiPR-Cycle appeared within bacterial cells.

Encoding probes 58-70 (SEQ ID NO: 72-84), as shown in Table 5, and encoding probes 81-112, as shown in Table 7 below, were used in this example Amplifier probes 7-10 (SEQ ID NO: 129-132), as shown in Table 2, were used in this example. Readout probes 1-10 (SEQ ID NO: 25-34), as shown in Table 3, were used in this example.

TABLE 7
Encoding probes used in Example 6.

| SEQ ID | | |
|---|---|---|
| NO: | Probe Name | Sequence |
| 97 | Encoding Probe | CGTCAGGTGAGCATCTTACATCTATTCGGCGCTCCACAGTTCC |
| | 81 | GGATTT |
| 98 | Encoding Probe | CGTCAGGTGAGCATCTTACATTTGTGCTTACCTTGCGGGCCAA |
| | 82 | CATCCA |
| 99 | Encoding Probe | CGTCAGGTGAGCATCTTACATCGGTCCAGTACCGCGCGGCTGA |
| | 83 | AATCAT |
| 100 | Encoding Probe | CGTCAGGTGAGCATCTTACATCGCTCGTGATTAGCGCCGTGGC |
| | 84 | CTGATT |
| 101 | Encoding Probe | CGTCAGGTGAGCATCTTACATGCGACAGCGTGTACCACAGCGG |
| | 85 | ATGGTT |
| 102 | Encoding Probe | CGTCAGGTGAGCATCTTACATAGTTACAGAACTGGCGATCGTT |
| | 86 | CGGCGT |
| 103 | Encoding Probe | CGTCAGGTGAGCATCTTACATTAACATTGGCACCATGCCGTGG |
| | 87 | GTTTCA |
| 104 | Encoding Probe | CGTCAGGTGAGCATCTTACATCGGTCTTCGCTATTACGCCAGC |
| | 88 | TGGCGA |
| 105 | Encoding Probe | CGTCAGGTGAGCATCTTACATTAGACACTCGGGTGATTACGAT |
| | 89 | CGCGCT |
| 106 | Encoding Probe | CGTCAGGTGAGCATCTTACATAGGAGATAACTGCCGTCACTCC |
| | 90 | AGCGCA |
| 107 | Encoding Probe | CGTCAGGTGAGCATCTTACATAAATTTGATGGACCATTICGGC |
| | 91 | ACCGCC |
| 108 | Encoding Probe | CGTCAGGTGAGCATCTTACATCTAGTGCCGCAAGGCGATTAAG |
| | 92 | TTGGGT |
| 109 | Encoding Probe | CGTCAGGTGAGCATCTTACATCAGTGACAATGGCAGATCCCAG |
| | 93 | CGGTCA |
| 110 | Encoding Probe | CGTCAGGTGAGCATCTTACATTTTATGCCGCTCATCCGCCACA |
| | 94 | TATCCT |
| 111 | Encoding Probe | CGTCAGGTGAGCATCTTACATAGGAGTGCCACCATCCAGTGCA |
| | 95 | GGAACT |
| 112 | Encoding Probe | CGTCAGGTGAGCATCTTACATTGATCGATGGTTCGCCCGGATA |
| | 96 | AACGGA |
| 113 | Encoding Probe | CGTCAGGTGAGCATCTTACATCCGGGTGTGCAGTTCAACCACT |
| | 97 | GCACGA |
| 114 | Encoding Probe | CGTCAGGTGAGCATCTTACATTGGATCTCACCGTGCCCATCAA |
| | 98 | TCCGGT |
| 115 | Encoding Probe | CGTCAGGTGAGCATCTTACATTTAGCGCTCAGGTCAAATTCAG |
| | 99 | ACGGCA |
| 116 | Encoding Probe | CGTCAGGTGAGCATCTTACATCAGAGGAAGATCGCACTCCAGC |
| | 100 | CAGCTT |
| 117 | Encoding Probe | CGTCAGGTGAGCATCTTACATCCGTGATGTTGAACTGGAAGTC |
| | 101 | GCCGCG |
| 118 | Encoding Probe | CGTCAGGTGAGCATCTTACATCCGGCATCGTAACCGTGCATCT |
| | 102 | GCCAGT |
| 119 | Encoding Probe | CGTCAGGTGAGCATCTTACATAGCCGCAGCTCGCCGTACATCT |
| | 103 | GAACTT |
| 120 | Encoding Probe | CGTCAGGTGAGCATCTTACATCCCCCATAATTCAATTCGCGCG |
| | 104 | TCCCGC |
| 121 | Encoding Probe | CGTCAGGTGAGCATCTTACATGCATGCACCACAGATGAAACGC |
| | 105 | CGAGTT |
| 122 | Encoding Probe | CGTCAGGTGAGCATCTTACATTTAAATTCGCGTCTGGCCTTCCT |
| | 106 | GTAGC |
| 123 | Encoding Probe | CGTCAGGTGAGCATCTTACATGAAGGAAATCGCTGATTTGCGT |
| | 107 | GGTCGG |
| 124 | Encoding Probe | CGTCAGGTGAGCATCTTACATTGAACGCGTACCGTTAGCCAGA |
| | 108 | GTTGTC |
| 125 | Encoding Probe | CGTCAGGTGAGCATCTTACATCGGTTCATACTGTACCGGGCGG |
| | 109 | GAAGGA |
| 126 | Encoding Probe | CGTCAGGTGAGCATCTTACATGAGAGCTGGAATTCCGCCGATA |
| | 110 | CTGACG |
| 127 | Encoding Probe | CGTCAGGTGAGCATCTTACATCGCACTGATCCACCCAGTCCCA |
| | 111 | GACGAA |
| 128 | Encoding Probe | CGTCAGGTGAGCATCTTACATTAGTGTGAAAGAAAGCCTGACT |
| | 112 | GGCGGT |

### Example 7. Detecting Stress Response Genes in Bacterial Cells

Another important application for HiPR-Cycle is to observe how changes in environmental conditions change bacterial gene expression, including how the bacteria respond to stresses.

To test the ability of HiPR-Cycle to detect stress response in bacteria, we created a stress response panel (all genes encoded with same initiator) to detect bacterial stress response to heat. The panel included the following genes: ibpA, ipbB, hslJ, hslR, hspQ, yedK, rpoS, recA, and rssB (each with 7 to 12 probes per gene), each pooled at equimolar proportion.

To generate a stress response, E. coli (ATCC 25922) were cultured in tryptic soy broth. Prior to reaching the logarithmic phase of growth E. coli were moved from a 37° C. incubator to a 53° C. water bath for a prescribed amount of time (15 to 60 minutes); a negative control remained at 37° C. for the entirety of the experiment. At the conclusion of the exposure, the bacteria were immediately fixed in 2% formaldehyde.

To detect stress response and bacterial taxonomy, 400 nM of each encoding probe in the stress response panel (corresponding to readout probe 9) and 400 nM of a 16S/23S rRNA panel (corresponding to readout probe 2) were used, respectively. The results of the experiment are shown in FIGS. 9A-9C. The rRNA signal (gather from excitation with 561 nm laser) was used to segment bacteria and determine cellular boundaries. The stress response signal for each bacterium was then measured as the mean intensity of 423 nm emission from 405 nm excitation within each cell. FIG. 9 shows an example field of view after processing, which shows a noticeably heightened signal from the stress response genes in some cells after heat shock, but not in cells that were not shocked. A comparison of mean intensity distributions showed that, on average, the mean intensity increases by two- to three-fold for the sample receiving a heat shock.

Encoding probes 46-57 (SEQ ID NO: 60-71), as shown in Table 5, and encoding probes 113-221, as shown in Table 8 below, were used in this example Amplifier probes 1-2 and 11-14 (SEQ ID NO: 21-22 and 242-245), as shown in Table 2, were used in this example. Readout probes 1-10 (SEQ ID NO: 25-34), as shown in Table 3, were used in this example.

TABLE 8
Encoding probes used in Example 7.

| SEQ ID | | |
|---|---|---|
| NO: | Probe Name | Sequence |
| 133 | Encoding Probe | GCTCGACGTTCCTTTGCAACAGAAAGCGCAACAAGCGCGGC |
| | 113 | TACTTT |
| 134 | Encoding Probe | GCTCGACGTTCCTTTGCAACACAGTGTTTCGCGGTCGCCAG |
| | 114 | CGTTAA |
| 135 | Encoding Probe | GCTCGACGTTCCTTTGCAACAGATAAGCGGTTACACATGCT |
| | 115 | GCCGGA |
| 136 | Encoding Probe | GCTCGACGTTCCTTTGCAACACCATCGCGGTCAGATCCACT |
| | 116 | TGTGCA |
| 137 | Encoding Probe | GCTCGACGTTCCTTTGCAACAGACGTCACGGGCTTACCGTT |
| | 117 | TACGCT |
| 138 | Encoding Probe | GCTCGACGTTCCTTTGCAACAAATGCGCACATCATACGGGT |
| | 118 | CATTGC |
| 139 | Encoding Probe | GCTCGACGTTCCTTTGCAACACGAGTAGCTGTTCTGGCGTT |
| | 119 | ACAGCA |
| 140 | Encoding Probe | GCTCGACGTTCCTTTGCAACAAGATCATACAGCAAGGCTGC |
| | 120 | CTCGCT |
| 141 | Encoding Probe | GCTCGACGTTCCTTTGCAACAGCATCGTCATTTCCCTGGCG |
| | 121 | CAGAGT |
| 142 | Encoding Probe | GCTCGACGTTCCTTTGCAACACCCGCGACGCTGTTCAGTAA |
| | 122 | TCGCCT |
| 143 | Encoding Probe | GCTCGACGTTCCTTTGCAACAATTAAACGGGCAGCCCATAG |
| | 123 | CCATTT |
| 144 | Encoding Probe | GCTCGACGTTCCTTTGCAACAAATCCGCCTTCAATCATTTC |
| | 124 | ACGGGC |
| 145 | Encoding Probe | GCTCGACGTTCCTTTGCAACAACATTAAATCGTAACAGGTC |
| | 125 | GCGGCG |
| 146 | Encoding Probe | GCTCGACGTTCCTTTGCAACACTCTTGTTTGCGGATGGTCT |
| | 126 | GCGCCA |
| 147 | Encoding Probe | GCTCGACGTTCCTTTGCAACACGCAAGCTCGTCATTCACCG |
| | 127 | CCAGCT |
| 148 | Encoding Probe | GCTCGACGTTCCTTTGCAACATGGTAACAGGGAATGGCGGA |
| | 128 | CCTGCT |
| 149 | Encoding Probe | GCTCGACGTTCCTTTGCAACATATAACCGGGTCGATATCCA |
| | 129 | CGACCA |
| 150 | Encoding Probe | GCTCGACGTTCCTTTGCAACATTATCGCTACTTAGCTGGGC |
| | 130 | TTCAGC |
| 151 | Encoding Probe | GCTCGACGTTCCTTTGCAACAGGACCATCACCACGTGATAC |
| | 131 | CACGGA |
| 152 | Encoding Probe | GCTCGACGTTCCTTTGCAACACCAAGGTATGAACCGGTAGG |
| | 132 | CCGTTA |
| 153 | Encoding Probe | GCTCGACGTTCCTTTGCAACAGTACCATGGATGGCTGTTCA |
| | 133 | GGATGT |
| 154 | Encoding Probe | GCTCGACGTTCCTTTGCAACATTGCGATCCATTGACGCATC |
| | 134 | AGTGGG |
| 155 | Encoding Probe | GCTCGACGTTCCTTTGCAACAACTTTCATAAGCCCTTGATG |
| | 135 | CAGCCA |
| 156 | Encoding Probe | GCTCGACGTTCCTTTGCAACATGGAGCCACAGCGATAGCAA |
| | 136 | TGCGGT |
| 157 | Encoding Probe | GCTCGACGTTCCTTTGCAACATTCTTGCGTTCAGCGATGCC |
| | 137 | CTGGTA |
| 158 | Encoding Probe | GCTCGACGTTCCTTTGCAACATGGACCAGCAGATTATCCTG |
| | 138 | GGCGGT |
| 159 | Encoding Probe | GCTCGACGTTCCTTTGCAACAAAGCGGAATCACGCGTTCGA |
| | 139 | GATCGA |
| 160 | Encoding Probe | GCTCGACGTTCCTTTGCAACAAATTACGGAGGGTAGCCGCC |
| | 140 | ATTACT |
| 161 | Encoding Probe | GCTCGACGTTCCTTTGCAACATCTCCATTCACCAGGTTAGC |
| | 141 | ACCACG |
| 162 | Encoding Probe | GCTCGACGTTCCTTTGCAACATTTCGGTCAAATCCAATAGC |
| | 142 | AGAACG |
| 163 | Encoding Probe | GCTCGACGTTCCTTTGCAACAAGATTTGGCTGCTCTGGCGT |
| | 143 | GCCTTT |
| 164 | Encoding Probe | GCTCGACGTTCCTTTGCAACAGACATAGCGATACGCTGCGC |
| | 144 | TGCGAT |
| 165 | Encoding Probe | GCTCGACGTTCCTTTGCAACAATTACCGTTTACGAAGGTTG |
| | 145 | CGCCAG |
| 166 | Encoding Probe | GCTCGACGTTCCTTTGCAACACGGAGCGCAAGGGTAATGCG |
| | 146 | GTAGTG |
| 167 | Encoding Probe | GCTCGACGTTCCTTTGCAACAAGTATGTTGTACGGCGGGAA |
| | 147 | GCTCTG |
| 168 | Encoding Probe | GCTCGACGTTCCTTTGCAACAAATAACATGCGACTGGTTGC |
| | 148 | GGCAGT |
| 169 | Encoding Probe | GCTCGACGTTCCTTTGCAACAGTGGCGTTTGGGATTGGGCA |
| | 149 | AAGCGT |
| 170 | Encoding Probe | GCTCGACGTTCCTTTGCAACATTAATCTTCCGCGAGAAACG |
| | 150 | CGAGGT |
| 171 | Encoding Probe | GCTCGACGTTCCTTTGCAACAAATATCAGCGGCGGTTTATC |
| | 151 | CCACCA |
| 172 | Encoding Probe | GCTCGACGTTCCTTTGCAACAACGTGGCACACAGCTTCTGG |
| | 152 | TAGCGA |
| 173 | Encoding Probe | GCTCGACGTTCCTTTGCAACACGGGCATCCATTCCCGCGCA |
| | 153 | GTTTCT |
| 174 | Encoding Probe | GCTCGACGTTCCTTTGCAACACGGCTGGTCTGCTGCTGCAG |
| | 154 | TGACAA |
| 175 | Encoding Probe | GCTCGACGTTCCTTTGCAACAATTAAACAGGTTGTCCATCA |
| | 155 | GCGCGA |
| 176 | Encoding Probe | GCTCGACGTTCCTTTGCAACAGTCCGGGCGGCGGTCATGAA |
| | 156 | TATCTA |
| 177 | Encoding Probe | GCTCGACGTTCCTTTGCAACATTGAGGTTGAATTAACTCCG |
| | 157 | CGCCCT |
| 178 | Encoding Probe | GCTCGACGTTCCTTTGCAACACAACGGGATCGTAGGGAATA |
| | 158 | TCGCGT |
| 179 | Encoding Probe | GCTCGACGTTCCTTTGCAACAGCGTCAAACGGTGTGCTACC |
| | 159 | TATCGC |
| 180 | Encoding Probe | GCTCGACGTTCCTTTGCAACAAAGAGCCAGGCAGTCGCATT |
| | 160 | CGCTTT |
| 181 | Encoding Probe | GCTCGACGTTCCTTTGCAACAAAGACCACTTTCACGCGGGT |
| | 161 | TTCGCT |
| 182 | Encoding Probe | GCTCGACGTTCCTTTGCAACATTAATCGACGCCCAGTTTAC |
| | 162 | GTGCGT |
| 183 | Encoding Probe | GCTCGACGTTCCTTTGCAACAGAGATCATACGTGCCGCAAG |
| | 163 | GCCCAT |
| 184 | Encoding Probe | GCTCGACGTTCCTTTGCAACATGGACGCGCCTGCTTTCTCG |
| | 164 | ATAAGC |
| 185 | Encoding Probe | GCTCGACGTTCCTTTGCAACACTACAGCAGCGTGTTGGACT |
| | 165 | GCTTCA |
| 186 | Encoding Probe | GCTCGACGTTCCTTTGCAACATCTAATCCGGCGTTGAGTTC |
| | 166 | GGGTTG |
| 187 | Encoding Probe | GCTCGACGTTCCTTTGCAACAGGGAGGTCAACCAGCTCGCC |
| | 167 | GTAGAA |
| 188 | Encoding Probe | GCTCGACGTTCCTTTGCAACACTAAACGTCTACTGCGCCAG |
| | 168 | AACGTG |
| 189 | Encoding Probe | GCTCGACGTTCCTTTGCAACAGGTGGCGCATGATGGAGCCT |
| | 169 | TTACCA |
| 190 | Encoding Probe | GCTCGACGTTCCTTTGCAACAACATTCTCAATCTGGCCCAG |
| | 170 | TGCTGC |
| 191 | Encoding Probe | GCTCGACGTTCCTTTGCAACAAAGTCGATCTCTTTCGCGGT |
| | 171 | TTCCGG |
| 192 | Encoding Probe | GCTCGACGTTCCTTTGCAACAGGTGCAAACCGAATCGACGT |
| | 172 | GCCAGT |
| 193 | Encoding Probe | GCTCGACGTTCCTTTGCAACAACATGAGAAGCGGAAACCAC |
| | 173 | GTTCCG |
| 194 | Encoding Probe | GCTCGACGTTCCTTTGCAACACGGCGTTCGATCGTCTGGCG |
| | 174 | AATCCA |
| 195 | Encoding Probe | GCTCGACGTTCCTTTGCAACAGGGTCTTCGATCAGGTCCAG |
| | 175 | CAACGC |
| 196 | Encoding Probe | GCTCGACGTTCCTTTGCAACAAGTAACTTCTCTACCGCGCG |
| | 176 | GATCAG |
| 197 | Encoding Probe | GCTCGACGTTCCTTTGCAACACACTGGCTCCCTGCGATAAC |
| | 177 | AGTTCC |
| 198 | Encoding Probe | GCTCGACGTTCCTTTGCAACACCCTGTCTACCGAGGTAATG |
| | 178 | CGCTCG |
| 199 | Encoding Probe | GCTCGACGTTCCTTTGCAACAAGATCTTCGGCCGTTAACAG |
| | 179 | TGGTGA |
| 200 | Encoding Probe | GCTCGACGTTCCTTTGCAACAGCTACAGCCATTTGACGATG |
| | 180 | CTCTGC |
| 201 | Encoding Probe | GCTCGACGTTCCTTTGCAACAGAAGCGTGGTATCTTCCGGA |
| | 181 | CCATTC |
| 202 | Encoding Probe | GCTCGACGTTCCTTTGCAACACTTTCGGCAAACGAATAGTA |
| | 182 | CGGGTT |
| 203 | Encoding Probe | GCTCGACGTTCCTTTGCAACAAAGGGCCAAATCGTTATCAC |
| | 183 | TGGGTT |
| 204 | Encoding Probe | GCTCGACGTTCCTTTGCAACAGTCACAACATCAAGCGCAGC |
| | 184 | CGACCA |
| 205 | Encoding Probe | GCTCGACGTTCCTTTGCAACATTAATCAAGCACCAGGCCGG |
| | 185 | GTTTGT |
| 206 | Encoding Probe | GCTCGACGTTCCTTTGCAACATTTACCACGAATCCAGAAGC |
| | 186 | GAGCGA |
| 207 | Encoding Probe | GCTCGACGTTCCTTTGCAACATCGCCGTTCATTCGTGGCAT |
| | 187 | CGCGAT |
| 208 | Encoding Probe | GCTCGACGTTCCTTTGCAACATCAACGCCATTATGTCCAGC |
| | 188 | TCGGGT |
| 209 | Encoding Probe | GCTCGACGTTCCTTTGCAACAAGTATAACGCGCCCAACTCT |
| | 189 | GGCAAC |
| 210 | Encoding Probe | GCTCGACGTTCCTTTGCAACATTTACCATCTCGCGCAAGCG |
| | 190 | ATTCAG |
| 211 | Encoding Probe | GCTCGACGTTCCTTTGCAACAATACAACCATTGCATCCCAG |
| | 191 | TCGCGA |
| 212 | Encoding Probe | GCTCGACGTTCCTTTGCAACAGCACGCATTCAGACCCGCAG |
| | 192 | AAACCA |
| 213 | Encoding Probe | GCTCGACGTTCCTTTGCAACAAGTACACCCAGACGTAACGC |
| | 193 | TTTGGC |
| 214 | Encoding Probe | GCTCGACGTTCCTTTGCAACATGGTGCTGAACCGGCGGTTG |
| | 194 | TAGTTC |
| 215 | Encoding Probe | GCTCGACGTTCCTTTGCAACAGACATTTGCACCTGATGTTC |
| | 195 | GCCGGT |
| 216 | Encoding Probe | GGGTGGTCGTCGAAGTCGTATAAGACGCGTTGGCGCGCAAA |
| | 196 | GCATTGAA |
| 217 | Encoding Probe | GGGTGGTCGTCGAAGTCGTATCGGTGCGCCTGTTGGCGAAT |
| | 197 | GTGCAACA |
| 218 | Encoding Probe | GGGTGGTCGTCGAAGTCGTATAAAAGCAAATGCGGTTTACC |
| | 198 | GTCGCGCA |
| 219 | Encoding Probe | GGGTGGTCGTCGAAGTCGTATTGATTGCGCAGGAACGTCGT |
| | 199 | TGATCGCA |
| 220 | Encoding Probe | GGGTGGTCGTCGAAGTCGTATGACATAAACGGCAGTGAGGC |
| | 200 | GCGGGTTT |
| 221 | Encoding Probe | GGGTGGTCGTCGAAGTCGTATTCTATCCCTTGCGCCTGGGT |
| | 201 | GTTTGCTT |
| 222 | Encoding Probe | GGGTGGTCGTCGAAGTCGTATAAATTTAATCAGCGGCGAAC |
| | 202 | GGGCGCTA |
| 223 | Encoding Probe | GGGTGGTCGTCGAAGTCGTATCGGTGCGTTTCATCGTCGTC |
| | 203 | TGCCGGAA |
| 224 | Encoding Probe | GGGTGGTCGTCGAAGTCGTATACGAACCGGAATGCCCGGCA |
| | 204 | ACTGTTCT |
| 225 | Encoding Probe | GGGTGGTCGTCGAAGTCGTATCGATCACGATCGAGACGCGA |
| | 205 | ACGCCATT |
| 226 | Encoding Probe | GGGTGGTCGTCGAAGTCGTATTTAACCACATCCTTAAACCC |
| | 206 | GGCACGCT |
| 227 | Encoding Probe | GGGTGGTCGTCGAAGTCGTATAAGACGGGCATCGCGTACCA |
| | 207 | GATCGTTA |
| 228 | Encoding Probe | GGGTGGTCGTCGAAGTCGTATCGAACGCACCAGGCGATAGC |
| | 208 | TTAAACGC |
| 229 | Encoding Probe | GGGTGGTCGTCGAAGTCGTATTACAACAGTCAGTCGTACCA |
| | 209 | CCGCCGAT |
| 230 | Encoding Probe | GGGTGGTCGTCGAAGTCGTATCCCCGCTTTCCAGCCCTTGC |
| | 210 | TGGCTAAT |
| 231 | Encoding Probe | GGGTGGTCGTCGAAGTCGTATAGCCGGTTATAACGAATCGC |
| | 211 | CCGACGCA |
| 232 | Encoding Probe | GGGTGGTCGTCGAAGTCGTATGTCATCCTCAAACAGCGCTA |
| | 212 | CCTGCTGC |
| 233 | Encoding Probe | GGGTGGTCGTCGAAGTCGTATAGGTGCAAGGTGGCTTCGTA |
| | 213 | ATCCAGCC |
| 234 | Encoding Probe | GGGTGGTCGTCGAAGTCGTATGGTGTGAGGAAAGACCGAAC |
| | 214 | TGCACGCT |
| 235 | Encoding Probe | GGGTGGTCGTCGAAGTCGTATGTGCCATGCCCAGTAACGGC |
| | 215 | ATCAGGTT |
| 236 | Encoding Probe | GGGTGGTCGTCGAAGTCGTATAGCGGCGACCAATCACCGCC |
| | 216 | TGAGTAAT |
| 237 | Encoding Probe | GGGTGGTCGTCGAAGTCGTATTACATGGCGGTACAGCCATT |
| | 217 | CGCTTACA |
| 238 | Encoding Probe | GGGTGGTCGTCGAAGTCGTATGTTTACGGCAACCACTATGA |
| | 218 | CCCAGCAG |
| 239 | Encoding Probe | GGGTGGTCGTCGAAGTCGTATTGGGCCACTGAACAGTTTGC |
| | 219 | TGTACCGT |
| 240 | Encoding Probe | GGGTGGTCGTCGAAGTCGTATTGCCCAGACTTTCTGTAACA |
| | 220 | GGGCCACT |
| 241 | Encoding Probe | GGGTGGTCGTCGAAGTCGTATTGCCCAGTGCGATATCCAGA |
| | 221 | TCGTTACC |

### Example 8. Split Initiator in HiPR-Cycle

A concern in using HiPR-Cycle to detect transcripts may be false initialization of amplifier chains from, for example, encoding probes that are incorrectly hybridized to targets, endogenous sequences with homology to initiators, or amplifiers that move out of the hairpin state to the “unraveled” state and initiate a reaction.

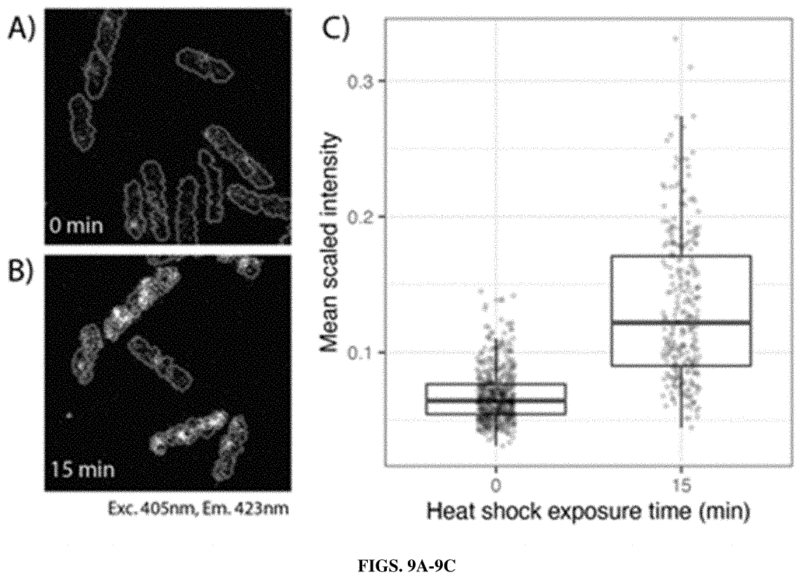

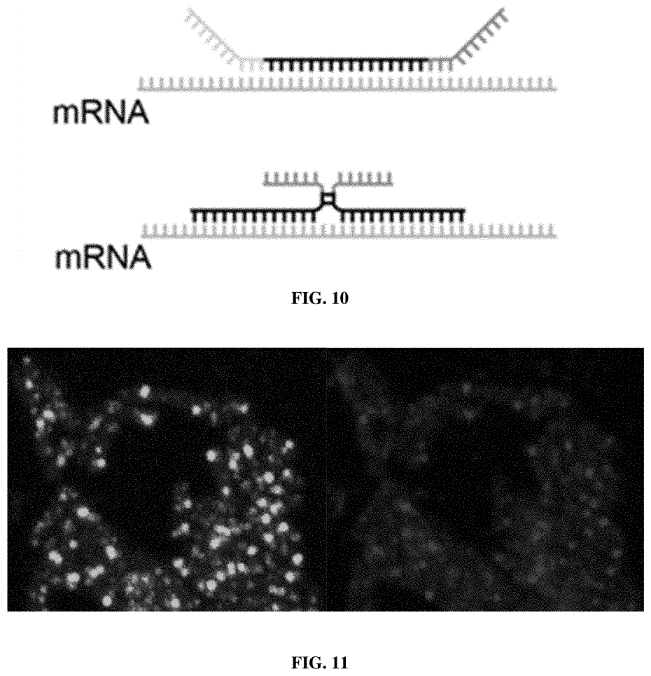

If the aforementioned problems are present, an option to solve this is using split initiators as primary probes. The probes split the initiator sequence into two separate probes with neighboring encoding regions. In order for initiation of the amplifiers to occur, both encoding probes forming the initiating complex must be bound as neighbors (see FIG. 10 for a schematic representation). These encoding probes can be made from two separate probes that have neighboring target regions. Together, these probes can create a continuous initiator sequence. HiPR-Cycle could employ a similar method to create encoding probes to remove background fluorescence from unintentional initiation. Probes would be added at the same concentration 10 nM to 2 μM. There would be two encoding probes per target with a single initiator. The amplifier design and concept as described above would not change.

### Example 9. Multiple Rounds of HiPR-Cycle

Another key application of HiPR-Cycle is the ability to perform measurements at high multiplexity with barcoding and spectral readouts. This allows us to theoretically detect 2^{d}−1 targets where d is the number of dyes used in an assay. The targets can be given barcodes (based on the encoding sequences) of d bits.

The addition of more rounds allows the barcode to be extended. For R rounds, the target multiplexity becomes (2^{d}−1)^{R} and allows for dR-bit barcodes.

For example, for a 10-bit system (using 10 dyes), one such code may be 0010011101. In a second round using the 10-bit system, the same code could be extended with additional bits, e.g. 1101011110. Thus, the full 2*10-bit code would include 20 bit, and in this example would be 00100111011101011110. In a 10-bit system with two rounds of HiPR-Cycle, we could use 20-bit barcodes and achieve 1,046,529 targets.

There are several ways that multiple rounds can be achieved.

One method includes repeating HiPR-Cycle, in its entirety, twice (or more) using two (or more) different sets of encoding probes, which could be accomplished as follows:
a. The encoding probes are encoded with between 1 and 10 initiators from a selection of 10 initiators. Each initiator corresponds to a unique set of amplifiers which all together hybridize a set of up 10 fluorescently-bound readout oligos.
b. HiPR-Cycle is performed (including imaging).
c. Encoding probes and readout probes are physically removed from the cells using a high-formamide stripping buffer (see below).
d. Another round of HiPR-Cycle is performed in its entirety.

Another method includes repeating HiPR-Cycle amplification/readout twice (or more) using two different sets of encoding probes. which could be accomplished as follows:
a. The encoding probes are encoded with between 1 and 20 initiators from a selection of 20 initiators. Each initiator corresponds to a unique set of amplifiers which all together hybridize a set of up 10 fluorescently-bound readout probes.
b. Here, only amplifiers/readout probes corresponding to a unique color in each round are used.
   i. For example, let's take a case where we only have 3 readout probes (red, blue, green [RGB]), but want to use 2 rounds of imaging. We could have codes such as 110+010 (RGB for each round where + signifies round break). Here only the first three digits can be read in a single round, and the second three can be read in a single round.
c. To perform this quickly, we can use the gel embedding strategy and bind the encoding probes into the gel substrate so that they are fixed in place (using label-IT) after they hybridize to gene targets.
d. We then perform the amplification/readout.
e. We then strip the probes using a high-formamide stripping buffer. This removes the amplifier and readout probes but does not remove the encoding probes.
f. A second amplifier/readout is performed with a unique set of amplifier probes.

Another method includes bleaching readout probes, which could be accomplished as follows:
a. Here encoding probes can contain many initiators, and many corresponding amplifier and readout probes. The main difference between other methods is that two readout probes may have the same fluorophore but different sequences.
b. We perform HiPR-Cycle and amplify all of the targets. Readout probes are collected into sets, each set has oligos with unique fluorescent dyes.
c. We add readout probes, mount samples and image according to our standard procedure.
d. A bleaching buffer (2×SSC, 2 mM VRC) is then placed on the specimen and high intensity/exposure laser (e.g. 647 nm at 100% intensity for 1 sec) is used to bleach probes.
e. The bleaching buffer is removed, specimen is washed, and the next set of readout probes is added to the buffer.

Procedure for Stripping Probes

The entire HiPR-Cycle procedure (fixation, digestion, encoding, amplification+readout, imaging) can be performed. Then, probes can be stripped (performed on 37° C. heat block with parafilm covering it) as follows: Cover glass is gently removed. 2×SSC is added to the samples and aspirated to remove imaging buffer. Stripping buffer (60% formamide) is added to the samples. Samples are incubated for 20 minutes. Stripping buffer is aspirated. 1×PBS is added, incubate for 15 minutes. Aspirate 1×PBS and replace with fresh 1×PBS for 15 minutes. Again, aspirate 1×PBS and replace with fresh 1×PBS for 15 minutes. Aspirate 1×PBS and add 2×SSC. Two optional steps can be (A) image samples again to ensure the signal is gone and (B) repeat amplification+readout steps to ensure the encoding probes are gone. Then, HiPR-Cycle is repeated.

### Example 9.1 HiPR-Cycle Amplification can be Expanded by Performing Multiple Rounds of Amplification

In this experiment, we showed that additional rounds of amplification can be performed to generate brighter signals.

Method

We cultured E. coli in the presence of cAMP and IPTG to generate high LacZ expression. The cell suspensions were fixed with 2% formaldehyde (90 minutes at room temperature), washed, and stored in 50% ethanol at −20° C.

For the experiment, cell suspensions were deposited on glass slides and treated with lysozyme (10 mg/mL) for 30 minutes at 37° C. to digest the cell wall. The slide was then washed with PBS (15 min., room temperature) and a pre-encoding buffer was added for 30 minutes (37° C.). A hybridization buffer with LacZ encoding probes (200 nM) and Eubacterium probes (1 μM) was added to the slides and incubated for 16 hours at 37° C. Following the encoding probe hybridization, cells were washed (wash buffer, 48° C. for 15 minutes; 5×SSCT, room temperature for 5 minutes) and a pre-amplification buffer was added for 30 minutes at room temperature.

Amplifier probes and corresponding readout probes were added to the slide for 5 hours at 30° C. The original amplification buffer was removed and a new amplification buffer with new amplifiers and readout probes that could expand off of the old product was added to the samples for 16 hours at 30° C. At the conclusion of amplification, the slides were washed in 2×SSC+Tween 20 at 42° C. for 15 minutes and mounted in ProLong Antifade.

Slides were imaged using a Zeiss i880 confocal in lambda mode with lasers set for 405 nm, 488 nm, 514 nm, 561 nm, and 633 nm excitation modes.

Results

Amplification for two separate rounds was detected by different colors, as shown in FIG. 11. The signal detected from Alexa-488 dye corresponded to round 1 while the signal detected from Alexa-546 dye corresponded to round 2.

Encoding probes 222-254, as shown in Table 9 below, were used in this example. Amplifier probes 15-18 (SEQ ID NO: 279-282), as shown in Table 2, were used in this example. Readout probes 1 and 9-10 (SEQ ID NO: 25 and 33-34), as shown in Table 3, were used in this example.

TABLE 9
Encoding probes used in Example 9.1.

| SEQ ID | | |
|---|---|---|
| NO: | Probe Name | Sequence |
| 246 | Encoding Probe | TAGAGTTGATAGAGGGAGAAGCTGCCTCCCGTAGGAGT |
| | 222 | |
| 247 | Encoding Probe | CGTCGGAGTGGGTTCAGTCTAGCCAAACCAGGCAAAGCGCCAT |
| | 223 | TCGCCA |
| 248 | Encoding Probe | CGTCGGAGTGGGTTCAGTCTAATTTGCGAACAGCGCACGGCGT |
| | 224 | TAAAGT |
| 249 | Encoding Probe | CGTCGGAGTGGGTTCAGTCTAGCCTTAACGCCGCGAATCAGCA |
| | 225 | ACGGCT |
| 250 | Encoding Probe | CGTCGGAGTGGGTTCAGTCTAGCTTAATTTCACCGCCGAAAGG |
| | 226 | CGCGGT |
| 251 | Encoding Probe | CGTCGGAGTGGGTTCAGTCTACATTCGCTTGCCACCGCAACAT |
| | 227 | CCACAT |
| 252 | Encoding Probe | CGTCGGAGTGGGTTCAGTCTAGGGTGCCACAAAGAAACCGTCA |
| | 228 | CCCGCA |
| 253 | Encoding Probe | CGTCGGAGTGGGTTCAGTCTACGCTGCAGCAGATGGCGATGGC |
| | 229 | TGGTTT |
| 254 | Encoding Probe | CGTCGGAGTGGGTTCAGTCTAACGAACAACGCCGCTTCGGCCT |
| | 230 | GGTAAT |
| 255 | Encoding Probe | CGTCGGAGTGGGTTCAGTCTACGATCTGACCATGCGGTCGCGT |
| | 231 | TTGGTT |
| 256 | Encoding Probe | CGTCGGAGTGGGTTCAGTCTAGCCAAACCGACGTCGCAGGCTT |
| | 232 | CTGCTT |
| 257 | Encoding Probe | CGTCGGAGTGGGTTCAGTCTACAAACCCATCGCGTGGGCATAT |
| | 233 | TCGCAA |
| 258 | Encoding Probe | CGTCGGAGTGGGTTCAGTCTACAGAATGCGGGTCGCTTCACTT |
| | 234 | ACGCCA |
| 259 | Encoding Probe | CGTCGGAGTGGGTTCAGTCTAAACTAATCAGCACCGCGTCGGC |
| | 235 | AAGTGT |
| 260 | Encoding Probe | CGTCGGAGTGGGTTCAGTCTACTATTCGGCGCTCCACAGTTCC |
| | 236 | GGATTT |
| 261 | Encoding Probe | CGTCGGAGTGGGTTCAGTCTATTGTGCTTACCTTGCGGGCCAA |
| | 237 | CATCCA |
| 262 | Encoding Probe | CGTCGGAGTGGGTTCAGTCTACGGTCCAGTACCGCGCGGCTGA |
| | 238 | AATCAT |
| 263 | Encoding Probe | CGTCGGAGTGGGTTCAGTCTACGCTCGTGATTAGCGCCGTGGC |
| | 239 | CTGATT |
| 264 | Encoding Probe | CGTCGGAGTGGGTTCAGTCTAGCGACAGCGTGTACCACAGCGG |
| | 240 | ATGGTT |
| 265 | Encoding Probe | CGTCGGAGTGGGTTCAGTCTAAGTTACAGAACTGGCGATCGTT |
| | 241 | CGGCGT |
| 266 | Encoding Probe | CGTCGGAGTGGGTTCAGTCTATAACATTGGCACCATGCCGTGG |
| | 242 | GTTTCA |
| 267 | Encoding Probe | CGTCGGAGTGGGTTCAGTCTACGGTCTTCGCTATTACGCCAGC |
| | 243 | TGGCGA |
| 268 | Encoding Probe | CGTCGGAGTGGGTTCAGTCTATAGACACTCGGGTGATTACGAT |
| | 244 | CGCGCT |
| 269 | Encoding Probe | CGTCGGAGTGGGTTCAGTCTAAGGAGATAACTGCCGTCACTCC |
| | 245 | AGCGCA |
| 270 | Encoding Probe | CGTCGGAGTGGGTTCAGTCTAAAATTTGATGGACCATTTCGGC |
| | 246 | ACCGCC |
| 271 | Encoding Probe | CGTCGGAGTGGGTTCAGTCTACTAGTGCCGCAAGGCGATTAAG |
| | 247 | TTGGGT |
| 272 | Encoding Probe | CGTCGGAGTGGGTTCAGTCTACAGTGACAATGGCAGATCCCAG |
| | 248 | CGGTCA |
| 273 | Encoding Probe | CGTCGGAGTGGGTTCAGTCTATTTATGCCGCTCATCCGCCACA |
| | 249 | TATCCT |
| 274 | Encoding Probe | CGTCGGAGTGGGTTCAGTCTAAGGAGTGCCACCATCCAGTGCA |
| | 250 | GGAACT |
| 275 | Encoding Probe | CGTCGGAGTGGGTTCAGTCTATGATCGATGGTTCGCCCGGATA |
| | 251 | AACGGA |
| 276 | Encoding Probe | CGTCGGAGTGGGTTCAGTCTACCGGGTGTGCAGTTCAACCACT |
| | 252 | GCACGA |
| 277 | Encoding Probe | CGTCGGAGTGGGTTCAGTCTATGGATCTCACCGTGCCCATCAA |
| | 253 | TCCGGT |
| 278 | Encoding Probe | CGTCGGAGTGGGTTCAGTCTATTAGCGCTCAGGTCAAATTCAG |
| | 254 | ACGGCA |

### Example 10. Double Amplifier in HiPR-Cycle

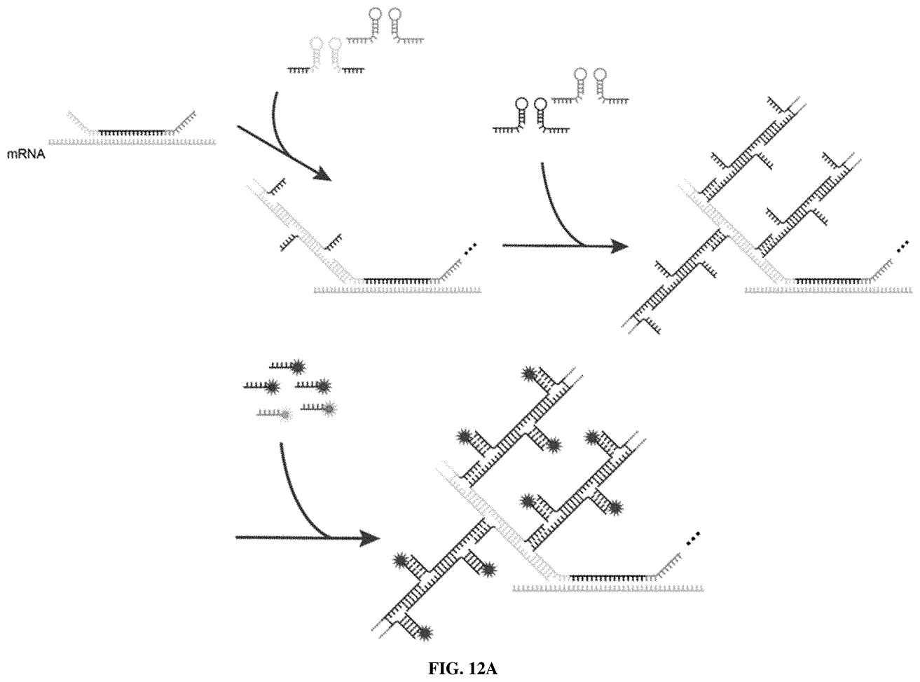

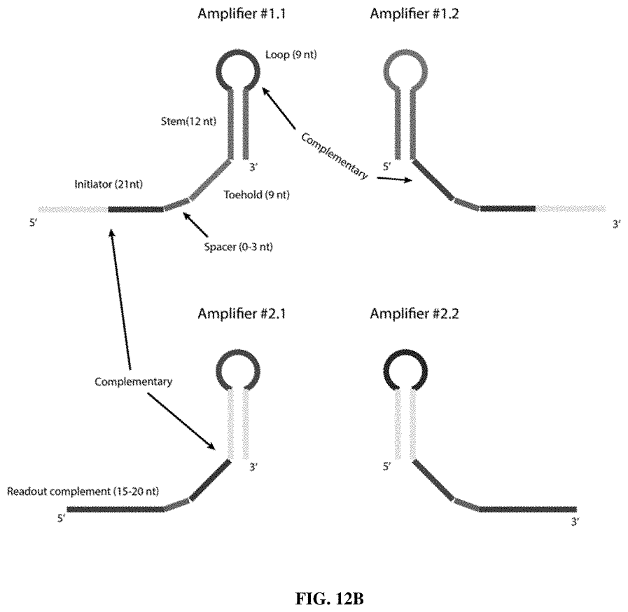

Another key application of HiPR-Cycle is the ability to perform measurements at high multiplexity with barcoding and spectral readouts, which allows us to theoretically detect 2^{d}−1 targets where d is the number of dyes used in an assay. The targets can be given barcodes (based on the encoding sequences) of d bits. FIG. 12A shows a schematic of this.

HiPR-Cycle boosts signals for specific targets, if amplification is not sufficient, further rounds can be used to amplify off of amplifiers. For n rounds, this requires n amplifier pairs and if the signal from a single round of a single target is some coefficient S, then the theoretical signal amplification from n rounds is Sn.

For example, take the case of two rounds of amplification and a single target. An encoding probe will include initiator A₁, the first set of amplifiers H_{1,1} and H_{2,1} will be triggered by A₁. Importantly, rather than a readout sequence H_{1,1} will have an overhanging amplifier A₂. A₂ will trigger a second set of amplifiers H_{1,2} and H_{2,2} which contain readout landing pads. The process is shown above. An example of the design is shown in FIG. 12B. An exemplary experiment employing this method is described in Example 10.1

### Example 10.1 HiPR-Cycle Amplification can be Expanded by Performing Multiple Rounds of Branched Amplification

In this experiment, we show that additional rounds of amplification can be performed to generate brighter signals using an exponentially growing (branched) amplification strategy.

Method

We cultured E. coli in the presence of cAMP and IPTG to generate high LacZ expression. The cell suspensions were fixed with 2% formaldehyde (90 minutes at room temperature), washed, and stored in 50% ethanol at −20° C.

For the experiment, cell suspensions were deposited on glass slides and treated with lysozyme (10 mg/mL) for 30 minutes at 37° C. to digest the cell wall. The slide was then washed with PBS (15 min., room temperature) and a pre-encoding buffer was added for 30 minutes (37° C.). A hybridization buffer with LacZ encoding probes (200 nM) and Eubacterium probes (1 μM) was added to the slides and incubated for 16 hours at 37° C. Following the encoding probe hybridization, cells were washed (wash buffer, 48° C. for 15 minutes; 5×SSCT, room temperature for 5 minutes) and a pre-amplification buffer was added for 30 minutes at room temperature.

First-stage amplifier probes were added to the slide for 5 hours at 30° C. The original amplification buffer was removed and a new amplification buffer with second-stage amplifiers and readout probes that could expand off of the old product was added to the samples for 16 hours at 30° C. At the conclusion of amplification, the slides were washed in 2×SSC+Tween 20 at 42° C. for 15 minutes and mounted in ProLong Antifade. Slides were imaged using a Zeiss i880 confocal in lambda mode with lasers set for 405 nm, 488 nm, 514 nm, 561 nm, and 633 nm excitation modes.

Results

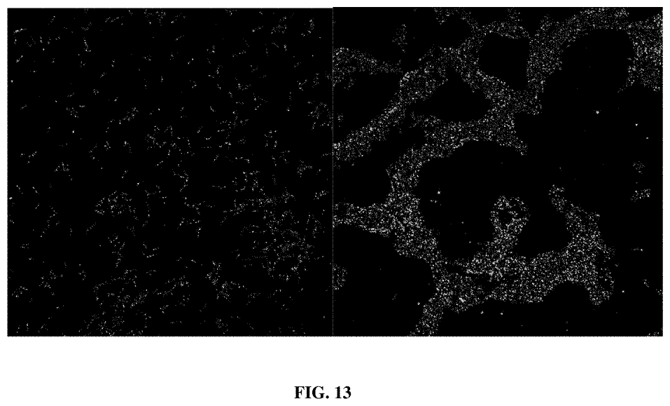

As shown in FIG. 13, the signal in samples with branched amplification was much brighter than those undergoing standard amplification.

Encoding probes 222-254 (SEQ ID NO: 246-278), as shown in Table 9, were used in this example. Amplifier probes 19-22 (SEQ ID NO: 283-286), as shown in Table 2, were used in this example. Readout probes 9-10 (SEQ ID NO: 33-34), as shown in Table 3, were used in this example.

### Example 11. Use of Gel Embedding, Clearing, and/or Physical Expansion of Specimen

Improvements to imaging resolution can be boosted, not only by increasing the magnification on a microscope, but also by physically expanding the observed specimen. This technique has been termed expansion microscopy and is compatible with single molecule FISH methods. For imaging applications in bacteria and other microorganisms, combining sample expansion approaches with RNA detection could enhance the ability to identify and quantify molecules, such as RNA, within cells by increasing the physical distance between them. Moreover, covalently embedding target molecules of a sample within a gel matrix makes it possible to “clear” (digest/remove) other biomolecules (proteins, lipids, etc.) which would otherwise contribute to light scattering and background autofluorescence.

To embed a sample within a gel in a way that preserves the RNA target locations, we can first chemically modify nucleic acids with LabelX (described below), which adds an acryloyl group to guanine nucleotides. This modification enables target RNA molecules to be incorporated into the polyacrylamide gel matrix. To embed the labeled samples within a gel matrix, we can perfuse (incubate) our preserved/fixed specimen with “monomer” solution (Stock X for expandable gels, or Stock Z for non-expandable gels, described below). We then can add initiator reagents to induce the polymerization of polyacrylamide and formation of a gel. Before expansion, proteins within the specimen can be digested to facilitate clearing of unwanted biomolecules from the specimen and enable isotropic expansion of the sample material. The entire matrix and embedded specimen is then expanded by adding water to the gel. Because small molecules, such DNA probes or amplifiers, can freely diffuse in and out of the gel, HiPR-Cycle can be directly performed on the sample to target RNA molecules directly integrated into the gel matrix before or after the expansion process.

Reagents

LabelX Solution

Label-IT amine is resuspended at 1 mg/mL using the commercial resuspension buffer and mixed. Resuspended Label-ITis then reacted with the AcX/DMSO stock solution at equal mass ratio (e.g. 10 μL of AcX/DMSO stock (at 10 mg/ml) added to 100 μL of Label-IT solution. The reaction is carried out overnight at RT with gentle agitation.

MelphaX

MelphaX can be used instead of LabelX solution to label DNA and RNA within cells for matrix integration. Melphalan (Cayman Chemicals, 16665) is dissolved in anhydrous DMSO (sigma) to 2.5 mg/ml in anhydrous DMSO (Sigma). Acryloyl-X, SE (Thermo Fisher, 20770) is dissolved in anhydrous DMSO to 10 mg/mL. To create MelphaX, Melphalan stock is combined Acryloyl-X 4:1 respectively, SE stock and incubated overnight at room temperature with shaking to make MelphaX (2 mg/ml). Aliquots can be stored at −20° C. in a desiccated environment. Working solution is prepared by diluting MelphaX stock to 1 mg/ml by MOPS buffer (20 mM, pH 7.7)

| | | | Final |
|---|---|---|---|
| | Stock solution | | concentration |
| Monomer solution | concentration (g/100 | Amount | (g/100 mL |
| (“Stock X”) | mL solution) | (mL) | solution) |
| Sodium acrylate | 38 (33 wt % due to | 2.25 | 8.6 |
| | higher density) | | |
| Acrylamide | 50 | 0.5 | 2.5 |
| N,N′- | 2 | 0.75 | 0.15 |
| Methylenebisacrylamide | | | |
| Sodium chloride | 29.2 (5M) | 4 | 11.7 |
| PBS 10X stock | 10X | 1 | 1X |
| Water | | 0.9 | |
| Total | | 9.4 | |

| Non-Expanding | | | |
|---|---|---|---|
| Monomer solution | Stock solution | Amount | Final |
| (“Stock Z”) | concentration | (mL) | concentration |
| Bis-Acrylamide | 2% | 2 | 0.05% |
| Acrylamide | 40% | 2 | 4% |
| Sodium chloride | 5M | 0.6 | 0.3M |
| Tris-HCl (pH 8) | 1M | 0.6 | 60 mM |
| Water | | 4.8 | |
| Total | | 10 | |

| MOPS stock solution | Stock solution concentration |
|---|---|
| (200 mM) | (g/100 mL solution) |
| MOPS | 4.18 |

| Gelling | Stock solution concentration | Amount | Final concentration |
|---|---|---|---|
| solution | (g/100 mL solution) | (μL) | (g/100 mL solution) |
| Stock X | NA | 188 | NA |
| TEMED | 10 | 4 | 0.2 |
| APS | 10 | 4 | 0.2 |
| Water | | 4 | |
| Total | | 200 | |

| | | |
|---|---|---|
| | | Final concentration |
| Digestion buffer | Amount | (/100 mL solution) |

| | | | | |
|---|---|---|---|---|
| Triton-X | 2.50 | g | 0.50 | g |
| EDTA | 0.146 | g | 0.027 | g |
| Tris (1M) aqueous | 25 | mL | 5 | mL |
| solution, pH 8 | | | | |
| NaCl | 23.38 | g | 4.67 | g |

| | | |
|---|---|---|
| Water | Add up to a total | |
| | volume of 500 mL | |
| Proteinase K | 1:100 dilution | 800 units |
| (800 units/mL) | | (=8 units/mL) |
| Total | 500 | |

HiPR-Cycle Reagents
probe hybridization buffer: 10% formamide (range: 5%-50%), 5× sodium chloride sodium citrate (SSC), 10% dextran sulfate, 0.1% Tween 20, 9 mM citric acid (pH 6.0; can be omitted), 50 μg/mL heparin (can be omitted), and 1×Denhardt's solution.
10% probe wash buffer: 10% formamide (range: 5%-50%), 5× sodium chloride sodium citrate (SSC), 9 mM citric acid (pH 6.0; can be omitted), 0.1% Tween 20, and 50 μg/mL heparin (can be omitted).
Amplification buffer: 5× sodium chloride sodium citrate (SSC), 0.1% Tween 20 (or other anionic detergent), and 10% dextran sulfate.
5×SSCT: 5× sodium chloride sodium citrate (SSC) and 0.1% Tween 20
50% dextran sulfate
10 mg/mL Lysozyme

Prepping Bacterial Cells for Embedding

Starting with 100 μL fixed frozen stocks of log-phase growth E. coli, pellet cells (10000×G for 5 mins), resuspend in 100 μL 10 mg/mL Lysozyme and incubate at 37° C. for 30 min to 12 hrs, wash cells 2× with 1×PBS, wash cells 1× with 20 mM MOPS pH 7.7, pellet cells, re-suspend in 100 μL LabelX or MelphaX Solution and incubate at 20-37° C. overnight.

Polymer Synthesis (Gelation):

Before gelation prepare gel casting chamber by placing two coverslides on glass slide with a square gap between them (note sides can remain exposed to air). Then, pellet and wash labeled cells 2× with 1×PBS, pellet and re-suspend cells in 50 μL monomer solution, incubate at RT for 1 minute before proceeding.

Initiate polymerization by adding APS and TEMED to 0.2% w/w final Immediately pipette solution into gel cast and add a coverslip to the top. Transfer specimen (in casting setup) to a humidified incubator set to 22-42° C. (e.g., 37° C.). Wait 1-2 hrs for gelation to occur.

As an alternative to performing preparation and polymer synthesis (gelation) of bacteria in solution, the above steps can be performed on (fixed) bacteria plated on a flat surface (e.g. glass slide). A benefit of this approach is that bacteria will be embedded within the gel in the same plane (closest to the glass surface). To perform steps on a glass slide, a silicone gasket with small chambers can be placed on the glass to contain bacteria and reaction volumes. Fixed bacteria are placed into a well and allowed to dry. Lysozyme treatment and subsequent washes are performed on the dried bacteria within the well. After washes, the bacteria are allowed to dry on the glass and the silicone gasket is removed. A gel casting chamber is then assembled as described, with the dried bacteria in the center. The gelation mix is prepared as described above, but in the absence of the bacteria/sample. Acting quickly, the gelation mix is pipetted onto the specimen located within the casting chamber and the sample is moved to a humidified incubator set to 22-42° C. (for example at 37° C.) and allowed to solidify for 1-2 hrs.

Non Expanding Gelation: To embed samples within non-expandable polyacrylamide gels, replace Stock X (described above) can be used instead of Stock Z (described above) during polymer synthesis steps.

Proteinase K Digestion

Dilute proteinase K (final concentration between 1 U/mL and 200 U/mL; optimal 8 U/mL) in digestion buffer, once gels have solidified, carefully remove coverslip lid and chamber walls, place microscope slide with gel on top into petri-dish containing enough volume (about 5 mL) of digestion buffer to cover gel, leave in digestion buffer for 3-24 hrs (e.g., 12 hrs) at 37° C. Note: After digestion, gels can be stored in 1×PBS, or other saline solutions (such as 5×SSC) until expansion.

HiPR-Cycle on Gelled Samples:

Gently transfer gel into 24-well glass bottom plate, or any other suitable container with a lid. Smaller containers reduce reagent requirements. Containers with glass bottoms are desirable because they enable direct imaging without the need to move gels.

Add encoding buffer (without probes) to samples within wells (30 min at 37° C.). Add 300 μL encoding buffer (with probes) to samples and incubate for 2 to 24 hours at 25-45° C. (for example 3 hrs at 37° C.). Wash gels 3× with excess volume of wash buffer (e.g., 500 μL for 24-well plates; 5 mins each at room temp). Wash with 5×SSCT (5 min at RT).

Start amplifier snap-cool procedure. Place each amplifier oligo (about 5 μL/sample) in its own tube of strip tube. Heat to 95° C. for 2 minutes on a PCR block. Remove and let cool at RT for 30 min. Pool snap-cooled amplifier. Then, add amplification buffer (without probes to sample). 30 min at RT.

Combined Amplification and Readout Probe Binding.

To gels within 24-well dish add the following Mixture:

| Reagent | 1X volume (μL) |
|---|---|
| Amp Buffer | 260 |
| Amplifier Pool | 20 |
| Readout Buffer w/probes; (RB10, R2 1:1) | 20 |

Place the sample in a covered box to allow for amplification. Allow amplification to proceed for 2-24 hrs at 20-40° C. (e.g., 12 hrs at 25° C.). Remove amp buffer. Wash 3× with 5×SSCT for 5 min at RT.

Expansion and Imaging

At this point the samples can be imaged (pre and/or post expansion). To expand the samples, wash with 0.05×SSCT for 10 minutes.

The expansion factor can be tuned by altering the salt concentration; imaging can also be performed in regular PBS for about 2× expansion.

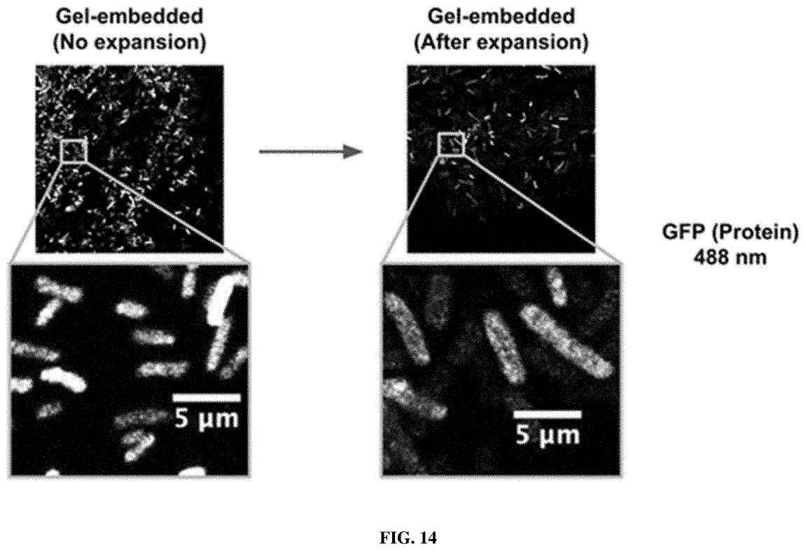

We examined whether E. coli cells expand uniformly when embedded in a swellable gel matrix (FIG. 14). Fixed, GFP expressing E. coli cells were embedded in either non-expanding (left), or swellable poly-acrylamide gels and GFP signal was imaged using a 488 nm excitation laser. After protease digestion for both gels, expansion of gel on the right was performed by washing the sodium-acrylate-containing gel in low salt solutions (0.05×SSC). The gel on the left does not contain sodium acrylate, and is thus non-swelling.

### Example 12. Multiple Rounds of HiPR-Cycle on Gel Embedded Specimen

As described in Example 9 and Example 9.1, multiple rounds of HiPR-Cycle can be performed directly on gel-embedded specimen. In the basic approach, both encoding and amplifier probes would be stripped out of the gel embedded specimen using high formamide washes solutions (described in Example 9). The fluorophore bleaching approach (also described in Example 9) could similarly be applied to gel-embedded specimen.

Alternatively, integrating nucleic acids directly into a gel matrix (either expandable or non-swelling) offers another approach for performing multiple rounds of HiPR-Cycle. Here encoding probe hybridization can be performed on the specimen prior to treatment with LabelX or MelphaX, which would chemically modify encoding probes directly, enabling their integration into the gel matrix with their locations relative to target genes preserved. In this scheme, because the encoding probes are covalently integrated within the gel matrix, they would be resistant to any stripping solutions applied to the specimen. Therefore, only one round of encoding hybridization would be needed, and multiple rounds of amplification and stripping could be used to sequentially create and image amplified signals.

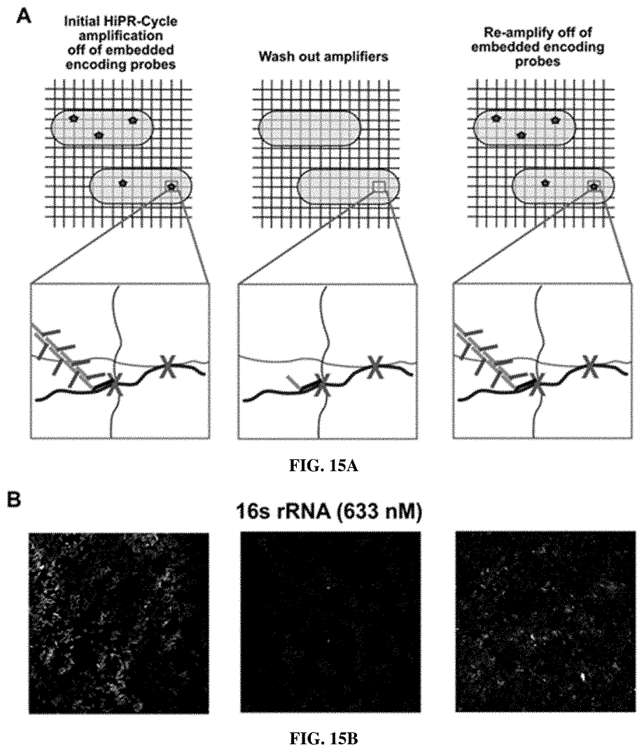

We have shown that rather than using a stripping buffer with a high concentration of formamide, a hypotonic solution (e.g. water) can be used to break down amplification products and remove HiPR-Cycle signal for a target gene. Finally, by re-performing the HiPR-Cycle amplification reaction we were able to restore the fluorescent signal (FIGS. 15A-15B).

Encoding probes 58-70 (SEQ ID NO: 72-84), as shown in Table 5, were used in this example. Amplifier probes 1-2 (SEQ ID NO: 21-22), as shown in Table 2, were used in this example. Readout probes 1-10 (SEQ ID NO: 25-34), as shown in Table 3, were used in this example.

### Example 13. Encoding Conditions in HiPR-Cycle Imaging Assay

This example shows that we are able to use the rRNA of bacteria as indicators of different conditions using different barcodes/colors. For example, and E. coli exposed two separate drugs can be encoded as 100 when exposed to drug 1 and 010 when exposed to drug 2. The two samples can then be mixed together in a single assay to examine gene expression changes from different conditions. This technique is valuable for screening purposes because it allows the user to highly multiplex the imaging and sample processing steps after encoding.

Methods

E. coli cells were separately grown and harvested under three separate heat stress conditions. Two samples were considered non-heat stressed and were grown at either 30° C. (Sample 1) or 37° C. (Sample 2) and harvested at mid-log growth phase (˜0D600=0.5). Sample 3 was grown at 30° C. until mid-log phase growth, at which point it was heated to 46° C. for 5 minutes to induce heat stress response and then collected. We separately performed HiPR-Cycle encoding probe hybridization on cells from each sample, targeting the same three transcript species within each sample: 16S/23S rRNA, atpD mRNA and clpB mRNA. In order to label cells by sample, we used 16s rRNA encoding probes with a distinct initiator to encode cells from each condition. Sample 1 (no HS 30° C.) cells were labeled so that they would fluoresce upon 488 nm excitation, Sample 2 (no HS 37° C.) with 633 nm, and Sample 3 (5′ HS 46° C.) with 405 nm. The same encoding probes targeting atpD (561 nm excitation) and clpB (514 nm excitation) mRNAs were used for all three samples. After encoding hybridization, cells from each sample were mixed at approximately equal proportions and the sample mixture was plated on glass slides. HiPR-Cycle amplification was performed on the mixture of cells followed by imaging. Consistent with sample-based rRNA encoding, E. coli (ATCC 25922) cells only exhibit fluorescence in one of the three possible channels, with no cells showing blended signals (FIG. 16B). The clpB gene transcript is known to only become expressed under heat stress conditions and we almost exclusively detect signal within the Sample 3 (blue) cells (FIG. 16C-16D, yellow arrows). By contrast, expression of atpD, which is a housekeeping gene, could be detected in both heat-stressed cells (Sample 3, blue) and non-stressed cells (Sample 1, green) (FIG. 16C-16D, magenta arrows).

Encoding probes 255-348, as shown in Table 10 below, were used in this example. Amplifier probes 1-2, 17-18 and 23-28 (SEQ ID NO: 21-22, 281-282 and 381-386), as shown in Table 2, were used in this example. Readout probes 1-10 (SEQ ID NO: 25-34), as shown in Table 3, were used in this example.

TABLE 10
Encoding probes used in Example 13.

| SEQ ID | | |
|---|---|---|
| NO: | Probe Name | Sequence |
| 287 | Encoding Probe | GCTGGTCGGTGGATTGGATTTCCATGCGTCAAGGTGCGCAA |
| | 255 | AGGTGGTT |
| 288 | Encoding Probe | GCTGGTCGGTGGATTGGATTTTCAACGTTCACCTACGCCCG |
| | 256 | CAAACACA |
| 289 | Encoding Probe | GCTGGTCGGTGGATTGGATTTCTTTTCGACGTCAACTACGG |
| | 257 | CGCCGATT |
| 290 | Encoding Probe | GCTGGTCGGTGGATTGGATTTTGCAGCACGCGCTACCACCA |
| | 258 | GTTTGTCT |
| 291 | Encoding Probe | GCTGGTCGGTGGATTGGATTTGGCAGCTGCTGCTGAACTTC |
| | 259 | CAGCACCA |
| 292 | Encoding Probe | GCTGGTCGGTGGATTGGATTTTGGTCAAGAGCATCGTACAC |
| | 260 | GCGCGGTA |
| 293 | Encoding Probe | GCTGGTCGGTGGATTGGATTTCTTTCGCCCAACGCTCTTCT |
| | 261 | TCACCGAT |
| 294 | Encoding Probe | GCTGGTCGGTGGATTGGATTTCGGACGCGCAGTGTCGTAGT |
| | 262 | GTTCCTGA |
| 295 | Encoding Probe | GCTGGTCGGTGGATTGGATTTCTAACGTTCCTGCAGAACGC |
| | 263 | CCATCTCT |
| 296 | Encoding Probe | GCTGGTCGGTGGATTGGATTTTGATTACCGCCCTTAGCGAA |
| | 264 | CGGACACA |
| 297 | Encoding Probe | GCTGGTCGGTGGATTGGATTTCATACCGGAGTGCTCGATCG |
| | 265 | CGATGTTA |
| 298 | Encoding Probe | GCTGGTCGGTGGATTGGATTTCCGATACGGCCCAGCAGTGC |
| | 266 | GGATACTT |
| 299 | Encoding Probe | GCTGGTCGGTGGATTGGATTTACGTCCGGCAGGTGATCGTA |
| | 267 | TTCGCCTT |
| 300 | Encoding Probe | GCTGGTCGGTGGATTGGATTTGGGATTGCGATGGTACGCAC |
| | 268 | GATACCGC |
| 301 | Encoding Probe | GCTGGTCGGTGGATTGGATTTCAATTACCTACACCCGCACC |
| | 269 | ACCGAACA |
| 302 | Encoding Probe | GCTGGTCGGTGGATTGGATTTAGTTGTCGACCGGTTCACCC |
| | 270 | AGTACGTT |
| 303 | Encoding Probe | GCTGGTCGGTGGATTGGATTTGCCAGACGGGTCAGTCAAGT |
| | 271 | CATCCGCA |
| 304 | Encoding Probe | GCTGGTCGGTGGATTGGATTTATTAGCCACGGATGGTGTCT |
| | 272 | TTCAGGGA |
| 305 | Encoding Probe | GCTGGTCGGTGGATTGGATTTAGATTTACATCCAGACCGCG |
| | 273 | ACGCAGAC |
| 306 | Encoding Probe | GCTGGTCGGTGGATTGGATTTGGGAGAGACGCGATCTGACG |
| | 274 | GCTCAGTA |
| 307 | Encoding Probe | GCTGGTCGGTGGATTGGATTTCTTTACTTCTGCCACGAAGA |
| | 275 | ACGGCTGG |
| 308 | Encoding Probe | GCTGGTCGGTGGATTGGATTTCCGGGCTCGTTCATCTGGCC |
| | 276 | ATACACCA |
| 309 | Encoding Probe | GCTGGTCGGTGGATTGGATTTTTAACGTCACGACCTTCGTC |
| | 277 | ACGGAATT |
| 310 | Encoding Probe | GCTGGTCGGTGGATTGGATTTTATACGTTGGAGTCGGTCAT |
| | 278 | TTCGTGGT |
| 311 | Encoding Probe | GCTGGTCGGTGGATTGGATTTAGGACAGCTTCTTCGATGGA |
| | 279 | ACCGACCA |
| 312 | Encoding Probe | GCTGGTCGGTGGATTGGATTTCATCCGGCCAGGGTGTAACG |
| | 280 | ATAGATGT |
| 313 | Encoding Probe | GCTGGTCGGTGGATTGGATTTACTTACGGCCCAGAGTCGCT |
| | 281 | TTACCTAC |
| 314 | Encoding Probe | GCTGGTCGGTGGATTGGATTTATGTGCCTGTACGGAGGTGA |
| | 282 | TAGAACCG |
| 315 | Encoding Probe | GCTGGTCGGTGGATTGGATTTCTGAGTTCATCCATACCCAG |
| | 283 | GATGGCGA |
| 316 | Encoding Probe | GCTGGTCGGTGGATTGGATTTTCATTGACAGCTCTTCGTAG |
| | 284 | GAAGGTGC |
| 317 | Encoding Probe | GCTGGTCGGTGGATTGGATTTTCATCCTGATAACGTTGCAG |
| | 285 | GATGGACT |
| 318 | Encoding Probe | GCTGGTCGGTGGATTGGATTTATTCTTTGATACCGGTTTCC |
| | 286 | AGCAGTTC |
| 319 | Encoding Probe | CGTCGGAGTGGGTTCAGTCTATCATCGCCAGCGCCTTACAA |
| | 287 | AGCTCT |
| 320 | Encoding Probe | CGTCGGAGTGGGTTCAGTCTACGGTTCGAGCTGCGTTGCGG |
| | 288 | CTTCCA |
| 321 | Encoding Probe | CGTCGGAGTGGGTTCAGTCTAAAGACCACGCGCCAGTGCAG |
| | 289 | GTTTCA |
| 322 | Encoding Probe | CGTCGGAGTGGGTTCAGTCTAGCACGCTGCTGCAACAATTG |
| | 290 | CCGGGT |
| 323 | Encoding Probe | CGTCGGAGTGGGTTCAGTCTAGTCTACGCGGCGGCCTTTCA |
| | 291 | ACCCTT |
| 324 | Encoding Probe | CGTCGGAGTGGGTTCAGTCTATTAACGCTGCGCCAGACCTT |
| | 292 | CAACGA |
| 325 | Encoding Probe | CGTCGGAGTGGGTTCAGTCTACAGAAGCGTCGTGGCACCTA |
| | 293 | CGCAGT |
| 326 | Encoding Probe | CGTCGGAGTGGGTTCAGTCTACAGACCCACACGGCGAGCCT |
| | 294 | GTTCAA |
| 327 | Encoding Probe | CGTCGGAGTGGGTTCAGTCTACGCACGACGCACCGCTTCGG |
| | 295 | TCAGAT |
| 328 | Encoding Probe | CGTCGGAGTGGGTTCAGTCTAAAAATCCGGCAGCTGACGGT |
| | 296 | CAGCAA |
| 329 | Encoding Probe | CGTCGGAGTGGGTTCAGTCTAAAATCGCGCTCGCTTTCCAT |
| | 297 | CATGCG |
| 330 | Encoding Probe | CGTCGGAGTGGGTTCAGTCTAGAATACCCGCGCCGACCATG |
| | 298 | GTATGT |
| 331 | Encoding Probe | CGTCGGAGTGGGTTCAGTCTACTATGGGCATCGGCAAGAGC |
| | 299 | AAGCTG |
| 332 | Encoding Probe | CGTCGGAGTGGGTTCAGTCTAACGTTTCAGGATGTCGGCCA |
| | 300 | GCGTGC |
| 333 | Encoding Probe | CGTCGGAGTGGGTTCAGTCTAAGCATACGGACCATCGCCTC |
| | 301 | GTCGCT |
| 334 | Encoding Probe | CGTCGGAGTGGGTTCAGTCTAGCATGCAGCACCTGAATGGT |
| | 302 | ACGGCG |
| 335 | Encoding Probe | CGTCGGAGTGGGTTCAGTCTATTATTGCACATGGTGGTGCA |
| | 303 | GCTCGT |
| 336 | Encoding Probe | CGTCGGAGTGGGTTCAGTCTACTTTTGGTTGTCGTGCCCGA |
| | 304 | GTGCAA |
| 337 | Encoding Probe | CGTCGGAGTGGGTTCAGTCTAAACAGTAATGTTGGCGGTGG |
| | 305 | TCGCCC |
| 338 | Encoding Probe | CGTCGGAGTGGGTTCAGTCTAGGTATCGGGCGATTTGGATC |
| | 306 | CGCCAG |
| 339 | Encoding Probe | CGTCGGAGTGGGTTCAGTCTATCATTGCCTTGTTCGGCTCG |
| | 307 | TTCGGT |
| 340 | Encoding Probe | CGTCGGAGTGGGTTCAGTCTATTGGCGCACCAGATCCTGTG |
| | 308 | ATGGCT |
| 341 | Encoding Probe | CGTCGGAGTGGGTTCAGTCTATACGCTTACCACACCGAGCA |
| | 309 | CCAGCT |
| 342 | Encoding Probe | CGTCGGAGTGGGTTCAGTCTACTACGTTCACGCTTTCACCT |
| | 310 | CCACGC |
| 343 | Encoding Probe | CGTCGGAGTGGGTTCAGTCTAGTGCCGTTGGGCCGAGGAAC |
| | 311 | AGGAAT |
| 344 | Encoding Probe | GCTCGACGTTCCTTTGCAACACCTCAGTTAATGATAGTGTG |
| | 312 | TCGATTG |
| 345 | Encoding Probe | GCTCGACGTTCCTTTGCAACAGTGTCTCATCTCTGAAAACT |
| | 313 | TCCCAC |
| 346 | Encoding Probe | GCTCGACGTTCCTTTGCAACACCACGTCAATGAGCAAAGGT |
| | 314 | AAAT |
| 347 | Encoding Probe | GCTCGACGTTCCTTTGCAACAGATACACACACTGATTCAGG |
| | 315 | CAGA |
| 348 | Encoding Probe | GCTCGACGTTCCTTTGCAACAACCTCAGTTAATGATAGTGT |
| | 316 | GTCGTTT |
| 349 | Encoding Probe | GCTCGACGTTCCTTTGCAACAGTATCATCTCTGAAAACTTC |
| | 317 | CGACC |
| 350 | Encoding Probe | GCTCGACGTTCCTTTGCAACATGCGTCACCCCATTAAGAGG |
| | 318 | CAGG |
| 351 | Encoding Probe | GCTCGACGTTCCTTTGCAACAGTAAGCTCACAATATGTGCA |
| | 319 | TTAAA |
| 352 | Encoding Probe | GCTCGACGTTCCTTTGCAACACTAAGTTAATGATAGTGTGT |
| | 320 | CGATTG |
| 353 | Encoding Probe | GCTCGACGTTCCTTTGCAACAAGGAAGGCACATTCTCATCT |
| | 321 | CACT |
| 354 | Encoding Probe | GCTCGACGTTCCTTTGCAACAGCGTCACCCCATTAAGAGGC |
| | 322 | TAGG |
| 355 | Encoding Probe | GCTCGACGTTCCTTTGCAACATAGGCTCACAATATGTGCAT |
| | 323 | TAAA |
| 356 | Encoding Probe | GCTCGACGTTCCTTTGCAACACCTCAGTTAATGATAGTGTG |
| | 324 | TCGTTT |
| 357 | Encoding Probe | CGGTCGGTGAGCATCTTCCATACCTCAGTTAATGATAGTGT |
| | 325 | GTCGATTG |
| 358 | Encoding Probe | CGGTCGGTGAGCATCTTCCATTGGAGCCTTGGTTTTCCGGA |
| | 326 | TTACG |
| 359 | Encoding Probe | CGGTCGGTGAGCATCTTCCATAGTGTCTCATCTCTGAAAAC |
| | 327 | TTCCCAC |
| 360 | Encoding Probe | CGGTCGGTGAGCATCTTCCATTGTCACCCCATTAAGAGGCT |
| | 328 | CCGTG |
| 361 | Encoding Probe | CGGTCGGTGAGCATCTTCCATACCACGTCAATGAGCAAAGG |
| | 329 | TAAAT |
| 362 | Encoding Probe | CGGTCGGTGAGCATCTTCCATTGTAAGCTCACAATATGTGC |
| | 330 | ATAAA |
| 363 | Encoding Probe | CGGTCGGTGAGCATCTTCCATAGATACACACACTGATTCAG |
| | 331 | GCAGA |
| 364 | Encoding Probe | CGGTCGGTGAGCATCTTCCATTAGTCTTGGTTTTCCGGATT |
| | 332 | TGGGA |
| 365 | Encoding Probe | CGGTCGGTGAGCATCTTCCATAACCTCAGTTAATGATAGTG |
| | 333 | TGTCGTTT |
| 366 | Encoding Probe | CGGTCGGTGAGCATCTTCCATTGAGCCTTGGTTTTCCGGAT |
| | 334 | TTCGG |
| 367 | Encoding Probe | CGGTCGGTGAGCATCTTCCATAGTATCATCTCTGAAAACTT |
| | 335 | CCGACC |
| 368 | Encoding Probe | CGGTCGGTGAGCATCTTCCATTGTGCTCAGCCTTGGTTTTC |
| | 336 | CGCTA |
| 369 | Encoding Probe | GTGGAGCGTCAGTACATGCTAATGCGTCACCCCATTAAGAG |
| | 337 | GCAGG |
| 370 | Encoding Probe | GTGGAGCGTCAGTACATGCTATCATGTCAATGAGCAAAGGT |
| | 338 | ATTAAGAA |
| 371 | Encoding Probe | GTGGAGCGTCAGTACATGCTAAGTAAGCTCACAATATGTGC |
| | 339 | ATTAAA |
| 372 | Encoding Probe | GTGGAGCGTCAGTACATGCTATGAAACTAACACACACACTG |
| | 340 | ATTGTC |
| 373 | Encoding Probe | GTGGAGCGTCAGTACATGCTAACTAAGTTAATGATAGTGTG |
| | 341 | TCGATTG |
| 374 | Encoding Probe | GTGGAGCGTCAGTACATGCTATGTGTCTCATCTCTGAAAAC |
| | 342 | TTCCGACC |
| 375 | Encoding Probe | GTGGAGCGTCAGTACATGCTAAAGGAAGGCACATTCTCATC |
| | 343 | TCACT |
| 376 | Encoding Probe | GTGGAGCGTCAGTACATGCTATCGTCACCCCATTAAGAGGC |
| | 344 | TCGGT |
| 377 | Encoding Probe | GTGGAGCGTCAGTACATGCTAAGCGTCACCCCATTAAGAGG |
| | 345 | CTAGG |
| 378 | Encoding Probe | GTGGAGCGTCAGTACATGCTATCATGTCAATGAGCAAAGGT |
| | 346 | ATTATGA |
| 379 | Encoding Probe | GTGGAGCGTCAGTACATGCTAATAGGCTCACAATATGTGCA |
| | 347 | TTAAA |
| 380 | Encoding Probe | GTGGAGCGTCAGTACATGCTATTGACACACACACTGATTCA |
| | 348 | GGGAG |

### Example 14. HiPR-Cycle Allows Detection of Unique and Shared Genes Across Multiple Taxa

In here, we show that HiPR-Cycle can be used to visualize and quantify gene expression in multiple taxa in a single field of view. While we have shown that we are capable of detecting multiple transcripts in a single taxa, this is the first evidence that a combined assay of HiPR-FISH and HiPR-Cycle probes identifies mRNA and rRNA in specific and broad use cases. An additional valuable piece of evidence in this experiment is the ability to detect antimicrobial resistant genes, as shown by the detection of bla mRNA in K. pneumoniae.

Method

To validate HiPR-Cycle's ability to detect multiple genes across multiple taxa, we performed HiPR-Cycle in a synthetic mixed community. The following bacteria under the following conditions were used: (1) E. coli (ATCC 25922) cultured in exponential growth phase and exposed to a large temperature shock (+16° C.) for 5 minutes; (2) carbapenem-resistant Klebsiella pneumoniae (ATCC BAA-1705) cultured under standard conditions; (3) Pseudomonas aeruginosa (ATCC 10145) cultured to the point it formed a biofilm. The three taxa were fixed in 2% formaldehyde and mixed in equal volumes. We targeted the rRNA of each taxa using HiPR-FISH probes containing a distinct readout for each taxa (Readout probe 4 for E. coli, Readout probe 6 for K. pneumoniae, and Readout probe 8 for P. aeruginosa.). We targeted several mRNAs including specific genes (bla(4) in K. pneumoniae with Readout probe 9, clpB in heat-shocked E. coli with Readout probe 1) and broad genes (me with Readout probe 7 and rho with Readout probe 5).

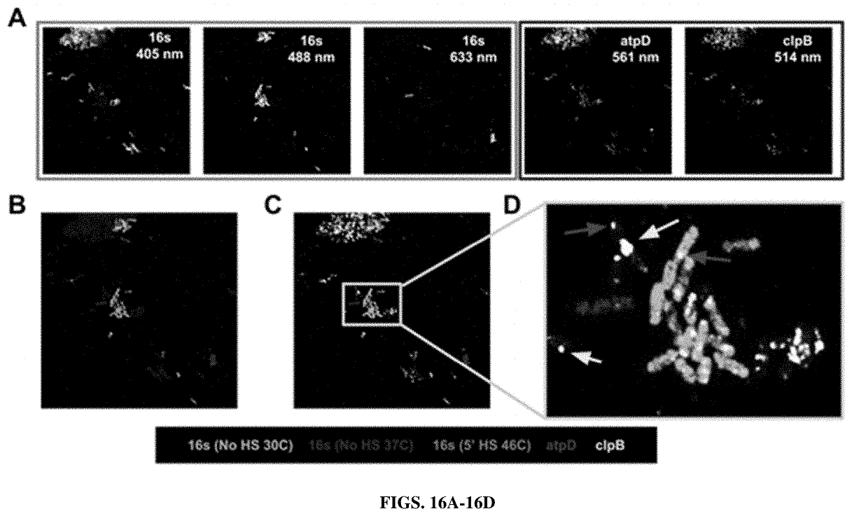

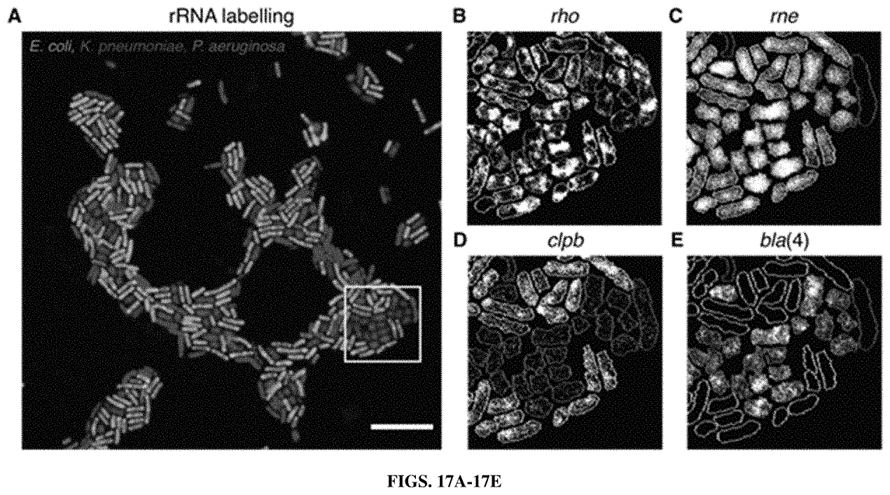

Encoding probes were added at a concentration of 400 nm and we performed encoding for 3 hours, followed by an overnight amplification/readout at 30° C. with amplifiers at a concentration of 200 nm and readouts at a concentration of 400 nm. Confocal imaging was performed using excitation wavelengths of 405 nm, 488 nm, 514 nm, 561 nm, and 633 nm. FIG. 17A shows a single field of view with all three microbial taxa in a single field of view. Bacteria were segmented based on the signal collected from rRNA-corresponding dyes (FIG. 17A), which showed strong, specific signal to their targeted taxa. House-keeping genes related to transcription and ribonuclease processing showed expression in multiple taxa. Specific gene expression was detected in heat-shocked E. coli (only taxa expressing clpB) and in carbapenem resistant K. pneumoniae (only expressing bla(4)).

FIGS. 17A-17E show a single field of view with all three microbial taxa in a single field of view. Bacteria were segmented based on the signal collected from rRNA-corresponding dyes (FIG. 17A). Signals from individual genes are shown in FIGS. 17B-17E; here, we either masked the signal within all segmented bacteria (FIGS. 17B-17D) or specifically masked K. pneumoniae for the purposes of illustrating bla(4) expression.

Encoding probes 349-513, as shown in Table 11 below, were used in this example. Amplifier probes 11-14, 17-18, and 23-24 (SEQ ID NO: 242-245, 281-282, and 381-382), as shown in Table 2, were used in this example. Readout probes 1-10 (SEQ ID NO: 25-34), as shown in Table 3, were used in this example.

TABLE 11
Encoding probes used in Example 14.

| SEQ | | |
|---|---|---|
| ID NO: | Probe Name | Sequence |
| 387 | Encoding Probe | TGTGATGGAAGTTAGAGGGTCCTCAGTTAATGATAGTGTGTCG |
| | 349 | ATTG |
| 388 | Encoding Probe | TGTGATGGAAGTTAGAGGGTGGAGCCTTGGTTTTCCGGATTAC |
| | 350 | G |
| 389 | Encoding Probe | TGTGATGGAAGTTAGAGGGTGTGTCTCATCTCTGAAAACTTCC |
| | 351 | CAC |
| 390 | Encoding Probe | TGTGATGGAAGTTAGAGGGTGTCACCCCATTAAGAGGCTCCGT |
| | 352 | G |
| 391 | Encoding Probe | TGTGATGGAAGTTAGAGGGTCCACGTCAATGAGCAAAGGTAAA |
| | 353 | T |
| 392 | Encoding Probe | TGTGATGGAAGTTAGAGGGTGTAAGCTCACAATATGTGCATAA |
| | 354 | A |
| 393 | Encoding Probe | TGTGATGGAAGTTAGAGGGTGATACACACACTGATTCAGGCAG |
| | 355 | A |
| 394 | Encoding Probe | TGTGATGGAAGTTAGAGGGTAGTCTTGGTTTTCCGGATTTGGG |
| | 356 | A |
| 395 | Encoding Probe | TGTGATGGAAGTTAGAGGGTACCTCAGTTAATGATAGTGTGTC |
| | 357 | GTTT |
| 396 | Encoding Probe | TGTGATGGAAGTTAGAGGGTGAGCCTTGGTTTTCCGGATTTCG |
| | 358 | G |
| 397 | Encoding Probe | TGTGATGGAAGTTAGAGGGTGTATCATCTCTGAAAACTTCCGA |
| | 359 | CC |
| 398 | Encoding Probe | TGTGATGGAAGTTAGAGGGTGTGCTCAGCCTTGGTTTTCCGCT |
| | 360 | A |
| 399 | Encoding Probe | TGTGATGGAAGTTAGAGGGTTGCGTCACCCCATTAAGAGGCAG |
| | 361 | G |
| 400 | Encoding Probe | TGTGATGGAAGTTAGAGGGTCATGTCAATGAGCAAAGGTATTA |
| | 362 | AGAA |
| 401 | Encoding Probe | TGTGATGGAAGTTAGAGGGTGTAAGCTCACAATATGTGCATTA |
| | 363 | AA |
| 402 | Encoding Probe | TGTGATGGAAGTTAGAGGGTGAAACTAACACACACACTGATTG |
| | 364 | TC |
| 403 | Encoding Probe | TGTGATGGAAGTTAGAGGGTCTAAGTTAATGATAGTGTGTCGA |
| | 365 | TTG |
| 404 | Encoding Probe | TGTGATGGAAGTTAGAGGGTGTGTCTCATCTCTGAAAACTTCC |
| | 366 | GACC |
| 405 | Encoding Probe | TGTGATGGAAGTTAGAGGGTAGGAAGGCACATTCTCATCTCAC |
| | 367 | T |
| 406 | Encoding Probe | TGTGATGGAAGTTAGAGGGTCGTCACCCCATTAAGAGGCTCGG |
| | 368 | T |
| 407 | Encoding Probe | TGTGATGGAAGTTAGAGGGTGCGTCACCCCATTAAGAGGCTAG |
| | 369 | G |
| 408 | Encoding Probe | TGTGATGGAAGTTAGAGGGTCATGTCAATGAGCAAAGGTATTA |
| | 370 | TGA |
| 409 | Encoding Probe | TGTGATGGAAGTTAGAGGGTTAGGCTCACAATATGTGCATTAA |
| | 371 | A |
| 410 | Encoding Probe | TGTGATGGAAGTTAGAGGGTTGACACACACACTGATTCAGGGA |
| | 372 | G |
| 411 | Encoding Probe | AGGTTAGGTTGAGAATAGGAGAGGCTCAGTAGTTTTGGATGCT |
| | 373 | CA |
| 412 | Encoding Probe | AGGTTAGGTTGAGAATAGGAAGACGCGTCACTTACGTGACACG |
| | 374 | GC |
| 413 | Encoding Probe | AGGTTAGGTTGAGAATAGGAGTGGAGGTGCTGGTAACTAAGCT |
| | 375 | G |
| 414 | Encoding Probe | AGGTTAGGTTGAGAATAGGACTAGTITTATGGGATTAGCTCCA |
| | 376 | GGA |
| 415 | Encoding Probe | AGGTTAGGTTGAGAATAGGAGAGGAAAGTTCTCAGCATGTCTT |
| | 377 | C |
| 416 | Encoding Probe | AGGTTAGGTTGAGAATAGGAACACCCATGCTCGGCACTTCTCC |
| | 378 | C |
| 417 | Encoding Probe | AGGTTAGGTTGAGAATAGGACGCGGTGTTTTTCACACCCATAC |
| | 379 | A |
| 418 | Encoding Probe | AGGTTAGGTTGAGAATAGGATGGCCAGAGTGATACATGAGGGC |
| | 380 | G |
| 419 | Encoding Probe | AGGTTAGGTTGAGAATAGGATGGCTATCTCCGAGCTTGATTTC |
| | 381 | G |
| 420 | Encoding Probe | AGGTTAGGTTGAGAATAGGAGGCACACAGGAAATTCCACCAAG |
| | 382 | G |
| 421 | Encoding Probe | AGGTTAGGTTGAGAATAGGAAAGATCCAACTTGCTGAACCAGG |
| | 383 | A |
| 422 | Encoding Probe | AGGTTAGGTTGAGAATAGGATGCGTCACCTAACAAGTAGGCAG |
| | 384 | G |
| 423 | Encoding Probe | AGGTTAGGTTGAGAATAGGACGTGTATTAACTTACTGCCCTTC |
| | 385 | GAG |
| 424 | Encoding Probe | AGGTTAGGTTGAGAATAGGAACAAGACAAAGTTTCTCGTGCAG |
| | 386 | G |
| 425 | Encoding Probe | AGGTTAGGTTGAGAATAGGAAAACTTCAAAGATCCTTTCGCCA |
| | 387 | T |
| 426 | Encoding Probe | AGGTTAGGTTGAGAATAGGAGCACGCTAAAATCAATGAAGCTA |
| | 388 | TT |
| 427 | Encoding Probe | AGGTTAGGTTGAGAATAGGACGATCTGATAGCGTGAGGTCCCT |
| | 389 | T |
| 428 | Encoding Probe | AGGTTAGGTTGAGAATAGGAATAATTCAGTACAAGATACCTAG |
| | 390 | GAAT |
| 429 | Encoding Probe | AGGTTAGGTTGAGAATAGGAAGGCGCTGAATCCAGGAGCAACG |
| | 391 | A |
| 430 | Encoding Probe | AGGTTAGGTTGAGAATAGGACAAAACGCTCTATGATCGTCAAT |
| | 392 | A |
| 431 | Encoding Probe | AGGTTAGGTTGAGAATAGGAGCAGTGTTTTTCACACCCATTGT |
| | 393 | GCA |
| 432 | Encoding Probe | AGGTTAGGTTGAGAATAGGACTGCGATCGGTTTTATGGGATAT |
| | 394 | C |
| 433 | Encoding Probe | AGGTTAGGTTGAGAATAGGAGGATCGACGTGTCTGTCTCGCTC |
| | 395 | A |
| 434 | Encoding Probe | AGGTTAGGTTGAGAATAGGAGGTGCAGTAACCAGAAGTACACC |
| | 396 | T |
| 435 | Encoding Probe | TTGGAGGTGTAGGGAGTAAAACCTCTTCGACTGGTCTCAGCAG |
| | 397 | G |
| 436 | Encoding Probe | TTGGAGGTGTAGGGAGTAAATGCAATCGATGAGGTTATTAACC |
| | 398 | TGTA |
| 437 | Encoding Probe | TTGGAGGTGTAGGGAGTAAACATCAGTCACACCCGAAGGTGCT |
| | 399 | AGG |
| 438 | Encoding Probe | TTGGAGGTGTAGGGAGTAAAGCAATCGATGAGGTTATTAACCT |
| | 400 | GTA |
| 439 | Encoding Probe | TTGGAGGTGTAGGGAGTAAACATCAGTCACACCCGAAGGTGCA |
| | 401 | GG |
| 440 | Encoding Probe | TTGGAGGTGTAGGGAGTAAAATGAGTCACACCCGAAGGTGCTA |
| | 402 | GG |
| 441 | Encoding Probe | TTGGAGGTGTAGGGAGTAAATCCCTTCACCTACACACCAGCGA |
| | 403 | CG |
| 442 | Encoding Probe | TTGGAGGTGTAGGGAGTAAATCCCTTCACCTACACACCAGCCA |
| | 404 | C |
| 443 | Encoding Probe | TTGGAGGTGTAGGGAGTAAATGACCGCAACCCCGGTGAGGGCG |
| | 405 | G |
| 444 | Encoding Probe | TTGGAGGTGTAGGGAGTAAAAGAGACTGGTCTCAGCTCCACGG |
| | 406 | C |
| 445 | Encoding Probe | TTGGAGGTGTAGGGAGTAAAATGAGTCACACCCGAAGGTGCAG |
| | 407 | G |
| 446 | Encoding Probe | TTGGAGGTGTAGGGAGTAAATGCGTCACACCCGAAGGTGCTAG |
| | 408 | G |
| 447 | Encoding Probe | TTGGAGGTGTAGGGAGTAAAGTGCTCAGCCTTGATTATCCGCT |
| | 409 | A |
| 448 | Encoding Probe | TTGGAGGTGTAGGGAGTAAACCACGTCAATCGATGAGGTTAAA |
| | 410 | T |
| 449 | Encoding Probe | TTGGAGGTGTAGGGAGTAAAAATAACCTCATCGCCTTCCTCAG |
| | 411 | G |
| 450 | Encoding Probe | TTGGAGGTGTAGGGAGTAAACCCACGTCAATCGATGAGGTTTA |
| | 412 | A |
| 451 | Encoding Probe | TTGGAGGTGTAGGGAGTAAACATCAGTCACACCCGAAGGTGGA |
| | 413 | G |
| 452 | Encoding Probe | TTGGAGGTGTAGGGAGTAAACCCTTCACCTACACACCAGCGAC |
| | 414 | G |
| 453 | Encoding Probe | CGGTCGGTGAGCATCTTCCATTCATCGCCAGCGCCTTACAAAG |
| | 415 | CTCT |
| 454 | Encoding Probe | CGGTCGGTGAGCATCTTCCATCGGTTCGAGCTGCGTTGCGGCT |
| | 416 | TCCA |
| 455 | Encoding Probe | CGGTCGGTGAGCATCTTCCATAAGACCACGCGCCAGTGCAGGT |
| | 417 | TTCA |
| 456 | Encoding Probe | CGGTCGGTGAGCATCTTCCATGCACGCTGCTGCAACAATTGCC |
| | 418 | GGGT |
| 457 | Encoding Probe | CGGTCGGTGAGCATCTTCCATGTCTACGCGGCGGCCTTTCAAC |
| | 419 | CCTT |
| 458 | Encoding Probe | CGGTCGGTGAGCATCTTCCATTTAACGCTGCGCCAGACCTTCA |
| | 420 | ACGA |
| 459 | Encoding Probe | CGGTCGGTGAGCATCTTCCATCAGAAGCGTCGTGGCACCTACG |
| | 421 | CAGT |
| 460 | Encoding Probe | CGGTCGGTGAGCATCTTCCATCAGACCCACACGGCGAGCCTGT |
| | 422 | TCAA |
| 461 | Encoding Probe | CGGTCGGTGAGCATCTTCCATCGCACGACGCACCGCTTCGGTC |
| | 423 | AGAT |
| 462 | Encoding Probe | CGGTCGGTGAGCATCTTCCATAAAATCCGGCAGCTGACGGTCA |
| | 424 | GCAA |
| 463 | Encoding Probe | CGGTCGGTGAGCATCTTCCATAAATCGCGCTCGCTTTCCATCA |
| | 425 | TGCG |
| 464 | Encoding Probe | CGGTCGGTGAGCATCTTCCATGAATACCCGCGCCGACCATGGT |
| | 426 | ATGT |
| 465 | Encoding Probe | CGGTCGGTGAGCATCTTCCATCTATGGGCATCGGCAAGAGCAA |
| | 427 | GCTG |
| 466 | Encoding Probe | CGGTCGGTGAGCATCTTCCATACGTTTCAGGATGTCGGCCAGC |
| | 428 | GTGC |
| 467 | Encoding Probe | CGGTCGGTGAGCATCTTCCATAGCATACGGACCATCGCCTCGT |
| | 429 | CGCT |
| 468 | Encoding Probe | CGGTCGGTGAGCATCTTCCATGCATGCAGCACCTGAATGGTAC |
| | 430 | GGCG |
| 469 | Encoding Probe | CGGTCGGTGAGCATCTTCCATTTATTGCACATGGTGGTGCAGC |
| | 431 | TCGT |
| 470 | Encoding Probe | CGGTCGGTGAGCATCTTCCATCTTTTGGTTGTCGTGCCCGAGT |
| | 432 | GCAA |
| 471 | Encoding Probe | CGGTCGGTGAGCATCTTCCATAACAGTAATGTTGGCGGTGGTC |
| | 433 | GCCC |
| 472 | Encoding Probe | CGGTCGGTGAGCATCTTCCATGGTATCGGGCGATTTGGATCCG |
| | 434 | CCAG |
| 473 | Encoding Probe | CGGTCGGTGAGCATCTTCCATTCATTGCCTTGTTCGGCTCGTT |
| | 435 | CGGT |
| 474 | Encoding Probe | CGGTCGGTGAGCATCTTCCATTTGGCGCACCAGATCCTGTGAT |
| | 436 | GGCT |
| 475 | Encoding Probe | CGGTCGGTGAGCATCTTCCATTACGCTTACCACACCGAGCACC |
| | 437 | AGCT |
| 476 | Encoding Probe | CGGTCGGTGAGCATCTTCCATCTACGTTCACGCTTTCACCTCC |
| | 438 | ACGC |
| 477 | Encoding Probe | CGGTCGGTGAGCATCTTCCATGTGCCGTTGGGCCGAGGAACAG |
| | 439 | GAAT |
| 478 | Encoding Probe | CGGTCGGTGAGCATCTTCCATCCGGCACCAACCAGACGAGACA |
| | 440 | CCGA |
| 479 | Encoding Probe | CGGTCGGTGAGCATCTTCCATGACGACGGCGACGATCCGGTCT |
| | 441 | TCAT |
| 480 | Encoding Probe | CGGTCGGTGAGCATCTTCCATATTCGCGATGGTGCAGTTCTTG |
| | 442 | CTCC |
| 481 | Encoding Probe | CGGTCGGTGAGCATCTTCCATGACGAAACGACGTTCCAGCGCA |
| | 443 | GCAT |
| 482 | Encoding Probe | GGGTGGTCGTCGAAGTCGTATCCGATGCGGGCGCGAGGTTTCG |
| | 444 | CTTT |
| 483 | Encoding Probe | GGGTGGTCGTCGAAGTCGTATAGATCGCGGCTTTCCACGCTTT |
| | 445 | CGCT |
| 484 | Encoding Probe | GGGTGGTCGTCGAAGTCGTATACCTCGGCGCGTCGGTTTCATC |
| | 446 | GCTT |
| 485 | Encoding Probe | GGGTGGTCGTCGAAGTCGTATACGTGGTTTCAGCAGGCTTGGC |
| | 447 | GGCA |
| 486 | Encoding Probe | GGGTGGTCGTCGAAGTCGTATAAGACCTTGCTGGCGTACTGCG |
| | 448 | GCAT |
| 487 | Encoding Probe | GGGTGGTCGTCGAAGTCGTATGTGTTCGCGCTGGAAAGCGGTC |
| | 449 | TCGA |
| 488 | Encoding Probe | GGGTGGTCGTCGAAGTCGTATGCCTGGCGTCTTCGCCGGAAGC |
| | 450 | TTCA |
| 489 | Encoding Probe | GGGTGGTCGTCGAAGTCGTATCGGACGTTCGCCGGACGCTTCC |
| | 451 | TTGA |
| 490 | Encoding Probe | GGGTGGTCGTCGAAGTCGTATCAATGCTTCTACGGCTTCGCTG |
| | 452 | GCGA |
| 491 | Encoding Probe | GGGTGGTCGTCGAAGTCGTATACCTTTCGCTGGCAGCCTCGCT |
| | 453 | GGTA |
| 492 | Encoding Probe | GGGTGGTCGTCGAAGTCGTATACTTGGCGTTGCGCTTCTCGTT |
| | 454 | GAGC |
| 493 | Encoding Probe | GGGTGGTCGTCGAAGTCGTATGTATTCGTAGCTGGTCTGGCCG |
| | 455 | GCAA |
| 494 | Encoding Probe | GGGTGGTCGTCGAAGTCGTATACCCCGTCGACCAGTGCAACAC |
| | 456 | GCAA |
| 495 | Encoding Probe | GGGTGGTCGTCGAAGTCGTATAGCAGGCTGGGTTCGACGCGAG |
| | 457 | TGAT |
| 496 | Encoding Probe | GGGTGGTCGTCGAAGTCGTATCGTACTTCTTCGGCCGCTTCCG |
| | 458 | TTGC |
| 497 | Encoding Probe | GGGTGGTCGTCGAAGTCGTATGCGAGCTCGTTGCGCTCTTCGC |
| | 459 | CTTC |
| 498 | Encoding Probe | GGGTGGTCGTCGAAGTCGTATCGCCCCTTGTTGCCGCGCTCTT |
| | 460 | CCTT |
| 499 | Encoding Probe | GGGTGGTCGTCGAAGTCGTATGCGGCGCGGGTACGCAGTTCGA |
| | 461 | TCTT |
| 500 | Encoding Probe | GGGTGGTCGTCGAAGTCGTATCTGTTCACCAGGCCCTGGAACA |
| | 462 | GGCT |
| 501 | Encoding Probe | GGGTGGTCGTCGAAGTCGTATCCTTGGCGCGGATGATGACGTT |
| | 463 | GCTT |
| 502 | Encoding Probe | GGGTGGTCGTCGAAGTCGTATTACTCGATGGAAACCAGGGCCT |
| | 464 | CGGT |
| 503 | Encoding Probe | GGGTGGTCGTCGAAGTCGTATCGATCTTCAGTGGCTTGCGCGA |
| | 465 | CCAG |
| 504 | Encoding Probe | GGGTGGTCGTCGAAGTCGTATCCGTCGACCGGTGCTTCGGTGT |
| | 466 | GTTG |
| 505 | Encoding Probe | GGGTGGTCGTCGAAGTCGTATTGCTCGGCCTGGGCGATTTCTT |
| | 467 | CAGC |
| 506 | Encoding Probe | GCTCGACGTTCCTTTGCAACACCTTGCGGTGGTTGCCGGTCGT |
| | 468 | GTTT |
| 507 | Encoding Probe | GCTCGACGTTCCTTTGCAACACGCAGCCAGCACAGCGGCAGCA |
| | 469 | AGAA |
| 508 | Encoding Probe | GCTCGACGTTCCTTTGCAACACGCATGAGGTATCGCGCGCATC |
| | 470 | GCCT |
| 509 | Encoding Probe | GCTCGACGTTCCTTTGCAACATCGTCCGCCACCGTCATGCCTG |
| | 471 | TTGT |
| 510 | Encoding Probe | GCTCGACGTTCCTTTGCAACAGTTTCAACAAACTGCTGCCGCT |
| | 472 | GCGG |
| 511 | Encoding Probe | GCTCGACGTTCCTTTGCAACAGCTTGACGGCCTCGCTGTGCTT |
| | 473 | GTCA |
| 512 | Encoding Probe | GCTCGACGTTCCTTTGCAACAATCACGGCCAACACAATAGGTG |
| | 474 | CGCG |
| 513 | Encoding Probe | GCTCGACGTTCCTTTGCAACATCGTGCACAGTGGGAAGCGCTC |
| | 475 | CTCA |
| 514 | Encoding Probe | GCTCGACGTTCCTTTGCAACACTTTGGTTCCGCGACGAGGTTG |
| | 476 | GTCA |
| 515 | Encoding Probe | GCTCGACGTTCCTTTGCAACAATGAGTTGCGCCTGAGCCGGTA |
| | 477 | TCCA |
| 516 | Encoding Probe | GCTCGACGTTCCTTTGCAACAAAATGTAAGCTTTCCGTCACGG |
| | 478 | CGCG |
| 517 | Encoding Probe | GCTCGACGTTCCTTTGCAACACAAATCACTGTATTGCACGGCG |
| | 479 | GCCG |
| 518 | Encoding Probe | GCTCGACGTTCCTTTGCAACATACGGTGACCACGGAACCAGCG |
| | 480 | CATT |
| 519 | Encoding Probe | GCTCGACGTTCCTTTGCAACAGCCGCCGCCCAACTCCTTCAGC |
| | 481 | AACA |
| 520 | Encoding Probe | GCTCGACGTTCCTTTGCAACAGTATTGCCGTGCCATACACTCC |
| | 482 | GCAG |
| 521 | Encoding Probe | GCTCGACGTTCCTTTGCAACAGGGAGCGGTCCAGACGGAACGT |
| | 483 | GGTA |
| 522 | Encoding Probe | GCTCGACGTTCCTTTGCAACACATACGGATGGGTGTGTCCAGC |
| | 484 | AAGC |
| 523 | Encoding Probe | GCTCGACGTTCCTTTGCAACAGCTTGGAGCCGCCAAAGTCCTG |
| | 485 | TTCG |
| 524 | Encoding Probe | GCTCGACGTTCCTTTGCAACAGCTTAGAGCGCATGAAGGCCGT |
| | 486 | CAGC |
| 525 | Encoding Probe | GCTCGACGTTCCTTTGCAACACAATTGTCTCCGACTGCCCAGT |
| | 487 | CTGC |
| 526 | Encoding Probe | GCTCGACGTTCCTTTGCAACAGGGAATCCCTCGAGCGCGAGTC |
| | 488 | TAGC |
| 527 | Encoding Probe | GCTCGACGTTCCTTTGCAACAGGTGCGGCCATGAGAGACAAGA |
| | 489 | CAGC |
| 528 | Encoding Probe | CGTCGGAGTGGGTTCAGTCTACGCAGAGTTCGCGTGCAGCGGC |
| | 490 | GTTA |
| 529 | Encoding Probe | CGTCGGAGTGGGTTCAGTCTATGGAGTGCGGAGGTTGAAACGG |
| | 491 | CGGA |
| 530 | Encoding Probe | CGTCGGAGTGGGTTCAGTCTATGCTGACGGGATGCCGGCTCGT |
| | 492 | CAAA |
| 531 | Encoding Probe | CGTCGGAGTGGGTTCAGTCTACAAACGTGCCGCACCAAAGAAA |
| | 493 | CGCT |
| 532 | Encoding Probe | CGTCGGAGTGGGTTCAGTCTAACAAGGCACGCGCCAGACGGGT |
| | 494 | AATA |
| 533 | Encoding Probe | CGTCGGAGTGGGTTCAGTCTATCCAGCTGTCTGCGGAGCGGAG |
| | 495 | GAAT |
| 534 | Encoding Probe | CGTCGGAGTGGGTTCAGTCTAACAAGTCGATAGCCGGGAAGAC |
| | 496 | GCGT |
| 535 | Encoding Probe | CGTCGGAGTGGGTTCAGTCTATCCCCAGATCCAGTACGCGAGC |
| | 497 | GGTT |
| 536 | Encoding Probe | CGTCGGAGTGGGTTCAGTCTACCTTACGTAGATGTCGTCAGGG |
| | 498 | CCGG |
| 537 | Encoding Probe | CGTCGGAGTGGGTTCAGTCTATCCTGCAGTTCCATGTTACCGG |
| | 499 | TGCC |
| 538 | Encoding Probe | CGTCGGAGTGGGTTCAGTCTAATCAACCGTTACCACGCTCCAT |
| | 500 | ACGC |
| 539 | Encoding Probe | CGTCGGAGTGGGTTCAGTCTAAGTGCAGCGCAAAGTAACGTTC |
| | 501 | ACCC |
| 540 | Encoding Probe | CGTCGGAGTGGGTTCAGTCTAAGACGAACAGGATCTTGTTACG |
| | 502 | CGCG |
| 541 | Encoding Probe | CGTCGGAGTGGGTTCAGTCTAGTACCTGCAGTATCTCCAGTAC |
| | 503 | GCCG |
| 542 | Encoding Probe | CGTCGGAGTGGGTTCAGTCTAGGTTCGCATCAATCTCACCCAT |
| | 504 | CGGA |
| 543 | Encoding Probe | CGTCGGAGTGGGTTCAGTCTAGGAGCTTACGCATACGAGCCAG |
| | 505 | GTTT |
| 544 | Encoding Probe | CGTCGGAGTGGGTTCAGTCTATAATTCTGCAGCTCTTCCTGAG |
| | 506 | TGGT |
| 545 | Encoding Probe | CGTCGGAGTGGGTTCAGTCTAAGTTTCGTCTTGGTCATTGCCA |
| | 507 | GCTT |
| 546 | Encoding Probe | CGTCGGAGTGGGTTCAGTCTAATTTCAGCTCAGAAACCGGCGT |
| | 508 | ATTC |
| 547 | Encoding Probe | CGTCGGAGTGGGTTCAGTCTACAACAACCAGACGTTTCGCTTT |
| | 509 | CTCA |
| 548 | Encoding Probe | CGTCGGAGTGGGTTCAGTCTAAAGCGGCTTGTCGTAGTTAACT |
| | 510 | TCGT |
| 549 | Encoding Probe | CGTCGGAGTGGGTTCAGTCTAGCTGCGCTTCATCATATCGAAG |
| | 511 | AAGT |
| 550 | Encoding Probe | CGTCGGAGTGGGTTCAGTCTATTTCTCTTCGTAGATAACTTCG |
| | 512 | TCCA |
| 551 | Encoding Probe | CGTCGGAGTGGGTTCAGTCTATGATTACCGGAAGCCGGAACCA |
| | 513 | CGGT |

### Example 15. HiPR-Cycle Allows Detection of Unique and Shared Genes Across Multiple Taxa

This example shows that HiPR-Cycle is easily adaptable to profile mammalian gene expression in FFPE tissue. This can have a wide range of applications including and beyond microbiome studies and microbiology. Importantly, the expression of the targeted gene, muc2, seems to be isolated to a small fraction cells which aligns with what is known about colonic goblet cells, which highly express this gene.

Method

We examined the possibility to detect gene expression of host-related genes while simultaneously profiling the taxa of several bacteria present in the healthy mouse colon. Encoding probes were designed to target the expression of mucin transcripts (muc2), which are highly expressed in mucus-producing goblet cells present in the mouse gastrointestinal tract. Additionally, HiPR-FISH encoding probes were used to detect several species of bacteria. A specimen (formalin-fixed paraffin-embedded mouse colon slice on a microscope slide) was first washed in xylene and ethanol in ethanol before HiPR-Cycle was performed. Encoding hybridization was performed at 37° C. for 24 hours, followed by an amplification/readout step performed at 30° C. for 15 hours. After amplification: (1) off target signal was removed with heated (42° C.) washes, (2) nuclei were stained with DAPI (1:50000 in 5×SSC), and (3) the tissue was cleared with Vector Laboratories' TrueVIEW Autofluorescence Quenching Kit. Mounted slides were then imaged on the confocal microscope.

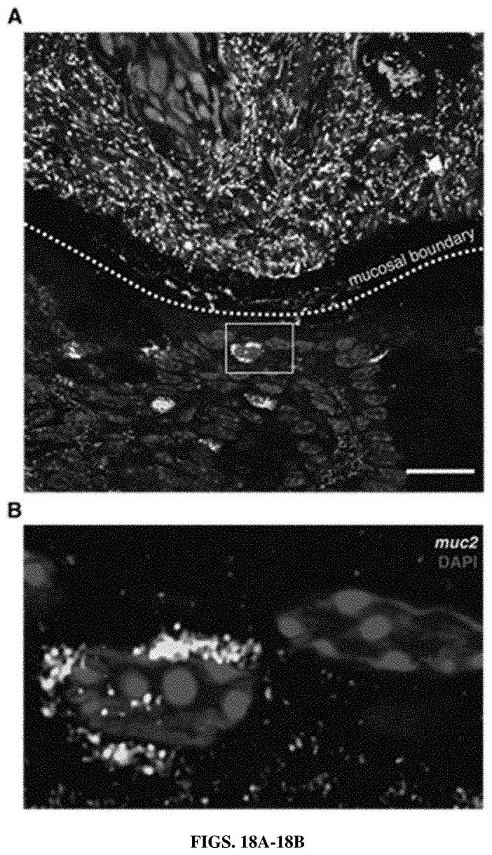

We detected high muc2 gene expression is a small fraction of cells, which agreed with past reviews and work showing goblet cell fractions do not exceed 1 in 6 intestinal epithelial cells (Kim Y S, Ho S B. Intestinal goblet cells and mucins in health and disease: recent insights and progress. Curr Gastroenterol Rep. 2010; 12(5):319-330) (FIG. 18A). Super-resolution microscopy revealed the punctate structure of gene expression, as one would see performing standard FISH on mammalian cells using standard microscopy (FIG. 18B). Importantly, the bacterial taxa were detectable with standard HiPR-FISH probes, showing the capability to combine HiPR-FISH and HiPR-Cycle probes in tissue in a single assay.

Encoding probes 514-531, as shown in Table 11 below, were used in this example. Amplifier probes 17-18 (SEQ ID NO: 281-282), as shown in Table 2, were used in this example. Readout probes 1-10 (SEQ ID NO: 25-34), as shown in Table 3, were used in this example

TABLE 12
Encoding probes used in Example 15.

| SEQ ID | | |
|---|---|---|
| NO: | Probe Name | Sequence |
| 552 | Encoding Probe 514 | CGTCGGAGTGGGTTCAGTCTACTGGTCCAGAGTCACAAAA |
| | | AGCTGCATGAC |
| 553 | Encoding Probe 515 | CGTCGGAGTGGGTTCAGTCTACAGTTGCACGTGTCATATT |
| | | TGCACCTCTTG |
| 554 | Encoding Probe 516 | CGTCGGAGTGGGTTCAGTCTAGTACATGAAGATCTGTGAG |
| | | CTTGGGCAAGC |
| 555 | Encoding Probe 517 | CGTCGGAGTGGGTTCAGTCTAAAGACCATGGTGGTAGCAG |
| | | GAACACTTAGC |
| 556 | Encoding Probe 518 | CGTCGGAGTGGGTTCAGTCTATGCAGATCTCATCAGTGGG |
| | | AACAGATCCTC |
| 557 | Encoding Probe 519 | CGTCGGAGTGGGTTCAGTCTATCTTCCCTTCATCTGGATG |
| | | GCATTCGATTT |
| 558 | Encoding Probe 520 | CGTCGGAGTGGGTTCAGTCTAGAGTGGTAGTTTCCGTTGG |
| | | AACAGTGAAGG |
| 559 | Encoding Probe 521 | CGTCGGAGTGGGTTCAGTCTAGTTCACCTGGGCCGTAGAA |
| | | CATCTCATTCA |
| 560 | Encoding Probe 522 | CGTCGGAGTGGGTTCAGTCTACAGTTACACAGCCACCAGG |
| | | TCTCATTAACC |
| 561 | Encoding Probe 523 | CGTCGGAGTGGGTTCAGTCTAACAGTTACCCTGGTAACTG |
| | | TAGTAGAGCCC |
| 562 | Encoding Probe 524 | CGTCGGAGTGGGTTCAGTCTAGGCATCACAGTGGTAGTTG |
| | | TCAATGTAGAC |
| 563 | Encoding Probe 525 | CGTCGGAGTGGGTTCAGTCTAGGAGATGTTCACCACAATG |
| | | TTGATGCCAGA |
| 564 | Encoding Probe 526 | CGTCGGAGTGGGTTCAGTCTATTCACAGTCAGAGATGATC |
| | | TTCCCACTGGG |
| 565 | Encoding Probe 527 | CGTCGGAGTGGGTTCAGTCTACACATGTCTTCACACAGAC |
| | | GTCATAGCCAG |
| 566 | Encoding Probe 528 | CGTCGGAGTGGGTTCAGTCTATCACTTCGAATCCCAACAA |
| | | ACATGTGGGGC |
| 567 | Encoding Probe 529 | CGTCGGAGTGGGTTCAGTCTACTCCCCAGGCTTCAGAATA |
| | | ATGTACTGCTG |
| 568 | Encoding Probe 530 | CGTCGGAGTGGGTTCAGTCTAAATCACTTGGGTTGAAGTC |
| | | GGGACAGGTGA |
| 569 | Encoding Probe 531 | CGTCGGAGTGGGTTCAGTCTAAGTACATGGCAAAAGTCCC |
| | | ACAGGACCCAA |

### Example 16: HiPR-Cycle can be Used to Detect Multiple Genes in Tissue Samples

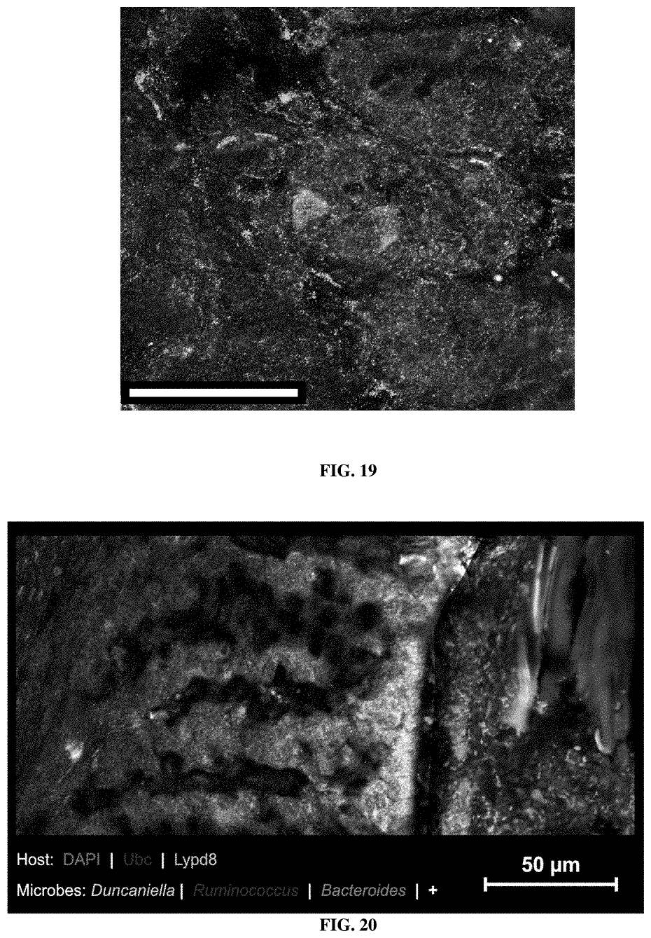

This experiment allowed us to show the ability to detect the expression of multiple genes in tissue using HiPR-Cycle, for example, to detect uncommon cell types like Gcg-expressing enteroendocrine cells, as shown in FIG. 19.

Method

Fresh frozen colon tissue was embedded in Tissue-Tek O.C.T. compound and sectioned at a thickness of 10 microns at −19° C. onto Ultrastick glass slides. Following sectioning, sections were covered with 2% formaldehyde and incubated in a chemical fume hood for 90 minutes at room temperature. Following fixation, samples were rinsed with 1×PBS, three times to remove the fixative. Specimens were placed in mailers with 70% ethanol and chilled to 4° C. for four hours to permeabilize the cell membrane.

After fixation, we added 10 μg/ml lysozyme to digest bacterial cell walls and incubated the sections for 30 minutes at 37° C.; sections were then washed with 1×PBS for 15 minutes at room temperature. We then added pre-encoding buffer to samples for 30 minutes at 37° C. Encoding buffer containing probes for Gcg (readout probe 10 [R10]), Gsn (readout probe 1 and 3 [R1+R3]), Aqp4 (readout probe 2 [R2]), Pam (readout probe 4 [R4]), Krt8 (readout probe 5 [R5]), Prdx1 (readout probe 6 [R6]), Col3a (readout probe 7 [R7]), Atp5a1 (readout probe 8 [R8]), and Kif3a (readout probe 9 [R9]) (at a concentration of 400 nM per gene pool) were added to specimens. The specimens were incubated overnight (16 hours) at 37° C.

The following day, specimens were washed in HiPR-FISH wash buffer for 15 minutes at 48° C. followed by 5×SSC+Tween 20 (5 minutes, room temperature) to remove unbound encoding probes. A pre-amplification buffer was added to specimens for 30 minutes at room temperature. During this incubation, amplifier probes were annealed by heating to 95° C. for 2 minutes and allowed to cool to room temperature for 30 minutes. An amplification buffer was then prepared by adding ten pairs of amplifier probes (90 nM each; stocks of probes at 9 μM). Amplification proceeded at 30° C., overnight (20 hours).

The following day, specimens were washed with 2×SSC+Tween 20 at 42° C. for 15 minutes. We then performed a readout step, where amplification buffer with all 10 readout probes (400 nM each) was added to specimens and incubated for 90 minutes at room temperature in the dark. Specimens were again washed with 2×SSC+Tween 20 at 42° C. for 15 minutes. Mammalian tissue was then cleared using TruView (Vector Laboratories) according to manufacturer instructions. Following the manufacturer-described wash step, specimens were incubated in 5×SSC+DAPI (1:1,000,000 dilution) for two minutes. The specimens were then mounted in Prolong Antifade.

Slides were imaged using a Zeiss i880 confocal in lambda mode with lasers set for 405 nm, 488 nm, 514 nm, 561 nm, and 633 nm excitation modes.

Encoding probes 222 and 570-756, as shown in Table 13 below, were used in this example. Amplifier probes 7-10, 17-18, and 27-36 (SEQ ID NO: 129-132, 281-282, 385-386, and 795-802), as shown in Table 2, were used in this example. Readout probes 1-10 (SEQ ID NO: 25-34), as shown in Table 3, were used in this example.

TABLE 13
Encoding probes used in Example 16.

| SEQ ID | | |
|---|---|---|
| NO: | Probe Name | Sequence |
| 246 | Encoding | TAGAGTTGATAGAGGGAGAAGCTGCCTCCCGTAGGAGT |
| | Probe 222 | |
| 570 | Encoding | CGTCGGAGTGGGTTCAGTCTAGTCCATGCCTCTCAAATTCAT |
| | Probe 532 | CATGACG |
| 571 | Encoding | CGTCGGAGTGGGTTCAGTCTACCGCCTCCAAGTAAGAACTCA |
| | Probe 533 | CATCACT |
| 572 | Encoding | CGTCGGAGTGGGTTCAGTCTACGTCCAGCATTATAAGCAATC |
| | Probe 534 | CAGC |
| 573 | Encoding | CGTCGGAGTGGGTTCAGTCTAAAAGTCTTCATTCATCTCATC |
| | Probe 535 | AGGGTC |
| 574 | Encoding | CGTCGGAGTGGGTTCAGTCTAACACAGTGATCTTGGTTTGAA |
| | Probe 536 | TCAGCC |
| 575 | Encoding | CGTCGGAGTGGGTTCAGTCTAGTCCCAAGCAATGAATTCCTT |
| | Probe 537 | TGCTG |
| 576 | Encoding | CGTCGGAGTGGGTTCAGTCTACCAGCTCATCTCGTCAGAGAA |
| | Probe 538 | GGA |
| 577 | Encoding | CGTCGGAGTGGGTTCAGTCTAAGACCTCTGTGTCTTGAAGGG |
| | Probe 539 | CG |
| 578 | Encoding | CGTCGGAGTGGGTTCAGTCTAGGGTGGTGGCAAGATTATCCA |
| | Probe 540 | GAATGG |
| 579 | Encoding | CGTCGGAGTGGGTTCAGTCTATTAGGCGACTTCTTCTGGGAA |
| | Probe 541 | GT |
| 580 | Encoding | CGTCGGAGTGGGTTCAGTCTAAAGCTCTTGGTGTTCATCAAC |
| | Probe 542 | CACTG |
| 581 | Encoding | CGTCGGAGTGGGTTCAGTCTATCACCAGGTATTTGCTGTAGT |
| | Probe 543 | CGCT |
| 582 | Encoding | CGTCGGAGTGGGTTCAGTCTAAGAGGGAAGCTGGGAATGATC |
| | Probe 544 | TGG |
| 583 | Encoding | CGTCGGAGTGGGTTCAGTCTAACCTGAATGTGCCCTGTGAGT |
| | Probe 545 | GG |
| 584 | Encoding | GCTCGACGTTCCTTTGCAACAGTGTTGGAAGAGGCGGATACT |
| | Probe 546 | GG |
| 585 | Encoding | GCTCGACGTTCCTTTGCAACATCGCACACCACTGATAGATAT |
| | Probe 547 | TGTTTCCCA |
| 586 | Encoding | GCTCGACGTTCCTTTGCAACAGTGCTCCTTTCTTGTACTTAA |
| | Probe 548 | GTCCAGACTTG |
| 587 | Encoding | GCTCGACGTTCCTTTGCAACAGAACAGCCTTTCAAATTTGTT |
| | Probe 549 | GCTGC |
| 588 | Encoding | GCTCGACGTTCCTTTGCAACACAACTTGAAGAACTGCTTAAA |
| | Probe 550 | GAGAGGG |
| 589 | Encoding | GCTCGACGTTCCTTTGCAACAGACCACAGTCTTTAGGATGAC |
| | Probe 551 | ATAGGC |
| 590 | Encoding | GCTCGACGTTCCTTTGCAACAACCCGGTAGTTGTACAGAATG |
| | Probe 552 | ATGTAGCT |
| 591 | Encoding | GCTCGACGTTCCTTTGCAACATTCCCCTGCCTAACGACTGTG |
| | Probe 553 | ATG |
| 592 | Encoding | GCTCGACGTTCCTTTGCAACACGGACCCTTGTAGATGATCAT |
| | Probe 554 | GGGC |
| 593 | Encoding | GCTCGACGTTCCTTTGCAACAGTGAGGTACCTCAGTAGCACG |
| | Probe 555 | GAC |
| 594 | Encoding | GCTCGACGTTCCTTTGCAACAGGCTGAAGAAGTCTCCATAGA |
| | Probe 556 | GGTTGGG |
| 595 | Encoding | GCTCGACGTTCCTTTGCAACATCACGCCATTGTTGAAACTGT |
| | Probe 557 | CCC |
| 596 | Encoding | GCTCGACGTTCCTTTGCAACAGGGTGCCAGTTGTAGATGATC |
| | Probe 558 | TGTC |
| 597 | Encoding | GCTCGACGTTCCTTTGCAACACGGACTAGGGAGACTGACATG |
| | Probe 559 | CTACCTG |
| 598 | Encoding | GCTCGACGTTCCTTTGCAACAGAGTGGGAGAACTGAAACCTG |
| | Probe 560 | GG |
| 599 | Encoding | GCTCGACGTTCCTTTGCAACATAGCAGTTGCACAGTAAAGAT |
| | Probe 561 | GGC |
| 600 | Encoding | GCTCGACGTTCCTTTGCAACATTCGCATCGTTGGAGTTCAGA |
| | Probe 562 | GC |
| 601 | Encoding | GCTCGACGTTCCTTTGCAACACTCGATGGGCATCCATCTTCT |
| | Probe 563 | TGTC |
| 602 | Encoding | GCTCGACGTTCCTTTGCAACAGGCATTTGCTGGATCTGTCTC |
| | Probe 564 | GATGTAC |
| 603 | Encoding | GCTCGACGTTCCTTTGCAACATAAGGGTACCACGTGTTTGAA |
| | Probe 565 | TCCA |
| 604 | Encoding | GCTCGACGTTCCTTTGCAACATATCTGCAGATTCCCATTCCT |
| | Probe 566 | CAG |
| 605 | Encoding | GCTCGACGTTCCTTTGCAACAGACGCACCTTGTTGGAACCTT |
| | Probe 567 | CA |
| 606 | Encoding | GCTCGACGTTCCTTTGCAACAAGATCAGACACGTGTACTTGA |
| | Probe 568 | GCA |
| 607 | Encoding | GCTCGACGTTCCTTTGCAACATGGGTTGGAGACCTTGTAGAG |
| | Probe 569 | CTTG |
| 608 | Encoding | GCACATGCTCCGTGTAGAATATCGCACACCACTGATAGATAT |
| | Probe 570 | TGTTTCCCA |
| 609 | Encoding | GCACATGCTCCGTGTAGAATAGTGCTCCTTTCTTGTACTTAA |
| | Probe 571 | GTCCAGACTTG |
| 610 | Encoding | GCACATGCTCCGTGTAGAATAGAACAGCCTTTCAAATTTGTT |
| | Probe 572 | GCTGC |
| 611 | Encoding | GCACATGCTCCGTGTAGAATACAACTTGAAGAACTGCTTAAA |
| | Probe 573 | GAGAGGG |
| 612 | Encoding | GCACATGCTCCGTGTAGAATAGACCACAGTCTTTAGGATGAC |
| | Probe 574 | ATAGGC |
| 613 | Encoding | GCACATGCTCCGTGTAGAATAACCCGGTAGTTGTACAGAATG |
| | Probe 575 | ATGTAGCT |
| 614 | Encoding | GCACATGCTCCGTGTAGAATATTCCCCTGCCTAACGACTGTG |
| | Probe 576 | ATG |
| 615 | Encoding | GCACATGCTCCGTGTAGAATACGGACCCTTGTAGATGATCAT |
| | Probe 577 | GGGC |
| 616 | Encoding | GCACATGCTCCGTGTAGAATAGTGAGGTACCTCAGTAGCACG |
| | Probe 578 | GAC |
| 617 | Encoding | GCACATGCTCCGTGTAGAATAGGCTGAAGAAGTCTCCATAGA |
| | Probe 579 | GGTTGGG |
| 618 | Encoding | GCACATGCTCCGTGTAGAATATCACGCCATTGTTGAAACTGT |
| | Probe 580 | CCC |
| 619 | Encoding | GCACATGCTCCGTGTAGAATAGGGTGCCAGTTGTAGATGATC |
| | Probe 581 | TGTC |
| 620 | Encoding | GCACATGCTCCGTGTAGAATACGGACTAGGGAGACTGACATG |
| | Probe 582 | CTACCTG |
| 621 | Encoding | GCACATGCTCCGTGTAGAATAGAGTGGGAGAACTGAAACCTG |
| | Probe 583 | GG |
| 622 | Encoding | GCACATGCTCCGTGTAGAATATAGCAGTTGCACAGTAAAGAT |
| | Probe 584 | GGC |
| 623 | Encoding | GCACATGCTCCGTGTAGAATACTCGATGGGCATCCATCTTCT |
| | Probe 585 | TGTC |
| 624 | Encoding | GCACATGCTCCGTGTAGAATAGGCATTTGCTGGATCTGTCTC |
| | Probe 586 | GATGTAC |
| 625 | Encoding | GCACATGCTCCGTGTAGAATATAAGGGTACCACGTGTTTGAA |
| | Probe 587 | TCCA |
| 626 | Encoding | GCACATGCTCCGTGTAGAATATATCTGCAGATTCCCATTCCT |
| | Probe 588 | CAG |
| 627 | Encoding | GCACATGCTCCGTGTAGAATAGACGCACCTTGTTGGAACCTT |
| | Probe 589 | CA |
| 628 | Encoding | GCACATGCTCCGTGTAGAATATTCGCATCGTTGGAGTTCAGA |
| | Probe 590 | GCA |
| 629 | Encoding | GCACATGCTCCGTGTAGAATAAGATCAGACACGTGTACTTGA |
| | Probe 591 | GCA |
| 630 | Encoding | GCACATGCTCCGTGTAGAATATGGGTTGGAGACCTTGTAGAG |
| | Probe 592 | CTTG |
| 631 | Encoding | GCACATGCTCCGTGTAGAATAGAAGCCTTTCCAAACAAAGAT |
| | Probe 593 | TTTCCCA |
| 632 | Encoding | GCTGGTCGGTGGATTGGATTTGTCGACAGAAGACATACTCAT |
| | Probe 594 | AAAGGGCA |
| 633 | Encoding | GCTGGTCGGTGGATTGGATTTGATGTCATACGGAAGACAATA |
| | Probe 595 | CCTCTCC |
| 634 | Encoding | GCTGGTCGGTGGATTGGATTTGTGCGAGCAAAACAAAGATAA |
| | Probe 596 | GCGTG |
| 635 | Encoding | GCTGGTCGGTGGATTGGATTTCTTCCAGTAACATCAGTTCGT |
| | Probe 597 | TTGGAATC |
| 636 | Encoding | GCTGGTCGGTGGATTGGATTTTGTGCTGGCAAAAATAGTGAA |
| | Probe 598 | CACCAA |
| 637 | Encoding | GCTGGTCGGTGGATTGGATTTTTGTGGAAAGTGATTATTAAC |
| | Probe 599 | TCCACCAGG |
| 638 | Encoding | GCTGGTCGGTGGATTGGATTTTTAGATGTAGAAGACGGACTT |
| | Probe 600 | AGCG |
| 639 | Encoding | GCTGGTCGGTGGATTGGATTTGGTGTTTCCCATGATAACTGC |
| | Probe 601 | GGGT |
| 640 | Encoding | GCTGGTCGGTGGATTGGATTTGTGCCCAGTTTATGGTGGATC |
| | Probe 602 | CCA |
| 641 | Encoding | GCTGGTCGGTGGATTGGATTTTACCTGGCTCCAGTATAATTG |
| | Probe 603 | ATTGCAAAC |
| 642 | Encoding | GCTGGTCGGTGGATTGGATTTGAACAGGATCAAGTCTTCCGT |
| | Probe 604 | CTCC |
| 643 | Encoding | GCTGGTCGGTGGATTGGATTTAGGACGGTCAATGTCAATCAC |
| | Probe 605 | ATGC |
| 644 | Encoding | GCTGGTCGGTGGATTGGATTTTAAGCAACGGAAAATCCAATT |
| | Probe 606 | GCTAAAG |
| 645 | Encoding | GCTGGTCGGTGGATTGGATTTGTCCGGTGAGGTTTCCATGAA |
| | Probe 607 | CCG |
| 646 | Encoding | GCTGGTCGGTGGATTGGATTTGCTTGCTGATCTTTCGTGTGC |
| | Probe 608 | AC |
| 647 | Encoding | GCTGGTCGGTGGATTGGATTTCCACCAACCCAATATATCCAG |
| | Probe 609 | TGGTTT |
| 648 | Encoding | GCTGGTCGGTGGATTGGATTTTCGAATGCTGAGTCCAAAGCA |
| | Probe 610 | AAGG |
| 649 | Encoding | GCTGGTCGGTGGATTGGATTTACTGTCCAGACTCCTTTGAAA |
| | Probe 611 | GCCA |
| 650 | Encoding | GCTGGTCGGTGGATTGGATTTCTTGGCTTCCTTAAGGCGACG |
| | Probe 612 | TT |
| 651 | Encoding | GCTGGTCGGTGGATTGGATTTGTTTCCTCCAACCACACTGGG |
| | Probe 613 | AG |
| 652 | Encoding | GCTGGTCGGTGGATTGGATTTGTAGATGCTCTCTCTACTGCA |
| | Probe 614 | GGAATG |
| 653 | Encoding | GCTGGTCGGTGGATTGGATTTAGGTCCACCTCCATGTAGCTC |
| | Probe 615 | CC |
| 654 | Encoding | GCTGGTCGGTGGATTGGATTTAAGTGCTGAGACTGCCTTCCA |
| | Probe 616 | GA |
| 655 | Encoding | GCTGGTCGGTGGATTGGATTTCCCAGATGAGGACCATGTCCA |
| | Probe 617 | CAGG |
| 656 | Encoding | CGCATACCGGCCTATGGACTAAGTCTACCTTACCTAAATGGT |
| | Probe 618 | GAGTATGGAC |
| 657 | Encoding | CGCATACCGGCCTATGGACTAGTACCGTGACCCAATAATTTC |
| | Probe 619 | CATCTGTA |
| 658 | Encoding | CGCATACCGGCCTATGGACTATTACCGAAGAATCAATAGGAG |
| | Probe 620 | TGATGGG |
| 659 | Encoding | CGCATACCGGCCTATGGACTAAAAGGCTGCTGTATTAAAGCA |
| | Probe 621 | CTCTTTTC |
| 660 | Encoding | CGCATACCGGCCTATGGACTAGTCCTTGGAAAGTAGTGAATA |
| | Probe 622 | GAAATCACCCTG |
| 661 | Encoding | CGCATACCGGCCTATGGACTAGAAGCCCAGGCATATAGAATA |
| | Probe 623 | TTGGCTT |
| 662 | Encoding | CGCATACCGGCCTATGGACTAGTAGCGGTAAATAAAACAGAT |
| | Probe 624 | TCTTGCC |
| 663 | Encoding | CGCATACCGGCCTATGGACTACGGAGCCTTTTTAACTGATCG |
| | Probe 625 | ATGCTC |
| 664 | Encoding | CGCATACCGGCCTATGGACTATTACCAACACCTTTAGGGAGC |
| | Probe 626 | CG |
| 665 | Encoding | CGCATACCGGCCTATGGACTACTTCTTGCTGTCAAAAGAGTT |
| | Probe 627 | TCCATCC |
| 666 | Encoding | CGCATACCGGCCTATGGACTAAAGTGGGATAGTTCTGAACAT |
| | Probe 628 | ATCTGGAG |
| 667 | Encoding | CGCATACCGGCCTATGGACTATTTTGTCAGAATTCACTACTT |
| | Probe 629 | TCTCTCCTGG |
| 668 | Encoding | CGCATACCGGCCTATGGACTAATTCCAGAAGGGTTGTAATGA |
| | Probe 630 | GAACCA |
| 669 | Encoding | CGCATACCGGCCTATGGACTATTTGAGGAAACCTGGTATATA |
| | Probe 631 | TGAGATTGC |
| 670 | Encoding | CGCATACCGGCCTATGGACTATCTAGCCACAATATCATGAGG |
| | Probe 632 | CATGT |
| 671 | Encoding | CGCATACCGGCCTATGGACTAAGGCCACTGGAAAAGTTCATC |
| | Probe 633 | ACAAATC |
| 672 | Encoding | CGCATACCGGCCTATGGACTAGAATGTTTTCTGTTTCTTTGT |
| | Probe 634 | GATGCCC |
| 673 | Encoding | CGCATACCGGCCTATGGACTAGAAGCGAACTGGTTTGAAAAC |
| | Probe 635 | ATCT |
| 674 | Encoding | CGCATACCGGCCTATGGACTACAACCACTGGGTAAAAAGCCT |
| | Probe 636 | GTGG |
| 675 | Encoding | CGCATACCGGCCTATGGACTAGGTAGGCATTCATTGGAAAAT |
| | Probe 637 | GATCTGG |
| 676 | Encoding | CGCATACCGGCCTATGGACTACTCGCTTGAAGTCAATCACGA |
| | Probe 638 | AGGC |
| 677 | Encoding | CGCATACCGGCCTATGGACTAGCAGTCAGACTCTTTAGGTGT |
| | Probe 639 | GACCC |
| 678 | Encoding | CGCATACCGGCCTATGGACTACAAGCAGTAACCATCTGACAC |
| | Probe 640 | GAAG |
| 679 | Encoding | CGCATACCGGCCTATGGACTATAATCAGCTTTATTTGGGTCA |
| | Probe 641 | ATGACCA |
| 680 | Encoding | GCTCCGTTACGCGTTTAAGTCAACGGATATCCCAGATAGGAA |
| | Probe 642 | ATTCCTCCT |
| 681 | Encoding | GCTCCGTTACGCGTTTAAGTCACCTGGTCTTCGTATGAATGC |
| | Probe 643 | TCATGTT |
| 682 | Encoding | GCTCCGTTACGCGTTTAAGTCAGCGACGGGTCTCTAGTTCCC |
| | Probe 644 | TG |
| 683 | Encoding | GCTCCGTTACGCGTTTAAGTCAGGCCCATAGGATGAACTCAG |
| | Probe 645 | TCCTC |
| 684 | Encoding | GCTCCGTTACGCGTTTAAGTCTGATGGACACGACATCAGAAG |
| | Probe 646 | ACTC |
| 685 | Encoding | GCTCCGTTACGCGTTTAAGTCACGGGCTGAAAGTGTTGGATC |
| | Probe 647 | CC |
| 686 | Encoding | GCTCCGTTACGCGTTTAAGTCTCGTGAAGACGGAGGTTCGAG |
| | Probe 648 | AG |
| 687 | Encoding | GCTCCGTTACGCGTTTAAGTCCGGAGAGGATTAGGGCTGATC |
| | Probe 649 | CTC |
| 688 | Encoding | GCTCCGTTACGCGTTTAAGTCTTTATACAACTGAATTGGGTT |
| | Probe 650 | TGGATGG |
| 689 | Encoding | GCTCCGTTACGCGTTTAAGTCCGAGCTTCTAGACGGTGGGAC |
| | Probe 651 | AG |
| 690 | Encoding | GCTCCGTTACGCGTTTAAGTCACTTGGACATGGTGAAGTCTG |
| | Probe 652 | GAG |
| 691 | Encoding | GCTCCGTTACGCGTTTAAGTCTCCAGCTCATTCCGTAGCTGA |
| | Probe 653 | AGC |
| 692 | Encoding | GCTCCGTTACGCGTTTAAGTCCGGTTTGAGGGCTTCAATCTC |
| | Probe 654 | CGC |
| 693 | Encoding | GCTCCGTTACGCGTTTAAGTCAACCGGTTCATCTCGGAGATC |
| | Probe 655 | TCTG |
| 694 | Encoding | GCTCCGTTACGCGTTTAAGTCGAACCCATCTCGGGTTTCAAT |
| | Probe 656 | CTTCT |
| 695 | Encoding | GCTCCGTTACGCGTTTAAGTCTGACTGCCATACAGCTGTCTC |
| | Probe 657 | CC |
| 696 | Encoding | GCTCCGTTACGCGTTTAAGTCCCCCATCCTTAATGGCCATCT |
| | Probe 658 | CCC |
| 697 | Encoding | GCTCCGTTACGCGTTTAAGTCGTTGGACTTCAGTGGCCATTC |
| | Probe 659 | AC |
| 698 | Encoding | GCTCCGTTACGCGTTTAAGTCTCCTGGTGATCTCGATGTCCA |
| | Probe 660 | GG |
| 699 | Encoding | GCAACCCGAGCATTCGTATGATTTTTCATCGGCTCTATCACT |
| | Probe 661 | GAAAG |
| 700 | Encoding | GCAACCCGAGCATTCGTATGATCGTGGACACACTTCACCATG |
| | Probe 662 | TTT |
| 701 | Encoding | GCAACCCGAGCATTCGTATGAGTAAACAGCTGTAGCTTTGAA |
| | Probe 663 | GTTGG |
| 702 | Encoding | GCAACCCGAGCATTCGTATGATTTTTCATCGGCTCTATCACT |
| | Probe 664 | GAAAG |
| 703 | Encoding | GCAACCCGAGCATTCGTATGATCGTGGACACACTTCACCATG |
| | Probe 665 | TTT |
| 704 | Encoding | GCAACCCGAGCATTCGTATGAGTAAACAGCTGTAGCTTTGAA |
| | Probe 666 | GTTGG |
| 705 | Encoding | GCAACCCGAGCATTCGTATGAGTGCTACCTAGGAACTTAACT |
| | Probe 667 | GTAGCTCAATC |
| 706 | Encoding | GCAACCCGAGCATTCGTATGAGTATTCCTGAAGACATCTTGC |
| | Probe 668 | TATCAGC |
| 707 | Encoding | GCAACCCGAGCATTCGTATGATTGCAGCACTAGAAAAGTAGA |
| | Probe 669 | TCCTGAGAG |
| 708 | Encoding | GCAACCCGAGCATTCGTATGAGTCCTCAGACCATTACCAATA |
| | Probe 670 | GTGGAAGAA |
| 709 | Encoding | GCAACCCGAGCATTCGTATGATGTGACACTAGGCATTTATCC |
| | Probe 671 | TAAGAAGCA |
| 710 | Encoding | GCAACCCGAGCATTCGTATGATAACCTGGCAGATTAAATAGG |
| | Probe 672 | CAGGTAG |
| 711 | Encoding | GCAACCCGAGCATTCGTATGAGATGCCTCACATATACATCAA |
| | Probe 673 | GTTCTAGATCAG |
| 712 | Encoding | GCAACCCGAGCATTCGTATGATAGCTGTCTCAAAAGTTAAGA |
| | Probe 674 | GACTGGAGA |
| 713 | Encoding | GCAACCCGAGCATTCGTATGACCCGCTTTGGAGATAGTTCAG |
| | Probe 675 | CTGTG |
| 714 | Encoding | GCAACCCGAGCATTCGTATGATCGACGAGTAGAGTACAAGAG |
| | Probe 676 | TTTCTTCTG |
| 715 | Encoding | GCAACCCGAGCATTCGTATGATTTGGCCAACTATGGTAATCC |
| | Probe 677 | ATGC |
| 716 | Encoding | GCAACCCGAGCATTCGTATGACAACAGCAACAAGTAAAACAG |
| | Probe 678 | AATACCAGG |
| 717 | Encoding | GCAACCCGAGCATTCGTATGACTTTCACCAAGTTCTCAGAAT |
| | Probe 679 | TCACGTT |
| 718 | Encoding | GCAACCCGAGCATTCGTATGAAACCCTTCAGTTCACTAGTAC |
| | Probe 680 | TGAGATTACAG |
| 719 | Encoding | GCAACCCGAGCATTCGTATGAATTACATTTCCAAGATTAGAG |
| | Probe 681 | TGGTCTGTG |
| 720 | Encoding | GCAACCCGAGCATTCGTATGAGACTGACCAGGAAGTATAAGC |
| | Probe 682 | CAGATACTT |
| 721 | Encoding | GCAACCCGAGCATTCGTATGAGACCCTGGCAGAAAAATGGTC |
| | Probe 683 | CAGT |
| 722 | Encoding | GCAACCCGAGCATTCGTATGATGTTGGCAATAGGGTTATAAC |
| | Probe 684 | AAGGC |
| 723 | Encoding | CGTCAGGTGAGCATCTTACATCGACCTCATCACAGATTATGT |
| | Probe 685 | CATCGCA |
| 724 | Encoding | CGTCAGGTGAGCATCTTACATGTCACTTGACATCATATGAGT |
| | Probe 686 | CGAATTGGG |
| 725 | Encoding | CGTCAGGTGAGCATCTTACATGTACAGGGCTTATAAGACTCT |
| | Probe 687 | CTATTTGTCC |
| 726 | Encoding | CGTCAGGTGAGCATCTTACATCCCTGCGATATCTATGATGGG |
| | Probe 688 | TAGTCTCAT |
| 727 | Encoding | CGTCAGGTGAGCATCTTACATCCGTTCCAGACATCTCTAGAC |
| | Probe 689 | TCATAGGAC |
| 728 | Encoding | CGTCAGGTGAGCATCTTACATAGTGTGTTGATCTTGAAATCC |
| | Probe 690 | ATTGGATCG |
| 729 | Encoding | CGTCAGGTGAGCATCTTACATCCGCCCTTGCTCCTATTAGTC |
| | Probe 691 | CAGGG |
| 730 | Encoding | CGTCAGGTGAGCATCTTACATGTACCTCTAGAACTGTGTAAG |
| | Probe 692 | TGAATTTGC |
| 731 | Encoding | CGTCAGGTGAGCATCTTACATTAAGCACAACATTCTCCAAAT |
| | Probe 693 | GGGATC |
| 732 | Encoding | CGTCAGGTGAGCATCTTACATCGAGCATCCCAATTCATCTAC |
| | Probe 694 | GTTGGA |
| 733 | Encoding | CGTCAGGTGAGCATCTTACATGAAGCGTGTTTGATATTCAAA |
| | Probe 695 | GACTGTC |
| 734 | Encoding | CGTCAGGTGAGCATCTTACATTTGCACCATTCATAGATTCTC |
| | Probe 696 | CAAACCA |
| 735 | Encoding | CGTCAGGTGAGCATCTTACATCCAGGCAGAATTTTAGGTCTC |
| | Probe 697 | TGCA |
| 736 | Encoding | CGTCAGGTGAGCATCTTACATAGTCAGACACATATTTGACAT |
| | Probe 698 | GGTTCTG |
| 737 | Encoding | CGTCAGGTGAGCATCTTACATCCCCACCATCATTTCCTTTAG |
| | Probe 699 | GACCAG |
| 738 | Encoding | CGTCAGGTGAGCATCTTACATAACGACATGATTCACAGATTC |
| | Probe 700 | CAGGG |
| 739 | Encoding | CGTCAGGTGAGCATCTTACATGACCTGTGCCAAAATAAGAGT |
| | Probe 701 | GGGA |
| 740 | Encoding | CGTCAGGTGAGCATCTTACATGGACGAGGTCCAGCTATACCT |
| | Probe 702 | GG |
| 741 | Encoding | CGTCAGGTGAGCATCTTACATACACCTTTGATGCCATTAGAG |
| | Probe 703 | CCA |
| 742 | Encoding | CGTCAGGTGAGCATCTTACATGGTGTATTCTCCACTCTTGAG |
| | Probe 704 | TTCGGG |
| 743 | Encoding | CGTCAGGTGAGCATCTTACATGACGTGGTCCAATAGGACCTG |
| | Probe 705 | GAT |
| 744 | Encoding | CGTCAGGTGAGCATCTTACATAAGCCCAGTGTGTTTAGTACA |
| | Probe 706 | GCC |
| 745 | Encoding | CGTCAGGTGAGCATCTTACATCTCCCCTCAGATCCTCTTTCA |
| | Probe 707 | CCTC |
| 746 | Encoding | CGTCAGGTGAGCATCTTACATAAGCCCAGTTTCCATGTTACA |
| | Probe 708 | GAATACT |
| 747 | Encoding | TGCCTCCGTCTGAGTATTCCTCGGGTTGTCTCTAAAGTACTC |
| | Probe 709 | TCCCATGG |
| 748 | Encoding | TGCCTCCGTCTGAGTATTCCTCTAAGATCGTCATAGATGATC |
| | Probe 710 | AAAGCGT |
| 749 | Encoding | TGCCTCCGTCTGAGTATTCCTGACGCTGATAACGTGAGACAA |
| | Probe 711 | GAAAGC |
| 750 | Encoding | TGCCTCCGTCTGAGTATTCCTTTGGTCTCCTTCTTTAATTAG |
| | Probe 712 | CTTGTCATTCCC |
| 751 | Encoding | TGCCTCCGTCTGAGTATTCCTGTGGACAACTCCAACATTGTC |
| | Probe 713 | GGGT |
| 752 | Encoding | TGCCTCCGTCTGAGTATTCCTCTCGCATGGAGATTTCTTGCA |
| | Probe 714 | CCA |
| 753 | Encoding | TGCCTCCGTCTGAGTATTCCTAAACCCATCAGACCTGATATT |
| | Probe 715 | GCCC |
| 754 | Encoding | TGCCTCCGTCTGAGTATTCCTTTAAACATTTGTTGGAATGTA |
| | Probe 716 | AGCGGAC |
| 755 | Encoding | TGCCTCCGTCTGAGTATTCCTCTGCCTTTCCATCAATAGCAT |
| | Probe 717 | TACCGAGG |
| 756 | Encoding | TGCCTCCGTCTGAGTATTCCTTATCCCTTTAAGCCTGAAGAG |
| | Probe 718 | AATTCC |
| 757 | Encoding | TGCCTCCGTCTGAGTATTCCTGAGTGCTTGAACATTCCTCAG |
| | Probe 719 | CC |
| 758 | Encoding | TGCCTCCGTCTGAGTATTCCTGAGCTCGAGAATGGAGGACAT |
| | Probe 720 | CTCA |
| 759 | Encoding | TGCCTCCGTCTGAGTATTCCTTGGTGTTCTTCAATAGCCATG |
| | Probe 721 | GGAGA |
| 760 | Encoding | TGCCTCCGTCTGAGTATTCCTTCGCGGAGTAGGGAGCCAAGT |
| | Probe 722 | ACT |
| 761 | Encoding | TGCCTCCGTCTGAGTATTCCTACCGCTCCAGTTTGTCAAGAT |
| | Probe 723 | AACCC |
| 762 | Encoding | TGCCTCCGTCTGAGTATTCCTAAGAATGACTGGTAAGGCAGT |
| | Probe 724 | CAAAGAG |
| 763 | Encoding | TGCCTCCGTCTGAGTATTCCTGAGCTCCAGGTCAACAGACGT |
| | Probe 725 | GTC |
| 764 | Encoding | TGCCTCCGTCTGAGTATTCCTCACCCAGTCTTCTGAAGTCGA |
| | Probe 726 | GTGTTAG |
| 765 | Encoding | TGCCTCCGTCTGAGTATTCCTTCCGCGGATGCCTTTATAGAA |
| | Probe 727 | CAATTCTG |
| 766 | Encoding | TGCCTCCGTCTGAGTATTCCTTGGAAAGGAATCGTTCATCTT |
| | Probe 728 | GGCTG |
| 767 | Encoding | TGCCTCCGTCTGAGTATTCCTGGTTCGTTGAAACGTTTCTGG |
| | Probe 729 | TTGA |
| 768 | Encoding | TGCCTCCGTCTGAGTATTCCTGGCCATCCGTCAGTCTCTTCA |
| | Probe 730 | CC |
| 769 | Encoding | TGCCTCCGTCTGAGTATTCCTGGGCAAAGCATTTTTGGAGAC |
| | Probe 731 | CAGTCC |
| 770 | Encoding | TGCCTCCGTCTGAGTATTCCTCGTCTCGCGCAATACCATCAC |
| | Probe 732 | CA |
| 771 | Encoding | GTGTGTGCCAGGATGATCAATGACTGCTCGTTCATATTAGTC |
| | Probe 733 | GCACC |
| 772 | Encoding | GTGTGTGCCAGGATGATCAATGATGAGTCATGATTCTATCCA |
| | Probe 734 | TGTCGTCAG |
| 773 | Encoding | GTGTGTGCCAGGATGATCAATAGAGGGTCTGATCTTTACCCA |
| | Probe 735 | AAAGGTC |
| 774 | Encoding | GTGTGTGCCAGGATGATCAATAATCTGAAGTAAAGAATCAAT |
| | Probe 736 | TACGGTCTCAGG |
| 775 | Encoding | GTGTGTGCCAGGATGATCAATGAGCAAGTATGACACTCGAAC |
| | Probe 737 | CAAAAACC |
| 776 | Encoding | GTGTGTGCCAGGATGATCAATGAAGCAGACTGGTATATTTTT |
| | Probe 738 | CCTCGATGT |
| 777 | Encoding | GTGTGTGCCAGGATGATCAATAGTCTGCACTCTATAGTAATT |
| | Probe 739 | GTAAAGATGGCG |
| 778 | Encoding | GTGTGTGCCAGGATGATCAATAGTGGCCTTTCTTTAACCTCT |
| | Probe 740 | AACCTC |
| 779 | Encoding | GTGTGTGCCAGGATGATCAATGTGCACCTTCACATTATCGCA |
| | Probe 741 | GCTTTC |
| 780 | Encoding | GTGTGTGCCAGGATGATCAATCACTAGGCCACACATTTTAGC |
| | Probe 742 | TGCC |
| 781 | Encoding | GTGTGTGCCAGGATGATCAATACTCCAAATATGTGAGCAAAT |
| | Probe 743 | GAGTTGGG |
| 782 | Encoding | GTGTGTGCCAGGATGATCAATAACGGTCCTCATTGATTCTCG |
| | Probe 744 | CTTT |
| 783 | Encoding | GTGTGTGCCAGGATGATCAATTCAGGACGTAGTTTTCAATCA |
| | Probe 745 | TTTCCTG |
| 784 | Encoding | GTGTGTGCCAGGATGATCAATGCCTCTTATGGACGGTGATAG |
| | Probe 746 | TTCCC |
| 785 | Encoding | GTGTGTGCCAGGATGATCAATAGTGCATGGTCCAGACTTTCT |
| | Probe 747 | TTAATTTCTTGG |
| 786 | Encoding | GTGTGTGCCAGGATGATCAATACCGAAAGGTCGACTTCAAAT |
| | Probe 748 | GGGTC |
| 787 | Encoding | GTGTGTGCCAGGATGATCAATTCCGACTGGAGTTTGTTTCCT |
| | Probe 749 | CATGTT |
| 788 | Encoding | GTGTGTGCCAGGATGATCAATGTCCTCTTCGATTTCTTTCTG |
| | Probe 750 | AAACTGGC |
| 789 | Encoding | GTGTGTGCCAGGATGATCAATGAACCAGAACAGAATCAATAA |
| | Probe 751 | TTGGTCTTGCAG |
| 790 | Encoding | GTGTGTGCCAGGATGATCAATTGATCTCTCTCTCATTGAGAG |
| | Probe 752 | GCCG |
| 791 | Encoding | GTGTGTGCCAGGATGATCAATAAAGAGTTCCCTCCTAAGGAG |
| | Probe 753 | TCCTG |
| 792 | Encoding | GTGTGTGCCAGGATGATCAATATACGAAAGTAAATGTCTTTG |
| | Probe 754 | GAGGTTC |
| 793 | Encoding | GTGTGTGCCAGGATGATCAATTCATATCAATGATGAGCATCT |
| | Probe 755 | GAAGACGG |
| 794 | Encoding | GTGTGTGCCAGGATGATCAATACGCCAGGGTAGACAATGAAA |
| | Probe 756 | GGTT |

### Example 17: HiPR-Cycle can Detect Genes in Communication with Microbiome

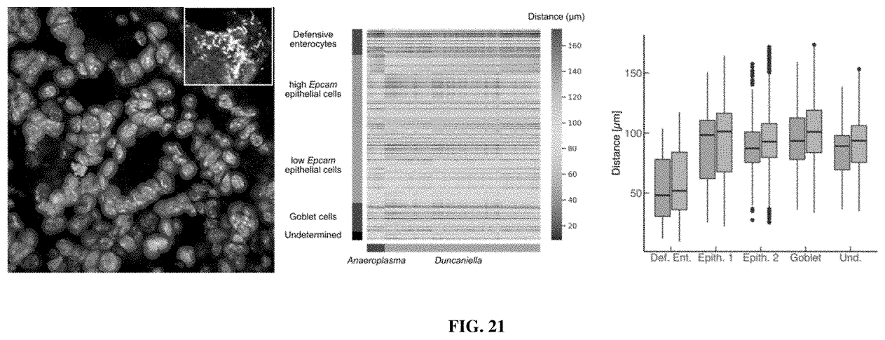

In this experiment we show the ability to detect the expression of multiple genes in relation to the microbial taxa (FIG. 20). Lypd8 is a gene that's known to confer resistance to the adhesion of gram-positive bacteria in the intestine.

Method

Fresh frozen colon tissue was embedded in Tissue-Tek O.C.T. compounds and sectioned at a thickness of 10 microns at −19° C. onto Ultrastick glass slides. Following sectioning, sections were covered with 2% formaldehyde and incubated in a chemical fume hood for 90 minutes at room temperature. Following fixation, samples were rinsed with 1×PBS, three times to remove the fixative. Specimens were placed in mailers with 70% ethanol and chilled to 4° C. for four hours to permeabilize the cell membrane.

After fixation, we added 10 μg/ml lysozyme to digest bacterial cell walls and incubated the sections for 30 minutes at 37° C.; sections were then washed with 1×PBS for 15 minutes at room temperature. We then added pre-encoding buffer to samples for 30 minutes at 37° C. An encoding buffer was synthesized to include probes for: mRNA from Ceacam20 (readout probe 3 [R3]), Myh11 (readout probe 10 [R10]), Lypd8 (readout probe 1 [R1]), Cd52 (readout probe 2 [R2]), Ubc (readout probe 7 [R7]), and Acta2 (readout probe 9 [R9]) (at a concentration of 400 nM per gene pool); 16S rRNA from Duncaniella (readout probe 1 [R1]), Bacteroides (readout probe 2 [R2]), Turicibacter (readout probe 3 [R3]), Akkermansia (readout probe 6 [R6]), Ruminococcus (readout probe 7 [R7]), Enterococcus (readout probe 8 [R8]), Anaeroplasma (readout probe 10 [R10]), and broad Eubacterium (readout probe 9 [R9]) (at a concentration of 200 nM per pool). The encoding probes were added to specimens. The specimens were incubated overnight (16 hours) at 37° C.

The following day, specimens were washed in HiPR-FISH wash buffer for 15 minutes at 48° C. followed by 5×SSC+Tween 20 (5 minutes, room temperature) to remove unbound encoding probes. A pre-amplification buffer was added to specimens for 30 minutes at room temperature. During this incubation, amplifier probes were annealed by heating to 95° C. for 2 minutes and allowed to cool to room temperature for 30 minutes. An amplification buffer was prepared by adding ten pairs of amplifier probes (90 nM each; stocks of probes at 9 μM) and readout probes (400 nM each) and incubated overnight at 30° C. at room temperature in the dark. Specimens were again washed with 2×SSC+Tween 20 at 42° C. for 15 minutes. Mammalian tissue was then cleared using TruView (Vector Laboratories) according to manufacturer instructions. Following the manufacturer-described wash step, specimens were incubated in 5×SSC+DAPI (1:1,000,000 dilution) for two minutes. The specimens were then mounted in Prolong Antifade.

Slides were imaged using a Zeiss widefield epifluorescence microscope (20Z air objective) with channels (excitation plus filters) set for dyes present in the experiment.

Encoding probes 757-928, as shown in Table 14 below, were used in this example. Amplifier probes 7-10, 29-30, and 39-44 (SEQ ID NO: 129-132, 795-796, 805-806, and 979-982), as shown in Table 2, were used in this example. Readout probes 1-10 (SEQ ID NO: 25-34), as shown in Table 3, were used in this example.

TABLE 14
Encoding probes used in Example 17.

| SEQ | Probe | |
|---|---|---|
| ID NO: | Name | Sequence |
| 246 | Encoding | TAGAGTTGATAGAGGGAGAAGCTGCCTCCCGTAGGAGT |
| | Probe 222 | |
| 807 | Encoding | TGTGGAGGGATTGAAGGATACGTGAGTTAGCCGATGCTTTTAGA |
| | Probe 757 | |
| 808 | Encoding | TGTGGAGGGATTGAAGGATATCAGGTCCGTGTCTCAGTACCTCA |
| | Probe 758 | |
| 809 | Encoding | TGTGGAGGGATTGAAGGATATCGATCCAGCTTCATGGAGTCCCC |
| | Probe 759 | |
| 810 | Encoding | TGTGGAGGGATTGAAGGATATGCTGTCGCCCCGGACGTAAGCCG |
| | Probe 760 | |
| 811 | Encoding | TGTGGAGGGATTGAAGGATACTGTTGAGCCCGGGTAAGGTTGGA |
| | Probe 761 | |
| 812 | Encoding | TGTGGAGGGATTGAAGGATATGGGCATTCATCGTTTACTGCCAC |
| | Probe 762 | |
| 813 | Encoding | TGTGGAGGGATTGAAGGATATGGGCATTCCGCGTACTTCTCTAG |
| | Probe 763 | |
| 814 | Encoding | TGTGGAGGGATTGAAGGATAGTCTGCATACGTGTTACTCACGGC |
| | Probe 764 | |
| 815 | Encoding | ATAGGAAATGGTGGTAGTGTATGGCGAATCCAGCTTCACGATCA |
| | Probe 765 | |
| 816 | Encoding | ATAGGAAATGGTGGTAGTGTCGTCTCTCTAGAGTCCTCAGCTAC |
| | Probe 766 | |
| 817 | Encoding | ATAGGAAATGGTGGTAGTGTGATCCCGAGTTATCGGCAGGTACC |
| | Probe 767 | |
| 818 | Encoding | ATAGGAAATGGTGGTAGTGTTCTCCCTATCCATCGAAGGTTACC |
| | Probe 768 | |
| 819 | Encoding | ATAGGAAATGGTGGTAGTGTCCTACGCTCGGATCCTCCGTAAAT |
| | Probe 769 | |
| 820 | Encoding | ATAGGAAATGGTGGTAGTGTGGAACTGTACTCAAGACAGCCTCA |
| | Probe 770 | |
| 821 | Encoding | ATAGGAAATGGTGGTAGTGTCGTCAGCGAGTATTCATCGTTATG |
| | Probe 771 | |
| 822 | Encoding | ATAGGAAATGGTGGTAGTGTGTGTTGTCTTACGACTATACTGTTAG |
| | Probe 772 | G |
| 823 | Encoding | AGAGTGAGTAGTAGTGGAGTAAGTCTTCTGCACTCAAGTCATGG |
| | Probe 773 | |
| 824 | Encoding | AGAGTGAGTAGTAGTGGAGTGGCGGCTCATCCATCAGTGATCGG |
| | Probe 774 | |
| 825 | Encoding | AGAGTGAGTAGTAGTGGAGTTACCCGCAGGCTCATCCATCACAC |
| | Probe 775 | |
| 826 | Encoding | AGAGTGAGTAGTAGTGGAGTGTAGGTACCGTCAATTGATAGTGTA |
| | Probe 776 | |
| 827 | Encoding | AGAGTGAGTAGTAGTGGAGTAGCCCCCAACACTTAGCACTCTAG |
| | Probe 777 | |
| 828 | Encoding | AGAGTGAGTAGTAGTGGAGTGGAAATCCGAACTGAGATTGGGAA |
| | Probe 778 | |
| 829 | Encoding | AGAGTGAGTAGTAGTGGAGTAAGACTTCATGTAGGCGAGTTCGT |
| | Probe 779 | |
| 830 | Encoding | AGAGTGAGTAGTAGTGGAGTCCTCCCTCTATCTCTAGAGGCCAG |
| | Probe 780 | |
| 831 | Encoding | TTGGAGGTGTAGGGAGTAAAGGAGACGGCTGACTCCTATAATCC |
| | Probe 781 | |
| 832 | Encoding | TTGGAGGTGTAGGGAGTAAATGAAATCATCTAGGCAAGCTCCGA |
| | Probe 782 | |
| 833 | Encoding | TTGGAGGTGTAGGGAGTAAAAGTCTCTAGACATGCGTCTAGACA |
| | Probe 783 | |
| 834 | Encoding | TTGGAGGTGTAGGGAGTAAAAAATCACTACCAACAGAGCTTATG |
| | Probe 784 | |
| 835 | Encoding | TTGGAGGTGTAGGGAGTAAAGGCGGCTTTCACATCAGACTTATACT |
| | Probe 785 | |
| 836 | Encoding | TTGGAGGTGTAGGGAGTAAAAGTGTCAGTTGCAGACCAGAGTCG |
| | Probe 786 | |
| 837 | Encoding | TTGGAGGTGTAGGGAGTAAAACGCACCTGTCTCAGCGTCCCGCT |
| | Probe 787 | |
| 838 | Encoding | TTGGAGGTGTAGGGAGTAAAGAATGATGGCAACTAATGACATCC |
| | Probe 788 | |
| 839 | Encoding | AGGGTGTGTTTGTAAAGGGTGGAGGCATAAGGGCCATACTGTGG |
| | Probe 789 | |
| 840 | Encoding | AGGGTGTGTTTGTAAAGGGTGCGCCGAAGAGTCGCATGCTTAGT |
| | Probe 790 | |
| 841 | Encoding | AGGGTGTGTTTGTAAAGGGTGCAACTGCCAGGACTACAGGGCAT |
| | Probe 791 | |
| 842 | Encoding | AGGGTGTGTTTGTAAAGGGTCGCTGGCTTCAGATACTTCGGCAC |
| | Probe 792 | |
| 843 | Encoding | AGGGTGTGTTTGTAAAGGGTCACCCTCTCGATATCTACGCAAAA |
| | Probe 793 | |
| 844 | Encoding | AGGGTGTGTTTGTAAAGGGTGTTCGAAGACCTTCATTCCCCGTG |
| | Probe 794 | |
| 845 | Encoding | AGGGTGTGTTTGTAAAGGGTCAACCCCATTGTGAATGATTCTGCT |
| | Probe 795 | |
| 846 | Encoding | AGGGTGTGTTTGTAAAGGGTTTATTCGATACTATGCGGTATTTTA |
| | Probe 796 | |
| 847 | Encoding | AGGTTAGGTTGAGAATAGGAACGCGCGGGCCCATCTCATAGGCC |
| | Probe 797 | |
| 848 | Encoding | AGGTTAGGTTGAGAATAGGAATACCCTCTATGAGGCAGGTTCGG |
| | Probe 798 | |
| 849 | Encoding | AGGTTAGGTTGAGAATAGGAACTACCCCTACACCTAGTATTCTAG |
| | Probe 799 | |
| 850 | Encoding | AGGTTAGGTTGAGAATAGGAAGTTACAGTCCAGAGAATCGCGAA |
| | Probe 800 | |
| 851 | Encoding | AGGTTAGGTTGAGAATAGGAATCACTCCAGCTTCATGTAGGGCT |
| | Probe 801 | |
| 852 | Encoding | AGGTTAGGTTGAGAATAGGATGTCCGAACTGAGACAAGTTTAAC |
| | Probe 802 | |
| 853 | Encoding | AGGTTAGGTTGAGAATAGGAACGCACCTGTCTTCCTGTCCCGCT |
| | Probe 803 | |
| 854 | Encoding | AGGTTAGGTTGAGAATAGGAAGCCCTGAAGACAGAGCTTTAGTT |
| | Probe 804 | |
| 855 | Encoding | GATGATGTAGTAGTAAGGGTAAAAATGCAGTCTGAAGGTTGTCG |
| | Probe 805 | |
| 856 | Encoding | GATGATGTAGTAGTAAGGGTCGACCCTCTCTCAGAGGCAGGAAC |
| | Probe 806 | |
| 857 | Encoding | GATGATGTAGTAGTAAGGGTCATGTCATTATCGTCCCTGATATTT |
| | Probe 807 | |
| 858 | Encoding | GATGATGTAGTAGTAAGGGTCGCCCCATCTCTGAGCGAATTAGA |
| | Probe 808 | |
| 859 | Encoding | GATGATGTAGTAGTAAGGGTGATCCTCTCTCAGAGGCAGGTACG |
| | Probe 809 | |
| 860 | Encoding | GATGATGTAGTAGTAAGGGTTCGTCTGATCTGCGATTACTAGCATA |
| | Probe 810 | A |
| 861 | Encoding | GATGATGTAGTAGTAAGGGTGTCCCTCTAGAGTGCTCTTGCCAT |
| | Probe 811 | |
| 862 | Encoding | GATGATGTAGTAGTAAGGGTAAACCTATCTCTAGGCTATGCAATG |
| | Probe 812 | |
| 863 | Encoding | GCACATGCTCCGTGTAGAATAGACACCCTTTAGAAACCAATGGAC |
| | Probe 813 | ACC |
| 864 | Encoding | GCACATGCTCCGTGTAGAATAGTTGGTTGGGAACTCGATATTCGGT |
| | Probe 814 | |
| 865 | Encoding | GCACATGCTCCGTGTAGAATAACCCACCTGGTATGGAGTCTGCC |
| | Probe 815 | |
| 866 | Encoding | GCACATGCTCCGTGTAGAATAAGTGCATTAGTGTTACAGTAGAAA |
| | Probe 816 | ACTGCC |
| 867 | Encoding | GCACATGCTCCGTGTAGAATATCACCAGCTTGATTGATACAGGATC |
| | Probe 817 | AGG |
| 868 | Encoding | GCACATGCTCCGTGTAGAATAGTTGCACCTTAGAGGAGTGCTCATG |
| | Probe 818 | |
| 869 | Encoding | GCACATGCTCCGTGTAGAATAGTGAGCATCGATTGTGAATGTTCCT |
| | Probe 819 | GT |
| 870 | Encoding | GCACATGCTCCGTGTAGAATAAAGATGCGCTCATTCAACACAAGC |
| | Probe 820 | |
| 871 | Encoding | GCACATGCTCCGTGTAGAATATCCCTGAGAGAAGGATCACTGTCCA |
| | Probe 821 | |
| 872 | Encoding | GCACATGCTCCGTGTAGAATAGAGCAGGAGACATAGAGGATGACT |
| | Probe 822 | GGA |
| 873 | Encoding | GCACATGCTCCGTGTAGAATATTGCAGGATCCCGATAACGATGC |
| | Probe 823 | |
| 874 | Encoding | GCACATGCTCCGTGTAGAATAAGTCACTCATAGGGCCCAATATCAT |
| | Probe 824 | C |
| 875 | Encoding | GCACATGCTCCGTGTAGAATACTCGCAGTGTTACTTCTCTGAACCT |
| | Probe 825 | |
| 876 | Encoding | GCACATGCTCCGTGTAGAATAAGTCTTTTTCAAGCACTTGGACTTT |
| | Probe 826 | GAC |
| 877 | Encoding | GCACATGCTCCGTGTAGAATATCAGCTCTGATTGACTGTGAGGGT |
| | Probe 827 | |
| 878 | Encoding | GCACATGCTCCGTGTAGAATACCAGAGGGTCTTGTTATCTGCAGAC |
| | Probe 828 | AG |
| 879 | Encoding | GCACATGCTCCGTGTAGAATAGGAGGGTGATTTCCACTTGGTCG |
| | Probe 829 | |
| 880 | Encoding | GCACATGCTCCGTGTAGAATACTCCTTGTACAGCTGTAAATGCCCT |
| | Probe 830 | |
| 881 | Encoding | GCACATGCTCCGTGTAGAATAAGTGGGTTCTGTTTTGGGATGACAG |
| | Probe 831 | T |
| 882 | Encoding | GCACATGCTCCGTGTAGAATAGTAACACAGTCATTGGTTTGTTGTG |
| | Probe 832 | C |
| 883 | Encoding | GCACATGCTCCGTGTAGAATATAAGCCTCAATAGTGCTGACCAC |
| | Probe 833 | |
| 884 | Encoding | GCACATGCTCCGTGTAGAATACCGACCTGTACATGCCCTCATGTTC |
| | Probe 834 | |
| 885 | Encoding | GCACATGCTCCGTGTAGAATAACGTGCACTTCACACAGGTAAGAC |
| | Probe 835 | C |
| 886 | Encoding | GCACATGCTCCGTGTAGAATAACCGGTGATCTTGCAGTAAAGACTA |
| | Probe 836 | TGGT |
| 887 | Encoding | TGAACTCGGCGGGTTAGGAATCGAGAAGGACAGATGATACTACCT |
| | Probe 837 | TCAAGATG |
| 888 | Encoding | TGAACTCGGCGGGTTAGGAATCTCGAGTGATGTCACATTGTCATTT |
| | Probe 838 | AGCG |
| 889 | Encoding | TGAACTCGGCGGGTTAGGAATTTTGCCATTGGATAAAAATGTGTAG |
| | Probe 839 | CTG |
| 890 | Encoding | TGAACTCGGCGGGTTAGGAATATATGGCACCTACAATGTAACCAG |
| | Probe 840 | T |
| 891 | Encoding | TGAACTCGGCGGGTTAGGAATCCCGCCCTCTGAGTTTGCTCTTGA |
| | Probe 841 | |
| 892 | Encoding | TGAACTCGGCGGGTTAGGAATAGAGCTAGGTTGGTTGTCAAGTCG |
| | Probe 842 | C |
| 893 | Encoding | TGAACTCGGCGGGTTAGGAATGTCCTCTGAGATCATAGACTCATGC |
| | Probe 843 | TTG |
| 894 | Encoding | TGAACTCGGCGGGTTAGGAATTCGCTGTCGAATAGCCCTAGACTTT |
| | Probe 844 | TCC |
| 895 | Encoding | TGAACTCGGCGGGTTAGGAATAGAGATCATCCATAACCAAGATGT |
| | Probe 845 | CATCC |
| 896 | Encoding | TGAACTCGGCGGGTTAGGAATGTACCCGTCCAACTTTGATACGTGG |
| | Probe 846 | |
| 897 | Encoding | TGAACTCGGCGGGTTAGGAATCCTGCAATGGCTTTACTTTGGTGAA |
| | Probe 847 | GAG |
| 898 | Encoding | TGAACTCGGCGGGTTAGGAATACGAGCTTCTTCTTAGAGTCTGAGA |
| | Probe 848 | GC |
| 899 | Encoding | TGAACTCGGCGGGTTAGGAATACTCCTTCTCTAGCTGTAGTTTCTG |
| | Probe 849 | TCT |
| 900 | Encoding | TGAACTCGGCGGGTTAGGAATCAACCAGCACTAGAAATGCATCCA |
| | Probe 850 | G |
| 901 | Encoding | TGAACTCGGCGGGTTAGGAATGAGGCGAATCTTCTTTAGGGCATTG |
| | Probe 851 | TTT |
| 902 | Encoding | TGAACTCGGCGGGTTAGGAATCGGTTGCGTACTCTATCACTCATGG |
| | Probe 852 | C |
| 903 | Encoding | TGAACTCGGCGGGTTAGGAATGGTCGATCTTTTCTGAGTAGATGGG |
| | Probe 853 | TAGG |
| 904 | Encoding | TGAACTCGGCGGGTTAGGAATTCCTCCTCTTGTAGGTCTGAGATAT |
| | Probe 854 | GGC |
| 905 | Encoding | TGAACTCGGCGGGTTAGGAATGGGCCGTTCGGAAGAAGATTTTGC |
| | Probe 855 | TC |
| 906 | Encoding | TGAACTCGGCGGGTTAGGAATCAGTTCACGATCTTGTAGCATGCTT |
| | Probe 856 | C |
| 907 | Encoding | TGAACTCGGCGGGTTAGGAATAGATCATTGAAGCCCATGATAGAC |
| | Probe 857 | ATGG |
| 908 | Encoding | TGAACTCGGCGGGTTAGGAATCAGCAAGGCCTTGTTTACACGGC |
| | Probe 858 | |
| 909 | Encoding | TGAACTCGGCGGGTTAGGAATACTCATCAGTAATTTTCAGGTCTCG |
| | Probe 859 | TTCC |
| 910 | Encoding | TGAACTCGGCGGGTTAGGAATAGGTGGAACATCTCATCATCTTGTG |
| | Probe 860 | C |
| 911 | Encoding | GCTCGACGTTCCTTTGCAACACCCACAGGAATTGACAAGTAGAGTT |
| | Probe 861 | GCT |
| 912 | Encoding | GCTCGACGTTCCTTTGCAACACACGGTTGCGTACATTCAACCGAC |
| | Probe 862 | |
| 913 | Encoding | GCTCGACGTTCCTTTGCAACAGTGAGGTGCTATTGTACGTATAACA |
| | Probe 863 | CTGTG |
| 914 | Encoding | GCTCGACGTTCCTTTGCAACATGTGTCTAACTGGAAGGTATATGAA |
| | Probe 864 | GAGAAAGG |
| 915 | Encoding | GCTCGACGTTCCTTTGCAACAGTCAAAAGCCATGGATTTAGCTTCT |
| | Probe 865 | CAG |
| 916 | Encoding | GCTCGACGTTCCTTTGCAACATAACAGGGAGTCTACAACTGTGATG |
| | Probe 866 | G |
| 917 | Encoding | GCTCGACGTTCCTTTGCAACAGTTGCAACACTTTAATAAAGGAGAT |
| | Probe 867 | ACTGTGGC |
| 918 | Encoding | GCTCGACGTTCCTTTGCAACACGGCCAAACATATCAGAACAGATTG |
| | Probe 868 | TCTAGACC |
| 919 | Encoding | GCTCGACGTTCCTTTGCAACAGACCATGTCAGTTACATTCTGTGTTC |
| | Probe 869 | CG |
| 920 | Encoding | GCTCGACGTTCCTTTGCAACAATCAGTGGAGTTGATAGAGGACTCC |
| | Probe 870 | AC |
| 921 | Encoding | GCTCGACGTTCCTTTGCAACATGTCATTGTTCTCCTTCGTAACACTT |
| | Probe 871 | TTGG |
| 922 | Encoding | GCTCGACGTTCCTTTGCAACATCTGTGTTTTGTTGTGACCATAGCAA |
| | Probe 872 | G |
| 923 | Encoding | GCTCGACGTTCCTTTGCAACACCGTGCCGAAGATAGAGGAGGTGA |
| | Probe 873 | |
| 924 | Encoding | GCTCGACGTTCCTTTGCAACACGGAGAGCCATTTACCATCTCCGC |
| | Probe 874 | |
| 925 | Encoding | GCTCGACGTTCCTTTGCAACACACGTGGTCGAGTTGGTATACTCAG |
| | Probe 875 | T |
| 926 | Encoding | GCTCGACGTTCCTTTGCAACAGCCGGGAAGTTTTGATGCCTGGTC |
| | Probe 876 | |
| 927 | Encoding | GCTCGACGTTCCTTTGCAACAGTCAACTTGTTCTGGTACACATGCA |
| | Probe 877 | |
| 928 | Encoding | GCTCGACGTTCCTTTGCAACACGAGTTCTCGGTGCAATTCGATGC |
| | Probe 878 | |
| 929 | Encoding | GCTCGACGTTCCTTTGCAACAGAATGGTCATCAAATAGATGGACAG |
| | Probe 879 | TGAAG |
| 930 | Encoding | GCTCGACGTTCCTTTGCAACACTTGGACTGCTCATTGCATTCAGTG |
| | Probe 880 | |
| 931 | Encoding | GCTCGACGTTCCTTTGCAACATTCTGTTGGTGATTGGTGGGATAGT |
| | Probe 881 | |
| 932 | Encoding | GCTCGACGTTCCTTTGCAACACCAGGCATTGCAGGACTCTCCTT |
| | Probe 882 | |
| 933 | Encoding | GCTCGACGTTCCTTTGCAACATGTAACTCTCCAACTGTTGTGTTCCC |
| | Probe 883 | |
| 934 | Encoding | GCTCGACGTTCCTTTGCAACATCGCCAGGAACCTCAGAACAGCA |
| | Probe 884 | |
| 935 | Encoding | CGTCGGAGTGGGTTCAGTCTATCTAGAGGCACATTAAGGTATTGGC |
| | Probe 885 | AAA |
| 936 | Encoding | CGTCGGAGTGGGTTCAGTCTACAGCCAAGGATCCTGTTTGTATCTG |
| | Probe 886 | AATCA |
| 937 | Encoding | CGTCGGAGTGGGTTCAGTCTATCCTGCTGTTTTTGTTAGTACCAGA |
| | Probe 887 | AGC |
| 938 | Encoding | CGTCGGAGTGGGTTCAGTCTAATCCAGCTTTGAGAGATGAGTTCAG |
| | Probe 888 | G |
| 939 | Encoding | CGTCGGAGTGGGTTCAGTCTAGTTCCAGAAGAATGATAGTGAGGA |
| | Probe 889 | AGAGG |
| 940 | Encoding | CGTCGGAGTGGGTTCAGTCTAGGACCTTGGATATCTGCTATCAACC |
| | Probe 890 | CT |
| 941 | Encoding | CGTCGGAGTGGGTTCAGTCTAAACGGATGTCTCTCGCTACTGATGG |
| | Probe 891 | |
| 942 | Encoding | CGTCGGAGTGGGTTCAGTCTACTTGCTCTTCATCCTGAAATCTTCCT |
| | Probe 892 | G |
| 943 | Encoding | CGTCGGAGTGGGTTCAGTCTAGGCCATCGATGATGGATGAGGCC |
| | Probe 893 | |
| 944 | Encoding | CGTCGGAGTGGGTTCAGTCTATCAATAACTTTATTGTGCCCTAGCT |
| | Probe 894 | GGG |
| 945 | Encoding | CGTCGGAGTGGGTTCAGTCTACGTGGCCACTTTGAACCTGGCTG |
| | Probe 895 | |
| 946 | Encoding | CGTCGGAGTGGGTTCAGTCTATTCAAGAGGAAACTGCAGGCACC |
| | Probe 896 | |
| 947 | Encoding | CGTCGGAGTGGGTTCAGTCTAGGCGCCATTGGCTGTCAACTTTAGC |
| | Probe 897 | |
| 948 | Encoding | CGTCAGGTGAGCATCTTACATCAGTTCCTGTTTGACCTTCTTGGTGG |
| | Probe 898 | TAACACTCCCTTCATGTAACGTCAGGTGAGC |
| 949 | Encoding | CGTCAGGTGAGCATCTTACATTAGTTTGCCTTGACATTCTCAATGG |
| | Probe 899 | TGTCACTGCCGTTCATGTAACGTCAGGTGAGC |
| 950 | Encoding | CGTCAGGTGAGCATCTTACATAGGTTGTCCTGGATCTTTGCCTTGA |
| | Probe 900 | CATTCTCTTACTTCATGTAACGTCAGGTGAGC |
| 951 | Encoding | CGTCAGGTGAGCATCTTACATTACGTTTTGCCTGTCAGGGTCTTCA |
| | Probe 901 | CAAAGATCACGTTCATGTAACGTCAGGTGAGC |
| 952 | Encoding | CGTCAGGTGAGCATCTTACATCACCAGGGTGGACTCTTTCTGGATG |
| | Probe 902 | TTGTAGTCACTTTCATGTAACGTCAGGTGAGC |
| 953 | Encoding | CGTCAGGTGAGCATCTTACATCCACTTCACAAAGATCTGCATCCCA |
| | Probe 903 | CCTCTGAGCGCTTCATGTAACGTCAGGTGAGC |
| 954 | Encoding | CGTCAGGTGAGCATCTTACATTACGTCTTGCCTGTCAGGGTCTTCA |
| | Probe 904 | CAAAGATCACGTTCATGTAACGTCAGGTGAGC |
| 955 | Encoding | GTGTGTGCCAGGATGATCAATCGGAGGGCTACAAGTTAAGGGTAG |
| | Probe 905 | CA |
| 956 | Encoding | GTGTGTGCCAGGATGATCAATTCCGGGCCACCCTATAATAAATGAT |
| | Probe 906 | TCTCA |
| 957 | Encoding | GTGTGTGCCAGGATGATCAATTACATGCCGTGTTCTATCGGATACT |
| | Probe 907 | TC |
| 958 | Encoding | GTGTGTGCCAGGATGATCAATGTGACGAAGCTCGTTATAGAAAGA |
| | Probe 908 | GTGG |
| 959 | Encoding | GTGTGTGCCAGGATGATCAATCCTCGTTGTTAGCATAGAGATCCTT |
| | Probe 909 | CC |
| 960 | Encoding | GTGTGTGCCAGGATGATCAATCAGCAGCACAATACCAGTTGTACGT |
| | Probe 910 | |
| 961 | Encoding | GTGTGTGCCAGGATGATCAATGGTACCATTACTCCCTGATGTCTGG |
| | Probe 911 | |
| 962 | Encoding | GTGTGTGCCAGGATGATCAATCGACGGCAGTAGTCACGAAGGAAT |
| | Probe 912 | AG |
| 963 | Encoding | GTGTGTGCCAGGATGATCAATAACGCCTTAGGGTTCAGTGGTGC |
| | Probe 913 | |
| 964 | Encoding | GTGTGTGCCAGGATGATCAATGGTGACAGAGTACTTGCGTTCTGGA |
| | Probe 914 | G |
| 965 | Encoding | GTGTGTGCCAGGATGATCAATATCGCACGTTGTGAGTCACACCA |
| | Probe 915 | |
| 966 | Encoding | GTGTGTGCCAGGATGATCAATGACACTCCATCCCAATGAAAGATG |
| | Probe 916 | G |
| 967 | Encoding | GTGTGTGCCAGGATGATCAATAAGATGAGGTAGTCGGTGAGATCT |
| | Probe 917 | C |
| 968 | Encoding | GTGTGTGCCAGGATGATCAATTAATTCAAAGTCCAGAGCTACATAG |
| | Probe 918 | C |
| 969 | Encoding | GTGTGTGCCAGGATGATCAATAGTGGCAGTTCGTAGCTCTTCTCC |
| | Probe 919 | |
| 970 | Encoding | GTGTGTGCCAGGATGATCAATCCCGGGACTTAGAAGCATTTGCGG |
| | Probe 920 | |
| 971 | Encoding | GTGTGTGCCAGGATGATCAATTACATGCTGTTATAGGTGGTTTCGT |
| | Probe 921 | G |
| 972 | Encoding | GTGTGTGCCAGGATGATCAATTCGCCTGGGAGCATCATCACCAG |
| | Probe 922 | |
| 973 | Encoding | GTGTGTGCCAGGATGATCAATGAACTCCTTGATGTCACGGACAATC |
| | Probe 923 | TC |
| 974 | Encoding | GTGTGTGCCAGGATGATCAATCGAGGAAGAGAGTCTCTGGGCAG |
| | Probe 924 | |
| 975 | Encoding | GTGTGTGCCAGGATGATCAATCGGTCTGTCAGCAGTGTCGGATG |
| | Probe 925 | |
| 976 | Encoding | GTGTGTGCCAGGATGATCAATTTCCGTTCGTTTCCAATGGTGATC |
| | Probe 926 | |
| 977 | Encoding | GTGTGTGCCAGGATGATCAATAGTGCGTCAGGATCCCTCTCTTG |
| | Probe 927 | |
| 978 | Encoding | GTGTGTGCCAGGATGATCAATCAGCAGAGGCATAGAGGGACAGC |
| | Probe 928 | |

### Example 18: HiPR-Cycle can Inform Maps of the Microbiome and Host Cells

In here, we show the ability to create spatial maps illustrating the distance between host cells and microbes (FIG. 21).

Method

Fresh frozen colon tissue was embedded in Tissue-Tek O.C.T. compound and sectioned at a thickness of 10 microns at −19° C. onto Ultrastick glass slides. Following sectioning, sections were covered with 2% formaldehyde and incubated in a chemical fume hood for 90 minutes at room temperature. Following fixation, samples were rinsed with 1×PBS, three times to remove the fixative. Specimens were placed in mailers with 70% ethanol and chilled to 4° C. for four hours to permeabilize the cell membrane.

After fixation, we added 10 μg/ml lysozyme to digest bacterial cell walls and incubated the sections for 30 minutes at 37° C.; sections were then washed with 1×PBS for 15 minutes at room temperature. We then added pre-encoding buffer to samples for 30 minutes at 37° C. An encoding buffer was synthesized to include probes for: mRNA from Hif1a (R3), Muc2 (R10), Sprr2a1 (R1), Epcam (R2), Mki67 (R7), and Acta2 (R9) (at a concentration of 400 nM per gene pool); 16S rRNA from Duncaniella (R1), Bacteroides (R2), Turicibacter (R3), Akkermansia (R6), Ruminococcus (R7), Enterococcus (R8), Anaeroplasma (R10), and broad Eubacterium (R9) (at a concentration of 200 nM per pool). The encoding probes were added to specimens. The specimens were incubated overnight (16 hours) at 37° C.

The following day, specimens were washed in HiPR-FISH wash buffer for 15 minutes at 48° C. followed by 5×SSC+Tween 20 (5 minutes, room temperature) to remove unbound encoding probes. A pre-amplification buffer was added to specimens for 30 minutes at room temperature. During this incubation, amplifier probes were annealed by heating to 95° C. for 2 minutes and allowed to cool to room temperature for 30 minutes. An amplification buffer was prepared by adding ten pairs of amplifier probes (90 nM each; stocks of probes at 9 μM) and readout probes (400 nM each) and incubated overnight at 30° C. at room temperature in the dark. Specimens were again washed with 2×SSC+Tween 20 at 42° C. for 15 minutes. Mammalian tissue was then cleared using TruView (Vector Laboratories) according to manufacturer instructions. Following the manufacturer-described wash step, specimens were incubated in 5×SSC+DAPI (1:1,000,000 dilution) for two minutes. The specimens were then mounted in Prolong Antifade.

Slides were imaged using a Zeiss i880 confocal in lambda mode with lasers set for 405 nm, 488 nm, 514 nm, 561 nm, and 633 nm excitation modes.

We processed the images (five matched .CZI files) using our standard HiPR-FISH pipeline to segment bacterial cells (using the maximum projection of all 95 collected channels), determine their spectral signature, and convert the spectra to a barcode to reveal the taxonomic identity (here, genus). Cell types were determined by examining the intensity corresponding to label transcripts (e.g. examining channels corresponding to high Alexa Fluor 488 signal. we determined cells containing R1-tagged transcripts and hence those with high Sprr2a1 expression).

We assigned a cell type to each identified mammalian cell and determined the centroid for each mammalian cell and each microbial cell. The distances between each mammalian cell and microbial cell were determined and used to assemble a heat map, as well as examine broader trends of association between cell types/states and microbes.

Encoding probes 757-796, 905-928 (SEQ ID NO: 807-846, 955-978), as shown in Table 14, and encoding probes 983-1084, as shown in Table 15 below, were used in this example. Amplifier probes 7-10, 17-18, 27-30, and 39-40 (SEQ ID NO: 129-132, 281-282, 385-386, 795-796, and 805-806), as shown in Table 2, were used in this example. Readout probes 1-10 (SEQ ID NO: 25-34), as shown in Table 3, were used in this example.

TABLE 15
Encoding probes used in Example 18.

| SEQ ID | Probe | |
|---|---|---|
| NO: | Name | Sequence |
| 983 | Encoding Probe | GCACATGCTCCGTGTAGAATATCACTGCATGCTAAATCGGAG |
| | 929 | GGTATT |
| 984 | Encoding Probe | GCACATGCTCCGTGTAGAATAGTAATCATGGTGAGTTTTGGT |
| | 930 | CAGATGA |
| 985 | Encoding Probe | GCACATGCTCCGTGTAGAATACCTAACGCTCAGTTAACTTGA |
| | 931 | TCCAAAG |
| 986 | Encoding Probe | GCACATGCTCCGTGTAGAATAGTIGTCGTGCTGAATAATACC |
| | 932 | ACTTACAAC |
| 987 | Encoding Probe | GCACATGCTCCGTGTAGAATACGAGCATCTCTAGACTTTTCT |
| | 933 | TTTCGACG |
| 988 | Encoding Probe | GCACATGCTCCGTGTAGAATAGTCCATCTCCAAATCTAAATC |
| | 934 | AGTGTCCTG |
| 989 | Encoding Probe | GCACATGCTCCGTGTAGAATAACAGTCCAGTTAGTTCAAACT |
| | 935 | GAGTTAACC |
| 990 | Encoding Probe | GCACATGCTCCGTGTAGAATAAAGCAGGAAAGTGACATAGT |
| | 936 | AGGTGCA |
| 991 | Encoding Probe | GCACATGCTCCGTGTAGAATATATACAGAAGCTTTATCAAGA |
| | 937 | TGTGAGC |
| 992 | Encoding Probe | GCACATGCTCCGTGTAGAATATCATTCAACCCAGACATATCC |
| | 938 | ACCTCT |
| 993 | Encoding Probe | GCACATGCTCCGTGTAGAATACCCCTTGGAGAATTGCTCTCT |
| | 939 | AATGGTGA |
| 994 | Encoding Probe | GCACATGCTCCGTGTAGAATAATCGGCTTTCAGATAAAAAC |
| | 940 | AGTCCATCTG |
| 995 | Encoding Probe | GCACATGCTCCGTGTAGAATAGAGTCAGGTGAACTTTGTCTA |
| | 941 | GTGCT |
| 996 | Encoding Probe | GCACATGCTCCGTGTAGAATATACGGAGCATTAACTTCACAA |
| | 942 | TCGTAACTG |
| 997 | Encoding Probe | GCACATGCTCCGTGTAGAATACCGAGCTTGTATCCTCTGATT |
| | 943 | CAACTTTGG |
| 998 | Encoding Probe | GCACATGCTCCGTGTAGAATAGACGGGCATGGTAAAAGAAA |
| | 944 | GTCCCA |
| 999 | Encoding Probe | GCACATGCTCCGTGTAGAATATTTGGAACGTAACTGGAAATC |
| | 945 | ATCATCC |
| 1000 | Encoding Probe | GCACATGCTCCGTGTAGAATAGTGACTGAGGTTGGTTACTGT |
| | 946 | TGGTAT |
| 1001 | Encoding Probe | GCACATGCTCCGTGTAGAATACGACCGTTCCATTCTGTTCAC |
| | 947 | TAGATATGA |
| 1002 | Encoding Probe | GCACATGCTCCGTGTAGAATAAAAGTCTGTCTGTTCTATGAC |
| | 948 | TCTCTTTCC |
| 1003 | Encoding Probe | GCACATGCTCCGTGTAGAATATAATTAATGCAACCTCTTGAT |
| | 949 | TCAGTGC |
| 1004 | Encoding Probe | GCACATGCTCCGTGTAGAATATTTGTTAGGAGTGTTTACGTT |
| | 950 | TTCCTGA |
| 1005 | Encoding Probe | GCACATGCTCCGTGTAGAATATCAGGTCGTTTCTTGAGGTAC |
| | 951 | TTGGG |
| 1006 | Encoding Probe | GCACATGCTCCGTGTAGAATAATTCCCATCAACTCAGTAATT |
| | 952 | CTTTCATCACAG |
| 1007 | Encoding Probe | CGTCGGAGTGGGTTCAGTCTACTGGTCCAGAGTCACAAAAA |
| | 953 | GCTGCATGAC |
| 1008 | Encoding Probe | CGTCGGAGTGGGTTCAGTCTACAGTTGCACGTGTCATATTTG |
| | 954 | CACCTCTTG |
| 1009 | Encoding Probe | CGTCGGAGTGGGTTCAGTCTAGTACATGAAGATCTGTGAGCT |
| | 955 | TGGGCAAGC |
| 1010 | Encoding Probe | CGTCGGAGTGGGTTCAGTCTAAAGACCATGGTGGTAGCAGG |
| | 956 | AACACTTAGC |
| 1011 | Encoding Probe | CGTCGGAGTGGGTTCAGTCTATGCAGATCTCATCAGTGGGAA |
| | 957 | CAGATCCTC |
| 1012 | Encoding Probe | CGTCGGAGTGGGTTCAGTCTATCTTCCCTTCATCTGGATGGC |
| | 958 | ATTCGATTT |
| 1013 | Encoding Probe | CGTCGGAGTGGGTTCAGTCTAGAGTGGTAGTTTCCGTTGGAA |
| | 959 | CAGTGAAGG |
| 1014 | Encoding Probe | CGTCGGAGTGGGTTCAGTCTAGTTCACCTGGGCCGTAGAACA |
| | 960 | TCTCATTCA |
| 1015 | Encoding Probe | CGTCGGAGTGGGTTCAGTCTACAGTTACACAGCCACCAGGTC |
| | 961 | TCATTAACC |
| 1016 | Encoding Probe | CGTCGGAGTGGGTTCAGTCTAACAGTTACCCTGGTAACTGTA |
| | 962 | GTAGAGCCC |
| 1017 | Encoding Probe | CGTCGGAGTGGGTTCAGTCTAGGCATCACAGTGGTAGTTGTC |
| | 963 | AATGTAGAC |
| 1018 | Encoding Probe | CGTCGGAGTGGGTTCAGTCTAGGAGATGTTCACCACAATGTT |
| | 964 | GATGCCAGA |
| 1019 | Encoding Probe | CGTCGGAGTGGGTTCAGTCTATTCACAGTCAGAGATGATCTT |
| | 965 | CCCACTGGG |
| 1020 | Encoding Probe | CGTCGGAGTGGGTTCAGTCTACACATGTCTTCACACAGACGT |
| | 966 | CATAGCCAG |
| 1021 | Encoding Probe | CGTCGGAGTGGGTTCAGTCTATCACTTCGAATCCCAACAAAC |
| | 967 | ATGTGGGGC |
| 1022 | Encoding Probe | CGTCGGAGTGGGTTCAGTCTACTCCCCAGGCTTCAGAATAAT |
| | 968 | GTACTGCTG |
| 1023 | Encoding Probe | CGTCGGAGTGGGTTCAGTCTAAATCACTTGGGTTGAAGTCGG |
| | 969 | GACAGGTGA |
| 1024 | Encoding Probe | CGTCGGAGTGGGTTCAGTCTAAGTACATGGCAAAAGTCCCA |
| | 970 | CAGGACCCAA |
| 1025 | Encoding Probe | GCTCGACGTTCCTTTGCAACAAGATGGAATCTGAGTAGTAGA |
| | 971 | TGTCTGCTT |
| 1026 | Encoding Probe | GCTCGACGTTCCTTTGCAACACATCAGGATTTTGTGAGATGT |
| | 972 | CAGAATAGC |
| 1027 | Encoding Probe | GCTCGACGTTCCTTTGCAACACACAGAACAGGCTTTATATGC |
| | 973 | TTTCTCGG |
| 1028 | Encoding Probe | GCTCGACGTTCCTTTGCAACAATACCCACTCGATATTTTATCT |
| | 974 | TTTGTCCTAGC |
| 1029 | Encoding Probe | GCTCGACGTTCCTTTGCAACATCAGCATGGACAATTACTGAA |
| | 975 | CTCTATAGC |
| 1030 | Encoding Probe | GCTCGACGTTCCTTTGCAACACCCGTCTGACAGTAACTATAC |
| | 976 | CATAGGTGACA |
| 1031 | Encoding Probe | GCTCGACGTTCCTTTGCAACAGTTGGATACCTATGGAAAAGG |
| | 977 | CAATTTGAC |
| 1032 | Encoding Probe | GCTCGACGTTCCTTTGCAACATAGCGGCTAAACATTTATCAC |
| | 978 | CTGTCA |
| 1033 | Encoding Probe | GCTCGACGTTCCTTTGCAACAAATACCATGTTATTCTGTAAA |
| | 979 | GACTGGC |
| 1034 | Encoding Probe | GCTCGACGTTCCTTTGCAACAACGTGGTAGTAAGACATCGTA |
| | 980 | CTTGAGT |
| 1035 | Encoding Probe | GCTCGACGTTCCTTTGCAACACCAGGCTGACTGATGATAGTG |
| | 981 | TTTTACC |
| 1036 | Encoding Probe | GCTCGACGTTCCTTTGCAACAAGAGCAGAAATCTGCTAGTGA |
| | 982 | TTGTCGTA |
| 1037 | Encoding Probe | GCTCGACGTTCCTTTGCAACATAATGCTGAGTAGTTATGAGA |
| | 983 | AAGTGTCAC |
| 1038 | Encoding Probe | GCTCGACGTTCCTTTGCAACAAACGAGATTCCTCTGTAATGT |
| | 984 | AAAGTCAGGG |
| 1039 | Encoding Probe | GCTCGACGTTCCTTTGCAACAAACGCTAGAATCTTGCCCTTA |
| | 985 | TTAACTTGC |
| 1040 | Encoding Probe | GCTCGACGTTCCTTTGCAACATAGCCCAGTAACTTCTTAACT |
| | 986 | CCACTATG |
| 1041 | Encoding Probe | GCTCGACGTTCCTTTGCAACACTTGCTAAGAATGAGGAAGGT |
| | 987 | CGTAATGT |
| 1042 | Encoding Probe | GCTCGACGTTCCTTTGCAACAGTGAGCTCAAAAGGTTCATGG |
| | 988 | AGAG |
| 1043 | Encoding Probe | GCTCGACGTTCCTTTGCAACATTCAGGTTTTGCCTAAATTTCT |
| | 989 | GCCTG |
| 1044 | Encoding Probe | GCTCGACGTTCCTTTGCAACAGAAGTGTGTTTCATGTCTCTCT |
| | 990 | GTGG |
| 1045 | Encoding Probe | GCTCGACGTTCCTTTGCAACAGTAGGGAGACAGGTAGAATTT |
| | 991 | TCATTGAAG |
| 1046 | Encoding Probe | GCTCGACGTTCCTTTGCAACATCCGGTATTGCTGATAATGAT |
| | 992 | GGAGAGCT |
| 1047 | Encoding Probe | GCTCGACGTTCCTTTGCAACATCTAGCTGTTTCTGCTATTTGA |
| | 993 | GAGCTT |
| 1048 | Encoding Probe | GCTCGACGTTCCTTTGCAACAGTATGCTCATAGCACACTACA |
| | 994 | GGAC |
| 1049 | Encoding Probe | GCTGGTCGGTGGATTGGATTTTTTATAGTAAGCCACATCAGC |
| | 995 | TATGTCCACGTC |
| 1050 | Encoding Probe | GCTGGTCGGTGGATTGGATTTGTCACTTGCTGTGAGTCATTT |
| | 996 | CTGCTTTCATCG |
| 1051 | Encoding Probe | GCTGGTCGGTGGATTGGATTTGCGGATGACTGCTAATGACAC |
| | 997 | CACCACAATGAC |
| 1052 | Encoding Probe | GCTGGTCGGTGGATTGGATTTAAATCATCAACGTAGTAAATC |
| | 998 | AGAGTCTGCCCG |
| 1053 | Encoding Probe | GCTGGTCGGTGGATTGGATTTACAGTACCATAGGAAGTACA |
| | 999 | CTGGCATTCACCA |
| 1054 | Encoding Probe | GCTGGTCGGTGGATTGGATTTGTCGTCCATGCTCTTAGAAGA |
| | 1000 | ATGGAACAGGGA |
| 1055 | Encoding Probe | GCTGGTCGGTGGATTGGATTTCGACTGATGGTCGTAGGGGCT |
| | 1001 | TTCTCTTTCTTT |
| 1056 | Encoding Probe | GCTGGTCGGTGGATTGGATTTACTTTCAGCTTATATCGAGAT |
| | 1002 | GTGAACGCCTCT |
| 1057 | Encoding Probe | GCTGGTCGGTGGATTGGATTTTCCCATTAAGCTCTCTGTGGA |
| | 1003 | TCTCACCCATCT |
| 1058 | Encoding Probe | GCTGGTCGGTGGATTGGATTTGTTCTTGTTGCCAGCTTGTAG |
| | 1004 | TTGTCACAGACA |
| 1059 | Encoding Probe | GCTGGTCGGTGGATTGGATTTAAGTGAGAAGAGTTTTGCATC |
| | 1005 | AGATCAATGGTG |
| 1060 | Encoding Probe | GCTGGTCGGTGGATTGGATTTGAGAGCCTTCTCATATTTTGC |
| | 1006 | TGATTTCTTCCT |
| 1061 | Encoding Probe | CGTCAGGTGAGCATCTTACATACGCTGATCTGCGTCTTTGAT |
| | 1007 | CATTTGTCCTCG |
| 1062 | Encoding Probe | CGTCAGGTGAGCATCTTACATAGACAGAGGTTTTACTGAGTC |
| | 1008 | TGGCTTTTGCTG |
| 1063 | Encoding Probe | CGTCAGGTGAGCATCTTACATAAGCCCTGGCATATGTATACG |
| | 1009 | TTTTGTCAAGGC |
| 1064 | Encoding Probe | CGTCAGGTGAGCATCTTACATCTGACGATTTTCAGGTGTTTC |
| | 1010 | ATCTTCACAGGC |
| 1065 | Encoding Probe | CGTCAGGTGAGCATCTTACATTATCTGGTGTCATGTGCTAGT |
| | 1011 | TTCTCAGAGTGA |
| 1066 | Encoding Probe | CGTCAGGTGAGCATCTTACATTCTTGTCCACCAAAGGATACA |
| | 1012 | CGTCTTCTCTTC |
| 1067 | Encoding Probe | CGTCAGGTGAGCATCTTACATCGAGATATCAACGGAACTAA |
| | 1013 | GACCAGGTAGGCC |
| 1068 | Encoding Probe | CGTCAGGTGAGCATCTTACATAGGAGATTCACAGAATGTCGT |
| | 1014 | CTGCCAGTAACA |
| 1069 | Encoding Probe | CGTCAGGTGAGCATCTTACATACACGGGCATCTTTGGGGTTT |
| | 1015 | TCTCAACAATAA |
| 1070 | Encoding Probe | CGTCAGGTGAGCATCTTACATAACGTTTTGACAACCACTAAT |
| | 1016 | GGGCCATTAGCA |
| 1071 | Encoding Probe | CGTCAGGTGAGCATCTTACATACAGTCATCTTCTGGCCCTAT |
| | 1017 | GGGTATGTGTAC |
| 1072 | Encoding Probe | CGTCAGGTGAGCATCTTACATCCGCCCGAGATGTAGATTTCT |
| | 1018 | TGGCTCCATCAT |
| 1073 | Encoding Probe | CGTCAGGTGAGCATCTTACATCAACCTCTAGACCTCAAGCAT |
| | 1019 | GTTCTTTTCCCA |
| 1074 | Encoding Probe | CGTCAGGTGAGCATCTTACATTCATGTATTCTGAGCTGCCTC |
| | 1020 | TTTAAACCTGCT |
| 1075 | Encoding Probe | CGTCAGGTGAGCATCTTACATTTACCTGGTTGTGGAGATCTC |
| | 1021 | AAGGACATTCTT |
| 1076 | Encoding Probe | CGTCAGGTGAGCATCTTACATTGTTCAGACTCTCCAGAGACT |
| | 1022 | CCTTTCTCTTCC |
| 1077 | Encoding Probe | CGTCAGGTGAGCATCTTACATTGATCAAGAGGTTGAACCCCA |
| | 1023 | TCCTTATGTGTC |
| 1078 | Encoding Probe | CGTCAGGTGAGCATCTTACATACATCTCGAAATGTCCCCCTT |
| | 1024 | AAATGTCTTGCT |
| 1079 | Encoding Probe | CGTCAGGTGAGCATCTTACATGTGAATGCTTCCATCTCTTGC |
| | 1025 | AGACTTCCTCTT |
| 1080 | Encoding Probe | CGTCAGGTGAGCATCTTACATGTGTGTCACTGAATCCTTGTT |
| | 1026 | ATGGCCTGATGT |
| 1081 | Encoding Probe | CGTCAGGTGAGCATCTTACATATGAGGGAGAGTTTGCATGG |
| | 1027 | CCTGTAGTAAATT |
| 1082 | Encoding Probe | CGTCAGGTGAGCATCTTACATAGGTTGGTTGGTTCCTCCTGC |
| | 1028 | CAGTTAAACTTA |
| 1083 | Encoding Probe | CGTCAGGTGAGCATCTTACATGGCAGGCCTCCTCTTATGTCC |
| | 1029 | TGTTGAATTTCC |
| 1084 | Encoding Probe | CGTCAGGTGAGCATCTTACATTGATTTTCCTTAGGTGTTTGTG |
| | 1030 | GCTGTCTGGTA |

### Example 19: HiPR-Cycle can be Performed Simultaneously Across Biological Kingdoms

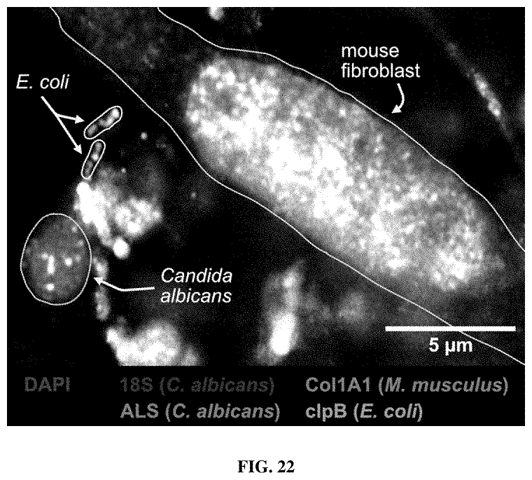

Here, as shown in FIG. 22, we show that signal amplification via HiPR-Cycle was not biased to detect genes of a particular kingdom (i.e. bacteria).

Method

Fixed suspensions of cells were prepared separately as follows:

E. coli (ATCC 25922) were cultured in suspension at 37° C. in a shaker for multiple passages. In the last passage, cells were incubated at 30° C. for one hour and then the tube containing cells were placed in a water bath at 46° C. for 5 minutes to induce heat shock. Following the shock, cells were placed on ice for 30 seconds and then immediately fixed in an equal volume of 2% formaldehyde for 90 minutes at room temperature. Pelleted cells were rinsed with 1×PBS and stored in 70% ethanol at −20° C.

Candida albicans cells were cultured on YM media plates for several passages at 30° C. Cells were pelleted and fixed in 2% formaldehyde for 60 minutes at room temperature in a rotator. Pelleted cells were rinsed with 1×PBS and resuspended in ice-cold Buffer B (1×PBS with 1.2 M sorbitol and 100 mM of potassium phosphate dibasic). 5 μL of zymolyase (per 1 mL of cell suspension) was added to the suspension and mixed by vortexing. The cells were incubated for 30 minutes at 30° C. to enable cell wall digestion. Cells were then washed with ice-cold Buffer B and stored in 70% ethanol at −20° C.

Mouse 3T3 fibroblast cells were cultured in Complete Growth Medium (DMEM+10% bovine calf serum+1× Penicillin and Streptomycin) in Petri dishes at 37° C. (5% CO₂). At collection, adherent cells were released from the plate using a Trypsin-EDTA solution and incubated for several minutes. Cells were then washed in 1×PBS before being fixed in 3.7% formaldehyde for 10 minutes at room temperature. Fixed stocks were washed in 1×PBS and resuspended in 70% ethanol at −20° C.

A mixture of fixed 3T3 cells, C. albicans cells, and heat-shocked E. coli were mixed in a 1:1:5 volume ratio and deposited on Ultrastick glass slides. We added 10 μg/ml lysozyme to digest bacterial cell walls and incubated the sections for 30 minutes at 37° C.; sections were then washed with 1×PBS for 15 minutes at room temperature. We then added pre-encoding buffer to samples for 30 minutes at 37° C. An encoding buffer was synthesized to include probes for: mRNA from C. albicans-specific ALS10 (R6), E. coli-specific clpB (R2), and murine-specific Vtn (R2) and Col1a1 (R6) (at a concentration of 400 nM per gene pool); 16S rRNA from E. coli (R4) and 18S rRNA from C. albicans (R1) (at a concentration of 2 μM per pool). The encoding probes were added to specimens. The specimens were incubated overnight (16 hours) at 37° C.

The following day, specimens were washed in HiPR-FISH wash buffer for 15 minutes at 48° C. followed by 5×SSC+Tween 20 (5 minutes, room temperature) to remove unbound encoding probes. A pre-amplification buffer was added to specimens for 30 minutes at room temperature. During this incubation, amplifier probes were annealed by heating to 95° C. for 2 minutes and allowed to cool to room temperature for 30 minutes. An amplification buffer was prepared by adding ten pairs of amplifier probes (60 nM each; stocks of probes at 3 μM) and readout probes (400 nM each) and incubated overnight at 30° C. at room temperature in the dark. Specimens were again washed with 2×SSC+Tween 20 at 42° C. for 15 minutes.

Encoding probes 287-311 (SEQ ID NO: 319-343), as shown in Table 10, and encoding probes 1031-1130, as shown in Table 16 below, were used in this example Amplifier probes 41-42 and 45-46 (SEQ ID NO: 979-980 and 1185-1186), as shown in Table 2, were used in this example. Readout probes 1-2 and 5-6 (SEQ ID NO: 25-26 and 29-30), as shown in Table 3, were used in this example.

TABLE 16
Encoding used in Example 19.

| SEQ ID | Probe | |
|---|---|---|
| NO: | Name | Sequence |
| 1085 | Encoding Probe | CGTCGGAGTGGGTTCAGTCTACCGGCACCAACCAGACGAGA |
| | 1031 | CACCGA |
| 1086 | Encoding Probe | CGTCGGAGTGGGTTCAGTCTAGACGACGGCGACGATCCGGT |
| | 1032 | CTTCAT |
| 1087 | Encoding Probe | CGTCGGAGTGGGTTCAGTCTAATTCGCGATGGTGCAGTTCTT |
| | 1033 | GCTCC |
| 1088 | Encoding Probe | CGTCGGAGTGGGTTCAGTCTAGACGAAACGACGTTCCAGCG |
| | 1034 | CAGCAT |
| 1089 | Encoding Probe | GCTCGACGTTCCTTTGCAACAACACTTAGTTTTGTCGCGGTT |
| | 1035 | AGGATCGAATGG |
| 1090 | Encoding Probe | GCTCGACGTTCCTTTGCAACACCAGGTGCAGTAACGGTAGTA |
| | 1036 | GCAGTGGTATAG |
| 1091 | Encoding Probe | GCTCGACGTTCCTTTGCAACAACAGAATGTATCTCCCGGACT |
| | 1037 | TGCACTAGTACC |
| 1092 | Encoding Probe | GCTCGACGTTCCTTTGCAACATTTCCCAAGAACAACCTTTAT |
| | 1038 | CACCTTCACAGC |
| 1093 | Encoding Probe | GCTCGACGTTCCTTTGCAACAAGGATCTTAATGTGAAAGGTG |
| | 1039 | CACGTTGCCAAT |
| 1094 | Encoding Probe | GCTCGACGTTCCTTTGCAACAACCAGCGGTGACAGTAGTAGT |
| | 1040 | GGTAGTGTAAGA |
| 1095 | Encoding Probe | GCTCGACGTTCCTTTGCAACAACCTAGTGGTGGTTGCGTAAG |
| | 1041 | ATTGAGACCAAT |
| 1096 | Encoding Probe | GCTCGACGTTCCTTTGCAACATCAATGGTTTGGTGGTTCTCTG |
| | 1042 | ATAATCACGGT |
| 1097 | Encoding Probe | GCTCGACGTTCCTTTGCAACAAGGTTGATGATAACTGAATCA |
| | 1043 | GTGCCACCTGGT |
| 1098 | Encoding Probe | GCTCGACGTTCCTTTGCAACAAGATCCAAATCAACAGAAGA |
| | 1044 | ACCAGTTCCACCT |
| 1099 | Encoding Probe | GCTCGACGTTCCTTTGCAACAACGAAGTGGTAAAGTGACAGT |
| | 1045 | ACCCAAAGCCTT |
| 1100 | Encoding Probe | GCTCGACGTTCCTTTGCAACATAAAAAACAGTATCAGTGTTT |
| | 1046 | CCTGGTGGTCCA |
| 1101 | Encoding Probe | GCTCGACGTTCCTTTGCAACATAACCAAGTTGGGGTTCCTGG |
| | 1047 | TCCCTTATAATT |
| 1102 | Encoding Probe | GCTCGACGTTCCTTTGCAACACTAAATAACTGAATCAGTTCC |
| | 1048 | ACCTGGAGGAGC |
| 1103 | Encoding Probe | GCTCGACGTTCCTTTGCAACATTCTGTTAGCGAATCCCATTGT |
| | 1049 | ACCAGATGTGT |
| 1104 | Encoding Probe | GCTCGACGTTCCTTTGCAACACAAGCGTAAGATTGAGACCAG |
| | 1050 | TACTCAGTGGTT |
| 1105 | Encoding Probe | GCTCGACGTTCCTTTGCAACAGATGAAGAAATGATAGGCGA |
| | 1051 | TGAAGCTTCCACG |
| 1106 | Encoding Probe | GCTCGACGTTCCTTTGCAACAATAGTGTTGTGGTGGAAGTAA |
| | 1052 | TTGTTCCTGTCC |
| 1107 | Encoding Probe | GCTCGACGTTCCTTTGCAACACCTTTGCGATTGAGATTGGTT |
| | 1053 | GGTTGATGTTGT |
| 1108 | Encoding Probe | GCTCGACGTTCCTTTGCAACATCTTGGGGATTGTAAAGTGGA |
| | 1054 | TTCTGTGGTTGT |
| 1109 | Encoding Probe | GCTCGACGTTCCTTTGCAACACCCTTTGGCAGTGGAACTTGT |
| | 1055 | ACAATGACAGTG |
| 1110 | Encoding Probe | GCTCGACGTTCCTTTGCAACATCATTTGGTCAGGTAGGAAGT |
| | 1056 | AGTCACACCAAC |
| 1111 | Encoding Probe | GCTCGACGTTCCTTTGCAACAAAGTCTAATGATAACCGAATC |
| | 1057 | AGTGCCACCTGG |
| 1112 | Encoding Probe | GCTCGACGTTCCTTTGCAACACCAGGTGCAGTTACAGTTGTG |
| | 1058 | GTTGTAGCAAAT |
| 1113 | Encoding Probe | GCTCGACGTTCCTTTGCAACAAAACATCATAGCCATAGGACA |
| | 1059 | TCTGGGAAGCAA |
| 1114 | Encoding Probe | GCTCGACGTTCCTTTGCAACACGCTTCACCACTTGATCCAGA |
| | 1060 | AGGACCTTGTTT |
| 1115 | Encoding Probe | GCTCGACGTTCCTTTGCAACAGTCGAGGACCAGCATCACCTT |
| | 1061 | TAACACCAGTAT |
| 1116 | Encoding Probe | GCTCGACGTTCCTTTGCAACAGACTTCTTGCAGTGATAGGTG |
| | 1062 | ATGTTCTGGGAG |
| 1117 | Encoding Probe | GCTCGACGTTCCTTTGCAACACGTGATACAGATCAAGCATAC |
| | 1063 | CTCGGGTTTCCA |
| 1118 | Encoding Probe | GCTCGACGTTCCTTTGCAACACAGCCTCGACTCCTACATCTT |
| | 1064 | CTGAGTTTGGTG |
| 1119 | Encoding Probe | GCTCGACGTTCCTTTGCAACAAGATGGTGGTTTTGTATTCGA |
| | 1065 | TGACTGTCTTGC |
| 1120 | Encoding Probe | GCTCGACGTTCCTTTGCAACAGTGCATCTTTACCAGGAGAAC |
| | 1066 | CATCAGCACCTT |
| 1121 | Encoding Probe | GCTCGACGTTCCTTTGCAACAGTGCATCTTGAGACTTCTCTTG |
| | 1067 | AGGTGGCTGAG |
| 1122 | Encoding Probe | GCTCGACGTTCCTTTGCAACACGCGATGTTCTCAATCTGCTG |
| | 1068 | ACTCAGGCTCTT |
| 1123 | Encoding Probe | GCTCGACGTTCCTTTGCAACACCCGTTGGGACAGTCCAGTTC |
| | 1069 | TTCATTGCATTG |
| 1124 | Encoding Probe | GCTCGACGTTCCTTTGCAACAGTGGATGACCCTTTATGCCTC |
| | 1070 | TGTCACCTTGTT |
| 1125 | Encoding Probe | GCTCGACGTTCCTTTGCAACAACTCCTGTCTCCATGTTGCAGT |
| | 1071 | AGACCTTGATG |
| 1126 | Encoding Probe | GCTCGACGTTCCTTTGCAACATGGCTTAGGCCATTGTGTATG |
| | 1072 | CAGCTGACTTCA |
| 1127 | Encoding Probe | GCTCGACGTTCCTTTGCAACACGACTCTCCAAACCAGACGTG |
| | 1073 | CTTCTTTTCCTT |
| 1128 | Encoding Probe | GCTCGACGTTCCTTTGCAACACCCCCAATGTCTAGTCCGAAT |
| | 1074 | TCCTGGTCTGGG |
| 1129 | Encoding Probe | GCTCGACGTTCCTTTGCAACACCCTTCTCCTTTGGCACCAGTG |
| | 1075 | TCTCCTTTGTT |
| 1130 | Encoding Probe | GCTCGACGTTCCTTTGCAACAGGCCTCTTCCAGTCAGAGTGG |
| | 1076 | CACATCTTGAGG |
| 1131 | Encoding Probe | GCTCGACGTTCCTTTGCAACAGTCGAAGACCAGGGAAGCCTC |
| | 1077 | TTTCTCCTCTCT |
| 1132 | Encoding Probe | GCTCGACGTTCCTTTGCAACACGCGTACCCTTAGGTCCAGGG |
| | 1078 | AATCCCATCACA |
| 1133 | Encoding Probe | GCTCGACGTTCCTTTGCAACAACGAGCCTTGGTTAGGGTCGA |
| | 1079 | TCCAGTACTCTC |
| 1134 | Encoding Probe | GCTCGACGTTCCTTTGCAACATCGAGGTCCATCAGCACCAGG |
| | 1080 | AGATCCTTTCTC |
| 1135 | Encoding Probe | GCTCGACGTTCCTTTGCAACACTGCTTGTACACCACGTTCAC |
| | 1081 | CAGGGAAACCTC |
| 1136 | Encoding Probe | GCTCGACGTTCCTTTGCAACAAGGCTTAGGACCAGCAGGACC |
| | 1082 | AGCATCTCCTTT |
| 1137 | Encoding Probe | CGTCGGAGTGGGTTCAGTCTAGAGTGATAGTAAGTGCAAAG |
| | 1083 | CTCGTCACACTGA |
| 1138 | Encoding Probe | CGTCGGAGTGGGTTCAGTCTAGGACGCTGGAGAACAAAGAG |
| | 1084 | AACCAGATTGAAC |
| 1139 | Encoding Probe | CGTCGGAGTGGGTTCAGTCTAAGGAGAGAAGAAATAGACGC |
| | 1085 | TCTGAATGGGCTC |
| 1140 | Encoding Probe | CGTCGGAGTGGGTTCAGTCTAGTTTAATCATCCTCTGGCATA |
| | 1086 | GTGAACACGTCC |
| 1141 | Encoding Probe | CGTCGGAGTGGGTTCAGTCTAAACAACAAGTAGGTCTTCCCC |
| | 1087 | TGACAGTTGATG |
| 1142 | Encoding Probe | CGTCGGAGTGGGTTCAGTCTAGGACAATGCCCCAGACATCTT |
| | 1088 | GGATAAGTTTGG |
| 1143 | Encoding Probe | CGTCGGAGTGGGTTCAGTCTACGGCCAGAAGAGGAGTTCGA |
| | 1089 | AAATGTTCTCCCA |
| 1144 | Encoding Probe | CGTCGGAGTGGGTTCAGTCTACGAGCATCAACATTGTCTGGT |
| | 1090 | ATGCCACTGAAG |
| 1145 | Encoding Probe | CGTCGGAGTGGGTTCAGTCTAAGCCCGGGTTCTAAGGTTGAC |
| | 1091 | TCGGTAGTATTT |
| 1146 | Encoding Probe | CGTCGGAGTGGGTTCAGTCTAACGTGCTGAAATTCGTACTCC |
| | 1092 | CAGTACTGCTTC |
| 1147 | Encoding Probe | CGTCGGAGTGGGTTCAGTCTACCCCTTTCCACTGCACAGTTC |
| | 1093 | TTCCTCTGGAAA |
| 1148 | Encoding Probe | CGTCGGAGTGGGTTCAGTCTAAGCGAAGGCAAAGAGGGACC |
| | 1094 | CATTCTTGAGATC |
| 1149 | Encoding Probe | CGTCGGAGTGGGTTCAGTCTACGCCTTTCGGCTTCTACGCTT |
| | 1095 | AGACTTCTGTTT |
| 1150 | Encoding Probe | CGTCGGAGTGGGTTCAGTCTAAGTCTGCCGTCTCATCTAGCT |
| | 1096 | CATAGCAGTACT |
| 1151 | Encoding Probe | CGTCGGAGTGGGTTCAGTCTACGTGAGTGGGATAAGGAGCC |
| | 1097 | AGTGACGTAGATG |
| 1152 | Encoding Probe | CGTCGGAGTGGGTTCAGTCTAAAGCTCAGGATCTAGGAAGG |
| | 1098 | CTGTCGGCTTTAG |
| 1153 | Encoding Probe | CGTCGGAGTGGGTTCAGTCTAAACGTATTGTTCTTGGGCTCC |
| | 1099 | TCCACGTAGTCA |
| 1154 | Encoding Probe | CGTCGGAGTGGGTTCAGTCTAACGCACCAGGGCTAGTATGA |
| | 1100 | AAAAGGGCCTCAG |
| 1155 | Encoding Probe | CGTCGGAGTGGGTTCAGTCTAAACAAGAAGTAGACCCTTTCC |
| | 1101 | CGGCCACTGTAA |
| 1156 | Encoding Probe | CGTCGGAGTGGGTTCAGTCTAGGCGCTGATGAATTGGGGTTC |
| | 1102 | TCTGGCTCCATC |
| 1157 | Encoding Probe | CGTCGGAGTGGGTTCAGTCTACTCGATTTAGGTCACCGGGTG |
| | 1103 | GAGAGGTGTTCT |
| 1158 | Encoding Probe | CGTCGGAGTGGGTTCAGTCTAGGGTTGATCAGTGGTGTCGGG |
| | 1104 | CCTTAGGATCTC |
| 1159 | Encoding Probe | CGTCGGAGTGGGTTCAGTCTAGGGCATCCTCAAAGCGCCAGT |
| | 1105 | ACTGACTACCCT |
| 1160 | Encoding Probe | CGTCGGAGTGGGTTCAGTCTACGGAATACTGAGCAATGGAG |
| | 1106 | CGTGGGTAGGGAG |
| 1161 | Encoding Probe | TGTGGAGGGATTGAAGGATAACTTCCCCGTGGTTGAGTCAA |
| | 1107 | AAAT |
| 1162 | Encoding Probe | TGTGGAGGGATTGAAGGATAACCCCAGACTTGGCCTTCCAAT |
| | 1108 | TGTAGG |
| 1163 | Encoding Probe | TGTGGAGGGATTGAAGGATAGAGTTCCAGAATGAGGTTGCC |
| | 1109 | AGG |
| 1164 | Encoding Probe | TGTGGAGGGATTGAAGGATAAGAGTTCCAGAATGAGGTTGC |
| | 1110 | CAGG |
| 1165 | Encoding Probe | TGTGGAGGGATTGAAGGATAAGGGTTCGCCATAAATGGCTA |
| | 1111 | CCGTC |
| 1166 | Encoding Probe | TGTGGAGGGATTGAAGGATATGACATCGACTTGGAGTCGAT |
| | 1112 | TCA |
| 1167 | Encoding Probe | TGTGGAGGGATTGAAGGATACGTTGACTACTGGCAGGATCA |
| | 1113 | ACCACTA |
| 1168 | Encoding Probe | TGTGGAGGGATTGAAGGATAACTTCCCCGTGGTTGAGTCAAT |
| | 1114 | AA |
| 1169 | Encoding Probe | TGTGGAGGGATTGAAGGATAGGATTCGCCATAAATGGCTAC |
| | 1115 | CGTC |
| 1170 | Encoding Probe | TGTGGAGGGATTGAAGGATAGTAACTTGGAGTCGATAGTCC |
| | 1116 | CAGA |
| 1171 | Encoding Probe | TGTGGAGGGATTGAAGGATATCGATGACTACTGGCAGGATC |
| | 1117 | AACCACTA |
| 1172 | Encoding Probe | TGTGGAGGGATTGAAGGATAAGTACCTCCCCTGAATCGGGA |
| | 1118 | TTCCC |
| 1173 | Encoding Probe | TGTGATGGAAGTTAGAGGGTAGTCTTGGTTTTCCGGATTTGG |
| | 1119 | GA |
| 1174 | Encoding Probe | TGTGATGGAAGTTAGAGGGTCATGTCAATGAGCAAAGGTAT |
| | 1120 | TAAGAA |
| 1175 | Encoding Probe | TGTGATGGAAGTTAGAGGGTCATGTCAATGAGCAAAGGTAT |
| | 1121 | TATGA |
| 1176 | Encoding Probe | TGTGATGGAAGTTAGAGGGTCGTCACCCCATTAAGAGGCTC |
| | 1122 | GGT |
| 1177 | Encoding Probe | TGTGATGGAAGTTAGAGGGTGAAACTAACACACACACTGAT |
| | 1123 | TGTC |
| 1178 | Encoding Probe | TGTGATGGAAGTTAGAGGGTGAGCCTTGGTTTTCCGGATTTC |
| | 1124 | GG |
| 1179 | Encoding Probe | TGTGATGGAAGTTAGAGGGTGGAGCCTTGGTTTTCCGGATTA |
| | 1125 | CG |
| 1180 | Encoding Probe | TGTGATGGAAGTTAGAGGGTGTAAGCTCACAATATGTGCAT |
| | 1126 | AAA |
| 1181 | Encoding Probe | TGTGATGGAAGTTAGAGGGTGTCACCCCATTAAGAGGCTCC |
| | 1127 | GTG |
| 1182 | Encoding Probe | TGTGATGGAAGTTAGAGGGTGTGCTCAGCCTTGGTTTTCCGC |
| | 1128 | TA |
| 1183 | Encoding Probe | TGTGATGGAAGTTAGAGGGTGTGTCTCATCTCTGAAAACTTC |
| | 1129 | CGACC |
| 1184 | Encoding Probe | TGTGATGGAAGTTAGAGGGTTGACACACACACTGATTCAGG |
| | 1130 | GAG |

### Example 20: HiPR-Cycle can be Performed with Readout Probe Exchange to Enable Ultrahigh Multiplexity Measurements of Gene Expression

Here we demonstrate that after amplification readout probes could be unbound and bound without disturbing the amplified structures.

Method

E. coli cultures (ATCC 25922) were established by incubating bacteria in suspension in tryptic soy media at 37° C. in a shaker. For one batch, on the final passage, media included 1 mM IPTG and cAMP to induce LacZ expression. For another batch, in the last passage, cells were incubated at 30° C. for one hour and then the tube containing cells were placed in a water bath at 46° C. for 5 minutes to induce heat shock. Following the shock, cells were placed on ice for 30 seconds. In both cases, suspensions were then immediately fixed in an equal volume of 2% formaldehyde for 90 minutes at room temperature. Pelleted cells were rinsed with 1×PBS and stored in 50% ethanol at −20° C.

Suspensions were mixed in roughly equal concentrations and deposited on glass coverslip. Lysozyme (10 mg/mL) was deposited on the coverslip for 30 minutes at 37° C. Cells were then washed with 1×PBS at room temperature for 15 minutes. The coverslip was subsequently dried by dunking in 100% ethanol. An encoding buffer containing gene encoding probes for LacZ and clpB (200 nM) and Eubacterium (200 nM) was deposited on cells and incubated overnight at 37° C. The following day the coverslip was washed with HiPR-FISH wash buffer at 48° C. for 15 minutes and the coverslip was again dried with amplification 100% ethanol.

A pre-amplification buffer (i.e. containing no probes) was added to the specimens and incubated for 30 minutes at 30° C. Amplifier probes corresponding to encoding probe initiators were annealed and added to amplification buffer. The pre-amplification buffer was aspirated and replaced with amplification buffer and incubated at 30° C. for overnight.

The following day, the coverslip was washed in 2×SSCT at 42° C. for 15 minutes. The coverslip was then mounted on an FCS2 (bioptechs) flow cell and attached to an Aria flow system (Fluigent) to deliver buffers while the setup was on the confocal microscope (Zeiss i880).

First, a readout buffer containing readout probes 9-11 (each at 400 nM) was flowed onto the cells and incubated for 1 hour at 37° C. The cells were then washed with HiPR-FISH wash buffer for 15 minutes at 42° C. Finally, 2×SSC solution was flowed onto the cells and they were imaged.

In the second round, a readout/exchange buffer containing readout probe 12 (400 nM) and exchange probes 1 and 2 (10 μM) was flowed onto the cells and incubated for 1 hour at 37° C. The cells were then washed with HiPR-FISH wash buffer for 15 minutes at 42° C. Finally, 2×SSC solution was flowed onto the cells and they were imaged.

In the third round, a readout/exchange buffer containing readout probe 11 and 10 (400 nM) and exchange probe 3 (10 μM) was flowed onto the cells and incubated for 1 hour at 37° C. The cells were then washed with HiPR-FISH wash buffer for 15 minutes at 42° C. Finally, 2×SSC solution was flowed onto the cells and they were imaged.

Results

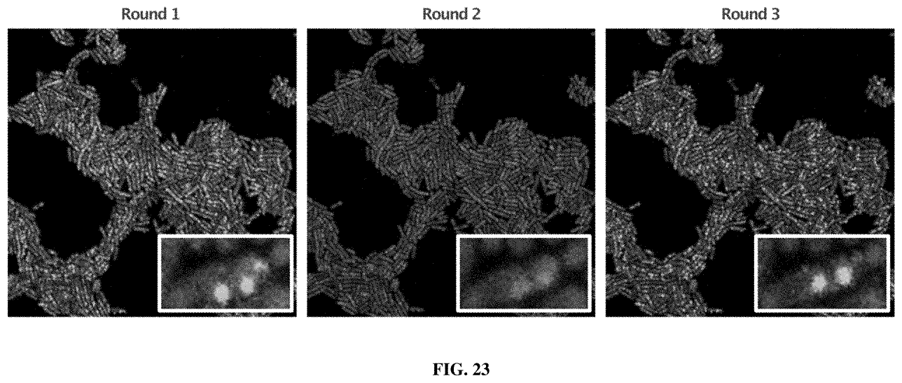

As seen in FIG. 23, we showed the ability to add and remove readout probes in different rounds without the loss of intensity; thus, the HiPR-Cycle amplification structures were not perturbed by probe exchange.

Encoding probes 287-311 (SEQ ID NO: 319-343), as shown in Table 10, 1031-1034 (SEQ ID NO: 1085-1088), and encoding probes 1131-1178, as shown in Table 17 below, were used in this example. Table 17 also contains the additional readout probes and exchange probes used in this example. Amplifier probes 17-18 and 47-50 (SEQ ID NO: 281-282 and 1240-1243), as shown in Table 2, were used in this example. Readout probes 9-10 (SEQ ID NO: 33-34), as shown in Table 3, were used in this example.

TABLE 17
Encoding, exchange, and additional readout probes used in
Example 20.

| SEQ ID | Probe | |
|---|---|---|
| NO: | Name | Sequence |
| 246 | Encoding Probe | TAGAGTTGATAGAGGGAGAAGCTGCCTCCCGTAGGAGT |
| | 222 | |
| 1187 | Encoding Probe | CTGGCACCGCTAAACCGTATTGCCAAACCAGGCAAAGCGC |
| | 1131 | CATTCGCCA |
| 1188 | Encoding Probe | CTGGCACCGCTAAACCGTATTATTTGCGAACAGCGCACGG |
| | 1132 | CGTTAAAGT |
| 1189 | Encoding Probe | CTGGCACCGCTAAACCGTATTGCCTTAACGCCGCGAATCA |
| | 1133 | GCAACGGCT |
| 1190 | Encoding Probe | CTGGCACCGCTAAACCGTATTGCTTAATTTCACCGCCGAAA |
| | 1134 | GGCGCGGT |
| 1191 | Encoding Probe | CTGGCACCGCTAAACCGTATTCATTCGCTTGCCACCGCAAC |
| | 1135 | ATCCACAT |
| 1192 | Encoding Probe | CTGGCACCGCTAAACCGTATTGGGTGCCACAAAGAAACCG |
| | 1136 | TCACCCGCA |
| 1193 | Encoding Probe | CTGGCACCGCTAAACCGTATTCGCTGCAGCAGATGGCGAT |
| | 1137 | GGCTGGTTT |
| 1194 | Encoding Probe | CTGGCACCGCTAAACCGTATTACGAACAACGCCGCTTCGG |
| | 1138 | CCTGGTAAT |
| 1195 | Encoding Probe | CTGGCACCGCTAAACCGTATTCGATCTGACCATGCGGTCGC |
| | 1139 | GTTTGGTT |
| 1196 | Encoding Probe | CTGGCACCGCTAAACCGTATTGCCAAACCGACGTCGCAGG |
| | 1140 | CTTCTGCTT |
| 1197 | Encoding Probe | CTGGCACCGCTAAACCGTATTCAAACCCATCGCGTGGGCA |
| | 1141 | TATTCGCAA |
| 1198 | Encoding Probe | CTGGCACCGCTAAACCGTATTCAGAATGCGGGTCGCTTCA |
| | 1142 | CTTACGCCA |
| 1199 | Encoding Probe | CTGGCACCGCTAAACCGTATTAACTAATCAGCACCGCGTC |
| | 1143 | GGCAAGTGT |
| 1200 | Encoding Probe | CTGGCACCGCTAAACCGTATTCTATTCGGCGCTCCACAGTT |
| | 1144 | CCGGATTT |
| 1201 | Encoding Probe | CTGGCACCGCTAAACCGTATTTTGTGCTTACCTTGCGGGCC |
| | 1145 | AACATCCA |
| 1202 | Encoding Probe | CTGGCACCGCTAAACCGTATTCGGTCCAGTACCGCGCGGC |
| | 1146 | TGAAATCAT |
| 1203 | Encoding Probe | CTGGCACCGCTAAACCGTATTCGCTCGTGATTAGCGCCGTG |
| | 1147 | GCCTGATT |
| 1204 | Encoding Probe | CTGGCACCGCTAAACCGTATTGCGACAGCGTGTACCACAG |
| | 1148 | CGGATGGTT |
| 1205 | Encoding Probe | CTGGCACCGCTAAACCGTATTAGTTACAGAACTGGCGATC |
| | 1149 | GTTCGGCGT |
| 1206 | Encoding Probe | CTGGCACCGCTAAACCGTATTTAACATTGGCACCATGCCGT |
| | 1150 | GGGTTTCA |
| 1207 | Encoding Probe | CTGGCACCGCTAAACCGTATTCGGTCTTCGCTATTACGCCA |
| | 1151 | GCTGGCGA |
| 1208 | Encoding Probe | CTGGCACCGCTAAACCGTATTTAGACACTCGGGTGATTAC |
| | 1152 | GATCGCGCT |
| 1209 | Encoding Probe | CTGGCACCGCTAAACCGTATTAGGAGATAACTGCCGTCAC |
| | 1153 | TCCAGCGCA |
| 1210 | Encoding Probe | CTGGCACCGCTAAACCGTATTAAATTTGATGGACCATTTCG |
| | 1154 | GCACCGCC |
| 1211 | Encoding Probe | CAACGATGCCCGTAGTTGACTGCCAAACCAGGCAAAGCGC |
| | 1155 | CATTCGCCA |
| 1212 | Encoding Probe | CAACGATGCCCGTAGTTGACTATTTGCGAACAGCGCACGG |
| | 1156 | CGTTAAAGT |
| 1213 | Encoding Probe | CAACGATGCCCGTAGTTGACTGCCTTAACGCCGCGAATCA |
| | 1157 | GCAACGGCT |
| 1214 | Encoding Probe | CAACGATGCCCGTAGTTGACTGCTTAATTTCACCGCCGAAA |
| | 1158 | GGCGCGGT |
| 1215 | Encoding Probe | CAACGATGCCCGTAGTTGACTCATTCGCTTGCCACCGCAAC |
| | 1159 | ATCCACAT |
| 1216 | Encoding Probe | CAACGATGCCCGTAGTTGACTGGGTGCCACAAAGAAACCG |
| | 1160 | TCACCCGCA |
| 1217 | Encoding Probe | CAACGATGCCCGTAGTTGACTCGCTGCAGCAGATGGCGAT |
| | 1161 | GGCTGGTTT |
| 1218 | Encoding Probe | CAACGATGCCCGTAGTTGACTACGAACAACGCCGCTTCGG |
| | 1162 | CCTGGTAAT |
| 1219 | Encoding Probe | CAACGATGCCCGTAGTTGACTCGATCTGACCATGCGGTCG |
| | 1163 | CGTTTGGTT |
| 1220 | Encoding Probe | CAACGATGCCCGTAGTTGACTGCCAAACCGACGTCGCAGG |
| | 1164 | CTTCTGCTT |
| 1221 | Encoding Probe | CAACGATGCCCGTAGTTGACTCAAACCCATCGCGTGGGCA |
| | 1165 | TATTCGCAA |
| 1222 | Encoding Probe | CAACGATGCCCGTAGTTGACTCAGAATGCGGGTCGCTTCA |
| | 1166 | CTTACGCCA |
| 1223 | Encoding Probe | CAACGATGCCCGTAGTTGACTAACTAATCAGCACCGCGTC |
| | 1167 | GGCAAGTGT |
| 1224 | Encoding Probe | CAACGATGCCCGTAGTTGACTCTATTCGGCGCTCCACAGTT |
| | 1168 | CCGGATTT |
| 1225 | Encoding Probe | CAACGATGCCCGTAGTTGACTTTGTGCTTACCTTGCGGGCC |
| | 1169 | AACATCCA |
| 1226 | Encoding Probe | CAACGATGCCCGTAGTTGACTCGGTCCAGTACCGCGCGGC |
| | 1170 | TGAAATCAT |
| 1227 | Encoding Probe | CAACGATGCCCGTAGTTGACTCGCTCGTGATTAGCGCCGTG |
| | 1171 | GCCTGATT |
| 1228 | Encoding Probe | CAACGATGCCCGTAGTTGACTGCGACAGCGTGTACCACAG |
| | 1172 | CGGATGGTT |
| 1229 | Encoding Probe | CAACGATGCCCGTAGTTGACTAGTTACAGAACTGGCGATC |
| | 1173 | GTTCGGCGT |
| 1230 | Encoding Probe | CAACGATGCCCGTAGTTGACTTAACATTGGCACCATGCCGT |
| | 1174 | GGGTTTCA |
| 1231 | Encoding Probe | CAACGATGCCCGTAGTTGACTCGGTCTTCGCTATTACGCCA |
| | 1175 | GCTGGCGA |
| 1232 | Encoding Probe | CAACGATGCCCGTAGTTGACTTAGACACTCGGGTGATTAC |
| | 1176 | GATCGCGCT |
| 1233 | Encoding Probe | CAACGATGCCCGTAGTTGACTAGGAGATAACTGCCGTCAC |
| | 1177 | TCCAGCGCA |
| 1234 | Encoding Probe | CAACGATGCCCGTAGTTGACTAAATTTGATGGACCATTTCG |
| | 1178 | GCACCGCC |
| 1235 | Readout probe 11 | /5Alex488N/CCCTTCTACTCAATTACCTCATCCC |
| 1236 | Readout probe 12 | /5Alex647N/CACCCTCATATCTATTACCCTCCCA |
| 1237 | Exchange probe | GATGATGTAGTAGTAAGGGT |
| | 1 | |
| 1238 | Exchange probe | GGGATGAGGTAATTGAGTAGAAGGG |
| | 2 | |
| 1239 | Exchange probe | TGGGAGGGTAATAGATATGAGGGTG |
| | 3 | |

### Example 21. HiPR-Cycle can Detect Proteins

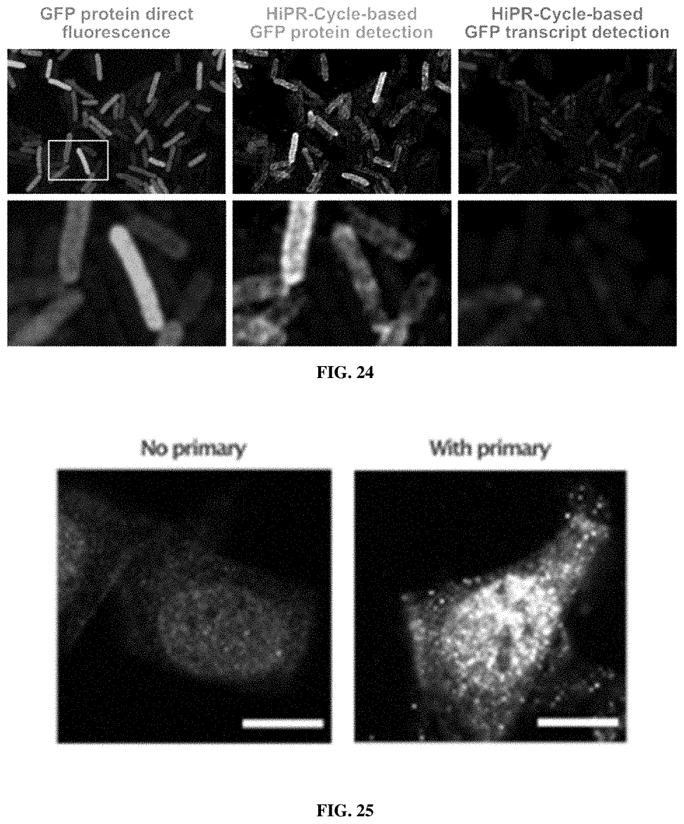

Here, we demonstrate the ability of HiPR-Cycle to measure molecular targets that extended beyond nucleic acids (FIG. 24).

Fixed GFP-expressing and non-GFP-expressing E. coli were mixed in a 1:1 ratio. The fixed E. coli stock was deposited onto a glass slide and lysozyme (10 μL of 10 mg/mL) was added to the slide and incubated at 37° C. for 15 minutes to digest the cell wall. The cells were washed twice with 1×PBS for 10 minutes at room temperature. Blocking buffer (5% bovine serum albumin (BSA) in PBS) was added to the slide for one hour at room temperature. Following blocking, we performed primary protein hybridization overnight at 4° C. with at a 1:500 dilution from stock. On the following day, slides were washed five times (5 minutes at room temperature, each) with PBST (PBS+0.1% Tween 20). A secondary antibody protein hybridization was performed with an initiator-conjugated protein for one hour at room temperature. At completion, we washed the slides with PBST three times (5 minutes at room temperature, each). Finally, the slides were re-fixed in 4% formaldehyde (Image-IT) for 10 minutes at room temperature and rinsed with 1×PBS.

Slides were then treated through the standard HiPR-Cycle assay. HiPR-Cycle encoding buffer containing probes for 16S rRNA and GFP mRNA (each pool at 80 nM and barcoded uniquely), was added to slides and incubated for 3 hours at 37° C. Samples were then washed with HiPR-Cycle wash buffer (5 minutes at 37° C., three times) and once with 5×SSC+Tween 20 for 5 minutes. A pre-amplification was performed (adding amplification buffer without HiPR-Cycle amplifier probes) for 30 minutes at room temperature, before adding amplification buffer with amplifier and readout probes corresponding to targets for amplification, and incubating at 30° C. overnight. Finally, slides were washed with 2×SSC+Tween 20 and incubated for 15 min at 37° C. before mourning with Prolong Antifade and a coverslip.

Slides were imaged using a Zeiss i880 confocal in the Airy Scan (super-resolution) mode with lasers set for 488 nm, 561 nm, and 633 nm excitation modes.

Encoding probes 71-80 (SEQ ID NO: 87-96), as shown in Table 6, and encoding probes 1179-1190, as shown in Table 18 below, were used in this example. Table 18 also contains the initiator sequence used to conjugate with the protein. Amplifier probes 7-8 (SEQ ID NO: 129-130), as shown in Table 2, were used in this example. Readout probes 7 and 9 (SEQ ID NO: 31 and 33), as shown in Table 3, were used in this example.

TABLE 18
Encoding and additional probes used in Example 21.

| SEQ ID | | |
|---|---|---|
| NO: | Probe Name | Sequence |
| 1244 | Encoding Probe 1179 | TAGAGTTGATAGAGGGAGAAAGTCTTGGTTTTCCGG |
| | | ATTTGGGA |
| 1245 | Encoding Probe 1180 | TAGAGTTGATAGAGGGAGAACATGTCAATGAGCAAA |
| | | GGTATTAAGAA |
| 1246 | Encoding Probe 1181 | TAGAGTTGATAGAGGGAGAACATGTCAATGAGCAAA |
| | | GGTATTATGA |
| 1247 | Encoding Probe 1182 | TAGAGTTGATAGAGGGAGAACGTCACCCCATTAAGA |
| | | GGCTCGGT |
| 1248 | Encoding Probe 1183 | TAGAGTTGATAGAGGGAGAAGAAACTAACACACACA |
| | | CTGATTGTC |
| 1249 | Encoding Probe 1184 | TAGAGTTGATAGAGGGAGAAGAGCCTTGGTTTTCCG |
| | | GATTTCGG |
| 1250 | Encoding Probe 1185 | TAGAGTTGATAGAGGGAGAAGGAGCCTTGGTTTTCC |
| | | GGATTACG |
| 1251 | Encoding Probe 1186 | TAGAGTTGATAGAGGGAGAAGTAAGCTCACAATATG |
| | | TGCATAAA |
| 1252 | Encoding Probe 1187 | TAGAGTTGATAGAGGGAGAAGTCACCCCATTAAGAG |
| | | GCTCCGTG |
| 1253 | Encoding Probe 1188 | TAGAGTTGATAGAGGGAGAAGTGCTCAGCCTTGGTT |
| | | TTCCGCTA |
| 1254 | Encoding Probe 1189 | TAGAGTTGATAGAGGGAGAAGTGTCTCATCTCTGAA |
| | | AACTTCCGACC |
| 1255 | Encoding Probe 1190 | TAGAGTTGATAGAGGGAGAATGACACACACACTGAT |
| | | TCAGGGAG |
| 1256 | Protein-bound | CGTCAGGTGAGCATCTTACAT/3AmMO/ |
| | initiator sequence | |

### Example 22. HiPR-Cycle can Detect Proteins

Here, we demonstrate the ability of HiPR-Cycle to measure molecular targets that extended beyond nucleic acids in mammalian cell types, as shown in FIG. 25.

Mouse 3T3 fibroblast cells were cultured in Complete Growth Medium (DMEM+10% bovine calf serum+1× Penicillin and Streptomycin) in Petri dishes at 37° C. (5% CO₂). At collection, adherent cells were released from the plate using a Trypsin-EDTA solution and incubated for several minutes. Cells were then washed in 1×PBS before being fixed in 3.7% formaldehyde for 10 minutes at room temperature. Fixed stocks were washed in 1×PBS and resuspended in 70% ethanol at −20° C.

The fixed 3T3 cells were deposited onto a glass slide and rinsed twice with 1×PBS. The cells were then permeabilized by adding Permeabilization Buffer (1×PBS with 0.1% Triton X-100). The slides were incubated for one hour at room temperature and then placed at 4° C. overnight. On the following day, the slides were washed with 1×PBS, twice, at room temperature. Blocking buffer (5% BSA in PBS) was deposited on the cells for one hour at room temperature. At the conclusion of blocking, the primary protein buffer was deposited on the cells and the slides were stored at 4° C., overnight. On the following day, specimens were washed with PBST (1×PBS with 0.1% Tween 20) for five minutes at room temperature (repeated four times). A secondary protein stain solution containing an initiator-conjugated protein (conc. 1 μg/mL in blocking buffer) was prepared and deposited on the cells. The slides were incubated for one hour at room temperature. Slides were washed with PBST for five minutes at room temperature (repeat two times).

Because no RNA molecules were targeted in this assay, we proceed to the amplification step. Samples were then washed once with 5×SSC+Tween 20 for 5 minutes. A pre-amplification was performed (adding amplification buffer without HiPR-Cycle amplifier probes) for 30 minutes at room temperature, before adding amplification buffer with amplifier and readout probes corresponding to targets for amplification, and incubating at 30° C. overnight. Slides were washed with 2×SSC+Tween 20 and incubated for 15 minutes at 42° C. Nuclei were stained with 5×SSC containing DAPI (20 ng/mL) and incubated for 5 minutes at room temperature in the dark. Prolong Antifade was added to each well with a coverslip to mount the samples.

Slides were imaged using a Zeiss i880 confocal in the lambda mode with lasers set for 488 nm, 561 nm, and 633 nm excitation modes.

Amplifier probes 45-46 (SEQ ID NO: 1185-1186), as shown in Table 2, were used in this example. Readout probes 1 and 6 (SEQ ID NO: 25 and 30), as shown in Table 3, were used in this example. The initiator sequence use in this example has the following sequence: GCTCGACGTTCCTTTGCAACA/3AmMO/(SEQ ID NO: 1257).

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

What is claimed is:

1. A method for analyzing a sample, comprising:
contacting at least one encoding probe with the sample to produce a first complex, wherein each encoding probe comprises a targeting sequence and an initiator sequence;
adding at least two different DNA amplifiers to the first complex to produce a second complex, wherein each DNA amplifier comprises an initiator complimentary sequence and a readout sequence; and
adding emissive readout probes to the second complex, wherein each emissive readout probe comprises a label and a complimentary sequence to the readout sequence of a corresponding DNA amplifier.

2. A method for analyzing a sample, comprising:
generating a set of probes, wherein each probe comprises:
(i) a targeting sequence;
(ii) at least one initiator sequence; and
(iii) at least two DNA amplifiers, wherein each DNA amplifier comprises an initiator complimentary sequence and a readout sequence;
contacting the set of probes with the sample to permit hybridization of the probes to nucleotides present in the sample to produce a complex;
adding a set of emissive readout probes to the complex, wherein each emissive readout probe comprises a label and a sequence complimentary to the readout sequence of a corresponding DNA amplifier;
detecting the emissive readout probes in the sample;
determining the spectra of “signal” and assigning them to a bacterium; and
decoding the spectra into a single, targeted transcript through means of signal deconvolution, error correction, comparison to reference standards

3. The method of claim 1 or 2, wherein the sample is at least one of a cell, a cell suspension, a tissue biopsy, a tissue specimen, urine, stool, blood, serum, plasma, bone biopsies, bone marrow, respiratory specimens, sputum, induced sputum, tracheal aspirates, bronchoalveolar lavage fluid, sweat, saliva, tears, ocular fluid, cerebral spinal fluid, pericardial fluid, pleural fluid, peritoneal fluid, placenta, amnion, pus, nasal swabs, nasopharyngeal swabs, oropharyngeal swabs, ocular swabs, skin swabs, wound swabs, mucosal swabs, buccal swabs, vaginal swabs, vulvar swabs, nails, nail scrapings, hair follicles, corneal scrapings, gavage fluids, gargle fluids, abscess fluids, wastewater, or plant biopsies.

4. The method of claim 3, wherein the sample is a cell.

5. The method of claim 4, wherein the cell is a bacterial or eukaryotic cell.

6. The method of claim 3, wherein the sample comprises a plurality of cells.

7. The method of claim 4, wherein each cell comprises a specific targeting sequence.

8. The method of claim 1 or 2, wherein the targeting sequence targets at least one of messenger RNA (mRNA), micro RNA (miRNA), long non-coding RNA (lncRNA), ribosomal RNA (rRNA), small interfering RNA (siRNA), transfer RNA (tRNA), Crispr RNA (crRNA), trans-activating cirspr RNA (tracrRNA), mitochondria RNA, Intronic RNA, viral mRNA, viral genomic RNA, environmental RNA, double-stranded RNA (dsRNA), small nuclear RNA (snRNA), small nucleolar (snoRNA), piwi-interacting RNA (piRNA), genomic DNA, synthetic DNA, DNA, plasmid DNA, a plasmid, viral DNA, retroviral DNA, environmental DNA, extracellular DNA, a protein, a small molecule, or an antigenic target.

9. The method of claim 8, wherein the target is mRNA.

10. The method of claim 8, wherein the target is rRNA.

11. The method of claim 8, wherein the target is mRNA and rRNA.

12. The method of claim 1 or 2, wherein the encoding probe comprises the initiator sequence on the 5′ end and/or the 3′ end.

13. The method of claim 12, wherein the encoding probe comprises an initiator sequence on the 5′ end and an initiator sequence on the 3′ end.

14. The method of claim 13, wherein the two initiator sequences have different sequences.

15. The method of claim 13, wherein the two initiator sequences have the same sequence.

16. The method of claim 1 or 2, wherein the encoding probe comprises two fractional initiator sequences.

17. The method of claim 1 or 2, wherein one of the two DNA amplifiers comprises, from 5′ to 3′, a readout sequence (R.1), a toehold sequence (T.1), a stem sequence (S.1), a loop sequence (L.1), and a complement stem sequence (cS.1).

18. The method of claim 1 or 2, wherein one of the two DNA amplifiers comprises, from 5′ to 3′, a stem sequence (S.2), a loop sequence (L.2), a complement stem sequence (cS.2), a toehold sequence (T.2), and a readout sequence (R.2).

19. The method of claim 17 or 18, wherein the two DNA amplifiers further comprise a spacer sequence, wherein the spacer sequence is about 1 to 3 nucleotides long.

20. The method of any one of claims 17 to 19, wherein the toehold sequence (T.1) is a sequence complementary to the loop sequence (L.2) of the other DNA amplifier.

21. The method of any one of claims 17 to 20, wherein the loop sequence (L.1) is a sequence complementary to the toehold sequence (T.2) of the other DNA amplifier.

22. The method of any one of claims 17 to 21, wherein the readout sequence of each DNA amplifier is the same sequence.

23. The method of any one of claims 17 to 21, wherein the readout sequence of each DNA amplifier is the different.

24. The method of claim 1 or 2, wherein the method comprises adding four DNA amplifiers.

25. The method of claim 24, wherein one of the four DNA amplifiers comprises, from 5′ to 3′ a amplifier initiator sequence (HI.1), a toehold sequence (T.1), a stem sequence (S.1), a loop sequence (L.1), and a complement stem sequence (cS.1).

26. The method of claim 24, wherein one of the four DNA amplifiers comprises, from 5′ to 3′ a stem sequence (S.2), a loop sequence (L.2), complement stem sequence (cS.2), a toehold sequence (T.2), and an amplifier initiator sequence (HI.2).

27. The method of claim 24, wherein one of the four DNA amplifiers comprises, from 5′ to 3′, a readout sequence (R.1-2), a toehold sequence (T.1-2), a stem sequence (S.1-2), a loop sequence (L.1-2), and a complement stem sequence (cS.1-2).

28. The method of claim 24, wherein one of the four DNA amplifiers comprises, from 5′ to 3′, a stem sequence (S.2-1), a loop sequence (L.2-1), a complement stem sequence (cS.2-1), a toehold sequence (T.2-1), and a readout sequence (R.2-1).

29. The method of any one of claims 24-28, wherein the four DNA amplifiers further comprise a spacer sequence, wherein the spacer sequence is about 1 to 3 nucleotides long.

30. The method of any one of claims 25-29, wherein the amplifier initiator sequence (HI.1) is a sequence complementary to the loop sequence (L.1-2 or L.2-1) of one of the other DNA amplifiers comprising the readout sequence.

31. The method of any one of claims 25-30, wherein the toehold sequence (T.1) is a sequence complementary to the loop sequence (L.2) of the other DNA amplifier comprising the amplifier initiator sequence.

32. The method of any one of claims 25-31, wherein the loop sequence (L.1) is a sequence complementary to the toehold sequence (T.2) of the other DNA amplifier comprising the amplifier initiator sequence.

33. The method of claim 1 or 2, wherein the emissive readout probe comprises a label on the 5′ or 3′ end.

34. The method of claim 1 or 2, wherein the emissive readout probe comprises a label on the 5′ end and a label on the 3′ end.

35. The method of claim 34, wherein the labels are the same.

36. The method of claim 34, wherein the labels are different.

37. The method of any one of claims 33-36, wherein the label is Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, Rox, Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740.

38. The method of claim 1 or 2, wherein the label is imaged using widefield microscopy, point scanning confocal microscopy, spinning disk confocal microscopy, lattice lightsheet microscopy, or light field microscopy.

39. The method of claim 38, wherein the detection strategy used is channel, spectral, channel and fluorescence lifetime, or spectral and fluorescence lifetime.

40. The method of claim 1 or 2, wherein the sample is on an analyzing platform, wherein the analyzing platform is a microscope slide, at least one chamber, at least one microfluidic device, at least one well, at least one plate, or at least one filter membrane.

41. A method for analyzing a cell, comprising:
contacting at least one encoding probe with the cell to produce a first complex, wherein each encoding probe comprises an mRNA targeting sequence and an initiator sequence;
adding two different DNA amplifiers to the first complex to produce a second complex, wherein each DNA amplifier comprises an initiator complimentary sequence and a readout sequence; and
adding two emissive readout probes to the second complex, wherein each emissive readout probe comprises a fluorophore and a complimentary sequence to the readout sequence of a corresponding DNA amplifier.

42. A construct comprising:
a targeting sequence that is complementary to a region of interest on a DNA/RNA sequence;
a first initiator sequence;
a second initiator sequence that is different from the first initiator sequence;
a first amplifier sequence comprising a readout sequence on the 5′ end of the sequence;
a second amplifier sequence comprising a readout sequence on the 3′ end of the sequence, wherein the second amplifier sequence is different from the first amplifier sequence; and
an emissive readout sequence comprising a sequence complimentary to the readout sequence of the first and/or second amplifier sequences and a label on the 5′ and/or 3′ end of the complimentary sequence.

43. The construct of claim 42, wherein the region of interest on a nucleotide is at least one of messenger RNA (mRNA), micro RNA (miRNA), long non coding RNA (lncRNA), ribosomal RNA (rRNA), small interfering RNA (siRNA), transfer RNA (tRNA), Crispr RNA (crRNA), trans-activating cirspr RNA (tracrRNA), mitochondria RNA, intronic RNA, viral mRNA, viral genomic RNA, environmental RNA, double-stranded RNA (dsRNA), small nuclear RNA (snRNA), small nucleolar (snoRNA), piwi-interacting RNA (piRNA), genomic DNA, synthetic DNA, DNA, plasmid DNA, a plasmid, viral DNA, retroviral DNA, environmental DNA, extracellular DNA, a protein, a small molecule, or an antigen.

44. The construct of claim 43, wherein the region of interest on a nucleotide is mRNA.

45. The construct of claim 43, wherein the region of interest on a nucleotide is rRNA.

46. The construct of claim 43, wherein the region of interest on a nucleotide is mRNA and rRNA.

47. The construct of claim 42, wherein the first initiator sequence is to the 5′ end of the targeting sequence.

48. The construct of claim 42, wherein the second initiator sequence is to the 3′ end of the targeting sequence.

49. The construct of any one of claims 42-48, wherein the first amplifier comprises, from 5′ to 3′, a readout sequence (R.1), a toehold sequence (T.1), a stem sequence (S.1), a loop sequence (L.1), and a complement stem sequence (cS.1).

50. The construct of any one of claims 42-49, wherein the second amplifier comprises, from 5′ to 3′, a stem sequence (S.2), a loop sequence (L.2), a complement stem sequence (cS.2), a toehold sequence (T.2), and a readout sequence (R.2).

51. The construct of any one of claims 42-50, wherein the each amplifier further comprises a spacer sequence, wherein the spacer sequence is about 1 to 3 nucleotides long.

52. The construct of any one of claims 49-51, wherein the toehold sequence (T.1) of the first amplifier is a sequence complementary to the loop sequence (L.2) of the second amplifier.

53. The construct of any one of claims 49-51, wherein the loop sequence (L.1) of the first amplifier is a sequence complementary to the toehold sequence (T.2) of the second amplifier.

54. The construct of any one of claims 42-53, wherein the first and second amplifier have the same readout sequence.

55. The construct of any one of claims 42-53, wherein the first and second amplifier have different readout sequences.

56. The construct of any one of claims 42-55, wherein the emissive readout sequence comprises a sequence complimentary to the readout sequence of the first amplifier sequence.

57. The construct of any one of claims 42-56, wherein the emissive readout sequence comprises a sequence complimentary to the readout sequence of the second amplifier sequence.

58. The construct of any one of claims 42-57, wherein the emissive readout sequence comprises a label on the 5′ end of the complimentary sequence.

59. The construct of any one of claims 42-58, wherein the emissive readout sequence comprises a label on the 3′ end of the complimentary sequence.

60. The construct of any one of claims 42-59, wherein the emissive readout sequence comprises a label on the 5′ end and 3′ end of the complimentary sequence.

61. The construct of any one of claims 42-60, wherein the label is Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 561, Alexa Fluor 568, Alexa Fluor 594, Alexa Fluor 610, Alexa Fluor 633, Alexa Fluor 635, Alexa Fluor 647, Alexa Fluor 647-R-phycoerythrin, Alexa Fluor 660, Alexa Fluor 680, Alexa Fluor 680-allophycocyanin, Alexa Fluor 700, Alexa Fluor 750, Alexa Fluor 790, Alexa Fluor Plus 405, Alexa Fluor Plus 488, Alexa Fluor Plus 555, Alexa Fluor Plus 594, Alexa Fluor Plus 647, Alexa Fluor Plus 680, Alexa Fluor Plus 750, Alexa Fluor Plus 800, Pacific Blue, Pacific Green, Rhodamine Red X, DyLight 485-LS, DyLight-510-LS, DyLight 515-LS, DyLight 521-LS, Hydroxycoumarin, methoxycoumarin, Cy2, FAM, Fluorescein FITC, R-phycoerythrin (PE), Tamara, Cy3.5 581, Rox, Red 613, Texas Red, Cy5, Cy5.5, Cy7, Allophycocyanin, ATTO 430LS, ATTO 490LS, ATTO 390, ATTO 425, Cyan 500 NHS-Ester, ATTO 465, ATTO 488, ATTO 495, ATTO Rho110, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740.

62. A construct comprising:
a targeting sequence that is a region of interest on a nucleotide;
a first initiator sequence;
a second initiator sequence that is different from the first initiator sequence;
a first amplifier sequence comprising a third initiator sequence;
a second amplifier sequence comprising a fourth initiator sequence;
a third amplifier sequence comprising a readout sequence on the 5′ end of the sequence;
a fourth amplifier sequence comprising a readout sequence on the 3′ end of the sequence, wherein the first, second, third, and fourth amplifier sequences are different from each other; and
an emissive readout sequence comprising a sequence complimentary to the readout sequence of the third and/or fourth amplifier sequences and a label on the 5′ and/or 3′ end of the complimentary sequence.

63. A library of constructs comprising a plurality of barcoded probes, wherein each barcoded probe comprises:
a targeting sequence that is a region of interest on a nucleotide;
at least one initiator sequence;
two DNA amplifiers, wherein each DNA amplifier comprises a readout sequence; and
an emissive readout probe, wherein each emissive readout probe comprises a label and a sequence complimentary to the readout sequence of a corresponding DNA amplifier.
wherein at least two barcoded probes of the plurality of barcoded probes include targeting sequences that is specific to different regions of interest.

64. A library of constructs comprising a plurality of barcoded probes, wherein each barcoded probe comprises:
a targeting sequence that is a region of interest on a nucleotide;
a first initiator sequence;
a first and a second DNA amplifier, wherein each first and second DNA amplifier comprises a second initiator sequence
a third and a fourth DNA amplifier, wherein each third and fourth DNA amplifier comprises a readout sequence; and
an emissive readout probe, wherein each emissive readout probe comprises a label and a sequence complimentary to the readout sequence of a corresponding third and/or fourth DNA amplifier;
wherein at least two barcoded probes of the plurality of barcoded probes include targeting sequences that are specific to different regions of interest.


SEQUENCE LISTING (1257 sequences)

SEQ ID NO: 1 - DNA, 46 bp, synthetic construct
  source 1..46: mol_type = other DNA
tgccatgtgtaatcccagcagcacagctcgacgttcctttgcaaca

SEQ ID NO: 2 - DNA, 47 bp, synthetic construct
  source 1..47: mol_type = other DNA
tgtggtctctcttttcgttgggaagagctcgacgttcctttgcaaca

SEQ ID NO: 3 - DNA, 45 bp, synthetic construct
  source 1..45: mol_type = other DNA
agggcagattgtgtggacaggttagctcgacgttcctttgcaaca

SEQ ID NO: 4 - DNA, 46 bp, synthetic construct
  source 1..46: mol_type = other DNA
ggtaaaaggacagggccatcgcgttgctcgacgttcctttgcaaca

SEQ ID NO: 5 - DNA, 47 bp, synthetic construct
  source 1..47: mol_type = other DNA
ggtctgctagttgaacgcttccaagagctcgacgttcctttgcaaca

SEQ ID NO: 6 - DNA, 47 bp, synthetic construct
  source 1..47: mol_type = other DNA
tcaaacttgacttcagcacgtgtgaagctcgacgttcctttgcaaca

SEQ ID NO: 7 - DNA, 44 bp, synthetic construct
  source 1..44: mol_type = other DNA
accttcgggcatggcactctactgctcgacgttcctttgcaaca

SEQ ID NO: 8 - DNA, 46 bp, synthetic construct
  source 1..46: mol_type = other DNA
tctcgcaaagcattgaacaccaattgctcgacgttcctttgcaaca

SEQ ID NO: 9 - DNA, 46 bp, synthetic construct
  source 1..46: mol_type = other DNA
agtgacaagtgttggccatggatgtgctcgacgttcctttgcaaca

SEQ ID NO: 10 - DNA, 46 bp, synthetic construct
  source 1..46: mol_type = other DNA
tgttgcatcaccttcaccctctggtgctcgacgttcctttgcaaca

SEQ ID NO: 11 - DNA, 46 bp, synthetic construct
  source 1..46: mol_type = other DNA
tgccatgtgtaatcccagcagcacagctaccaccacgacatgtact

SEQ ID NO: 12 - DNA, 47 bp, synthetic construct
  source 1..47: mol_type = other DNA
tgtggtctctcttttcgttgggaagagctaccaccacgacatgtact

SEQ ID NO: 13 - DNA, 45 bp, synthetic construct
  source 1..45: mol_type = other DNA
agggcagattgtgtggacaggttagctaccaccacgacatgtact

SEQ ID NO: 14 - DNA, 46 bp, synthetic construct
  source 1..46: mol_type = other DNA
ggtaaaaggacagggccatcgcgttgctaccaccacgacatgtact

SEQ ID NO: 15 - DNA, 47 bp, synthetic construct
  source 1..47: mol_type = other DNA
ggtctgctagttgaacgcttccaagagctaccaccacgacatgtact

SEQ ID NO: 16 - DNA, 47 bp, synthetic construct
  source 1..47: mol_type = other DNA
tcaaacttgacttcagcacgtgtgaagctaccaccacgacatgtact

SEQ ID NO: 17 - DNA, 44 bp, synthetic construct
  source 1..44: mol_type = other DNA
accttcgggcatggcactctactgctaccaccacgacatgtact

SEQ ID NO: 18 - DNA, 46 bp, synthetic construct
  source 1..46: mol_type = other DNA
tctcgcaaagcattgaacaccaattgctaccaccacgacatgtact

SEQ ID NO: 19 - DNA, 46 bp, synthetic construct
  source 1..46: mol_type = other DNA
agtgacaagtgttggccatggatgtgctaccaccacgacatgtact

SEQ ID NO: 20 - DNA, 46 bp, synthetic construct
  source 1..46: mol_type = other DNA
tgttgcatcaccttcaccctctggtgctaccaccacgacatgtact

SEQ ID NO: 21 - DNA, 63 bp, synthetic construct
  source 1..63: mol_type = other DNA
agggtgtgtttgtaaagggtttgttgcaaaggaacgtcgagctgtaatggtgctcgacgttcc

SEQ ID NO: 22 - DNA, 63 bp, synthetic construct
  source 1..63: mol_type = other DNA
gctcgacgttcctttgcaacaggaacgtcgagcaccattacatagggtgtgtttgtaaagggt

SEQ ID NO: 23 - DNA, 63 bp, synthetic construct
  source 1..63: mol_type = other DNA
tagagttgatagagggagaatagtacatgtcgtggtggtagcttgtatgaagctaccaccacg

SEQ ID NO: 24 - DNA, 63 bp, synthetic construct
  source 1..63: mol_type = other DNA
gctaccaccacgacatgtactcgtggtggtagcttcatacaattagagttgatagagggagaa

SEQ ID NO: 25 - DNA, 20 bp, synthetic construct
  source 1..20: mol_type = other DNA
  misc_feature 1: note = Alexa Fluor 488 (NHS Ester)
tatccttcaatccctccaca

SEQ ID NO: 26 - DNA, 20 bp, synthetic construct
  source 1..20: mol_type = other DNA
  misc_feature 1: note = Alexa Fluor 546 NHS ester
acactaccaccatttcctat

SEQ ID NO: 27 - DNA, 20 bp, synthetic construct
  source 1..20: mol_type = other DNA
  misc_feature 1: note = ROX (carboxy-X-rhodamine) NHS ester
  misc_feature 20: note = ROX (carboxy-X-rhodamine) NHS ester
actccactactactcactct

SEQ ID NO: 28 - DNA, 20 bp, synthetic construct
  source 1..20: mol_type = other DNA
  misc_feature 1: note = Pacific Green dye
accctctaacttccatcaca

SEQ ID NO: 29 - DNA, 20 bp, synthetic construct
  source 1..20: mol_type = other DNA
  misc_feature 1: note = Pacific Blue dye
accacaacccattcctttca

SEQ ID NO: 30 - DNA, 20 bp, synthetic construct
  source 1..20: mol_type = other DNA
  misc_feature 1: note = Atto610
tttactccctacacctccaa

SEQ ID NO: 31 - DNA, 20 bp, synthetic construct
  source 1..20: mol_type = other DNA
  misc_feature 1: note = Alexa Fluor 647 NHS Ester
accctttacaaacacaccct

SEQ ID NO: 32 - DNA, 20 bp, synthetic construct
  source 1..20: mol_type = other DNA
  misc_feature 1: note = DyLight 510-LS NHS Ester
  misc_feature 20: note = DyLigh 510-LS NHS Ester
tcctattctcaacctaacct

SEQ ID NO: 33 - DNA, 20 bp, synthetic construct
  source 1..20: mol_type = other DNA
  misc_feature 1: note = Alexa Fluor 405 NHS Ester
ttctccctctatcaactcta

SEQ ID NO: 34 - DNA, 20 bp, synthetic construct
  source 1..20: mol_type = other DNA
  misc_feature 1: note = Alexa Fluor 532 NHS Ester
  misc_feature 20: note = Alexa Fluor 532 NHS Ester
acccttactactacatcatc

SEQ ID NO: 35 - DNA, 47 bp, synthetic construct
  source 1..47: mol_type = other DNA
agggtgtgtttgtaaagggtacctcagttaatgatagtgtgtcgttt

SEQ ID NO: 36 - DNA, 44 bp, synthetic construct
  source 1..44: mol_type = other DNA
agggtgtgtttgtaaagggtaggaaggcacattctcatctcact

SEQ ID NO: 37 - DNA, 44 bp, synthetic construct
  source 1..44: mol_type = other DNA
agggtgtgtttgtaaagggtccacgtcaatgagcaaaggtaaat

SEQ ID NO: 38 - DNA, 47 bp, synthetic construct
  source 1..47: mol_type = other DNA
agggtgtgtttgtaaagggtcctcagttaatgatagtgtgtcgattg

SEQ ID NO: 39 - DNA, 46 bp, synthetic construct
  source 1..46: mol_type = other DNA
agggtgtgtttgtaaagggtcctcagttaatgatagtgtgtcgttt

SEQ ID NO: 40 - DNA, 46 bp, synthetic construct
  source 1..46: mol_type = other DNA
agggtgtgtttgtaaagggtctaagttaatgatagtgtgtcgattg

SEQ ID NO: 41 - DNA, 44 bp, synthetic construct
  source 1..44: mol_type = other DNA
agggtgtgtttgtaaagggtgatacacacactgattcaggcaga

SEQ ID NO: 42 - DNA, 44 bp, synthetic construct
  source 1..44: mol_type = other DNA
agggtgtgtttgtaaagggtgcgtcaccccattaagaggctagg

SEQ ID NO: 43 - DNA, 45 bp, synthetic construct
  source 1..45: mol_type = other DNA
agggtgtgtttgtaaagggtgtaagctcacaatatgtgcattaaa

SEQ ID NO: 44 - DNA, 45 bp, synthetic construct
  source 1..45: mol_type = other DNA
agggtgtgtttgtaaagggtgtatcatctctgaaaacttccgacc

SEQ ID NO: 45 - DNA, 46 bp, synthetic construct
  source 1..46: mol_type = other DNA
agggtgtgtttgtaaagggtgtgtctcatctctgaaaacttcccac

SEQ ID NO: 46 - DNA, 44 bp, synthetic construct
  source 1..44: mol_type = other DNA
agggtgtgtttgtaaagggttaggctcacaatatgtgcattaaa

SEQ ID NO: 47 - DNA, 44 bp, synthetic construct
  source 1..44: mol_type = other DNA
agggtgtgtttgtaaagggttgcgtcaccccattaagaggcagg

SEQ ID NO: 48 - DNA, 44 bp, synthetic construct
  source 1..44: mol_type = other DNA
ataggaaatggtggtagtgtagtcttggttttccggatttggga

SEQ ID NO: 49 - DNA, 47 bp, synthetic construct
  source 1..47: mol_type = other DNA
ataggaaatggtggtagtgtcatgtcaatgagcaaaggtattaagaa

SEQ ID NO: 50 - DNA, 46 bp, synthetic construct
  source 1..46: mol_type = other DNA
ataggaaatggtggtagtgtcatgtcaatgagcaaaggtattatga

SEQ ID NO: 51 - DNA, 44 bp, synthetic construct
  source 1..44: mol_type = other DNA
ataggaaatggtggtagtgtcgtcaccccattaagaggctcggt

SEQ ID NO: 52 - DNA, 45 bp, synthetic construct
  source 1..45: mol_type = other DNA
ataggaaatggtggtagtgtgaaactaacacacacactgattgtc

SEQ ID NO: 53 - DNA, 44 bp, synthetic construct
  source 1..44: mol_type = other DNA
ataggaaatggtggtagtgtgagccttggttttccggatttcgg

SEQ ID NO: 54 - DNA, 44 bp, synthetic construct
  source 1..44: mol_type = other DNA
ataggaaatggtggtagtgtggagccttggttttccggattacg

SEQ ID NO: 55 - DNA, 44 bp, synthetic construct
  source 1..44: mol_type = other DNA
ataggaaatggtggtagtgtgtaagctcacaatatgtgcataaa

SEQ ID NO: 56 - DNA, 44 bp, synthetic construct
  source 1..44: mol_type = other DNA
ataggaaatggtggtagtgtgtcaccccattaagaggctccgtg

SEQ ID NO: 57 - DNA, 44 bp, synthetic construct
  source 1..44: mol_type = other DNA
ataggaaatggtggtagtgtgtgctcagccttggttttccgcta

SEQ ID NO: 58 - DNA, 47 bp, synthetic construct
  source 1..47: mol_type = other DNA
ataggaaatggtggtagtgtgtgtctcatctctgaaaacttccgacc

SEQ ID NO: 59 - DNA, 44 bp, synthetic construct
  source 1..44: mol_type = other DNA
ataggaaatggtggtagtgttgacacacacactgattcagggag

SEQ ID NO: 60 - DNA, 45 bp, synthetic construct
  source 1..45: mol_type = other DNA
cgtcaggtgagcatcttacatagtcttggttttccggatttggga

SEQ ID NO: 61 - DNA, 48 bp, synthetic construct
  source 1..48: mol_type = other DNA
cgtcaggtgagcatcttacatcatgtcaatgagcaaaggtattaagaa

SEQ ID NO: 62 - DNA, 47 bp, synthetic construct
  source 1..47: mol_type = other DNA
cgtcaggtgagcatcttacatcatgtcaatgagcaaaggtattatga

SEQ ID NO: 63 - DNA, 45 bp, synthetic construct
  source 1..45: mol_type = other DNA
cgtcaggtgagcatcttacatcgtcaccccattaagaggctcggt

SEQ ID NO: 64 - DNA, 46 bp, synthetic construct
  source 1..46: mol_type = other DNA
cgtcaggtgagcatcttacatgaaactaacacacacactgattgtc

SEQ ID NO: 65 - DNA, 45 bp, synthetic construct
  source 1..45: mol_type = other DNA
cgtcaggtgagcatcttacatgagccttggttttccggatttcgg

SEQ ID NO: 66 - DNA, 45 bp, synthetic construct
  source 1..45: mol_type = other DNA
cgtcaggtgagcatcttacatggagccttggttttccggattacg

SEQ ID NO: 67 - DNA, 45 bp, synthetic construct
  source 1..45: mol_type = other DNA
cgtcaggtgagcatcttacatgtaagctcacaatatgtgcataaa

SEQ ID NO: 68 - DNA, 45 bp, synthetic construct
  source 1..45: mol_type = other DNA
cgtcaggtgagcatcttacatgtcaccccattaagaggctccgtg

SEQ ID NO: 69 - DNA, 45 bp, synthetic construct
  source 1..45: mol_type = other DNA
cgtcaggtgagcatcttacatgtgctcagccttggttttccgcta

SEQ ID NO: 70 - DNA, 48 bp, synthetic construct
  source 1..48: mol_type = other DNA
cgtcaggtgagcatcttacatgtgtctcatctctgaaaacttccgacc

SEQ ID NO: 71 - DNA, 45 bp, synthetic construct
  source 1..45: mol_type = other DNA
cgtcaggtgagcatcttacattgacacacacactgattcagggag

SEQ ID NO: 72 - DNA, 48 bp, synthetic construct
  source 1..48: mol_type = other DNA
gctcgacgttcctttgcaacaacctcagttaatgatagtgtgtcgttt

SEQ ID NO: 73 - DNA, 45 bp, synthetic construct
  source 1..45: mol_type = other DNA
gctcgacgttcctttgcaacaaggaaggcacattctcatctcact

SEQ ID NO: 74 - DNA, 45 bp, synthetic construct
  source 1..45: mol_type = other DNA
gctcgacgttcctttgcaacaccacgtcaatgagcaaaggtaaat

SEQ ID NO: 75 - DNA, 48 bp, synthetic construct
  source 1..48: mol_type = other DNA
gctcgacgttcctttgcaacacctcagttaatgatagtgtgtcgattg

SEQ ID NO: 76 - DNA, 47 bp, synthetic construct
  source 1..47: mol_type = other DNA
gctcgacgttcctttgcaacacctcagttaatgatagtgtgtcgttt

SEQ ID NO: 77 - DNA, 47 bp, synthetic construct
  source 1..47: mol_type = other DNA
gctcgacgttcctttgcaacactaagttaatgatagtgtgtcgattg

SEQ ID NO: 78 - DNA, 45 bp, synthetic construct
  source 1..45: mol_type = other DNA
gctcgacgttcctttgcaacagatacacacactgattcaggcaga

SEQ ID NO: 79 - DNA, 45 bp, synthetic construct
  source 1..45: mol_type = other DNA
gctcgacgttcctttgcaacagcgtcaccccattaagaggctagg

SEQ ID NO: 80 - DNA, 46 bp, synthetic construct
  source 1..46: mol_type = other DNA
gctcgacgttcctttgcaacagtaagctcacaatatgtgcattaaa

SEQ ID NO: 81 - DNA, 46 bp, synthetic construct
  source 1..46: mol_type = other DNA
gctcgacgttcctttgcaacagtatcatctctgaaaacttccgacc

SEQ ID NO: 82 - DNA, 47 bp, synthetic construct
  source 1..47: mol_type = other DNA
gctcgacgttcctttgcaacagtgtctcatctctgaaaacttcccac

SEQ ID NO: 83 - DNA, 45 bp, synthetic construct
  source 1..45: mol_type = other DNA
gctcgacgttcctttgcaacataggctcacaatatgtgcattaaa

SEQ ID NO: 84 - DNA, 45 bp, synthetic construct
  source 1..45: mol_type = other DNA
gctcgacgttcctttgcaacatgcgtcaccccattaagaggcagg

SEQ ID NO: 85 - DNA, 63 bp, synthetic construct
  source 1..63: mol_type = other DNA
ataggaaatggtggtagtgttatgtaagatgctcacctgacgttcatgtaacgtcaggtgagc

SEQ ID NO: 86 - DNA, 63 bp, synthetic construct
  source 1..63: mol_type = other DNA
cgtcaggtgagcatcttacatgctcacctgacgttacatgaatataggaaatggtggtagtgt

SEQ ID NO: 87 - DNA, 46 bp, synthetic construct
  source 1..46: mol_type = other DNA
tgccatgtgtaatcccagcagcacacgtcaggtgagcatcttacat

SEQ ID NO: 88 - DNA, 47 bp, synthetic construct
  source 1..47: mol_type = other DNA
tgtggtctctcttttcgttgggaagacgtcaggtgagcatcttacat

SEQ ID NO: 89 - DNA, 45 bp, synthetic construct
  source 1..45: mol_type = other DNA
agggcagattgtgtggacaggttacgtcaggtgagcatcttacat

SEQ ID NO: 90 - DNA, 46 bp, synthetic construct
  source 1..46: mol_type = other DNA
ggtaaaaggacagggccatcgcgttcgtcaggtgagcatcttacat

SEQ ID NO: 91 - DNA, 47 bp, synthetic construct
  source 1..47: mol_type = other DNA
ggtctgctagttgaacgcttccaagacgtcaggtgagcatcttacat

SEQ ID NO: 92 - DNA, 47 bp, synthetic construct
  source 1..47: mol_type = other DNA
tcaaacttgacttcagcacgtgtgaacgtcaggtgagcatcttacat

SEQ ID NO: 93 - DNA, 44 bp, synthetic construct
  source 1..44: mol_type = other DNA
accttcgggcatggcactctactcgtcaggtgagcatcttacat

SEQ ID NO: 94 - DNA, 46 bp, synthetic construct
  source 1..46: mol_type = other DNA
tctcgcaaagcattgaacaccaattcgtcaggtgagcatcttacat

SEQ ID NO: 95 - DNA, 46 bp, synthetic construct
  source 1..46: mol_type = other DNA
agtgacaagtgttggccatggatgtcgtcaggtgagcatcttacat

SEQ ID NO: 96 - DNA, 46 bp, synthetic construct
  source 1..46: mol_type = other DNA
tgttgcatcaccttcaccctctggtcgtcaggtgagcatcttacat

SEQ ID NO: 97 - DNA, 49 bp, synthetic construct
  source 1..49: mol_type = other DNA
cgtcaggtgagcatcttacatctattcggcgctccacagttccggattt

SEQ ID NO: 98 - DNA, 49 bp, synthetic construct
  source 1..49: mol_type = other DNA
cgtcaggtgagcatcttacatttgtgcttaccttgcgggccaacatcca

SEQ ID NO: 99 - DNA, 49 bp, synthetic construct
  source 1..49: mol_type = other DNA
cgtcaggtgagcatcttacatcggtccagtaccgcgcggctgaaatcat

SEQ ID NO: 100 - DNA, 49 bp, synthetic construct
  source 1..49: mol_type = other DNA
cgtcaggtgagcatcttacatcgctcgtgattagcgccgtggcctgatt

SEQ ID NO: 101 - DNA, 49 bp, synthetic construct
  source 1..49: mol_type = other DNA
cgtcaggtgagcatcttacatgcgacagcgtgtaccacagcggatggtt

SEQ ID NO: 102 - DNA, 49 bp, synthetic construct
  source 1..49: mol_type = other DNA
cgtcaggtgagcatcttacatagttacagaactggcgatcgttcggcgt

SEQ ID NO: 103 - DNA, 49 bp, synthetic construct
  source 1..49: mol_type = other DNA
cgtcaggtgagcatcttacattaacattggcaccatgccgtgggtttca

SEQ ID NO: 104 - DNA, 49 bp, synthetic construct
  source 1..49: mol_type = other DNA
cgtcaggtgagcatcttacatcggtcttcgctattacgccagctggcga

SEQ ID NO: 105 - DNA, 49 bp, synthetic construct
  source 1..49: mol_type = other DNA
cgtcaggtgagcatcttacattagacactcgggtgattacgatcgcgct

SEQ ID NO: 106 - DNA, 49 bp, synthetic construct
  source 1..49: mol_type = other DNA
cgtcaggtgagcatcttacataggagataactgccgtcactccagcgca

SEQ ID NO: 107 - DNA, 49 bp, synthetic construct
  source 1..49: mol_type = other DNA
cgtcaggtgagcatcttacataaatttgatggaccatttcggcaccgcc

SEQ ID NO: 108 - DNA, 49 bp, synthetic construct
  source 1..49: mol_type = other DNA
cgtcaggtgagcatcttacatctagtgccgcaaggcgattaagttgggt

SEQ ID NO: 109 - DNA, 49 bp, synthetic construct
  source 1..49: mol_type = other DNA
cgtcaggtgagcatcttacatcagtgacaatggcagatcccagcggtca

SEQ ID NO: 110 - DNA, 49 bp, synthetic construct
  source 1..49: mol_type = other DNA
cgtcaggtgagcatcttacattttatgccgctcatccgccacatatcct

SEQ ID NO: 111 - DNA, 49 bp, synthetic construct
  source 1..49: mol_type = other DNA
cgtcaggtgagcatcttacataggagtgccaccatccagtgcaggaact

SEQ ID NO: 112 - DNA, 49 bp, synthetic construct
  source 1..49: mol_type = other DNA
cgtcaggtgagcatcttacattgatcgatggttcgcccggataaacgga

SEQ ID NO: 113 - DNA, 49 bp, synthetic construct
  source 1..49: mol_type = other DNA
cgtcaggtgagcatcttacatccgggtgtgcagttcaaccactgcacga

SEQ ID NO: 114 - DNA, 49 bp, synthetic construct
  source 1..49: mol_type = other DNA
cgtcaggtgagcatcttacattggatctcaccgtgcccatcaatccggt

SEQ ID NO: 115 - DNA, 49 bp, synthetic construct
  source 1..49: mol_type = other DNA
cgtcaggtgagcatcttacatttagcgctcaggtcaaattcagacggca

SEQ ID NO: 116 - DNA, 49 bp, synthetic construct
  source 1..49: mol_type = other DNA
cgtcaggtgagcatcttacatcagaggaagatcgcactccagccagctt

SEQ ID NO: 117 - DNA, 49 bp, synthetic construct
  source 1..49: mol_type = other DNA
cgtcaggtgagcatcttacatccgtgatgttgaactggaagtcgccgcg

SEQ ID NO: 118 - DNA, 49 bp, synthetic construct
  source 1..49: mol_type = other DNA
cgtcaggtgagcatcttacatccggcatcgtaaccgtgcatctgccagt

SEQ ID NO: 119 - DNA, 49 bp, synthetic construct
  source 1..49: mol_type = other DNA
cgtcaggtgagcatcttacatagccgcagctcgccgtacatctgaactt

SEQ ID NO: 120 - DNA, 49 bp, synthetic construct
  source 1..49: mol_type = other DNA
cgtcaggtgagcatcttacatcccccataattcaattcgcgcgtcccgc

SEQ ID NO: 121 - DNA, 49 bp, synthetic construct
  source 1..49: mol_type = other DNA
cgtcaggtgagcatcttacatgcatgcaccacagatgaaacgccgagtt

SEQ ID NO: 122 - DNA, 49 bp, synthetic construct
  source 1..49: mol_type = other DNA
cgtcaggtgagcatcttacatttaaattcgcgtctggccttcctgtagc

SEQ ID NO: 123 - DNA, 49 bp, synthetic construct
  source 1..49: mol_type = other DNA
cgtcaggtgagcatcttacatgaaggaaatcgctgatttgcgtggtcgg

SEQ ID NO: 124 - DNA, 49 bp, synthetic construct
  source 1..49: mol_type = other DNA
cgtcaggtgagcatcttacattgaacgcgtaccgttagccagagttgtc

SEQ ID NO: 125 - DNA, 49 bp, synthetic construct
  source 1..49: mol_type = other DNA
cgtcaggtgagcatcttacatcggttcatactgtaccgggcgggaagga

SEQ ID NO: 126 - DNA, 49 bp, synthetic construct
  source 1..49: mol_type = other DNA
cgtcaggtgagcatcttacatgagagctggaattccgccgatactgacg

SEQ ID NO: 127 - DNA, 49 bp, synthetic construct
  source 1..49: mol_type = other DNA
cgtcaggtgagcatcttacatcgcactgatccacccagtcccagacgaa

SEQ ID NO: 128 - DNA, 49 bp, synthetic construct
  source 1..49: mol_type = other DNA
cgtcaggtgagcatcttacattagtgtgaaagaaagcctgactggcggt

SEQ ID NO: 129 - DNA, 63 bp, synthetic construct
  source 1..63: mol_type = other DNA
agggtgtgtttgtaaagggttatgtaagatgctcacctgacgttcatgtaacgtcaggtgagc

SEQ ID NO: 130 - DNA, 63 bp, synthetic construct
  source 1..63: mol_type = other DNA
cgtcaggtgagcatcttacatgctcacctgacgttacatgaatagggtgtgtttgtaaagggt

SEQ ID NO: 131 - DNA, 63 bp, synthetic construct
  source 1..63: mol_type = other DNA
tgtggagggattgaaggatattgttgcaaaggaacgtcgagctgtaatggtgctcgacgttcc

SEQ ID NO: 132 - DNA, 63 bp, synthetic construct
  source 1..63: mol_type = other DNA
gctcgacgttcctttgcaacaggaacgtcgagcaccattacattgtggagggattgaaggata

SEQ ID NO: 133 - DNA, 47 bp, synthetic construct
  source 1..47: mol_type = other DNA
gctcgacgttcctttgcaacagaaagcgcaacaagcgcggctacttt

SEQ ID NO: 134 - DNA, 47 bp, synthetic construct
  source 1..47: mol_type = other DNA
gctcgacgttcctttgcaacacagtgtttcgcggtcgccagcgttaa

SEQ ID NO: 135 - DNA, 47 bp, synthetic construct
  source 1..47: mol_type = other DNA
gctcgacgttcctttgcaacagataagcggttacacatgctgccgga

SEQ ID NO: 136 - DNA, 47 bp, synthetic construct
  source 1..47: mol_type = other DNA
gctcgacgttcctttgcaacaccatcgcggtcagatccacttgtgca

SEQ ID NO: 137 - DNA, 47 bp, synthetic construct
  source 1..47: mol_type = other DNA
gctcgacgttcctttgcaacagacgtcacgggcttaccgtttacgct

SEQ ID NO: 138 - DNA, 47 bp, synthetic construct
  source 1..47: mol_type = other DNA
gctcgacgttcctttgcaacaaatgcgcacatcatacgggtcattgc

SEQ ID NO: 139 - DNA, 47 bp, synthetic construct
  source 1..47: mol_type = other DNA
gctcgacgttcctttgcaacacgagtagctgttctggcgttacagca

SEQ ID NO: 140 - DNA, 47 bp, synthetic construct
  source 1..47: mol_type = other DNA
gctcgacgttcctttgcaacaagatcatacagcaaggctgcctcgct

SEQ ID NO: 141 - DNA, 47 bp, synthetic construct
  source 1..47: mol_type = other DNA
gctcgacgttcctttgcaacagcatcgtcatttccctggcgcagagt

SEQ ID NO: 142 - DNA, 47 bp, synthetic construct
  source 1..47: mol_type = other DNA
gctcgacgttcctttgcaacacccgcgacgctgttcagtaatcgcct

SEQ ID NO: 143 - DNA, 47 bp, synthetic construct
  source 1..47: mol_type = other DNA
gctcgacgttcctttgcaacaattaaacgggcagcccatagccattt

SEQ ID NO: 144 - DNA, 47 bp, synthetic construct
  source 1..47: mol_type = other DNA
gctcgacgttcctttgcaacaaatccgccttcaatcatttcacgggc

SEQ ID NO: 145 - DNA, 47 bp, synthetic construct
  source 1..47: mol_type = other DNA
gctcgacgttcctttgcaacaacattaaatcgtaacaggtcgcggcg

SEQ ID NO: 146 - DNA, 47 bp, synthetic construct
  source 1..47: mol_type = other DNA
gctcgacgttcctttgcaacactcttgtttgcggatggtctgcgcca

SEQ ID NO: 147 - DNA, 47 bp, synthetic construct
  source 1..47: mol_type = other DNA
gctcgacgttcctttgcaacacgcaagctcgtcattcaccgccagct

SEQ ID NO: 148 - DNA, 47 bp, synthetic construct
  source 1..47: mol_type = other DNA
gctcgacgttcctttgcaacatggtaacagggaatggcggacctgct

SEQ ID NO: 149 - DNA, 47 bp, synthetic construct
  source 1..47: mol_type = other DNA
gctcgacgttcctttgcaacatataaccgggtcgatatccacgacca

SEQ ID NO: 150 - DNA, 47 bp, synthetic construct
  source 1..47: mol_type = other DNA
gctcgacgttcctttgcaacattatcgctacttagctgggcttcagc

SEQ ID NO: 151 - DNA, 47 bp, synthetic construct
  source 1..47: mol_type = other DNA
gctcgacgttcctttgcaacaggaccatcaccacgtgataccacgga

SEQ ID NO: 152 - DNA, 47 bp, synthetic construct
  source 1..47: mol_type = other DNA
gctcgacgttcctttgcaacaccaaggtatgaaccggtaggccgtta

SEQ ID NO: 153 - DNA, 47 bp, synthetic construct
  source 1..47: mol_type = other DNA
gctcgacgttcctttgcaacagtaccatggatggctgttcaggatgt

SEQ ID NO: 154 - DNA, 47 bp, synthetic construct
  source 1..47: mol_type = other DNA
gctcgacgttcctttgcaacattgcgatccattgacgcatcagtggg

SEQ ID NO: 155 - DNA, 47 bp, synthetic construct
  source 1..47: mol_type = other DNA
gctcgacgttcctttgcaacaactttcataagcccttgatgcagcca

SEQ ID NO: 156 - DNA, 47 bp, synthetic construct
  source 1..47: mol_type = other DNA
gctcgacgttcctttgcaacatggagccacagcgatagcaatgcggt

SEQ ID NO: 157 - DNA, 47 bp, synthetic construct
  source 1..47: mol_type = other DNA
gctcgacgttcctttgcaacattcttgcgttcagcgatgccctggta

SEQ ID NO: 158 - DNA, 47 bp, synthetic construct
  source 1..47: mol_type = other DNA
gctcgacgttcctttgcaacatggaccagcagattatcctgggcggt

SEQ ID NO: 159 - DNA, 47 bp, synthetic construct
  source 1..47: mol_type = other DNA
gctcgacgttcctttgcaacaaagcggaatcacgcgttcgagatcga

SEQ ID NO: 160 - DNA, 47 bp, synthetic construct
  source 1..47: mol_type = other DNA
gctcgacgttcctttgcaacaaattacggagggtagccgccattact

SEQ ID NO: 161 - DNA, 47 bp, synthetic construct
  source 1..47: mol_type = other DNA
gctcgacgttcctttgcaacatctccattcaccaggttagcaccacg

SEQ ID NO: 162 - DNA, 47 bp, synthetic construct
  source 1..47: mol_type = other DNA
gctcgacgttcctttgcaacatttcggtcaaatccaatagcagaacg

SEQ ID NO: 163 - DNA, 47 bp, synthetic construct
  source 1..47: mol_type = other DNA
gctcgacgttcctttgcaacaagatttggctgctctggcgtgccttt

SEQ ID NO: 164 - DNA, 47 bp, synthetic construct
  source 1..47: mol_type = other DNA
gctcgacgttcctttgcaacagacatagcgatacgctgcgctgcgat

SEQ ID NO: 165 - DNA, 47 bp, synthetic construct
  source 1..47: mol_type = other DNA
gctcgacgttcctttgcaacaattaccgtttacgaaggttgcgccag

SEQ ID NO: 166 - DNA, 47 bp, synthetic construct
  source 1..47: mol_type = other DNA
gctcgacgttcctttgcaacacggagcgcaagggtaatgcggtagtg

SEQ ID NO: 167 - DNA, 47 bp, synthetic construct
  source 1..47: mol_type = other DNA
gctcgacgttcctttgcaacaagtatgttgtacggcgggaagctctg

SEQ ID NO: 168 - DNA, 47 bp, synthetic construct
  source 1..47: mol_type = other DNA
gctcgacgttcctttgcaacaaataacatgcgactggttgcggcagt

SEQ ID NO: 169 - DNA, 47 bp, synthetic construct
  source 1..47: mol_type = other DNA
gctcgacgttcctttgcaacagtggcgtttgggattgggcaaagcgt

SEQ ID NO: 170 - DNA, 47 bp, synthetic construct
  source 1..47: mol_type = other DNA
gctcgacgttcctttgcaacattaatcttccgcgagaaacgcgaggt

SEQ ID NO: 171 - DNA, 47 bp, synthetic construct
  source 1..47: mol_type = other DNA
gctcgacgttcctttgcaacaaatatcagcggcggtttatcccacca

SEQ ID NO: 172 - DNA, 47 bp, synthetic construct
  source 1..47: mol_type = other DNA
gctcgacgttcctttgcaacaacgtggcacacagcttctggtagcga

SEQ ID NO: 173 - DNA, 47 bp, synthetic construct
  source 1..47: mol_type = other DNA
gctcgacgttcctttgcaacacgggcatccattcccgcgcagtttct

SEQ ID NO: 174 - DNA, 47 bp, synthetic construct
  source 1..47: mol_type = other DNA
gctcgacgttcctttgcaacacggctggtctgctgctgcagtgacaa

SEQ ID NO: 175 - DNA, 47 bp, synthetic construct
  source 1..47: mol_type = other DNA
gctcgacgttcctttgcaacaattaaacaggttgtccatcagcgcga

SEQ ID NO: 176 - DNA, 47 bp, synthetic construct
  source 1..47: mol_type = other DNA
gctcgacgttcctttgcaacagtccgggcggcggtcatgaatatcta

SEQ ID NO: 177 - DNA, 47 bp, synthetic construct
  source 1..47: mol_type = other DNA
gctcgacgttcctttgcaacattgaggttgaattaactccgcgccct

SEQ ID NO: 178 - DNA, 47 bp, synthetic construct
  source 1..47: mol_type = other DNA
gctcgacgttcctttgcaacacaacgggatcgtagggaatatcgcgt

SEQ ID NO: 179 - DNA, 47 bp, synthetic construct
  source 1..47: mol_type = other DNA
gctcgacgttcctttgcaacagcgtcaaacggtgtgctacctatcgc

SEQ ID NO: 180 - DNA, 47 bp, synthetic construct
  source 1..47: mol_type = other DNA
gctcgacgttcctttgcaacaaagagccaggcagtcgcattcgcttt

SEQ ID NO: 181 - DNA, 47 bp, synthetic construct
  source 1..47: mol_type = other DNA
gctcgacgttcctttgcaacaaagaccactttcacgcgggtttcgct

SEQ ID NO: 182 - DNA, 47 bp, synthetic construct
  source 1..47: mol_type = other DNA
gctcgacgttcctttgcaacattaatcgacgcccagtttacgtgcgt

SEQ ID NO: 183 - DNA, 47 bp, synthetic construct
  source 1..47: mol_type = other DNA
gctcgacgttcctttgcaacagagatcatacgtgccgcaaggcccat

SEQ ID NO: 184 - DNA, 47 bp, synthetic construct
  source 1..47: mol_type = other DNA
gctcgacgttcctttgcaacatggacgcgcctgctttctcgataagc

SEQ ID NO: 185 - DNA, 47 bp, synthetic construct
  source 1..47: mol_type = other DNA
gctcgacgttcctttgcaacactacagcagcgtgttggactgcttca

SEQ ID NO: 186 - DNA, 47 bp, synthetic construct
  source 1..47: mol_type = other DNA
gctcgacgttcctttgcaacatctaatccggcgttgagttcgggttg

SEQ ID NO: 187 - DNA, 47 bp, synthetic construct
  source 1..47: mol_type = other DNA
gctcgacgttcctttgcaacagggaggtcaaccagctcgccgtagaa

SEQ ID NO: 188 - DNA, 47 bp, synthetic construct
  source 1..47: mol_type = other DNA
gctcgacgttcctttgcaacactaaacgtctactgcgccagaacgtg

SEQ ID NO: 189 - DNA, 47 bp, synthetic construct
  source 1..47: mol_type = other DNA
gctcgacgttcctttgcaacaggtggcgcatgatggagcctttacca

SEQ ID NO: 190 - DNA, 47 bp, synthetic construct
  source 1..47: mol_type = other DNA
gctcgacgttcctttgcaacaacattctcaatctggcccagtgctgc

SEQ ID NO: 191 - DNA, 47 bp, synthetic construct
  source 1..47: mol_type = other DNA
gctcgacgttcctttgcaacaaagtcgatctctttcgcggtttccgg

SEQ ID NO: 192 - DNA, 47 bp, synthetic construct
  source 1..47: mol_type = other DNA
gctcgacgttcctttgcaacaggtgcaaaccgaatcgacgtgccagt

SEQ ID NO: 193 - DNA, 47 bp, synthetic construct
  source 1..47: mol_type = other DNA
gctcgacgttcctttgcaacaacatgagaagcggaaaccacgttccg

SEQ ID NO: 194 - DNA, 47 bp, synthetic construct
  source 1..47: mol_type = other DNA
gctcgacgttcctttgcaacacggcgttcgatcgtctggcgaatcca

SEQ ID NO: 195 - DNA, 47 bp, synthetic construct
  source 1..47: mol_type = other DNA
gctcgacgttcctttgcaacagggtcttcgatcaggtccagcaacgc

SEQ ID NO: 196 - DNA, 47 bp, synthetic construct
  source 1..47: mol_type = other DNA
gctcgacgttcctttgcaacaagtaacttctctaccgcgcggatcag

SEQ ID NO: 197 - DNA, 47 bp, synthetic construct
  source 1..47: mol_type = other DNA
gctcgacgttcctttgcaacacactggctccctgcgataacagttcc

SEQ ID NO: 198 - DNA, 47 bp, synthetic construct
  source 1..47: mol_type = other DNA
gctcgacgttcctttgcaacaccctgtctaccgaggtaatgcgctcg

SEQ ID NO: 199 - DNA, 47 bp, synthetic construct
  source 1..47: mol_type = other DNA
gctcgacgttcctttgcaacaagatcttcggccgttaacagtggtga

SEQ ID NO: 200 - DNA, 47 bp, synthetic construct
  source 1..47: mol_type = other DNA
gctcgacgttcctttgcaacagctacagccatttgacgatgctctgc

SEQ ID NO: 201 - DNA, 47 bp, synthetic construct
  source 1..47: mol_type = other DNA
gctcgacgttcctttgcaacagaagcgtggtatcttccggaccattc

SEQ ID NO: 202 - DNA, 47 bp, synthetic construct
  source 1..47: mol_type = other DNA
gctcgacgttcctttgcaacactttcggcaaacgaatagtacgggtt

SEQ ID NO: 203 - DNA, 47 bp, synthetic construct
  source 1..47: mol_type = other DNA
gctcgacgttcctttgcaacaaagggccaaatcgttatcactgggtt

SEQ ID NO: 204 - DNA, 47 bp, synthetic construct
  source 1..47: mol_type = other DNA
gctcgacgttcctttgcaacagtcacaacatcaagcgcagccgacca

SEQ ID NO: 205 - DNA, 47 bp, synthetic construct
  source 1..47: mol_type = other DNA
gctcgacgttcctttgcaacattaatcaagcaccaggccgggtttgt

SEQ ID NO: 206 - DNA, 47 bp, synthetic construct
  source 1..47: mol_type = other DNA
gctcgacgttcctttgcaacatttaccacgaatccagaagcgagcga

SEQ ID NO: 207 - DNA, 47 bp, synthetic construct
  source 1..47: mol_type = other DNA
gctcgacgttcctttgcaacatcgccgttcattcgtggcatcgcgat

SEQ ID NO: 208 - DNA, 47 bp, synthetic construct
  source 1..47: mol_type = other DNA
gctcgacgttcctttgcaacatcaacgccattatgtccagctcgggt

SEQ ID NO: 209 - DNA, 47 bp, synthetic construct
  source 1..47: mol_type = other DNA
gctcgacgttcctttgcaacaagtataacgcgcccaactctggcaac

SEQ ID NO: 210 - DNA, 47 bp, synthetic construct
  source 1..47: mol_type = other DNA
gctcgacgttcctttgcaacatttaccatctcgcgcaagcgattcag

SEQ ID NO: 211 - DNA, 47 bp, synthetic construct
  source 1..47: mol_type = other DNA
gctcgacgttcctttgcaacaatacaaccattgcatcccagtcgcga

SEQ ID NO: 212 - DNA, 47 bp, synthetic construct
  source 1..47: mol_type = other DNA
gctcgacgttcctttgcaacagcacgcattcagacccgcagaaacca

SEQ ID NO: 213 - DNA, 47 bp, synthetic construct
  source 1..47: mol_type = other DNA
gctcgacgttcctttgcaacaagtacacccagacgtaacgctttggc

SEQ ID NO: 214 - DNA, 47 bp, synthetic construct
  source 1..47: mol_type = other DNA
gctcgacgttcctttgcaacatggtgctgaaccggcggttgtagttc

SEQ ID NO: 215 - DNA, 47 bp, synthetic construct
  source 1..47: mol_type = other DNA
gctcgacgttcctttgcaacagacatttgcacctgatgttcgccggt

SEQ ID NO: 216 - DNA, 49 bp, synthetic construct
  source 1..49: mol_type = other DNA
gggtggtcgtcgaagtcgtataagacgcgttggcgcgcaaagcattgaa

SEQ ID NO: 217 - DNA, 49 bp, synthetic construct
  source 1..49: mol_type = other DNA
gggtggtcgtcgaagtcgtatcggtgcgcctgttggcgaatgtgcaaca

SEQ ID NO: 218 - DNA, 49 bp, synthetic construct
  source 1..49: mol_type = other DNA
gggtggtcgtcgaagtcgtataaaagcaaatgcggtttaccgtcgcgca

SEQ ID NO: 219 - DNA, 49 bp, synthetic construct
  source 1..49: mol_type = other DNA
gggtggtcgtcgaagtcgtattgattgcgcaggaacgtcgttgatcgca

SEQ ID NO: 220 - DNA, 49 bp, synthetic construct
  source 1..49: mol_type = other DNA
gggtggtcgtcgaagtcgtatgacataaacggcagtgaggcgcgggttt

SEQ ID NO: 221 - DNA, 49 bp, synthetic construct
  source 1..49: mol_type = other DNA
gggtggtcgtcgaagtcgtattctatcccttgcgcctgggtgtttgctt

SEQ ID NO: 222 - DNA, 49 bp, synthetic construct
  source 1..49: mol_type = other DNA
gggtggtcgtcgaagtcgtataaatttaatcagcggcgaacgggcgcta

SEQ ID NO: 223 - DNA, 49 bp, synthetic construct
  source 1..49: mol_type = other DNA
gggtggtcgtcgaagtcgtatcggtgcgtttcatcgtcgtctgccggaa

SEQ ID NO: 224 - DNA, 49 bp, synthetic construct
  source 1..49: mol_type = other DNA
gggtggtcgtcgaagtcgtatacgaaccggaatgcccggcaactgttct

SEQ ID NO: 225 - DNA, 49 bp, synthetic construct
  source 1..49: mol_type = other DNA
gggtggtcgtcgaagtcgtatcgatcacgatcgagacgcgaacgccatt

SEQ ID NO: 226 - DNA, 49 bp, synthetic construct
  source 1..49: mol_type = other DNA
gggtggtcgtcgaagtcgtatttaaccacatccttaaacccggcacgct

SEQ ID NO: 227 - DNA, 49 bp, synthetic construct
  source 1..49: mol_type = other DNA
gggtggtcgtcgaagtcgtataagacgggcatcgcgtaccagatcgtta

SEQ ID NO: 228 - DNA, 49 bp, synthetic construct
  source 1..49: mol_type = other DNA
gggtggtcgtcgaagtcgtatcgaacgcaccaggcgatagcttaaacgc

SEQ ID NO: 229 - DNA, 49 bp, synthetic construct
  source 1..49: mol_type = other DNA
gggtggtcgtcgaagtcgtattacaacagtcagtcgtaccaccgccgat

SEQ ID NO: 230 - DNA, 49 bp, synthetic construct
  source 1..49: mol_type = other DNA
gggtggtcgtcgaagtcgtatccccgctttccagcccttgctggctaat

SEQ ID NO: 231 - DNA, 49 bp, synthetic construct
  source 1..49: mol_type = other DNA
gggtggtcgtcgaagtcgtatagccggttataacgaatcgcccgacgca

SEQ ID NO: 232 - DNA, 49 bp, synthetic construct
  source 1..49: mol_type = other DNA
gggtggtcgtcgaagtcgtatgtcatcctcaaacagcgctacctgctgc

SEQ ID NO: 233 - DNA, 49 bp, synthetic construct
  source 1..49: mol_type = other DNA
gggtggtcgtcgaagtcgtataggtgcaaggtggcttcgtaatccagcc

SEQ ID NO: 234 - DNA, 49 bp, synthetic construct
  source 1..49: mol_type = other DNA
gggtggtcgtcgaagtcgtatggtgtgaggaaagaccgaactgcacgct

SEQ ID NO: 235 - DNA, 49 bp, synthetic construct
  source 1..49: mol_type = other DNA
gggtggtcgtcgaagtcgtatgtgccatgcccagtaacggcatcaggtt

SEQ ID NO: 236 - DNA, 49 bp, synthetic construct
  source 1..49: mol_type = other DNA
gggtggtcgtcgaagtcgtatagcggcgaccaatcaccgcctgagtaat

SEQ ID NO: 237 - DNA, 49 bp, synthetic construct
  source 1..49: mol_type = other DNA
gggtggtcgtcgaagtcgtattacatggcggtacagccattcgcttaca

SEQ ID NO: 238 - DNA, 49 bp, synthetic construct
  source 1..49: mol_type = other DNA
gggtggtcgtcgaagtcgtatgtttacggcaaccactatgacccagcag

SEQ ID NO: 239 - DNA, 49 bp, synthetic construct
  source 1..49: mol_type = other DNA
gggtggtcgtcgaagtcgtattgggccactgaacagtttgctgtaccgt

SEQ ID NO: 240 - DNA, 49 bp, synthetic construct
  source 1..49: mol_type = other DNA
gggtggtcgtcgaagtcgtattgcccagactttctgtaacagggccact

SEQ ID NO: 241 - DNA, 49 bp, synthetic construct
  source 1..49: mol_type = other DNA
gggtggtcgtcgaagtcgtattgcccagtgcgatatccagatcgttacc

SEQ ID NO: 242 - DNA, 63 bp, synthetic construct
  source 1..63: mol_type = other DNA
tagagttgatagagggagaattgttgcaaaggaacgtcgagctgtaatggtgctcgacgttcc

SEQ ID NO: 243 - DNA, 63 bp, synthetic construct
  source 1..63: mol_type = other DNA
gctcgacgttcctttgcaacaggaacgtcgagcaccattacattagagttgatagagggagaa

SEQ ID NO: 244 - DNA, 63 bp, synthetic construct
  source 1..63: mol_type = other DNA
agggtgtgtttgtaaagggttatacgacttcgacgaccacccaacttgaatgggtggtcgtcg

SEQ ID NO: 245 - DNA, 63 bp, synthetic construct
  source 1..63: mol_type = other DNA
gggtggtcgtcgaagtcgtatcgacgaccacccattcaagtttagggtgtgtttgtaaagggt

SEQ ID NO: 246 - DNA, 38 bp, synthetic construct
  source 1..38: mol_type = other DNA
tagagttgatagagggagaagctgcctcccgtaggagt

SEQ ID NO: 247 - DNA, 49 bp, synthetic construct
  source 1..49: mol_type = other DNA
cgtcggagtgggttcagtctagccaaaccaggcaaagcgccattcgcca

SEQ ID NO: 248 - DNA, 49 bp, synthetic construct
  source 1..49: mol_type = other DNA
cgtcggagtgggttcagtctaatttgcgaacagcgcacggcgttaaagt

SEQ ID NO: 249 - DNA, 49 bp, synthetic construct
  source 1..49: mol_type = other DNA
cgtcggagtgggttcagtctagccttaacgccgcgaatcagcaacggct

SEQ ID NO: 250 - DNA, 49 bp, synthetic construct
  source 1..49: mol_type = other DNA
cgtcggagtgggttcagtctagcttaatttcaccgccgaaaggcgcggt

SEQ ID NO: 251 - DNA, 49 bp, synthetic construct
  source 1..49: mol_type = other DNA
cgtcggagtgggttcagtctacattcgcttgccaccgcaacatccacat

SEQ ID NO: 252 - DNA, 49 bp, synthetic construct
  source 1..49: mol_type = other DNA
cgtcggagtgggttcagtctagggtgccacaaagaaaccgtcacccgca

SEQ ID NO: 253 - DNA, 49 bp, synthetic construct
  source 1..49: mol_type = other DNA
cgtcggagtgggttcagtctacgctgcagcagatggcgatggctggttt

SEQ ID NO: 254 - DNA, 49 bp, synthetic construct
  source 1..49: mol_type = other DNA
cgtcggagtgggttcagtctaacgaacaacgccgcttcggcctggtaat

SEQ ID NO: 255 - DNA, 49 bp, synthetic construct
  source 1..49: mol_type = other DNA
cgtcggagtgggttcagtctacgatctgaccatgcggtcgcgtttggtt

SEQ ID NO: 256 - DNA, 49 bp, synthetic construct
  source 1..49: mol_type = other DNA
cgtcggagtgggttcagtctagccaaaccgacgtcgcaggcttctgctt

SEQ ID NO: 257 - DNA, 49 bp, synthetic construct
  source 1..49: mol_type = other DNA
cgtcggagtgggttcagtctacaaacccatcgcgtgggcatattcgcaa

SEQ ID NO: 258 - DNA, 49 bp, synthetic construct
  source 1..49: mol_type = other DNA
cgtcggagtgggttcagtctacagaatgcgggtcgcttcacttacgcca

SEQ ID NO: 259 - DNA, 49 bp, synthetic construct
  source 1..49: mol_type = other DNA
cgtcggagtgggttcagtctaaactaatcagcaccgcgtcggcaagtgt

SEQ ID NO: 260 - DNA, 49 bp, synthetic construct
  source 1..49: mol_type = other DNA
cgtcggagtgggttcagtctactattcggcgctccacagttccggattt

SEQ ID NO: 261 - DNA, 49 bp, synthetic construct
  source 1..49: mol_type = other DNA
cgtcggagtgggttcagtctattgtgcttaccttgcgggccaacatcca

SEQ ID NO: 262 - DNA, 49 bp, synthetic construct
  source 1..49: mol_type = other DNA
cgtcggagtgggttcagtctacggtccagtaccgcgcggctgaaatcat

SEQ ID NO: 263 - DNA, 49 bp, synthetic construct
  source 1..49: mol_type = other DNA
cgtcggagtgggttcagtctacgctcgtgattagcgccgtggcctgatt

SEQ ID NO: 264 - DNA, 49 bp, synthetic construct
  source 1..49: mol_type = other DNA
cgtcggagtgggttcagtctagcgacagcgtgtaccacagcggatggtt

SEQ ID NO: 265 - DNA, 49 bp, synthetic construct
  source 1..49: mol_type = other DNA
cgtcggagtgggttcagtctaagttacagaactggcgatcgttcggcgt

SEQ ID NO: 266 - DNA, 49 bp, synthetic construct
  source 1..49: mol_type = other DNA
cgtcggagtgggttcagtctataacattggcaccatgccgtgggtttca

SEQ ID NO: 267 - DNA, 49 bp, synthetic construct
  source 1..49: mol_type = other DNA
cgtcggagtgggttcagtctacggtcttcgctattacgccagctggcga

SEQ ID NO: 268 - DNA, 49 bp, synthetic construct
  source 1..49: mol_type = other DNA
cgtcggagtgggttcagtctatagacactcgggtgattacgatcgcgct

SEQ ID NO: 269 - DNA, 49 bp, synthetic construct
  source 1..49: mol_type = other DNA
cgtcggagtgggttcagtctaaggagataactgccgtcactccagcgca

SEQ ID NO: 270 - DNA, 49 bp, synthetic construct
  source 1..49: mol_type = other DNA
cgtcggagtgggttcagtctaaaatttgatggaccatttcggcaccgcc

SEQ ID NO: 271 - DNA, 49 bp, synthetic construct
  source 1..49: mol_type = other DNA
cgtcggagtgggttcagtctactagtgccgcaaggcgattaagttgggt

SEQ ID NO: 272 - DNA, 49 bp, synthetic construct
  source 1..49: mol_type = other DNA
cgtcggagtgggttcagtctacagtgacaatggcagatcccagcggtca

SEQ ID NO: 273 - DNA, 49 bp, synthetic construct
  source 1..49: mol_type = other DNA
cgtcggagtgggttcagtctatttatgccgctcatccgccacatatcct

SEQ ID NO: 274 - DNA, 49 bp, synthetic construct
  source 1..49: mol_type = other DNA
cgtcggagtgggttcagtctaaggagtgccaccatccagtgcaggaact

SEQ ID NO: 275 - DNA, 49 bp, synthetic construct
  source 1..49: mol_type = other DNA
cgtcggagtgggttcagtctatgatcgatggttcgcccggataaacgga

SEQ ID NO: 276 - DNA, 49 bp, synthetic construct
  source 1..49: mol_type = other DNA
cgtcggagtgggttcagtctaccgggtgtgcagttcaaccactgcacga

SEQ ID NO: 277 - DNA, 49 bp, synthetic construct
  source 1..49: mol_type = other DNA
cgtcggagtgggttcagtctatggatctcaccgtgcccatcaatccggt

SEQ ID NO: 278 - DNA, 49 bp, synthetic construct
  source 1..49: mol_type = other DNA
cgtcggagtgggttcagtctattagcgctcaggtcaaattcagacggca

SEQ ID NO: 279 - DNA, 63 bp, synthetic construct
  source 1..63: mol_type = other DNA
tgtggagggattgaaggatattagactgaacccactccgacgatctgtcttcgtcggagtggg

SEQ ID NO: 280 - DNA, 63 bp, synthetic construct
  source 1..63: mol_type = other DNA
cgtcggagtgggttcagtctacccactccgacgaagacagatttgtggagggattgaaggata

SEQ ID NO: 281 - DNA, 63 bp, synthetic construct
  source 1..63: mol_type = other DNA
gatgatgtagtagtaagggtttagactgaacccactccgacgatctgtcttcgtcggagtggg

SEQ ID NO: 282 - DNA, 63 bp, synthetic construct
  source 1..63: mol_type = other DNA
cgtcggagtgggttcagtctacccactccgacgaagacagattgatgatgtagtagtaagggt

SEQ ID NO: 283 - DNA, 64 bp, synthetic construct
  source 1..64: mol_type = other DNA
tgaactcggcgggttaggaatttagactgaacccactccgacgatctgtcttcgtcggagtggg

SEQ ID NO: 284 - DNA, 64 bp, synthetic construct
  source 1..64: mol_type = other DNA
cgtcggagtgggttcagtctacccactccgacgaagacagattctaaggttttgaactcggcgg

SEQ ID NO: 285 - DNA, 64 bp, synthetic construct
  source 1..64: mol_type = other DNA
gatgatgtagtagtaagggtttattcctaacccgccgagttcactaaggttttgaactcggcgg

SEQ ID NO: 286 - DNA, 64 bp, synthetic construct
  source 1..64: mol_type = other DNA
tgaactcggcgggttaggaatccgccgagttcaaaaccttagttgatgatgtagtagtaagggt

SEQ ID NO: 287 - DNA, 49 bp, synthetic construct
  source 1..49: mol_type = other DNA
gctggtcggtggattggatttccatgcgtcaaggtgcgcaaaggtggtt

SEQ ID NO: 288 - DNA, 49 bp, synthetic construct
  source 1..49: mol_type = other DNA
gctggtcggtggattggattttcaacgttcacctacgcccgcaaacaca

SEQ ID NO: 289 - DNA, 49 bp, synthetic construct
  source 1..49: mol_type = other DNA
gctggtcggtggattggatttcttttcgacgtcaactacggcgccgatt

SEQ ID NO: 290 - DNA, 49 bp, synthetic construct
  source 1..49: mol_type = other DNA
gctggtcggtggattggattttgcagcacgcgctaccaccagtttgtct

SEQ ID NO: 291 - DNA, 49 bp, synthetic construct
  source 1..49: mol_type = other DNA
gctggtcggtggattggatttggcagctgctgctgaacttccagcacca

SEQ ID NO: 292 - DNA, 49 bp, synthetic construct
  source 1..49: mol_type = other DNA
gctggtcggtggattggattttggtcaagagcatcgtacacgcgcggta

SEQ ID NO: 293 - DNA, 49 bp, synthetic construct
  source 1..49: mol_type = other DNA
gctggtcggtggattggatttctttcgcccaacgctcttcttcaccgat

SEQ ID NO: 294 - DNA, 49 bp, synthetic construct
  source 1..49: mol_type = other DNA
gctggtcggtggattggatttcggacgcgcagtgtcgtagtgttcctga

SEQ ID NO: 295 - DNA, 49 bp, synthetic construct
  source 1..49: mol_type = other DNA
gctggtcggtggattggatttctaacgttcctgcagaacgcccatctct

SEQ ID NO: 296 - DNA, 49 bp, synthetic construct
  source 1..49: mol_type = other DNA
gctggtcggtggattggattttgattaccgcccttagcgaacggacaca

SEQ ID NO: 297 - DNA, 49 bp, synthetic construct
  source 1..49: mol_type = other DNA
gctggtcggtggattggatttcataccggagtgctcgatcgcgatgtta

SEQ ID NO: 298 - DNA, 49 bp, synthetic construct
  source 1..49: mol_type = other DNA
gctggtcggtggattggatttccgatacggcccagcagtgcggatactt

SEQ ID NO: 299 - DNA, 49 bp, synthetic construct
  source 1..49: mol_type = other DNA
gctggtcggtggattggatttacgtccggcaggtgatcgtattcgcctt

SEQ ID NO: 300 - DNA, 49 bp, synthetic construct
  source 1..49: mol_type = other DNA
gctggtcggtggattggatttgggattgcgatggtacgcacgataccgc

SEQ ID NO: 301 - DNA, 49 bp, synthetic construct
  source 1..49: mol_type = other DNA
gctggtcggtggattggatttcaattacctacacccgcaccaccgaaca

SEQ ID NO: 302 - DNA, 49 bp, synthetic construct
  source 1..49: mol_type = other DNA
gctggtcggtggattggatttagttgtcgaccggttcacccagtacgtt

SEQ ID NO: 303 - DNA, 49 bp, synthetic construct
  source 1..49: mol_type = other DNA
gctggtcggtggattggatttgccagacgggtcagtcaagtcatccgca

SEQ ID NO: 304 - DNA, 49 bp, synthetic construct
  source 1..49: mol_type = other DNA
gctggtcggtggattggatttattagccacggatggtgtctttcaggga

SEQ ID NO: 305 - DNA, 49 bp, synthetic construct
  source 1..49: mol_type = other DNA
gctggtcggtggattggatttagatttacatccagaccgcgacgcagac

SEQ ID NO: 306 - DNA, 49 bp, synthetic construct
  source 1..49: mol_type = other DNA
gctggtcggtggattggatttgggagagacgcgatctgacggctcagta

SEQ ID NO: 307 - DNA, 49 bp, synthetic construct
  source 1..49: mol_type = other DNA
gctggtcggtggattggatttctttacttctgccacgaagaacggctgg

SEQ ID NO: 308 - DNA, 49 bp, synthetic construct
  source 1..49: mol_type = other DNA
gctggtcggtggattggatttccgggctcgttcatctggccatacacca

SEQ ID NO: 309 - DNA, 49 bp, synthetic construct
  source 1..49: mol_type = other DNA
gctggtcggtggattggatttttaacgtcacgaccttcgtcacggaatt

SEQ ID NO: 310 - DNA, 49 bp, synthetic construct
  source 1..49: mol_type = other DNA
gctggtcggtggattggattttatacgttggagtcggtcatttcgtggt

SEQ ID NO: 311 - DNA, 49 bp, synthetic construct
  source 1..49: mol_type = other DNA
gctggtcggtggattggatttaggacagcttcttcgatggaaccgacca

SEQ ID NO: 312 - DNA, 49 bp, synthetic construct
  source 1..49: mol_type = other DNA
gctggtcggtggattggatttcatccggccagggtgtaacgatagatgt

SEQ ID NO: 313 - DNA, 49 bp, synthetic construct
  source 1..49: mol_type = other DNA
gctggtcggtggattggatttacttacggcccagagtcgctttacctac

SEQ ID NO: 314 - DNA, 49 bp, synthetic construct
  source 1..49: mol_type = other DNA
gctggtcggtggattggatttatgtgcctgtacggaggtgatagaaccg

SEQ ID NO: 315 - DNA, 49 bp, synthetic construct
  source 1..49: mol_type = other DNA
gctggtcggtggattggatttctgagttcatccatacccaggatggcga

SEQ ID NO: 316 - DNA, 49 bp, synthetic construct
  source 1..49: mol_type = other DNA
gctggtcggtggattggattttcattgacagctcttcgtaggaaggtgc

SEQ ID NO: 317 - DNA, 49 bp, synthetic construct
  source 1..49: mol_type = other DNA
gctggtcggtggattggattttcatcctgataacgttgcaggatggact

SEQ ID NO: 318 - DNA, 49 bp, synthetic construct
  source 1..49: mol_type = other DNA
gctggtcggtggattggatttattctttgataccggtttccagcagttc

SEQ ID NO: 319 - DNA, 47 bp, synthetic construct
  source 1..47: mol_type = other DNA
cgtcggagtgggttcagtctatcatcgccagcgccttacaaagctct

SEQ ID NO: 320 - DNA, 47 bp, synthetic construct
  source 1..47: mol_type = other DNA
cgtcggagtgggttcagtctacggttcgagctgcgttgcggcttcca

SEQ ID NO: 321 - DNA, 47 bp, synthetic construct
  source 1..47: mol_type = other DNA
cgtcggagtgggttcagtctaaagaccacgcgccagtgcaggtttca

SEQ ID NO: 322 - DNA, 47 bp, synthetic construct
  source 1..47: mol_type = other DNA
cgtcggagtgggttcagtctagcacgctgctgcaacaattgccgggt

SEQ ID NO: 323 - DNA, 47 bp, synthetic construct
  source 1..47: mol_type = other DNA
cgtcggagtgggttcagtctagtctacgcggcggcctttcaaccctt

SEQ ID NO: 324 - DNA, 47 bp, synthetic construct
  source 1..47: mol_type = other DNA
cgtcggagtgggttcagtctattaacgctgcgccagaccttcaacga

SEQ ID NO: 325 - DNA, 47 bp, synthetic construct
  source 1..47: mol_type = other DNA
cgtcggagtgggttcagtctacagaagcgtcgtggcacctacgcagt

SEQ ID NO: 326 - DNA, 47 bp, synthetic construct
  source 1..47: mol_type = other DNA
cgtcggagtgggttcagtctacagacccacacggcgagcctgttcaa

SEQ ID NO: 327 - DNA, 47 bp, synthetic construct
  source 1..47: mol_type = other DNA
cgtcggagtgggttcagtctacgcacgacgcaccgcttcggtcagat

SEQ ID NO: 328 - DNA, 47 bp, synthetic construct
  source 1..47: mol_type = other DNA
cgtcggagtgggttcagtctaaaaatccggcagctgacggtcagcaa

SEQ ID NO: 329 - DNA, 47 bp, synthetic construct
  source 1..47: mol_type = other DNA
cgtcggagtgggttcagtctaaaatcgcgctcgctttccatcatgcg

SEQ ID NO: 330 - DNA, 47 bp, synthetic construct
  source 1..47: mol_type = other DNA
cgtcggagtgggttcagtctagaatacccgcgccgaccatggtatgt

SEQ ID NO: 331 - DNA, 47 bp, synthetic construct
  source 1..47: mol_type = other DNA
cgtcggagtgggttcagtctactatgggcatcggcaagagcaagctg

SEQ ID NO: 332 - DNA, 47 bp, synthetic construct
  source 1..47: mol_type = other DNA
cgtcggagtgggttcagtctaacgtttcaggatgtcggccagcgtgc

SEQ ID NO: 333 - DNA, 47 bp, synthetic construct
  source 1..47: mol_type = other DNA
cgtcggagtgggttcagtctaagcatacggaccatcgcctcgtcgct

SEQ ID NO: 334 - DNA, 47 bp, synthetic construct
  source 1..47: mol_type = other DNA
cgtcggagtgggttcagtctagcatgcagcacctgaatggtacggcg

SEQ ID NO: 335 - DNA, 47 bp, synthetic construct
  source 1..47: mol_type = other DNA
cgtcggagtgggttcagtctattattgcacatggtggtgcagctcgt

SEQ ID NO: 336 - DNA, 47 bp, synthetic construct
  source 1..47: mol_type = other DNA
cgtcggagtgggttcagtctacttttggttgtcgtgcccgagtgcaa

SEQ ID NO: 337 - DNA, 47 bp, synthetic construct
  source 1..47: mol_type = other DNA
cgtcggagtgggttcagtctaaacagtaatgttggcggtggtcgccc

SEQ ID NO: 338 - DNA, 47 bp, synthetic construct
  source 1..47: mol_type = other DNA
cgtcggagtgggttcagtctaggtatcgggcgatttggatccgccag

SEQ ID NO: 339 - DNA, 47 bp, synthetic construct
  source 1..47: mol_type = other DNA
cgtcggagtgggttcagtctatcattgccttgttcggctcgttcggt

SEQ ID NO: 340 - DNA, 47 bp, synthetic construct
  source 1..47: mol_type = other DNA
cgtcggagtgggttcagtctattggcgcaccagatcctgtgatggct

SEQ ID NO: 341 - DNA, 47 bp, synthetic construct
  source 1..47: mol_type = other DNA
cgtcggagtgggttcagtctatacgcttaccacaccgagcaccagct

SEQ ID NO: 342 - DNA, 47 bp, synthetic construct
  source 1..47: mol_type = other DNA
cgtcggagtgggttcagtctactacgttcacgctttcacctccacgc

SEQ ID NO: 343 - DNA, 47 bp, synthetic construct
  source 1..47: mol_type = other DNA
cgtcggagtgggttcagtctagtgccgttgggccgaggaacaggaat

SEQ ID NO: 344 - DNA, 48 bp, synthetic construct; identical to SEQ ID NO: 75
  source 1..48: mol_type = other DNA

SEQ ID NO: 345 - DNA, 47 bp, synthetic construct; identical to SEQ ID NO: 82
  source 1..47: mol_type = other DNA

SEQ ID NO: 346 - DNA, 45 bp, synthetic construct; identical to SEQ ID NO: 74
  source 1..45: mol_type = other DNA

SEQ ID NO: 347 - DNA, 45 bp, synthetic construct; identical to SEQ ID NO: 78
  source 1..45: mol_type = other DNA

SEQ ID NO: 348 - DNA, 48 bp, synthetic construct; identical to SEQ ID NO: 72
  source 1..48: mol_type = other DNA

SEQ ID NO: 349 - DNA, 46 bp, synthetic construct; identical to SEQ ID NO: 81
  source 1..46: mol_type = other DNA

SEQ ID NO: 350 - DNA, 45 bp, synthetic construct; identical to SEQ ID NO: 84
  source 1..45: mol_type = other DNA

SEQ ID NO: 351 - DNA, 46 bp, synthetic construct; identical to SEQ ID NO: 80
  source 1..46: mol_type = other DNA

SEQ ID NO: 352 - DNA, 47 bp, synthetic construct; identical to SEQ ID NO: 77
  source 1..47: mol_type = other DNA

SEQ ID NO: 353 - DNA, 45 bp, synthetic construct; identical to SEQ ID NO: 73
  source 1..45: mol_type = other DNA

SEQ ID NO: 354 - DNA, 45 bp, synthetic construct; identical to SEQ ID NO: 79
  source 1..45: mol_type = other DNA

SEQ ID NO: 355 - DNA, 45 bp, synthetic construct; identical to SEQ ID NO: 83
  source 1..45: mol_type = other DNA

SEQ ID NO: 356 - DNA, 47 bp, synthetic construct; identical to SEQ ID NO: 76
  source 1..47: mol_type = other DNA

SEQ ID NO: 357 - DNA, 49 bp, synthetic construct
  source 1..49: mol_type = other DNA
cggtcggtgagcatcttccatacctcagttaatgatagtgtgtcgattg

SEQ ID NO: 358 - DNA, 46 bp, synthetic construct
  source 1..46: mol_type = other DNA
cggtcggtgagcatcttccattggagccttggttttccggattacg

SEQ ID NO: 359 - DNA, 48 bp, synthetic construct
  source 1..48: mol_type = other DNA
cggtcggtgagcatcttccatagtgtctcatctctgaaaacttcccac

SEQ ID NO: 360 - DNA, 46 bp, synthetic construct
  source 1..46: mol_type = other DNA
cggtcggtgagcatcttccattgtcaccccattaagaggctccgtg

SEQ ID NO: 361 - DNA, 46 bp, synthetic construct
  source 1..46: mol_type = other DNA
cggtcggtgagcatcttccataccacgtcaatgagcaaaggtaaat

SEQ ID NO: 362 - DNA, 46 bp, synthetic construct
  source 1..46: mol_type = other DNA
cggtcggtgagcatcttccattgtaagctcacaatatgtgcataaa

SEQ ID NO: 363 - DNA, 46 bp, synthetic construct
  source 1..46: mol_type = other DNA
cggtcggtgagcatcttccatagatacacacactgattcaggcaga

SEQ ID NO: 364 - DNA, 46 bp, synthetic construct
  source 1..46: mol_type = other DNA
cggtcggtgagcatcttccattagtcttggttttccggatttggga

SEQ ID NO: 365 - DNA, 49 bp, synthetic construct
  source 1..49: mol_type = other DNA
cggtcggtgagcatcttccataacctcagttaatgatagtgtgtcgttt

SEQ ID NO: 366 - DNA, 46 bp, synthetic construct
  source 1..46: mol_type = other DNA
cggtcggtgagcatcttccattgagccttggttttccggatttcgg

SEQ ID NO: 367 - DNA, 47 bp, synthetic construct
  source 1..47: mol_type = other DNA
cggtcggtgagcatcttccatagtatcatctctgaaaacttccgacc

SEQ ID NO: 368 - DNA, 46 bp, synthetic construct
  source 1..46: mol_type = other DNA
cggtcggtgagcatcttccattgtgctcagccttggttttccgcta

SEQ ID NO: 369 - DNA, 46 bp, synthetic construct
  source 1..46: mol_type = other DNA
gtggagcgtcagtacatgctaatgcgtcaccccattaagaggcagg

SEQ ID NO: 370 - DNA, 49 bp, synthetic construct
  source 1..49: mol_type = other DNA
gtggagcgtcagtacatgctatcatgtcaatgagcaaaggtattaagaa

SEQ ID NO: 371 - DNA, 47 bp, synthetic construct
  source 1..47: mol_type = other DNA
gtggagcgtcagtacatgctaagtaagctcacaatatgtgcattaaa

SEQ ID NO: 372 - DNA, 47 bp, synthetic construct
  source 1..47: mol_type = other DNA
gtggagcgtcagtacatgctatgaaactaacacacacactgattgtc

SEQ ID NO: 373 - DNA, 48 bp, synthetic construct
  source 1..48: mol_type = other DNA
gtggagcgtcagtacatgctaactaagttaatgatagtgtgtcgattg

SEQ ID NO: 374 - DNA, 49 bp, synthetic construct
  source 1..49: mol_type = other DNA
gtggagcgtcagtacatgctatgtgtctcatctctgaaaacttccgacc

SEQ ID NO: 375 - DNA, 46 bp, synthetic construct
  source 1..46: mol_type = other DNA
gtggagcgtcagtacatgctaaaggaaggcacattctcatctcact

SEQ ID NO: 376 - DNA, 46 bp, synthetic construct
  source 1..46: mol_type = other DNA
gtggagcgtcagtacatgctatcgtcaccccattaagaggctcggt

SEQ ID NO: 377 - DNA, 46 bp, synthetic construct
  source 1..46: mol_type = other DNA
gtggagcgtcagtacatgctaagcgtcaccccattaagaggctagg

SEQ ID NO: 378 - DNA, 48 bp, synthetic construct
  source 1..48: mol_type = other DNA
gtggagcgtcagtacatgctatcatgtcaatgagcaaaggtattatga

SEQ ID NO: 379 - DNA, 46 bp, synthetic construct
  source 1..46: mol_type = other DNA
gtggagcgtcagtacatgctaataggctcacaatatgtgcattaaa

SEQ ID NO: 380 - DNA, 46 bp, synthetic construct
  source 1..46: mol_type = other DNA
gtggagcgtcagtacatgctattgacacacacactgattcagggag

SEQ ID NO: 381 - DNA, 63 bp, synthetic construct
  source 1..63: mol_type = other DNA
tgtggagggattgaaggatatatggaagatgctcaccgaccgttcatgcaacggtcggtgagc

SEQ ID NO: 382 - DNA, 63 bp, synthetic construct
  source 1..63: mol_type = other DNA
cggtcggtgagcatcttccatgctcaccgaccgttgcatgaattgtggagggattgaaggata

SEQ ID NO: 383 - DNA, 63 bp, synthetic construct
  source 1..63: mol_type = other DNA
tgaaaggaatgggttgtggtttagcatgtactgacgctccacttcaaccaagtggagcgtcag

SEQ ID NO: 384 - DNA, 63 bp, synthetic construct
  source 1..63: mol_type = other DNA
gtggagcgtcagtacatgctactgacgctccacttggttgaattgaaaggaatgggttgtggt

SEQ ID NO: 385 - DNA, 63 bp, synthetic construct
  source 1..63: mol_type = other DNA
ataggaaatggtggtagtgttaaatccaatccaccgaccagcaattgagatgctggtcggtgg

SEQ ID NO: 386 - DNA, 63 bp, synthetic construct
  source 1..63: mol_type = other DNA
gctggtcggtggattggatttccaccgaccagcatctcaatttataggaaatggtggtagtgt

SEQ ID NO: 387 - DNA, 47 bp, synthetic construct
  source 1..47: mol_type = other DNA
tgtgatggaagttagagggtcctcagttaatgatagtgtgtcgattg

SEQ ID NO: 388 - DNA, 44 bp, synthetic construct
  source 1..44: mol_type = other DNA
tgtgatggaagttagagggtggagccttggttttccggattacg

SEQ ID NO: 389 - DNA, 46 bp, synthetic construct
  source 1..46: mol_type = other DNA
tgtgatggaagttagagggtgtgtctcatctctgaaaacttcccac

SEQ ID NO: 390 - DNA, 44 bp, synthetic construct
  source 1..44: mol_type = other DNA
tgtgatggaagttagagggtgtcaccccattaagaggctccgtg

SEQ ID NO: 391 - DNA, 44 bp, synthetic construct
  source 1..44: mol_type = other DNA
tgtgatggaagttagagggtccacgtcaatgagcaaaggtaaat

SEQ ID NO: 392 - DNA, 44 bp, synthetic construct
  source 1..44: mol_type = other DNA
tgtgatggaagttagagggtgtaagctcacaatatgtgcataaa

SEQ ID NO: 393 - DNA, 44 bp, synthetic construct
  source 1..44: mol_type = other DNA
tgtgatggaagttagagggtgatacacacactgattcaggcaga

SEQ ID NO: 394 - DNA, 44 bp, synthetic construct
  source 1..44: mol_type = other DNA
tgtgatggaagttagagggtagtcttggttttccggatttggga

SEQ ID NO: 395 - DNA, 47 bp, synthetic construct
  source 1..47: mol_type = other DNA
tgtgatggaagttagagggtacctcagttaatgatagtgtgtcgttt

SEQ ID NO: 396 - DNA, 44 bp, synthetic construct
  source 1..44: mol_type = other DNA
tgtgatggaagttagagggtgagccttggttttccggatttcgg

SEQ ID NO: 397 - DNA, 45 bp, synthetic construct
  source 1..45: mol_type = other DNA
tgtgatggaagttagagggtgtatcatctctgaaaacttccgacc

SEQ ID NO: 398 - DNA, 44 bp, synthetic construct
  source 1..44: mol_type = other DNA
tgtgatggaagttagagggtgtgctcagccttggttttccgcta

SEQ ID NO: 399 - DNA, 44 bp, synthetic construct
  source 1..44: mol_type = other DNA
tgtgatggaagttagagggttgcgtcaccccattaagaggcagg

SEQ ID NO: 400 - DNA, 47 bp, synthetic construct
  source 1..47: mol_type = other DNA
tgtgatggaagttagagggtcatgtcaatgagcaaaggtattaagaa

SEQ ID NO: 401 - DNA, 45 bp, synthetic construct
  source 1..45: mol_type = other DNA
tgtgatggaagttagagggtgtaagctcacaatatgtgcattaaa

SEQ ID NO: 402 - DNA, 45 bp, synthetic construct
  source 1..45: mol_type = other DNA
tgtgatggaagttagagggtgaaactaacacacacactgattgtc

SEQ ID NO: 403 - DNA, 46 bp, synthetic construct
  source 1..46: mol_type = other DNA
tgtgatggaagttagagggtctaagttaatgatagtgtgtcgattg

SEQ ID NO: 404 - DNA, 47 bp, synthetic construct
  source 1..47: mol_type = other DNA
tgtgatggaagttagagggtgtgtctcatctctgaaaacttccgacc

SEQ ID NO: 405 - DNA, 44 bp, synthetic construct
  source 1..44: mol_type = other DNA
tgtgatggaagttagagggtaggaaggcacattctcatctcact

SEQ ID NO: 406 - DNA, 44 bp, synthetic construct
  source 1..44: mol_type = other DNA
tgtgatggaagttagagggtcgtcaccccattaagaggctcggt

SEQ ID NO: 407 - DNA, 44 bp, synthetic construct
  source 1..44: mol_type = other DNA
tgtgatggaagttagagggtgcgtcaccccattaagaggctagg

SEQ ID NO: 408 - DNA, 46 bp, synthetic construct
  source 1..46: mol_type = other DNA
tgtgatggaagttagagggtcatgtcaatgagcaaaggtattatga

SEQ ID NO: 409 - DNA, 44 bp, synthetic construct
  source 1..44: mol_type = other DNA
tgtgatggaagttagagggttaggctcacaatatgtgcattaaa

SEQ ID NO: 410 - DNA, 44 bp, synthetic construct
  source 1..44: mol_type = other DNA
tgtgatggaagttagagggttgacacacacactgattcagggag

SEQ ID NO: 411 - DNA, 45 bp, synthetic construct
  source 1..45: mol_type = other DNA
aggttaggttgagaataggagaggctcagtagttttggatgctca

SEQ ID NO: 412 - DNA, 45 bp, synthetic construct
  source 1..45: mol_type = other DNA
aggttaggttgagaataggaagacgcgtcacttacgtgacacggc

SEQ ID NO: 413 - DNA, 44 bp, synthetic construct
  source 1..44: mol_type = other DNA
aggttaggttgagaataggagtggaggtgctggtaactaagctg

SEQ ID NO: 414 - DNA, 46 bp, synthetic construct
  source 1..46: mol_type = other DNA
aggttaggttgagaataggactagttttatgggattagctccagga

SEQ ID NO: 415 - DNA, 44 bp, synthetic construct
  source 1..44: mol_type = other DNA
aggttaggttgagaataggagaggaaagttctcagcatgtcttc

SEQ ID NO: 416 - DNA, 44 bp, synthetic construct
  source 1..44: mol_type = other DNA
aggttaggttgagaataggaacacccatgctcggcacttctccc

SEQ ID NO: 417 - DNA, 44 bp, synthetic construct
  source 1..44: mol_type = other DNA
aggttaggttgagaataggacgcggtgtttttcacacccataca

SEQ ID NO: 418 - DNA, 44 bp, synthetic construct
  source 1..44: mol_type = other DNA
aggttaggttgagaataggatggccagagtgatacatgagggcg

SEQ ID NO: 419 - DNA, 44 bp, synthetic construct
  source 1..44: mol_type = other DNA
aggttaggttgagaataggatggctatctccgagcttgatttcg

SEQ ID NO: 420 - DNA, 44 bp, synthetic construct
  source 1..44: mol_type = other DNA
aggttaggttgagaataggaggcacacaggaaattccaccaagg

SEQ ID NO: 421 - DNA, 44 bp, synthetic construct
  source 1..44: mol_type = other DNA
aggttaggttgagaataggaaagatccaacttgctgaaccagga

SEQ ID NO: 422 - DNA, 44 bp, synthetic construct
  source 1..44: mol_type = other DNA
aggttaggttgagaataggatgcgtcacctaacaagtaggcagg

SEQ ID NO: 423 - DNA, 46 bp, synthetic construct
  source 1..46: mol_type = other DNA
aggttaggttgagaataggacgtgtattaacttactgcccttcgag

SEQ ID NO: 424 - DNA, 44 bp, synthetic construct
  source 1..44: mol_type = other DNA
aggttaggttgagaataggaacaagacaaagtttctcgtgcagg

SEQ ID NO: 425 - DNA, 44 bp, synthetic construct
  source 1..44: mol_type = other DNA
aggttaggttgagaataggaaaacttcaaagatcctttcgccat

SEQ ID NO: 426 - DNA, 45 bp, synthetic construct
  source 1..45: mol_type = other DNA
aggttaggttgagaataggagcacgctaaaatcaatgaagctatt

SEQ ID NO: 427 - DNA, 44 bp, synthetic construct
  source 1..44: mol_type = other DNA
aggttaggttgagaataggacgatctgatagcgtgaggtccctt

SEQ ID NO: 428 - DNA, 47 bp, synthetic construct
  source 1..47: mol_type = other DNA
aggttaggttgagaataggaataattcagtacaagatacctaggaat

SEQ ID NO: 429 - DNA, 44 bp, synthetic construct
  source 1..44: mol_type = other DNA
aggttaggttgagaataggaaggcgctgaatccaggagcaacga

SEQ ID NO: 430 - DNA, 44 bp, synthetic construct
  source 1..44: mol_type = other DNA
aggttaggttgagaataggacaaaacgctctatgatcgtcaata

SEQ ID NO: 431 - DNA, 46 bp, synthetic construct
  source 1..46: mol_type = other DNA
aggttaggttgagaataggagcagtgtttttcacacccattgtgca

SEQ ID NO: 432 - DNA, 44 bp, synthetic construct
  source 1..44: mol_type = other DNA
aggttaggttgagaataggactgcgatcggttttatgggatatc

SEQ ID NO: 433 - DNA, 44 bp, synthetic construct
  source 1..44: mol_type = other DNA
aggttaggttgagaataggaggatcgacgtgtctgtctcgctca

SEQ ID NO: 434 - DNA, 44 bp, synthetic construct
  source 1..44: mol_type = other DNA
aggttaggttgagaataggaggtgcagtaaccagaagtacacct

SEQ ID NO: 435 - DNA, 44 bp, synthetic construct
  source 1..44: mol_type = other DNA
ttggaggtgtagggagtaaaacctcttcgactggtctcagcagg

SEQ ID NO: 436 - DNA, 47 bp, synthetic construct
  source 1..47: mol_type = other DNA
ttggaggtgtagggagtaaatgcaatcgatgaggttattaacctgta

SEQ ID NO: 437 - DNA, 46 bp, synthetic construct
  source 1..46: mol_type = other DNA
ttggaggtgtagggagtaaacatcagtcacacccgaaggtgctagg

SEQ ID NO: 438 - DNA, 46 bp, synthetic construct
  source 1..46: mol_type = other DNA
ttggaggtgtagggagtaaagcaatcgatgaggttattaacctgta

SEQ ID NO: 439 - DNA, 45 bp, synthetic construct
  source 1..45: mol_type = other DNA
ttggaggtgtagggagtaaacatcagtcacacccgaaggtgcagg

SEQ ID NO: 440 - DNA, 45 bp, synthetic construct
  source 1..45: mol_type = other DNA
ttggaggtgtagggagtaaaatgagtcacacccgaaggtgctagg

SEQ ID NO: 441 - DNA, 45 bp, synthetic construct
  source 1..45: mol_type = other DNA
ttggaggtgtagggagtaaatcccttcacctacacaccagcgacg

SEQ ID NO: 442 - DNA, 44 bp, synthetic construct
  source 1..44: mol_type = other DNA
ttggaggtgtagggagtaaatcccttcacctacacaccagccac

SEQ ID NO: 443 - DNA, 44 bp, synthetic construct
  source 1..44: mol_type = other DNA
ttggaggtgtagggagtaaatgaccgcaaccccggtgagggcgg

SEQ ID NO: 444 - DNA, 44 bp, synthetic construct
  source 1..44: mol_type = other DNA
ttggaggtgtagggagtaaaagagactggtctcagctccacggc

SEQ ID NO: 445 - DNA, 44 bp, synthetic construct
  source 1..44: mol_type = other DNA
ttggaggtgtagggagtaaaatgagtcacacccgaaggtgcagg

SEQ ID NO: 446 - DNA, 44 bp, synthetic construct
  source 1..44: mol_type = other DNA
ttggaggtgtagggagtaaatgcgtcacacccgaaggtgctagg

SEQ ID NO: 447 - DNA, 44 bp, synthetic construct
  source 1..44: mol_type = other DNA
ttggaggtgtagggagtaaagtgctcagccttgattatccgcta

SEQ ID NO: 448 - DNA, 44 bp, synthetic construct
  source 1..44: mol_type = other DNA
ttggaggtgtagggagtaaaccacgtcaatcgatgaggttaaat

SEQ ID NO: 449 - DNA, 44 bp, synthetic construct
  source 1..44: mol_type = other DNA
ttggaggtgtagggagtaaaaataacctcatcgccttcctcagg

SEQ ID NO: 450 - DNA, 44 bp, synthetic construct
  source 1..44: mol_type = other DNA
ttggaggtgtagggagtaaacccacgtcaatcgatgaggtttaa

SEQ ID NO: 451 - DNA, 44 bp, synthetic construct
  source 1..44: mol_type = other DNA
ttggaggtgtagggagtaaacatcagtcacacccgaaggtggag

SEQ ID NO: 452 - DNA, 44 bp, synthetic construct
  source 1..44: mol_type = other DNA
ttggaggtgtagggagtaaacccttcacctacacaccagcgacg

SEQ ID NO: 453 - DNA, 47 bp, synthetic construct
  source 1..47: mol_type = other DNA
cggtcggtgagcatcttccattcatcgccagcgccttacaaagctct

SEQ ID NO: 454 - DNA, 47 bp, synthetic construct
  source 1..47: mol_type = other DNA
cggtcggtgagcatcttccatcggttcgagctgcgttgcggcttcca

SEQ ID NO: 455 - DNA, 47 bp, synthetic construct
  source 1..47: mol_type = other DNA
cggtcggtgagcatcttccataagaccacgcgccagtgcaggtttca

SEQ ID NO: 456 - DNA, 47 bp, synthetic construct
  source 1..47: mol_type = other DNA
cggtcggtgagcatcttccatgcacgctgctgcaacaattgccgggt

SEQ ID NO: 457 - DNA, 47 bp, synthetic construct
  source 1..47: mol_type = other DNA
cggtcggtgagcatcttccatgtctacgcggcggcctttcaaccctt

SEQ ID NO: 458 - DNA, 47 bp, synthetic construct
  source 1..47: mol_type = other DNA
cggtcggtgagcatcttccatttaacgctgcgccagaccttcaacga

SEQ ID NO: 459 - DNA, 47 bp, synthetic construct
  source 1..47: mol_type = other DNA
cggtcggtgagcatcttccatcagaagcgtcgtggcacctacgcagt

SEQ ID NO: 460 - DNA, 47 bp, synthetic construct
  source 1..47: mol_type = other DNA
cggtcggtgagcatcttccatcagacccacacggcgagcctgttcaa

SEQ ID NO: 461 - DNA, 47 bp, synthetic construct
  source 1..47: mol_type = other DNA
cggtcggtgagcatcttccatcgcacgacgcaccgcttcggtcagat

SEQ ID NO: 462 - DNA, 47 bp, synthetic construct
  source 1..47: mol_type = other DNA
cggtcggtgagcatcttccataaaatccggcagctgacggtcagcaa

SEQ ID NO: 463 - DNA, 47 bp, synthetic construct
  source 1..47: mol_type = other DNA
cggtcggtgagcatcttccataaatcgcgctcgctttccatcatgcg

SEQ ID NO: 464 - DNA, 47 bp, synthetic construct
  source 1..47: mol_type = other DNA
cggtcggtgagcatcttccatgaatacccgcgccgaccatggtatgt

SEQ ID NO: 465 - DNA, 47 bp, synthetic construct
  source 1..47: mol_type = other DNA
cggtcggtgagcatcttccatctatgggcatcggcaagagcaagctg

SEQ ID NO: 466 - DNA, 47 bp, synthetic construct
  source 1..47: mol_type = other DNA
cggtcggtgagcatcttccatacgtttcaggatgtcggccagcgtgc

SEQ ID NO: 467 - DNA, 47 bp, synthetic construct
  source 1..47: mol_type = other DNA
cggtcggtgagcatcttccatagcatacggaccatcgcctcgtcgct

SEQ ID NO: 468 - DNA, 47 bp, synthetic construct
  source 1..47: mol_type = other DNA
cggtcggtgagcatcttccatgcatgcagcacctgaatggtacggcg

SEQ ID NO: 469 - DNA, 47 bp, synthetic construct
  source 1..47: mol_type = other DNA
cggtcggtgagcatcttccatttattgcacatggtggtgcagctcgt

SEQ ID NO: 470 - DNA, 47 bp, synthetic construct
  source 1..47: mol_type = other DNA
cggtcggtgagcatcttccatcttttggttgtcgtgcccgagtgcaa

SEQ ID NO: 471 - DNA, 47 bp, synthetic construct
  source 1..47: mol_type = other DNA
cggtcggtgagcatcttccataacagtaatgttggcggtggtcgccc

SEQ ID NO: 472 - DNA, 47 bp, synthetic construct
  source 1..47: mol_type = other DNA
cggtcggtgagcatcttccatggtatcgggcgatttggatccgccag

SEQ ID NO: 473 - DNA, 47 bp, synthetic construct
  source 1..47: mol_type = other DNA
cggtcggtgagcatcttccattcattgccttgttcggctcgttcggt

SEQ ID NO: 474 - DNA, 47 bp, synthetic construct
  source 1..47: mol_type = other DNA
cggtcggtgagcatcttccatttggcgcaccagatcctgtgatggct

SEQ ID NO: 475 - DNA, 47 bp, synthetic construct
  source 1..47: mol_type = other DNA
cggtcggtgagcatcttccattacgcttaccacaccgagcaccagct

SEQ ID NO: 476 - DNA, 47 bp, synthetic construct
  source 1..47: mol_type = other DNA
cggtcggtgagcatcttccatctacgttcacgctttcacctccacgc

SEQ ID NO: 477 - DNA, 47 bp, synthetic construct
  source 1..47: mol_type = other DNA
cggtcggtgagcatcttccatgtgccgttgggccgaggaacaggaat

SEQ ID NO: 478 - DNA, 47 bp, synthetic construct
  source 1..47: mol_type = other DNA
cggtcggtgagcatcttccatccggcaccaaccagacgagacaccga

SEQ ID NO: 479 - DNA, 47 bp, synthetic construct
  source 1..47: mol_type = other DNA
cggtcggtgagcatcttccatgacgacggcgacgatccggtcttcat

SEQ ID NO: 480 - DNA, 47 bp, synthetic construct
  source 1..47: mol_type = other DNA
cggtcggtgagcatcttccatattcgcgatggtgcagttcttgctcc

SEQ ID NO: 481 - DNA, 47 bp, synthetic construct
  source 1..47: mol_type = other DNA
cggtcggtgagcatcttccatgacgaaacgacgttccagcgcagcat

SEQ ID NO: 482 - DNA, 47 bp, synthetic construct
  source 1..47: mol_type = other DNA
gggtggtcgtcgaagtcgtatccgatgcgggcgcgaggtttcgcttt

SEQ ID NO: 483 - DNA, 47 bp, synthetic construct
  source 1..47: mol_type = other DNA
gggtggtcgtcgaagtcgtatagatcgcggctttccacgctttcgct

SEQ ID NO: 484 - DNA, 47 bp, synthetic construct
  source 1..47: mol_type = other DNA
gggtggtcgtcgaagtcgtatacctcggcgcgtcggtttcatcgctt

SEQ ID NO: 485 - DNA, 47 bp, synthetic construct
  source 1..47: mol_type = other DNA
gggtggtcgtcgaagtcgtatacgtggtttcagcaggcttggcggca

SEQ ID NO: 486 - DNA, 47 bp, synthetic construct
  source 1..47: mol_type = other DNA
gggtggtcgtcgaagtcgtataagaccttgctggcgtactgcggcat

SEQ ID NO: 487 - DNA, 47 bp, synthetic construct
  source 1..47: mol_type = other DNA
gggtggtcgtcgaagtcgtatgtgttcgcgctggaaagcggtctcga

SEQ ID NO: 488 - DNA, 47 bp, synthetic construct
  source 1..47: mol_type = other DNA
gggtggtcgtcgaagtcgtatgcctggcgtcttcgccggaagcttca

SEQ ID NO: 489 - DNA, 47 bp, synthetic construct
  source 1..47: mol_type = other DNA
gggtggtcgtcgaagtcgtatcggacgttcgccggacgcttccttga

SEQ ID NO: 490 - DNA, 47 bp, synthetic construct
  source 1..47: mol_type = other DNA
gggtggtcgtcgaagtcgtatcaatgcttctacggcttcgctggcga

SEQ ID NO: 491 - DNA, 47 bp, synthetic construct
  source 1..47: mol_type = other DNA
gggtggtcgtcgaagtcgtatacctttcgctggcagcctcgctggta

SEQ ID NO: 492 - DNA, 47 bp, synthetic construct
  source 1..47: mol_type = other DNA
gggtggtcgtcgaagtcgtatacttggcgttgcgcttctcgttgagc

SEQ ID NO: 493 - DNA, 47 bp, synthetic construct
  source 1..47: mol_type = other DNA
gggtggtcgtcgaagtcgtatgtattcgtagctggtctggccggcaa

SEQ ID NO: 494 - DNA, 47 bp, synthetic construct
  source 1..47: mol_type = other DNA
gggtggtcgtcgaagtcgtataccccgtcgaccagtgcaacacgcaa

SEQ ID NO: 495 - DNA, 47 bp, synthetic construct
  source 1..47: mol_type = other DNA
gggtggtcgtcgaagtcgtatagcaggctgggttcgacgcgagtgat

SEQ ID NO: 496 - DNA, 47 bp, synthetic construct
  source 1..47: mol_type = other DNA
gggtggtcgtcgaagtcgtatcgtacttcttcggccgcttccgttgc

SEQ ID NO: 497 - DNA, 47 bp, synthetic construct
  source 1..47: mol_type = other DNA
gggtggtcgtcgaagtcgtatgcgagctcgttgcgctcttcgccttc

SEQ ID NO: 498 - DNA, 47 bp, synthetic construct
  source 1..47: mol_type = other DNA
gggtggtcgtcgaagtcgtatcgccccttgttgccgcgctcttcctt

SEQ ID NO: 499 - DNA, 47 bp, synthetic construct
  source 1..47: mol_type = other DNA
gggtggtcgtcgaagtcgtatgcggcgcgggtacgcagttcgatctt

SEQ ID NO: 500 - DNA, 47 bp, synthetic construct
  source 1..47: mol_type = other DNA
gggtggtcgtcgaagtcgtatctgttcaccaggccctggaacaggct

SEQ ID NO: 501 - DNA, 47 bp, synthetic construct
  source 1..47: mol_type = other DNA
gggtggtcgtcgaagtcgtatccttggcgcggatgatgacgttgctt

SEQ ID NO: 502 - DNA, 47 bp, synthetic construct
  source 1..47: mol_type = other DNA
gggtggtcgtcgaagtcgtattactcgatggaaaccagggcctcggt

SEQ ID NO: 503 - DNA, 47 bp, synthetic construct
  source 1..47: mol_type = other DNA
gggtggtcgtcgaagtcgtatcgatcttcagtggcttgcgcgaccag

SEQ ID NO: 504 - DNA, 47 bp, synthetic construct
  source 1..47: mol_type = other DNA
gggtggtcgtcgaagtcgtatccgtcgaccggtgcttcggtgtgttg

SEQ ID NO: 505 - DNA, 47 bp, synthetic construct
  source 1..47: mol_type = other DNA
gggtggtcgtcgaagtcgtattgctcggcctgggcgatttcttcagc

SEQ ID NO: 506 - DNA, 47 bp, synthetic construct
  source 1..47: mol_type = other DNA
gctcgacgttcctttgcaacaccttgcggtggttgccggtcgtgttt

SEQ ID NO: 507 - DNA, 47 bp, synthetic construct
  source 1..47: mol_type = other DNA
gctcgacgttcctttgcaacacgcagccagcacagcggcagcaagaa

SEQ ID NO: 508 - DNA, 47 bp, synthetic construct
  source 1..47: mol_type = other DNA
gctcgacgttcctttgcaacacgcatgaggtatcgcgcgcatcgcct

SEQ ID NO: 509 - DNA, 47 bp, synthetic construct
  source 1..47: mol_type = other DNA
gctcgacgttcctttgcaacatcgtccgccaccgtcatgcctgttgt

SEQ ID NO: 510 - DNA, 47 bp, synthetic construct
  source 1..47: mol_type = other DNA
gctcgacgttcctttgcaacagtttcaacaaactgctgccgctgcgg

SEQ ID NO: 511 - DNA, 47 bp, synthetic construct
  source 1..47: mol_type = other DNA
gctcgacgttcctttgcaacagcttgacggcctcgctgtgcttgtca

SEQ ID NO: 512 - DNA, 47 bp, synthetic construct
  source 1..47: mol_type = other DNA
gctcgacgttcctttgcaacaatcacggccaacacaataggtgcgcg

SEQ ID NO: 513 - DNA, 47 bp, synthetic construct
  source 1..47: mol_type = other DNA
gctcgacgttcctttgcaacatcgtgcacagtgggaagcgctcctca

SEQ ID NO: 514 - DNA, 47 bp, synthetic construct
  source 1..47: mol_type = other DNA
gctcgacgttcctttgcaacactttggttccgcgacgaggttggtca

SEQ ID NO: 515 - DNA, 47 bp, synthetic construct
  source 1..47: mol_type = other DNA
gctcgacgttcctttgcaacaatgagttgcgcctgagccggtatcca

SEQ ID NO: 516 - DNA, 47 bp, synthetic construct
  source 1..47: mol_type = other DNA
gctcgacgttcctttgcaacaaaatgtaagctttccgtcacggcgcg

SEQ ID NO: 517 - DNA, 47 bp, synthetic construct
  source 1..47: mol_type = other DNA
gctcgacgttcctttgcaacacaaatcactgtattgcacggcggccg

SEQ ID NO: 518 - DNA, 47 bp, synthetic construct
  source 1..47: mol_type = other DNA
gctcgacgttcctttgcaacatacggtgaccacggaaccagcgcatt

SEQ ID NO: 519 - DNA, 47 bp, synthetic construct
  source 1..47: mol_type = other DNA
gctcgacgttcctttgcaacagccgccgcccaactccttcagcaaca

SEQ ID NO: 520 - DNA, 47 bp, synthetic construct
  source 1..47: mol_type = other DNA
gctcgacgttcctttgcaacagtattgccgtgccatacactccgcag

SEQ ID NO: 521 - DNA, 47 bp, synthetic construct
  source 1..47: mol_type = other DNA
gctcgacgttcctttgcaacagggagcggtccagacggaacgtggta

SEQ ID NO: 522 - DNA, 47 bp, synthetic construct
  source 1..47: mol_type = other DNA
gctcgacgttcctttgcaacacatacggatgggtgtgtccagcaagc

SEQ ID NO: 523 - DNA, 47 bp, synthetic construct
  source 1..47: mol_type = other DNA
gctcgacgttcctttgcaacagcttggagccgccaaagtcctgttcg

SEQ ID NO: 524 - DNA, 47 bp, synthetic construct
  source 1..47: mol_type = other DNA
gctcgacgttcctttgcaacagcttagagcgcatgaaggccgtcagc

SEQ ID NO: 525 - DNA, 47 bp, synthetic construct
  source 1..47: mol_type = other DNA
gctcgacgttcctttgcaacacaattgtctccgactgcccagtctgc

SEQ ID NO: 526 - DNA, 47 bp, synthetic construct
  source 1..47: mol_type = other DNA
gctcgacgttcctttgcaacagggaatccctcgagcgcgagtctagc

SEQ ID NO: 527 - DNA, 47 bp, synthetic construct
  source 1..47: mol_type = other DNA
gctcgacgttcctttgcaacaggtgcggccatgagagacaagacagc

SEQ ID NO: 528 - DNA, 47 bp, synthetic construct
  source 1..47: mol_type = other DNA
cgtcggagtgggttcagtctacgcagagttcgcgtgcagcggcgtta

SEQ ID NO: 529 - DNA, 47 bp, synthetic construct
  source 1..47: mol_type = other DNA
cgtcggagtgggttcagtctatggagtgcggaggttgaaacggcgga

SEQ ID NO: 530 - DNA, 47 bp, synthetic construct
  source 1..47: mol_type = other DNA
cgtcggagtgggttcagtctatgctgacgggatgccggctcgtcaaa

SEQ ID NO: 531 - DNA, 47 bp, synthetic construct
  source 1..47: mol_type = other DNA
cgtcggagtgggttcagtctacaaacgtgccgcaccaaagaaacgct

SEQ ID NO: 532 - DNA, 47 bp, synthetic construct
  source 1..47: mol_type = other DNA
cgtcggagtgggttcagtctaacaaggcacgcgccagacgggtaata

SEQ ID NO: 533 - DNA, 47 bp, synthetic construct
  source 1..47: mol_type = other DNA
cgtcggagtgggttcagtctatccagctgtctgcggagcggaggaat

SEQ ID NO: 534 - DNA, 47 bp, synthetic construct
  source 1..47: mol_type = other DNA
cgtcggagtgggttcagtctaacaagtcgatagccgggaagacgcgt

SEQ ID NO: 535 - DNA, 47 bp, synthetic construct
  source 1..47: mol_type = other DNA
cgtcggagtgggttcagtctatccccagatccagtacgcgagcggtt

SEQ ID NO: 536 - DNA, 47 bp, synthetic construct
  source 1..47: mol_type = other DNA
cgtcggagtgggttcagtctaccttacgtagatgtcgtcagggccgg

SEQ ID NO: 537 - DNA, 47 bp, synthetic construct
  source 1..47: mol_type = other DNA
cgtcggagtgggttcagtctatcctgcagttccatgttaccggtgcc

SEQ ID NO: 538 - DNA, 47 bp, synthetic construct
  source 1..47: mol_type = other DNA
cgtcggagtgggttcagtctaatcaaccgttaccacgctccatacgc

SEQ ID NO: 539 - DNA, 47 bp, synthetic construct
  source 1..47: mol_type = other DNA
cgtcggagtgggttcagtctaagtgcagcgcaaagtaacgttcaccc

SEQ ID NO: 540 - DNA, 47 bp, synthetic construct
  source 1..47: mol_type = other DNA
cgtcggagtgggttcagtctaagacgaacaggatcttgttacgcgcg

SEQ ID NO: 541 - DNA, 47 bp, synthetic construct
  source 1..47: mol_type = other DNA
cgtcggagtgggttcagtctagtacctgcagtatctccagtacgccg

SEQ ID NO: 542 - DNA, 47 bp, synthetic construct
  source 1..47: mol_type = other DNA
cgtcggagtgggttcagtctaggttcgcatcaatctcacccatcgga

SEQ ID NO: 543 - DNA, 47 bp, synthetic construct
  source 1..47: mol_type = other DNA
cgtcggagtgggttcagtctaggagcttacgcatacgagccaggttt

SEQ ID NO: 544 - DNA, 47 bp, synthetic construct
  source 1..47: mol_type = other DNA
cgtcggagtgggttcagtctataattctgcagctcttcctgagtggt

SEQ ID NO: 545 - DNA, 47 bp, synthetic construct
  source 1..47: mol_type = other DNA
cgtcggagtgggttcagtctaagtttcgtcttggtcattgccagctt

SEQ ID NO: 546 - DNA, 47 bp, synthetic construct
  source 1..47: mol_type = other DNA
cgtcggagtgggttcagtctaatttcagctcagaaaccggcgtattc

SEQ ID NO: 547 - DNA, 47 bp, synthetic construct
  source 1..47: mol_type = other DNA
cgtcggagtgggttcagtctacaacaaccagacgtttcgctttctca

SEQ ID NO: 548 - DNA, 47 bp, synthetic construct
  source 1..47: mol_type = other DNA
cgtcggagtgggttcagtctaaagcggcttgtcgtagttaacttcgt

SEQ ID NO: 549 - DNA, 47 bp, synthetic construct
  source 1..47: mol_type = other DNA
cgtcggagtgggttcagtctagctgcgcttcatcatatcgaagaagt

SEQ ID NO: 550 - DNA, 47 bp, synthetic construct
  source 1..47: mol_type = other DNA
cgtcggagtgggttcagtctatttctcttcgtagataacttcgtcca

SEQ ID NO: 551 - DNA, 47 bp, synthetic construct
  source 1..47: mol_type = other DNA
cgtcggagtgggttcagtctatgattaccggaagccggaaccacggt

SEQ ID NO: 552 - DNA, 51 bp, synthetic construct
  source 1..51: mol_type = other DNA
cgtcggagtgggttcagtctactggtccagagtcacaaaaagctgcatgac

SEQ ID NO: 553 - DNA, 51 bp, synthetic construct
  source 1..51: mol_type = other DNA
cgtcggagtgggttcagtctacagttgcacgtgtcatatttgcacctcttg

SEQ ID NO: 554 - DNA, 51 bp, synthetic construct
  source 1..51: mol_type = other DNA
cgtcggagtgggttcagtctagtacatgaagatctgtgagcttgggcaagc

SEQ ID NO: 555 - DNA, 51 bp, synthetic construct
  source 1..51: mol_type = other DNA
cgtcggagtgggttcagtctaaagaccatggtggtagcaggaacacttagc

SEQ ID NO: 556 - DNA, 51 bp, synthetic construct
  source 1..51: mol_type = other DNA
cgtcggagtgggttcagtctatgcagatctcatcagtgggaacagatcctc

SEQ ID NO: 557 - DNA, 51 bp, synthetic construct
  source 1..51: mol_type = other DNA
cgtcggagtgggttcagtctatcttcccttcatctggatggcattcgattt

SEQ ID NO: 558 - DNA, 51 bp, synthetic construct
  source 1..51: mol_type = other DNA
cgtcggagtgggttcagtctagagtggtagtttccgttggaacagtgaagg

SEQ ID NO: 559 - DNA, 51 bp, synthetic construct
  source 1..51: mol_type = other DNA
cgtcggagtgggttcagtctagttcacctgggccgtagaacatctcattca

SEQ ID NO: 560 - DNA, 51 bp, synthetic construct
  source 1..51: mol_type = other DNA
cgtcggagtgggttcagtctacagttacacagccaccaggtctcattaacc

SEQ ID NO: 561 - DNA, 51 bp, synthetic construct
  source 1..51: mol_type = other DNA
cgtcggagtgggttcagtctaacagttaccctggtaactgtagtagagccc

SEQ ID NO: 562 - DNA, 51 bp, synthetic construct
  source 1..51: mol_type = other DNA
cgtcggagtgggttcagtctaggcatcacagtggtagttgtcaatgtagac

SEQ ID NO: 563 - DNA, 51 bp, synthetic construct
  source 1..51: mol_type = other DNA
cgtcggagtgggttcagtctaggagatgttcaccacaatgttgatgccaga

SEQ ID NO: 564 - DNA, 51 bp, synthetic construct
  source 1..51: mol_type = other DNA
cgtcggagtgggttcagtctattcacagtcagagatgatcttcccactggg

SEQ ID NO: 565 - DNA, 51 bp, synthetic construct
  source 1..51: mol_type = other DNA
cgtcggagtgggttcagtctacacatgtcttcacacagacgtcatagccag

SEQ ID NO: 566 - DNA, 51 bp, synthetic construct
  source 1..51: mol_type = other DNA
cgtcggagtgggttcagtctatcacttcgaatcccaacaaacatgtggggc

SEQ ID NO: 567 - DNA, 51 bp, synthetic construct
  source 1..51: mol_type = other DNA
cgtcggagtgggttcagtctactccccaggcttcagaataatgtactgctg

SEQ ID NO: 568 - DNA, 51 bp, synthetic construct
  source 1..51: mol_type = other DNA
cgtcggagtgggttcagtctaaatcacttgggttgaagtcgggacaggtga

SEQ ID NO: 569 - DNA, 51 bp, synthetic construct
  source 1..51: mol_type = other DNA
cgtcggagtgggttcagtctaagtacatggcaaaagtcccacaggacccaa

SEQ ID NO: 570 - DNA, 49 bp, synthetic construct
  source 1..49: mol_type = other DNA
cgtcggagtgggttcagtctagtccatgcctctcaaattcatcatgacg

SEQ ID NO: 571 - DNA, 49 bp, synthetic construct
  source 1..49: mol_type = other DNA
cgtcggagtgggttcagtctaccgcctccaagtaagaactcacatcact

SEQ ID NO: 572 - DNA, 46 bp, synthetic construct
  source 1..46: mol_type = other DNA
cgtcggagtgggttcagtctacgtccagcattataagcaatccagc

SEQ ID NO: 573 - DNA, 48 bp, synthetic construct
  source 1..48: mol_type = other DNA
cgtcggagtgggttcagtctaaaagtcttcattcatctcatcagggtc

SEQ ID NO: 574 - DNA, 48 bp, synthetic construct
  source 1..48: mol_type = other DNA
cgtcggagtgggttcagtctaacacagtgatcttggtttgaatcagcc

SEQ ID NO: 575 - DNA, 47 bp, synthetic construct
  source 1..47: mol_type = other DNA
cgtcggagtgggttcagtctagtcccaagcaatgaattcctttgctg

SEQ ID NO: 576 - DNA, 45 bp, synthetic construct
  source 1..45: mol_type = other DNA
cgtcggagtgggttcagtctaccagctcatctcgtcagagaagga

SEQ ID NO: 577 - DNA, 44 bp, synthetic construct
  source 1..44: mol_type = other DNA
cgtcggagtgggttcagtctaagacctctgtgtcttgaagggcg

SEQ ID NO: 578 - DNA, 48 bp, synthetic construct
  source 1..48: mol_type = other DNA
cgtcggagtgggttcagtctagggtggtggcaagattatccagaatgg

SEQ ID NO: 579 - DNA, 44 bp, synthetic construct
  source 1..44: mol_type = other DNA
cgtcggagtgggttcagtctattaggcgacttcttctgggaagt

SEQ ID NO: 580 - DNA, 47 bp, synthetic construct
  source 1..47: mol_type = other DNA
cgtcggagtgggttcagtctaaagctcttggtgttcatcaaccactg

SEQ ID NO: 581 - DNA, 46 bp, synthetic construct
  source 1..46: mol_type = other DNA
cgtcggagtgggttcagtctatcaccaggtatttgctgtagtcgct

SEQ ID NO: 582 - DNA, 45 bp, synthetic construct
  source 1..45: mol_type = other DNA
cgtcggagtgggttcagtctaagagggaagctgggaatgatctgg

SEQ ID NO: 583 - DNA, 44 bp, synthetic construct
  source 1..44: mol_type = other DNA
cgtcggagtgggttcagtctaacctgaatgtgccctgtgagtgg

SEQ ID NO: 584 - DNA, 44 bp, synthetic construct
  source 1..44: mol_type = other DNA
gctcgacgttcctttgcaacagtgttggaagaggcggatactgg

SEQ ID NO: 585 - DNA, 51 bp, synthetic construct
  source 1..51: mol_type = other DNA
gctcgacgttcctttgcaacatcgcacaccactgatagatattgtttccca

SEQ ID NO: 586 - DNA, 53 bp, synthetic construct
  source 1..53: mol_type = other DNA
gctcgacgttcctttgcaacagtgctcctttcttgtacttaagtccagacttg

SEQ ID NO: 587 - DNA, 47 bp, synthetic construct
  source 1..47: mol_type = other DNA
gctcgacgttcctttgcaacagaacagcctttcaaatttgttgctgc

SEQ ID NO: 588 - DNA, 49 bp, synthetic construct
  source 1..49: mol_type = other DNA
gctcgacgttcctttgcaacacaacttgaagaactgcttaaagagaggg

SEQ ID NO: 589 - DNA, 48 bp, synthetic construct
  source 1..48: mol_type = other DNA
gctcgacgttcctttgcaacagaccacagtctttaggatgacataggc

SEQ ID NO: 590 - DNA, 50 bp, synthetic construct
  source 1..50: mol_type = other DNA
gctcgacgttcctttgcaacaacccggtagttgtacagaatgatgtagct

SEQ ID NO: 591 - DNA, 45 bp, synthetic construct
  source 1..45: mol_type = other DNA
gctcgacgttcctttgcaacattcccctgcctaacgactgtgatg

SEQ ID NO: 592 - DNA, 46 bp, synthetic construct
  source 1..46: mol_type = other DNA
gctcgacgttcctttgcaacacggacccttgtagatgatcatgggc

SEQ ID NO: 593 - DNA, 45 bp, synthetic construct
  source 1..45: mol_type = other DNA
gctcgacgttcctttgcaacagtgaggtacctcagtagcacggac

SEQ ID NO: 594 - DNA, 49 bp, synthetic construct
  source 1..49: mol_type = other DNA
gctcgacgttcctttgcaacaggctgaagaagtctccatagaggttggg

SEQ ID NO: 595 - DNA, 45 bp, synthetic construct
  source 1..45: mol_type = other DNA
gctcgacgttcctttgcaacatcacgccattgttgaaactgtccc

SEQ ID NO: 596 - DNA, 46 bp, synthetic construct
  source 1..46: mol_type = other DNA
gctcgacgttcctttgcaacagggtgccagttgtagatgatctgtc

SEQ ID NO: 597 - DNA, 49 bp, synthetic construct
  source 1..49: mol_type = other DNA
gctcgacgttcctttgcaacacggactagggagactgacatgctacctg

SEQ ID NO: 598 - DNA, 44 bp, synthetic construct
  source 1..44: mol_type = other DNA
gctcgacgttcctttgcaacagagtgggagaactgaaacctggg

SEQ ID NO: 599 - DNA, 45 bp, synthetic construct
  source 1..45: mol_type = other DNA
gctcgacgttcctttgcaacatagcagttgcacagtaaagatggc

SEQ ID NO: 600 - DNA, 44 bp, synthetic construct
  source 1..44: mol_type = other DNA
gctcgacgttcctttgcaacattcgcatcgttggagttcagagc

SEQ ID NO: 601 - DNA, 46 bp, synthetic construct
  source 1..46: mol_type = other DNA
gctcgacgttcctttgcaacactcgatgggcatccatcttcttgtc

SEQ ID NO: 602 - DNA, 49 bp, synthetic construct
  source 1..49: mol_type = other DNA
gctcgacgttcctttgcaacaggcatttgctggatctgtctcgatgtac

SEQ ID NO: 603 - DNA, 46 bp, synthetic construct
  source 1..46: mol_type = other DNA
gctcgacgttcctttgcaacataagggtaccacgtgtttgaatcca

SEQ ID NO: 604 - DNA, 45 bp, synthetic construct
  source 1..45: mol_type = other DNA
gctcgacgttcctttgcaacatatctgcagattcccattcctcag

SEQ ID NO: 605 - DNA, 44 bp, synthetic construct
  source 1..44: mol_type = other DNA
gctcgacgttcctttgcaacagacgcaccttgttggaaccttca

SEQ ID NO: 606 - DNA, 45 bp, synthetic construct
  source 1..45: mol_type = other DNA
gctcgacgttcctttgcaacaagatcagacacgtgtacttgagca

SEQ ID NO: 607 - DNA, 46 bp, synthetic construct
  source 1..46: mol_type = other DNA
gctcgacgttcctttgcaacatgggttggagaccttgtagagcttg

SEQ ID NO: 608 - DNA, 51 bp, synthetic construct
  source 1..51: mol_type = other DNA
gcacatgctccgtgtagaatatcgcacaccactgatagatattgtttccca

SEQ ID NO: 609 - DNA, 53 bp, synthetic construct
  source 1..53: mol_type = other DNA
gcacatgctccgtgtagaatagtgctcctttcttgtacttaagtccagacttg

SEQ ID NO: 610 - DNA, 47 bp, synthetic construct
  source 1..47: mol_type = other DNA
gcacatgctccgtgtagaatagaacagcctttcaaatttgttgctgc

SEQ ID NO: 611 - DNA, 49 bp, synthetic construct
  source 1..49: mol_type = other DNA
gcacatgctccgtgtagaatacaacttgaagaactgcttaaagagaggg

SEQ ID NO: 612 - DNA, 48 bp, synthetic construct
  source 1..48: mol_type = other DNA
gcacatgctccgtgtagaatagaccacagtctttaggatgacataggc

SEQ ID NO: 613 - DNA, 50 bp, synthetic construct
  source 1..50: mol_type = other DNA
gcacatgctccgtgtagaataacccggtagttgtacagaatgatgtagct

SEQ ID NO: 614 - DNA, 45 bp, synthetic construct
  source 1..45: mol_type = other DNA
gcacatgctccgtgtagaatattcccctgcctaacgactgtgatg

SEQ ID NO: 615 - DNA, 46 bp, synthetic construct
  source 1..46: mol_type = other DNA
gcacatgctccgtgtagaatacggacccttgtagatgatcatgggc

SEQ ID NO: 616 - DNA, 45 bp, synthetic construct
  source 1..45: mol_type = other DNA
gcacatgctccgtgtagaatagtgaggtacctcagtagcacggac

SEQ ID NO: 617 - DNA, 49 bp, synthetic construct
  source 1..49: mol_type = other DNA
gcacatgctccgtgtagaataggctgaagaagtctccatagaggttggg

SEQ ID NO: 618 - DNA, 45 bp, synthetic construct
  source 1..45: mol_type = other DNA
gcacatgctccgtgtagaatatcacgccattgttgaaactgtccc

SEQ ID NO: 619 - DNA, 46 bp, synthetic construct
  source 1..46: mol_type = other DNA
gcacatgctccgtgtagaatagggtgccagttgtagatgatctgtc

SEQ ID NO: 620 - DNA, 49 bp, synthetic construct
  source 1..49: mol_type = other DNA
gcacatgctccgtgtagaatacggactagggagactgacatgctacctg

SEQ ID NO: 621 - DNA, 44 bp, synthetic construct
  source 1..44: mol_type = other DNA
gcacatgctccgtgtagaatagagtgggagaactgaaacctggg

SEQ ID NO: 622 - DNA, 45 bp, synthetic construct
  source 1..45: mol_type = other DNA
gcacatgctccgtgtagaatatagcagttgcacagtaaagatggc

SEQ ID NO: 623 - DNA, 46 bp, synthetic construct
  source 1..46: mol_type = other DNA
gcacatgctccgtgtagaatactcgatgggcatccatcttcttgtc

SEQ ID NO: 624 - DNA, 49 bp, synthetic construct
  source 1..49: mol_type = other DNA
gcacatgctccgtgtagaataggcatttgctggatctgtctcgatgtac

SEQ ID NO: 625 - DNA, 46 bp, synthetic construct
  source 1..46: mol_type = other DNA
gcacatgctccgtgtagaatataagggtaccacgtgtttgaatcca

SEQ ID NO: 626 - DNA, 45 bp, synthetic construct
  source 1..45: mol_type = other DNA
gcacatgctccgtgtagaatatatctgcagattcccattcctcag

SEQ ID NO: 627 - DNA, 44 bp, synthetic construct
  source 1..44: mol_type = other DNA
gcacatgctccgtgtagaatagacgcaccttgttggaaccttca

SEQ ID NO: 628 - DNA, 45 bp, synthetic construct
  source 1..45: mol_type = other DNA
gcacatgctccgtgtagaatattcgcatcgttggagttcagagca

SEQ ID NO: 629 - DNA, 45 bp, synthetic construct
  source 1..45: mol_type = other DNA
gcacatgctccgtgtagaataagatcagacacgtgtacttgagca

SEQ ID NO: 630 - DNA, 46 bp, synthetic construct
  source 1..46: mol_type = other DNA
gcacatgctccgtgtagaatatgggttggagaccttgtagagcttg

SEQ ID NO: 631 - DNA, 49 bp, synthetic construct
  source 1..49: mol_type = other DNA
gcacatgctccgtgtagaatagaagcctttccaaacaaagattttccca

SEQ ID NO: 632 - DNA, 50 bp, synthetic construct
  source 1..50: mol_type = other DNA
gctggtcggtggattggatttgtcgacagaagacatactcataaagggca

SEQ ID NO: 633 - DNA, 49 bp, synthetic construct
  source 1..49: mol_type = other DNA
gctggtcggtggattggatttgatgtcatacggaagacaatacctctcc

SEQ ID NO: 634 - DNA, 47 bp, synthetic construct
  source 1..47: mol_type = other DNA
gctggtcggtggattggatttgtgcgagcaaaacaaagataagcgtg

SEQ ID NO: 635 - DNA, 50 bp, synthetic construct
  source 1..50: mol_type = other DNA
gctggtcggtggattggatttcttccagtaacatcagttcgtttggaatc

SEQ ID NO: 636 - DNA, 48 bp, synthetic construct
  source 1..48: mol_type = other DNA
gctggtcggtggattggattttgtgctggcaaaaatagtgaacaccaa

SEQ ID NO: 637 - DNA, 51 bp, synthetic construct
  source 1..51: mol_type = other DNA
gctggtcggtggattggatttttgtggaaagtgattattaactccaccagg

SEQ ID NO: 638 - DNA, 46 bp, synthetic construct
  source 1..46: mol_type = other DNA
gctggtcggtggattggatttttagatgtagaagacggacttagcg

SEQ ID NO: 639 - DNA, 46 bp, synthetic construct
  source 1..46: mol_type = other DNA
gctggtcggtggattggatttggtgtttcccatgataactgcgggt

SEQ ID NO: 640 - DNA, 45 bp, synthetic construct
  source 1..45: mol_type = other DNA
gctggtcggtggattggatttgtgcccagtttatggtggatccca

SEQ ID NO: 641 - DNA, 51 bp, synthetic construct
  source 1..51: mol_type = other DNA
gctggtcggtggattggattttacctggctccagtataattgattgcaaac

SEQ ID NO: 642 - DNA, 46 bp, synthetic construct
  source 1..46: mol_type = other DNA
gctggtcggtggattggatttgaacaggatcaagtcttccgtctcc

SEQ ID NO: 643 - DNA, 46 bp, synthetic construct
  source 1..46: mol_type = other DNA
gctggtcggtggattggatttaggacggtcaatgtcaatcacatgc

SEQ ID NO: 644 - DNA, 49 bp, synthetic construct
  source 1..49: mol_type = other DNA
gctggtcggtggattggattttaagcaacggaaaatccaattgctaaag

SEQ ID NO: 645 - DNA, 45 bp, synthetic construct
  source 1..45: mol_type = other DNA
gctggtcggtggattggatttgtccggtgaggtttccatgaaccg

SEQ ID NO: 646 - DNA, 44 bp, synthetic construct
  source 1..44: mol_type = other DNA
gctggtcggtggattggatttgcttgctgatctttcgtgtgcac

SEQ ID NO: 647 - DNA, 48 bp, synthetic construct
  source 1..48: mol_type = other DNA
gctggtcggtggattggatttccaccaacccaatatatccagtggttt

SEQ ID NO: 648 - DNA, 46 bp, synthetic construct
  source 1..46: mol_type = other DNA
gctggtcggtggattggattttcgaatgctgagtccaaagcaaagg

SEQ ID NO: 649 - DNA, 46 bp, synthetic construct
  source 1..46: mol_type = other DNA
gctggtcggtggattggatttactgtccagactcctttgaaagcca

SEQ ID NO: 650 - DNA, 44 bp, synthetic construct
  source 1..44: mol_type = other DNA
gctggtcggtggattggatttcttggcttccttaaggcgacgtt

SEQ ID NO: 651 - DNA, 44 bp, synthetic construct
  source 1..44: mol_type = other DNA
gctggtcggtggattggatttgtttcctccaaccacactgggag

SEQ ID NO: 652 - DNA, 48 bp, synthetic construct
  source 1..48: mol_type = other DNA
gctggtcggtggattggatttgtagatgctctctctactgcaggaatg

SEQ ID NO: 653 - DNA, 44 bp, synthetic construct
  source 1..44: mol_type = other DNA
gctggtcggtggattggatttaggtccacctccatgtagctccc

SEQ ID NO: 654 - DNA, 44 bp, synthetic construct
  source 1..44: mol_type = other DNA
gctggtcggtggattggatttaagtgctgagactgccttccaga

SEQ ID NO: 655 - DNA, 46 bp, synthetic construct
  source 1..46: mol_type = other DNA
gctggtcggtggattggatttcccagatgaggaccatgtccacagg

SEQ ID NO: 656 - DNA, 52 bp, synthetic construct
  source 1..52: mol_type = other DNA
cgcataccggcctatggactaagtctaccttacctaaatggtgagtatggac

SEQ ID NO: 657 - DNA, 50 bp, synthetic construct
  source 1..50: mol_type = other DNA
cgcataccggcctatggactagtaccgtgacccaataatttccatctgta

SEQ ID NO: 658 - DNA, 49 bp, synthetic construct
  source 1..49: mol_type = other DNA
cgcataccggcctatggactattaccgaagaatcaataggagtgatggg

SEQ ID NO: 659 - DNA, 50 bp, synthetic construct
  source 1..50: mol_type = other DNA
cgcataccggcctatggactaaaaggctgctgtattaaagcactcttttc

SEQ ID NO: 660 - DNA, 54 bp, synthetic construct
  source 1..54: mol_type = other DNA
cgcataccggcctatggactagtccttggaaagtagtgaatagaaatcaccctg

SEQ ID NO: 661 - DNA, 49 bp, synthetic construct
  source 1..49: mol_type = other DNA
cgcataccggcctatggactagaagcccaggcatatagaatattggctt

SEQ ID NO: 662 - DNA, 49 bp, synthetic construct
  source 1..49: mol_type = other DNA
cgcataccggcctatggactagtagcggtaaataaaacagattcttgcc

SEQ ID NO: 663 - DNA, 48 bp, synthetic construct
  source 1..48: mol_type = other DNA
cgcataccggcctatggactacggagcctttttaactgatcgatgctc

SEQ ID NO: 664 - DNA, 44 bp, synthetic construct
  source 1..44: mol_type = other DNA
cgcataccggcctatggactattaccaacacctttagggagccg

SEQ ID NO: 665 - DNA, 49 bp, synthetic construct
  source 1..49: mol_type = other DNA
cgcataccggcctatggactacttcttgctgtcaaaagagtttccatcc

SEQ ID NO: 666 - DNA, 50 bp, synthetic construct
  source 1..50: mol_type = other DNA
cgcataccggcctatggactaaagtgggatagttctgaacatatctggag

SEQ ID NO: 667 - DNA, 52 bp, synthetic construct
  source 1..52: mol_type = other DNA
cgcataccggcctatggactattttgtcagaattcactactttctctcctgg

SEQ ID NO: 668 - DNA, 48 bp, synthetic construct
  source 1..48: mol_type = other DNA
cgcataccggcctatggactaattccagaagggttgtaatgagaacca

SEQ ID NO: 669 - DNA, 51 bp, synthetic construct
  source 1..51: mol_type = other DNA
cgcataccggcctatggactatttgaggaaacctggtatatatgagattgc

SEQ ID NO: 670 - DNA, 47 bp, synthetic construct
  source 1..47: mol_type = other DNA
cgcataccggcctatggactatctagccacaatatcatgaggcatgt

SEQ ID NO: 671 - DNA, 49 bp, synthetic construct
  source 1..49: mol_type = other DNA
cgcataccggcctatggactaaggccactggaaaagttcatcacaaatc

SEQ ID NO: 672 - DNA, 49 bp, synthetic construct
  source 1..49: mol_type = other DNA
cgcataccggcctatggactagaatgttttctgtttctttgtgatgccc

SEQ ID NO: 673 - DNA, 46 bp, synthetic construct
  source 1..46: mol_type = other DNA
cgcataccggcctatggactagaagcgaactggtttgaaaacatct

SEQ ID NO: 674 - DNA, 46 bp, synthetic construct
  source 1..46: mol_type = other DNA
cgcataccggcctatggactacaaccactgggtaaaaagcctgtgg

SEQ ID NO: 675 - DNA, 49 bp, synthetic construct
  source 1..49: mol_type = other DNA
cgcataccggcctatggactaggtaggcattcattggaaaatgatctgg

SEQ ID NO: 676 - DNA, 46 bp, synthetic construct
  source 1..46: mol_type = other DNA
cgcataccggcctatggactactcgcttgaagtcaatcacgaaggc

SEQ ID NO: 677 - DNA, 47 bp, synthetic construct
  source 1..47: mol_type = other DNA
cgcataccggcctatggactagcagtcagactctttaggtgtgaccc

SEQ ID NO: 678 - DNA, 46 bp, synthetic construct
  source 1..46: mol_type = other DNA
cgcataccggcctatggactacaagcagtaaccatctgacacgaag

SEQ ID NO: 679 - DNA, 49 bp, synthetic construct
  source 1..49: mol_type = other DNA
cgcataccggcctatggactataatcagctttatttgggtcaatgacca

SEQ ID NO: 680 - DNA, 51 bp, synthetic construct
  source 1..51: mol_type = other DNA
gctccgttacgcgtttaagtcaacggatatcccagataggaaattcctcct

SEQ ID NO: 681 - DNA, 49 bp, synthetic construct
  source 1..49: mol_type = other DNA
gctccgttacgcgtttaagtcacctggtcttcgtatgaatgctcatgtt

SEQ ID NO: 682 - DNA, 44 bp, synthetic construct
  source 1..44: mol_type = other DNA
gctccgttacgcgtttaagtcagcgacgggtctctagttccctg

SEQ ID NO: 683 - DNA, 47 bp, synthetic construct
  source 1..47: mol_type = other DNA
gctccgttacgcgtttaagtcaggcccataggatgaactcagtcctc

SEQ ID NO: 684 - DNA, 46 bp, synthetic construct
  source 1..46: mol_type = other DNA
gctccgttacgcgtttaagtctgatggacacgacatcagaagactc

SEQ ID NO: 685 - DNA, 44 bp, synthetic construct
  source 1..44: mol_type = other DNA
gctccgttacgcgtttaagtcacgggctgaaagtgttggatccc

SEQ ID NO: 686 - DNA, 44 bp, synthetic construct
  source 1..44: mol_type = other DNA
gctccgttacgcgtttaagtctcgtgaagacggaggttcgagag

SEQ ID NO: 687 - DNA, 45 bp, synthetic construct
  source 1..45: mol_type = other DNA
gctccgttacgcgtttaagtccggagaggattagggctgatcctc

SEQ ID NO: 688 - DNA, 49 bp, synthetic construct
  source 1..49: mol_type = other DNA
gctccgttacgcgtttaagtctttatacaactgaattgggtttggatgg

SEQ ID NO: 689 - DNA, 44 bp, synthetic construct
  source 1..44: mol_type = other DNA
gctccgttacgcgtttaagtccgagcttctagacggtgggacag

SEQ ID NO: 690 - DNA, 45 bp, synthetic construct
  source 1..45: mol_type = other DNA
gctccgttacgcgtttaagtcacttggacatggtgaagtctggag

SEQ ID NO: 691 - DNA, 45 bp, synthetic construct
  source 1..45: mol_type = other DNA
gctccgttacgcgtttaagtctccagctcattccgtagctgaagc

SEQ ID NO: 692 - DNA, 45 bp, synthetic construct
  source 1..45: mol_type = other DNA
gctccgttacgcgtttaagtccggtttgagggcttcaatctccgc

SEQ ID NO: 693 - DNA, 46 bp, synthetic construct
  source 1..46: mol_type = other DNA
gctccgttacgcgtttaagtcaaccggttcatctcggagatctctg

SEQ ID NO: 694 - DNA, 47 bp, synthetic construct
  source 1..47: mol_type = other DNA
gctccgttacgcgtttaagtcgaacccatctcgggtttcaatcttct

SEQ ID NO: 695 - DNA, 44 bp, synthetic construct
  source 1..44: mol_type = other DNA
gctccgttacgcgtttaagtctgactgccatacagctgtctccc

SEQ ID NO: 696 - DNA, 45 bp, synthetic construct
  source 1..45: mol_type = other DNA
gctccgttacgcgtttaagtcccccatccttaatggccatctccc

SEQ ID NO: 697 - DNA, 44 bp, synthetic construct
  source 1..44: mol_type = other DNA
gctccgttacgcgtttaagtcgttggacttcagtggccattcac

SEQ ID NO: 698 - DNA, 44 bp, synthetic construct
  source 1..44: mol_type = other DNA
gctccgttacgcgtttaagtctcctggtgatctcgatgtccagg

SEQ ID NO: 699 - DNA, 47 bp, synthetic construct
  source 1..47: mol_type = other DNA
gcaacccgagcattcgtatgatttttcatcggctctatcactgaaag

SEQ ID NO: 700 - DNA, 45 bp, synthetic construct
  source 1..45: mol_type = other DNA
gcaacccgagcattcgtatgatcgtggacacacttcaccatgttt

SEQ ID NO: 701 - DNA, 47 bp, synthetic construct
  source 1..47: mol_type = other DNA
gcaacccgagcattcgtatgagtaaacagctgtagctttgaagttgg

SEQ ID NO: 702 - DNA, 47 bp, synthetic construct; identical to SEQ ID NO: 699
  source 1..47: mol_type = other DNA

SEQ ID NO: 703 - DNA, 45 bp, synthetic construct; identical to SEQ ID NO: 700
  source 1..45: mol_type = other DNA

SEQ ID NO: 704 - DNA, 47 bp, synthetic construct; identical to SEQ ID NO: 701
  source 1..47: mol_type = other DNA

SEQ ID NO: 705 - DNA, 53 bp, synthetic construct
  source 1..53: mol_type = other DNA
gcaacccgagcattcgtatgagtgctacctaggaacttaactgtagctcaatc

SEQ ID NO: 706 - DNA, 49 bp, synthetic construct
  source 1..49: mol_type = other DNA
gcaacccgagcattcgtatgagtattcctgaagacatcttgctatcagc

SEQ ID NO: 707 - DNA, 51 bp, synthetic construct
  source 1..51: mol_type = other DNA
gcaacccgagcattcgtatgattgcagcactagaaaagtagatcctgagag

SEQ ID NO: 708 - DNA, 51 bp, synthetic construct
  source 1..51: mol_type = other DNA
gcaacccgagcattcgtatgagtcctcagaccattaccaatagtggaagaa

SEQ ID NO: 709 - DNA, 51 bp, synthetic construct
  source 1..51: mol_type = other DNA
gcaacccgagcattcgtatgatgtgacactaggcatttatcctaagaagca

SEQ ID NO: 710 - DNA, 49 bp, synthetic construct
  source 1..49: mol_type = other DNA
gcaacccgagcattcgtatgataacctggcagattaaataggcaggtag

SEQ ID NO: 711 - DNA, 54 bp, synthetic construct
  source 1..54: mol_type = other DNA
gcaacccgagcattcgtatgagatgcctcacatatacatcaagttctagatcag

SEQ ID NO: 712 - DNA, 51 bp, synthetic construct
  source 1..51: mol_type = other DNA
gcaacccgagcattcgtatgatagctgtctcaaaagttaagagactggaga

SEQ ID NO: 713 - DNA, 47 bp, synthetic construct
  source 1..47: mol_type = other DNA
gcaacccgagcattcgtatgacccgctttggagatagttcagctgtg

SEQ ID NO: 714 - DNA, 51 bp, synthetic construct
  source 1..51: mol_type = other DNA
gcaacccgagcattcgtatgatcgacgagtagagtacaagagtttcttctg

SEQ ID NO: 715 - DNA, 46 bp, synthetic construct
  source 1..46: mol_type = other DNA
gcaacccgagcattcgtatgatttggccaactatggtaatccatgc

SEQ ID NO: 716 - DNA, 51 bp, synthetic construct
  source 1..51: mol_type = other DNA
gcaacccgagcattcgtatgacaacagcaacaagtaaaacagaataccagg

SEQ ID NO: 717 - DNA, 49 bp, synthetic construct
  source 1..49: mol_type = other DNA
gcaacccgagcattcgtatgactttcaccaagttctcagaattcacgtt

SEQ ID NO: 718 - DNA, 53 bp, synthetic construct
  source 1..53: mol_type = other DNA
gcaacccgagcattcgtatgaaacccttcagttcactagtactgagattacag

SEQ ID NO: 719 - DNA, 51 bp, synthetic construct
  source 1..51: mol_type = other DNA
gcaacccgagcattcgtatgaattacatttccaagattagagtggtctgtg

SEQ ID NO: 720 - DNA, 51 bp, synthetic construct
  source 1..51: mol_type = other DNA
gcaacccgagcattcgtatgagactgaccaggaagtataagccagatactt

SEQ ID NO: 721 - DNA, 46 bp, synthetic construct
  source 1..46: mol_type = other DNA
gcaacccgagcattcgtatgagaccctggcagaaaaatggtccagt

SEQ ID NO: 722 - DNA, 47 bp, synthetic construct
  source 1..47: mol_type = other DNA
gcaacccgagcattcgtatgatgttggcaatagggttataacaaggc

SEQ ID NO: 723 - DNA, 49 bp, synthetic construct
  source 1..49: mol_type = other DNA
cgtcaggtgagcatcttacatcgacctcatcacagattatgtcatcgca

SEQ ID NO: 724 - DNA, 51 bp, synthetic construct
  source 1..51: mol_type = other DNA
cgtcaggtgagcatcttacatgtcacttgacatcatatgagtcgaattggg

SEQ ID NO: 725 - DNA, 52 bp, synthetic construct
  source 1..52: mol_type = other DNA
cgtcaggtgagcatcttacatgtacagggcttataagactctctatttgtcc

SEQ ID NO: 726 - DNA, 51 bp, synthetic construct
  source 1..51: mol_type = other DNA
cgtcaggtgagcatcttacatccctgcgatatctatgatgggtagtctcat

SEQ ID NO: 727 - DNA, 51 bp, synthetic construct
  source 1..51: mol_type = other DNA
cgtcaggtgagcatcttacatccgttccagacatctctagactcataggac

SEQ ID NO: 728 - DNA, 51 bp, synthetic construct
  source 1..51: mol_type = other DNA
cgtcaggtgagcatcttacatagtgtgttgatcttgaaatccattggatcg

SEQ ID NO: 729 - DNA, 47 bp, synthetic construct
  source 1..47: mol_type = other DNA
cgtcaggtgagcatcttacatccgcccttgctcctattagtccaggg

SEQ ID NO: 730 - DNA, 51 bp, synthetic construct
  source 1..51: mol_type = other DNA
cgtcaggtgagcatcttacatgtacctctagaactgtgtaagtgaatttgc

SEQ ID NO: 731 - DNA, 48 bp, synthetic construct
  source 1..48: mol_type = other DNA
cgtcaggtgagcatcttacattaagcacaacattctccaaatgggatc

SEQ ID NO: 732 - DNA, 48 bp, synthetic construct
  source 1..48: mol_type = other DNA
cgtcaggtgagcatcttacatcgagcatcccaattcatctacgttgga

SEQ ID NO: 733 - DNA, 49 bp, synthetic construct
  source 1..49: mol_type = other DNA
cgtcaggtgagcatcttacatgaagcgtgtttgatattcaaagactgtc

SEQ ID NO: 734 - DNA, 49 bp, synthetic construct
  source 1..49: mol_type = other DNA
cgtcaggtgagcatcttacatttgcaccattcatagattctccaaacca

SEQ ID NO: 735 - DNA, 46 bp, synthetic construct
  source 1..46: mol_type = other DNA
cgtcaggtgagcatcttacatccaggcagaattttaggtctctgca

SEQ ID NO: 736 - DNA, 49 bp, synthetic construct
  source 1..49: mol_type = other DNA
cgtcaggtgagcatcttacatagtcagacacatatttgacatggttctg

SEQ ID NO: 737 - DNA, 48 bp, synthetic construct
  source 1..48: mol_type = other DNA
cgtcaggtgagcatcttacatccccaccatcatttcctttaggaccag

SEQ ID NO: 738 - DNA, 47 bp, synthetic construct
  source 1..47: mol_type = other DNA
cgtcaggtgagcatcttacataacgacatgattcacagattccaggg

SEQ ID NO: 739 - DNA, 46 bp, synthetic construct
  source 1..46: mol_type = other DNA
cgtcaggtgagcatcttacatgacctgtgccaaaataagagtggga

SEQ ID NO: 740 - DNA, 44 bp, synthetic construct
  source 1..44: mol_type = other DNA
cgtcaggtgagcatcttacatggacgaggtccagctatacctgg

SEQ ID NO: 741 - DNA, 45 bp, synthetic construct
  source 1..45: mol_type = other DNA
cgtcaggtgagcatcttacatacacctttgatgccattagagcca

SEQ ID NO: 742 - DNA, 48 bp, synthetic construct
  source 1..48: mol_type = other DNA
cgtcaggtgagcatcttacatggtgtattctccactcttgagttcggg

SEQ ID NO: 743 - DNA, 45 bp, synthetic construct
  source 1..45: mol_type = other DNA
cgtcaggtgagcatcttacatgacgtggtccaataggacctggat

SEQ ID NO: 744 - DNA, 45 bp, synthetic construct
  source 1..45: mol_type = other DNA
cgtcaggtgagcatcttacataagcccagtgtgtttagtacagcc

SEQ ID NO: 745 - DNA, 46 bp, synthetic construct
  source 1..46: mol_type = other DNA
cgtcaggtgagcatcttacatctcccctcagatcctctttcacctc

SEQ ID NO: 746 - DNA, 49 bp, synthetic construct
  source 1..49: mol_type = other DNA
cgtcaggtgagcatcttacataagcccagtttccatgttacagaatact

SEQ ID NO: 747 - DNA, 50 bp, synthetic construct
  source 1..50: mol_type = other DNA
tgcctccgtctgagtattcctcgggttgtctctaaagtactctcccatgg

SEQ ID NO: 748 - DNA, 49 bp, synthetic construct
  source 1..49: mol_type = other DNA
tgcctccgtctgagtattcctctaagatcgtcatagatgatcaaagcgt

SEQ ID NO: 749 - DNA, 48 bp, synthetic construct
  source 1..48: mol_type = other DNA
tgcctccgtctgagtattcctgacgctgataacgtgagacaagaaagc

SEQ ID NO: 750 - DNA, 54 bp, synthetic construct
  source 1..54: mol_type = other DNA
tgcctccgtctgagtattcctttggtctccttctttaattagcttgtcattccc

SEQ ID NO: 751 - DNA, 46 bp, synthetic construct
  source 1..46: mol_type = other DNA
tgcctccgtctgagtattcctgtggacaactccaacattgtcgggt

SEQ ID NO: 752 - DNA, 45 bp, synthetic construct
  source 1..45: mol_type = other DNA
tgcctccgtctgagtattcctctcgcatggagatttcttgcacca

SEQ ID NO: 753 - DNA, 46 bp, synthetic construct
  source 1..46: mol_type = other DNA
tgcctccgtctgagtattcctaaacccatcagacctgatattgccc

SEQ ID NO: 754 - DNA, 49 bp, synthetic construct
  source 1..49: mol_type = other DNA
tgcctccgtctgagtattcctttaaacatttgttggaatgtaagcggac

SEQ ID NO: 755 - DNA, 50 bp, synthetic construct
  source 1..50: mol_type = other DNA
tgcctccgtctgagtattcctctgcctttccatcaatagcattaccgagg

SEQ ID NO: 756 - DNA, 48 bp, synthetic construct
  source 1..48: mol_type = other DNA
tgcctccgtctgagtattccttatccctttaagcctgaagagaattcc

SEQ ID NO: 757 - DNA, 44 bp, synthetic construct
  source 1..44: mol_type = other DNA
tgcctccgtctgagtattcctgagtgcttgaacattcctcagcc

SEQ ID NO: 758 - DNA, 46 bp, synthetic construct
  source 1..46: mol_type = other DNA
tgcctccgtctgagtattcctgagctcgagaatggaggacatctca

SEQ ID NO: 759 - DNA, 47 bp, synthetic construct
  source 1..47: mol_type = other DNA
tgcctccgtctgagtattccttggtgttcttcaatagccatgggaga

SEQ ID NO: 760 - DNA, 45 bp, synthetic construct
  source 1..45: mol_type = other DNA
tgcctccgtctgagtattccttcgcggagtagggagccaagtact

SEQ ID NO: 761 - DNA, 47 bp, synthetic construct
  source 1..47: mol_type = other DNA
tgcctccgtctgagtattcctaccgctccagtttgtcaagataaccc

SEQ ID NO: 762 - DNA, 49 bp, synthetic construct
  source 1..49: mol_type = other DNA
tgcctccgtctgagtattcctaagaatgactggtaaggcagtcaaagag

SEQ ID NO: 763 - DNA, 45 bp, synthetic construct
  source 1..45: mol_type = other DNA
tgcctccgtctgagtattcctgagctccaggtcaacagacgtgtc

SEQ ID NO: 764 - DNA, 49 bp, synthetic construct
  source 1..49: mol_type = other DNA
tgcctccgtctgagtattcctcacccagtcttctgaagtcgagtgttag

SEQ ID NO: 765 - DNA, 50 bp, synthetic construct
  source 1..50: mol_type = other DNA
tgcctccgtctgagtattccttccgcggatgcctttatagaacaattctg

SEQ ID NO: 766 - DNA, 47 bp, synthetic construct
  source 1..47: mol_type = other DNA
tgcctccgtctgagtattccttggaaaggaatcgttcatcttggctg

SEQ ID NO: 767 - DNA, 46 bp, synthetic construct
  source 1..46: mol_type = other DNA
tgcctccgtctgagtattcctggttcgttgaaacgtttctggttga

SEQ ID NO: 768 - DNA, 44 bp, synthetic construct
  source 1..44: mol_type = other DNA
tgcctccgtctgagtattcctggccatccgtcagtctcttcacc

SEQ ID NO: 769 - DNA, 48 bp, synthetic construct
  source 1..48: mol_type = other DNA
tgcctccgtctgagtattcctgggcaaagcatttttggagaccagtcc

SEQ ID NO: 770 - DNA, 44 bp, synthetic construct
  source 1..44: mol_type = other DNA
tgcctccgtctgagtattcctcgtctcgcgcaataccatcacca

SEQ ID NO: 771 - DNA, 47 bp, synthetic construct
  source 1..47: mol_type = other DNA
gtgtgtgccaggatgatcaatgactgctcgttcatattagtcgcacc

SEQ ID NO: 772 - DNA, 51 bp, synthetic construct
  source 1..51: mol_type = other DNA
gtgtgtgccaggatgatcaatgatgagtcatgattctatccatgtcgtcag

SEQ ID NO: 773 - DNA, 49 bp, synthetic construct
  source 1..49: mol_type = other DNA
gtgtgtgccaggatgatcaatagagggtctgatctttacccaaaaggtc

SEQ ID NO: 774 - DNA, 54 bp, synthetic construct
  source 1..54: mol_type = other DNA
gtgtgtgccaggatgatcaataatctgaagtaaagaatcaattacggtctcagg

SEQ ID NO: 775 - DNA, 50 bp, synthetic construct
  source 1..50: mol_type = other DNA
gtgtgtgccaggatgatcaatgagcaagtatgacactcgaaccaaaaacc

SEQ ID NO: 776 - DNA, 51 bp, synthetic construct
  source 1..51: mol_type = other DNA
gtgtgtgccaggatgatcaatgaagcagactggtatatttttcctcgatgt

SEQ ID NO: 777 - DNA, 54 bp, synthetic construct
  source 1..54: mol_type = other DNA
gtgtgtgccaggatgatcaatagtctgcactctatagtaattgtaaagatggcg

SEQ ID NO: 778 - DNA, 48 bp, synthetic construct
  source 1..48: mol_type = other DNA
gtgtgtgccaggatgatcaatagtggcctttctttaacctctaacctc

SEQ ID NO: 779 - DNA, 48 bp, synthetic construct
  source 1..48: mol_type = other DNA
gtgtgtgccaggatgatcaatgtgcaccttcacattatcgcagctttc

SEQ ID NO: 780 - DNA, 46 bp, synthetic construct
  source 1..46: mol_type = other DNA
gtgtgtgccaggatgatcaatcactaggccacacattttagctgcc

SEQ ID NO: 781 - DNA, 50 bp, synthetic construct
  source 1..50: mol_type = other DNA
gtgtgtgccaggatgatcaatactccaaatatgtgagcaaatgagttggg

SEQ ID NO: 782 - DNA, 46 bp, synthetic construct
  source 1..46: mol_type = other DNA
gtgtgtgccaggatgatcaataacggtcctcattgattctcgcttt

SEQ ID NO: 783 - DNA, 49 bp, synthetic construct
  source 1..49: mol_type = other DNA
gtgtgtgccaggatgatcaattcaggacgtagttttcaatcatttcctg

SEQ ID NO: 784 - DNA, 47 bp, synthetic construct
  source 1..47: mol_type = other DNA
gtgtgtgccaggatgatcaatgcctcttatggacggtgatagttccc

SEQ ID NO: 785 - DNA, 54 bp, synthetic construct
  source 1..54: mol_type = other DNA
gtgtgtgccaggatgatcaatagtgcatggtccagactttctttaatttcttgg

SEQ ID NO: 786 - DNA, 47 bp, synthetic construct
  source 1..47: mol_type = other DNA
gtgtgtgccaggatgatcaataccgaaaggtcgacttcaaatgggtc

SEQ ID NO: 787 - DNA, 48 bp, synthetic construct
  source 1..48: mol_type = other DNA
gtgtgtgccaggatgatcaattccgactggagtttgtttcctcatgtt

SEQ ID NO: 788 - DNA, 50 bp, synthetic construct
  source 1..50: mol_type = other DNA
gtgtgtgccaggatgatcaatgtcctcttcgatttctttctgaaactggc

SEQ ID NO: 789 - DNA, 54 bp, synthetic construct
  source 1..54: mol_type = other DNA
gtgtgtgccaggatgatcaatgaaccagaacagaatcaataattggtcttgcag

SEQ ID NO: 790 - DNA, 46 bp, synthetic construct
  source 1..46: mol_type = other DNA
gtgtgtgccaggatgatcaattgatctctctctcattgagaggccg

SEQ ID NO: 791 - DNA, 47 bp, synthetic construct
  source 1..47: mol_type = other DNA
gtgtgtgccaggatgatcaataaagagttccctcctaaggagtcctg

SEQ ID NO: 792 - DNA, 49 bp, synthetic construct
  source 1..49: mol_type = other DNA
gtgtgtgccaggatgatcaatatacgaaagtaaatgtctttggaggttc

SEQ ID NO: 793 - DNA, 50 bp, synthetic construct
  source 1..50: mol_type = other DNA
gtgtgtgccaggatgatcaattcatatcaatgatgagcatctgaagacgg

SEQ ID NO: 794 - DNA, 46 bp, synthetic construct
  source 1..46: mol_type = other DNA
gtgtgtgccaggatgatcaatacgccagggtagacaatgaaaggtt

SEQ ID NO: 795 - DNA, 63 bp, synthetic construct
  source 1..63: mol_type = other DNA
agagtgagtagtagtggagtttattctacacggagcatgtgcatatcaaccgcacatgctccg

SEQ ID NO: 796 - DNA, 63 bp, synthetic construct
  source 1..63: mol_type = other DNA
gcacatgctccgtgtagaatacggagcatgtgcggttgatattagagtgagtagtagtggagt

SEQ ID NO: 797 - DNA, 60 bp, synthetic construct
  source 1..60: mol_type = other DNA
ctctaacttccatcacattagtccataggccggtatgcgagctttaaacgcataccggcc

SEQ ID NO: 798 - DNA, 60 bp, synthetic construct
  source 1..60: mol_type = other DNA
cgcataccggcctatggactaggccggtatgcgtttaaagcttaccctctaacttccatc

SEQ ID NO: 799 - DNA, 60 bp, synthetic construct
  source 1..60: mol_type = other DNA
acaacccattcctttcatgacttaaacgcgtaacggagcgatcttagagctccgttacgc

SEQ ID NO: 800 - DNA, 60 bp, synthetic construct
  source 1..60: mol_type = other DNA
gctccgttacgcgtttaagtcgcgtaacggagctctaagatctaccacaacccattcctt

SEQ ID NO: 801 - DNA, 60 bp, synthetic construct
  source 1..60: mol_type = other DNA
actccctacacctccaattcatacgaatgctcgggttgcttactaatcgcaacccgagca

SEQ ID NO: 802 - DNA, 60 bp, synthetic construct
  source 1..60: mol_type = other DNA
gcaacccgagcattcgtatgatgctcgggttgcgattagtaattttactccctacacctc

SEQ ID NO: 803 - DNA, 60 bp, synthetic construct
  source 1..60: mol_type = other DNA
ttaggttgagaataggataggaatactcagacggaggcaagaagcttttgcctccgtctg

SEQ ID NO: 804 - DNA, 60 bp, synthetic construct
  source 1..60: mol_type = other DNA
tgcctccgtctgagtattcctcagacggaggcaaaagcttcttaggttaggttgagaata

SEQ ID NO: 805 - DNA, 60 bp, synthetic construct
  source 1..60: mol_type = other DNA
agttgatagagggagaatattgatcatcctggcacacacagtaacattgtgtgtgccagg

SEQ ID NO: 806 - DNA, 60 bp, synthetic construct
  source 1..60: mol_type = other DNA
gtgtgtgccaggatgatcaatcctggcacacacaatgttactttagagttgatagaggga

SEQ ID NO: 807 - DNA, 44 bp, synthetic construct
  source 1..44: mol_type = other DNA
tgtggagggattgaaggatacgtgagttagccgatgcttttaga

SEQ ID NO: 808 - DNA, 44 bp, synthetic construct
  source 1..44: mol_type = other DNA
tgtggagggattgaaggatatcaggtccgtgtctcagtacctca

SEQ ID NO: 809 - DNA, 44 bp, synthetic construct
  source 1..44: mol_type = other DNA
tgtggagggattgaaggatatcgatccagcttcatggagtcccc

SEQ ID NO: 810 - DNA, 44 bp, synthetic construct
  source 1..44: mol_type = other DNA
tgtggagggattgaaggatatgctgtcgccccggacgtaagccg

SEQ ID NO: 811 - DNA, 44 bp, synthetic construct
  source 1..44: mol_type = other DNA
tgtggagggattgaaggatactgttgagcccgggtaaggttgga

SEQ ID NO: 812 - DNA, 44 bp, synthetic construct
  source 1..44: mol_type = other DNA
tgtggagggattgaaggatatgggcattcatcgtttactgccac

SEQ ID NO: 813 - DNA, 44 bp, synthetic construct
  source 1..44: mol_type = other DNA
tgtggagggattgaaggatatgggcattccgcgtacttctctag

SEQ ID NO: 814 - DNA, 44 bp, synthetic construct
  source 1..44: mol_type = other DNA
tgtggagggattgaaggatagtctgcatacgtgttactcacggc

SEQ ID NO: 815 - DNA, 44 bp, synthetic construct
  source 1..44: mol_type = other DNA
ataggaaatggtggtagtgtatggcgaatccagcttcacgatca

SEQ ID NO: 816 - DNA, 44 bp, synthetic construct
  source 1..44: mol_type = other DNA
ataggaaatggtggtagtgtcgtctctctagagtcctcagctac

SEQ ID NO: 817 - DNA, 44 bp, synthetic construct
  source 1..44: mol_type = other DNA
ataggaaatggtggtagtgtgatcccgagttatcggcaggtacc

SEQ ID NO: 818 - DNA, 44 bp, synthetic construct
  source 1..44: mol_type = other DNA
ataggaaatggtggtagtgttctccctatccatcgaaggttacc

SEQ ID NO: 819 - DNA, 44 bp, synthetic construct
  source 1..44: mol_type = other DNA
ataggaaatggtggtagtgtcctacgctcggatcctccgtaaat

SEQ ID NO: 820 - DNA, 44 bp, synthetic construct
  source 1..44: mol_type = other DNA
ataggaaatggtggtagtgtggaactgtactcaagacagcctca

SEQ ID NO: 821 - DNA, 44 bp, synthetic construct
  source 1..44: mol_type = other DNA
ataggaaatggtggtagtgtcgtcagcgagtattcatcgttatg

SEQ ID NO: 822 - DNA, 47 bp, synthetic construct
  source 1..47: mol_type = other DNA
ataggaaatggtggtagtgtgtgttgtcttacgactatactgttagg

SEQ ID NO: 823 - DNA, 44 bp, synthetic construct
  source 1..44: mol_type = other DNA
agagtgagtagtagtggagtaagtcttctgcactcaagtcatgg

SEQ ID NO: 824 - DNA, 44 bp, synthetic construct
  source 1..44: mol_type = other DNA
agagtgagtagtagtggagtggcggctcatccatcagtgatcgg

SEQ ID NO: 825 - DNA, 44 bp, synthetic construct
  source 1..44: mol_type = other DNA
agagtgagtagtagtggagttacccgcaggctcatccatcacac

SEQ ID NO: 826 - DNA, 45 bp, synthetic construct
  source 1..45: mol_type = other DNA
agagtgagtagtagtggagtgtaggtaccgtcaattgatagtgta

SEQ ID NO: 827 - DNA, 44 bp, synthetic construct
  source 1..44: mol_type = other DNA
agagtgagtagtagtggagtagcccccaacacttagcactctag

SEQ ID NO: 828 - DNA, 44 bp, synthetic construct
  source 1..44: mol_type = other DNA
agagtgagtagtagtggagtggaaatccgaactgagattgggaa

SEQ ID NO: 829 - DNA, 44 bp, synthetic construct
  source 1..44: mol_type = other DNA
agagtgagtagtagtggagtaagacttcatgtaggcgagttcgt

SEQ ID NO: 830 - DNA, 44 bp, synthetic construct
  source 1..44: mol_type = other DNA
agagtgagtagtagtggagtcctccctctatctctagaggccag

SEQ ID NO: 831 - DNA, 44 bp, synthetic construct
  source 1..44: mol_type = other DNA
ttggaggtgtagggagtaaaggagacggctgactcctataatcc

SEQ ID NO: 832 - DNA, 44 bp, synthetic construct
  source 1..44: mol_type = other DNA
ttggaggtgtagggagtaaatgaaatcatctaggcaagctccga

SEQ ID NO: 833 - DNA, 44 bp, synthetic construct
  source 1..44: mol_type = other DNA
ttggaggtgtagggagtaaaagtctctagacatgcgtctagaca

SEQ ID NO: 834 - DNA, 44 bp, synthetic construct
  source 1..44: mol_type = other DNA
ttggaggtgtagggagtaaaaaatcactaccaacagagcttatg

SEQ ID NO: 835 - DNA, 46 bp, synthetic construct
  source 1..46: mol_type = other DNA
ttggaggtgtagggagtaaaggcggctttcacatcagacttatact

SEQ ID NO: 836 - DNA, 44 bp, synthetic construct
  source 1..44: mol_type = other DNA
ttggaggtgtagggagtaaaagtgtcagttgcagaccagagtcg

SEQ ID NO: 837 - DNA, 44 bp, synthetic construct
  source 1..44: mol_type = other DNA
ttggaggtgtagggagtaaaacgcacctgtctcagcgtcccgct

SEQ ID NO: 838 - DNA, 44 bp, synthetic construct
  source 1..44: mol_type = other DNA
ttggaggtgtagggagtaaagaatgatggcaactaatgacatcc

SEQ ID NO: 839 - DNA, 44 bp, synthetic construct
  source 1..44: mol_type = other DNA
agggtgtgtttgtaaagggtggaggcataagggccatactgtgg

SEQ ID NO: 840 - DNA, 44 bp, synthetic construct
  source 1..44: mol_type = other DNA
agggtgtgtttgtaaagggtgcgccgaagagtcgcatgcttagt

SEQ ID NO: 841 - DNA, 44 bp, synthetic construct
  source 1..44: mol_type = other DNA
agggtgtgtttgtaaagggtgcaactgccaggactacagggcat

SEQ ID NO: 842 - DNA, 44 bp, synthetic construct
  source 1..44: mol_type = other DNA
agggtgtgtttgtaaagggtcgctggcttcagatacttcggcac

SEQ ID NO: 843 - DNA, 44 bp, synthetic construct
  source 1..44: mol_type = other DNA
agggtgtgtttgtaaagggtcaccctctcgatatctacgcaaaa

SEQ ID NO: 844 - DNA, 44 bp, synthetic construct
  source 1..44: mol_type = other DNA
agggtgtgtttgtaaagggtgttcgaagaccttcattccccgtg

SEQ ID NO: 845 - DNA, 45 bp, synthetic construct
  source 1..45: mol_type = other DNA
agggtgtgtttgtaaagggtcaaccccattgtgaatgattctgct

SEQ ID NO: 846 - DNA, 45 bp, synthetic construct
  source 1..45: mol_type = other DNA
agggtgtgtttgtaaagggtttattcgatactatgcggtatttta

SEQ ID NO: 847 - DNA, 44 bp, synthetic construct
  source 1..44: mol_type = other DNA
aggttaggttgagaataggaacgcgcgggcccatctcataggcc

SEQ ID NO: 848 - DNA, 44 bp, synthetic construct
  source 1..44: mol_type = other DNA
aggttaggttgagaataggaataccctctatgaggcaggttcgg

SEQ ID NO: 849 - DNA, 45 bp, synthetic construct
  source 1..45: mol_type = other DNA
aggttaggttgagaataggaactacccctacacctagtattctag

SEQ ID NO: 850 - DNA, 44 bp, synthetic construct
  source 1..44: mol_type = other DNA
aggttaggttgagaataggaagttacagtccagagaatcgcgaa

SEQ ID NO: 851 - DNA, 44 bp, synthetic construct
  source 1..44: mol_type = other DNA
aggttaggttgagaataggaatcactccagcttcatgtagggct

SEQ ID NO: 852 - DNA, 44 bp, synthetic construct
  source 1..44: mol_type = other DNA
aggttaggttgagaataggatgtccgaactgagacaagtttaac

SEQ ID NO: 853 - DNA, 44 bp, synthetic construct
  source 1..44: mol_type = other DNA
aggttaggttgagaataggaacgcacctgtcttcctgtcccgct

SEQ ID NO: 854 - DNA, 44 bp, synthetic construct
  source 1..44: mol_type = other DNA
aggttaggttgagaataggaagccctgaagacagagctttagtt

SEQ ID NO: 855 - DNA, 44 bp, synthetic construct
  source 1..44: mol_type = other DNA
gatgatgtagtagtaagggtaaaaatgcagtctgaaggttgtcg

SEQ ID NO: 856 - DNA, 44 bp, synthetic construct
  source 1..44: mol_type = other DNA
gatgatgtagtagtaagggtcgaccctctctcagaggcaggaac

SEQ ID NO: 857 - DNA, 45 bp, synthetic construct
  source 1..45: mol_type = other DNA
gatgatgtagtagtaagggtcatgtcattatcgtccctgatattt

SEQ ID NO: 858 - DNA, 44 bp, synthetic construct
  source 1..44: mol_type = other DNA
gatgatgtagtagtaagggtcgccccatctctgagcgaattaga

SEQ ID NO: 859 - DNA, 44 bp, synthetic construct
  source 1..44: mol_type = other DNA
gatgatgtagtagtaagggtgatcctctctcagaggcaggtacg

SEQ ID NO: 860 - DNA, 47 bp, synthetic construct
  source 1..47: mol_type = other DNA
gatgatgtagtagtaagggttcgtctgatctgcgattactagcataa

SEQ ID NO: 861 - DNA, 44 bp, synthetic construct
  source 1..44: mol_type = other DNA
gatgatgtagtagtaagggtgtccctctagagtgctcttgccat

SEQ ID NO: 862 - DNA, 45 bp, synthetic construct
  source 1..45: mol_type = other DNA
gatgatgtagtagtaagggtaaacctatctctaggctatgcaatg

SEQ ID NO: 863 - DNA, 48 bp, synthetic construct
  source 1..48: mol_type = other DNA
gcacatgctccgtgtagaatagacaccctttagaaaccaatggacacc

SEQ ID NO: 864 - DNA, 46 bp, synthetic construct
  source 1..46: mol_type = other DNA
gcacatgctccgtgtagaatagttggttgggaactcgatattcggt

SEQ ID NO: 865 - DNA, 44 bp, synthetic construct
  source 1..44: mol_type = other DNA
gcacatgctccgtgtagaataacccacctggtatggagtctgcc

SEQ ID NO: 866 - DNA, 51 bp, synthetic construct
  source 1..51: mol_type = other DNA
gcacatgctccgtgtagaataagtgcattagtgttacagtagaaaactgcc

SEQ ID NO: 867 - DNA, 49 bp, synthetic construct
  source 1..49: mol_type = other DNA
gcacatgctccgtgtagaatatcaccagcttgattgatacaggatcagg

SEQ ID NO: 868 - DNA, 46 bp, synthetic construct
  source 1..46: mol_type = other DNA
gcacatgctccgtgtagaatagttgcaccttagaggagtgctcatg

SEQ ID NO: 869 - DNA, 48 bp, synthetic construct
  source 1..48: mol_type = other DNA
gcacatgctccgtgtagaatagtgagcatcgattgtgaatgttcctgt

SEQ ID NO: 870 - DNA, 45 bp, synthetic construct
  source 1..45: mol_type = other DNA
gcacatgctccgtgtagaataaagatgcgctcattcaacacaagc

SEQ ID NO: 871 - DNA, 46 bp, synthetic construct
  source 1..46: mol_type = other DNA
gcacatgctccgtgtagaatatccctgagagaaggatcactgtcca

SEQ ID NO: 872 - DNA, 48 bp, synthetic construct
  source 1..48: mol_type = other DNA
gcacatgctccgtgtagaatagagcaggagacatagaggatgactgga

SEQ ID NO: 873 - DNA, 44 bp, synthetic construct
  source 1..44: mol_type = other DNA
gcacatgctccgtgtagaatattgcaggatcccgataacgatgc

SEQ ID NO: 874 - DNA, 47 bp, synthetic construct
  source 1..47: mol_type = other DNA
gcacatgctccgtgtagaataagtcactcatagggcccaatatcatc

SEQ ID NO: 875 - DNA, 46 bp, synthetic construct
  source 1..46: mol_type = other DNA
gcacatgctccgtgtagaatactcgcagtgttacttctctgaacct

SEQ ID NO: 876 - DNA, 49 bp, synthetic construct
  source 1..49: mol_type = other DNA
gcacatgctccgtgtagaataagtctttttcaagcacttggactttgac

SEQ ID NO: 877 - DNA, 45 bp, synthetic construct
  source 1..45: mol_type = other DNA
gcacatgctccgtgtagaatatcagctctgattgactgtgagggt

SEQ ID NO: 878 - DNA, 48 bp, synthetic construct
  source 1..48: mol_type = other DNA
gcacatgctccgtgtagaataccagagggtcttgttatctgcagacag

SEQ ID NO: 879 - DNA, 44 bp, synthetic construct
  source 1..44: mol_type = other DNA
gcacatgctccgtgtagaataggagggtgatttccacttggtcg

SEQ ID NO: 880 - DNA, 46 bp, synthetic construct
  source 1..46: mol_type = other DNA
gcacatgctccgtgtagaatactccttgtacagctgtaaatgccct

SEQ ID NO: 881 - DNA, 47 bp, synthetic construct
  source 1..47: mol_type = other DNA
gcacatgctccgtgtagaataagtgggttctgttttgggatgacagt

SEQ ID NO: 882 - DNA, 47 bp, synthetic construct
  source 1..47: mol_type = other DNA
gcacatgctccgtgtagaatagtaacacagtcattggtttgttgtgc

SEQ ID NO: 883 - DNA, 44 bp, synthetic construct
  source 1..44: mol_type = other DNA
gcacatgctccgtgtagaatataagcctcaatagtgctgaccac

SEQ ID NO: 884 - DNA, 46 bp, synthetic construct
  source 1..46: mol_type = other DNA
gcacatgctccgtgtagaataccgacctgtacatgccctcatgttc

SEQ ID NO: 885 - DNA, 46 bp, synthetic construct
  source 1..46: mol_type = other DNA
gcacatgctccgtgtagaataacgtgcacttcacacaggtaagacc

SEQ ID NO: 886 - DNA, 50 bp, synthetic construct
  source 1..50: mol_type = other DNA
gcacatgctccgtgtagaataaccggtgatcttgcagtaaagactatggt

SEQ ID NO: 887 - DNA, 53 bp, synthetic construct
  source 1..53: mol_type = other DNA
tgaactcggcgggttaggaatcgagaaggacagatgatactaccttcaagatg

SEQ ID NO: 888 - DNA, 50 bp, synthetic construct
  source 1..50: mol_type = other DNA
tgaactcggcgggttaggaatctcgagtgatgtcacattgtcatttagcg

SEQ ID NO: 889 - DNA, 49 bp, synthetic construct
  source 1..49: mol_type = other DNA
tgaactcggcgggttaggaattttgccattggataaaaatgtgtagctg

SEQ ID NO: 890 - DNA, 46 bp, synthetic construct
  source 1..46: mol_type = other DNA
tgaactcggcgggttaggaatatatggcacctacaatgtaaccagt

SEQ ID NO: 891 - DNA, 45 bp, synthetic construct
  source 1..45: mol_type = other DNA
tgaactcggcgggttaggaatcccgccctctgagtttgctcttga

SEQ ID NO: 892 - DNA, 46 bp, synthetic construct
  source 1..46: mol_type = other DNA
tgaactcggcgggttaggaatagagctaggttggttgtcaagtcgc

SEQ ID NO: 893 - DNA, 49 bp, synthetic construct
  source 1..49: mol_type = other DNA
tgaactcggcgggttaggaatgtcctctgagatcatagactcatgcttg

SEQ ID NO: 894 - DNA, 49 bp, synthetic construct
  source 1..49: mol_type = other DNA
tgaactcggcgggttaggaattcgctgtcgaatagccctagacttttcc

SEQ ID NO: 895 - DNA, 50 bp, synthetic construct
  source 1..50: mol_type = other DNA
tgaactcggcgggttaggaatagagatcatccataaccaagatgtcatcc

SEQ ID NO: 896 - DNA, 46 bp, synthetic construct
  source 1..46: mol_type = other DNA
tgaactcggcgggttaggaatgtacccgtccaactttgatacgtgg

SEQ ID NO: 897 - DNA, 49 bp, synthetic construct
  source 1..49: mol_type = other DNA
tgaactcggcgggttaggaatcctgcaatggctttactttggtgaagag

SEQ ID NO: 898 - DNA, 48 bp, synthetic construct
  source 1..48: mol_type = other DNA
tgaactcggcgggttaggaatacgagcttcttcttagagtctgagagc

SEQ ID NO: 899 - DNA, 49 bp, synthetic construct
  source 1..49: mol_type = other DNA
tgaactcggcgggttaggaatactccttctctagctgtagtttctgtct

SEQ ID NO: 900 - DNA, 46 bp, synthetic construct
  source 1..46: mol_type = other DNA
tgaactcggcgggttaggaatcaaccagcactagaaatgcatccag

SEQ ID NO: 901 - DNA, 49 bp, synthetic construct
  source 1..49: mol_type = other DNA
tgaactcggcgggttaggaatgaggcgaatcttctttagggcattgttt

SEQ ID NO: 902 - DNA, 47 bp, synthetic construct
  source 1..47: mol_type = other DNA
tgaactcggcgggttaggaatcggttgcgtactctatcactcatggc

SEQ ID NO: 903 - DNA, 50 bp, synthetic construct
  source 1..50: mol_type = other DNA
tgaactcggcgggttaggaatggtcgatcttttctgagtagatgggtagg

SEQ ID NO: 904 - DNA, 49 bp, synthetic construct
  source 1..49: mol_type = other DNA
tgaactcggcgggttaggaattcctcctcttgtaggtctgagatatggc

SEQ ID NO: 905 - DNA, 47 bp, synthetic construct
  source 1..47: mol_type = other DNA
tgaactcggcgggttaggaatgggccgttcggaagaagattttgctc

SEQ ID NO: 906 - DNA, 47 bp, synthetic construct
  source 1..47: mol_type = other DNA
tgaactcggcgggttaggaatcagttcacgatcttgtagcatgcttc

SEQ ID NO: 907 - DNA, 49 bp, synthetic construct
  source 1..49: mol_type = other DNA
tgaactcggcgggttaggaatagatcattgaagcccatgatagacatgg

SEQ ID NO: 908 - DNA, 44 bp, synthetic construct
  source 1..44: mol_type = other DNA
tgaactcggcgggttaggaatcagcaaggccttgtttacacggc

SEQ ID NO: 909 - DNA, 50 bp, synthetic construct
  source 1..50: mol_type = other DNA
tgaactcggcgggttaggaatactcatcagtaattttcaggtctcgttcc

SEQ ID NO: 910 - DNA, 47 bp, synthetic construct
  source 1..47: mol_type = other DNA
tgaactcggcgggttaggaataggtggaacatctcatcatcttgtgc

SEQ ID NO: 911 - DNA, 49 bp, synthetic construct
  source 1..49: mol_type = other DNA
gctcgacgttcctttgcaacacccacaggaattgacaagtagagttgct

SEQ ID NO: 912 - DNA, 45 bp, synthetic construct
  source 1..45: mol_type = other DNA
gctcgacgttcctttgcaacacacggttgcgtacattcaaccgac

SEQ ID NO: 913 - DNA, 51 bp, synthetic construct
  source 1..51: mol_type = other DNA
gctcgacgttcctttgcaacagtgaggtgctattgtacgtataacactgtg

SEQ ID NO: 914 - DNA, 54 bp, synthetic construct
  source 1..54: mol_type = other DNA
gctcgacgttcctttgcaacatgtgtctaactggaaggtatatgaagagaaagg

SEQ ID NO: 915 - DNA, 49 bp, synthetic construct
  source 1..49: mol_type = other DNA
gctcgacgttcctttgcaacagtcaaaagccatggatttagcttctcag

SEQ ID NO: 916 - DNA, 47 bp, synthetic construct
  source 1..47: mol_type = other DNA
gctcgacgttcctttgcaacataacagggagtctacaactgtgatgg

SEQ ID NO: 917 - DNA, 54 bp, synthetic construct
  source 1..54: mol_type = other DNA
gctcgacgttcctttgcaacagttgcaacactttaataaaggagatactgtggc

SEQ ID NO: 918 - DNA, 54 bp, synthetic construct
  source 1..54: mol_type = other DNA
gctcgacgttcctttgcaacacggccaaacatatcagaacagattgtctagacc

SEQ ID NO: 919 - DNA, 49 bp, synthetic construct
  source 1..49: mol_type = other DNA
gctcgacgttcctttgcaacagaccatgtcagttacattctgtgttccg

SEQ ID NO: 920 - DNA, 48 bp, synthetic construct
  source 1..48: mol_type = other DNA
gctcgacgttcctttgcaacaatcagtggagttgatagaggactccac

SEQ ID NO: 921 - DNA, 51 bp, synthetic construct
  source 1..51: mol_type = other DNA
gctcgacgttcctttgcaacatgtcattgttctccttcgtaacacttttgg

SEQ ID NO: 922 - DNA, 48 bp, synthetic construct
  source 1..48: mol_type = other DNA
gctcgacgttcctttgcaacatctgtgttttgttgtgaccatagcaag

SEQ ID NO: 923 - DNA, 45 bp, synthetic construct
  source 1..45: mol_type = other DNA
gctcgacgttcctttgcaacaccgtgccgaagatagaggaggtga

SEQ ID NO: 924 - DNA, 45 bp, synthetic construct
  source 1..45: mol_type = other DNA
gctcgacgttcctttgcaacacggagagccatttaccatctccgc

SEQ ID NO: 925 - DNA, 47 bp, synthetic construct
  source 1..47: mol_type = other DNA
gctcgacgttcctttgcaacacacgtggtcgagttggtatactcagt

SEQ ID NO: 926 - DNA, 45 bp, synthetic construct
  source 1..45: mol_type = other DNA
gctcgacgttcctttgcaacagccgggaagttttgatgcctggtc

SEQ ID NO: 927 - DNA, 46 bp, synthetic construct
  source 1..46: mol_type = other DNA
gctcgacgttcctttgcaacagtcaacttgttctggtacacatgca

SEQ ID NO: 928 - DNA, 45 bp, synthetic construct
  source 1..45: mol_type = other DNA
gctcgacgttcctttgcaacacgagttctcggtgcaattcgatgc

SEQ ID NO: 929 - DNA, 51 bp, synthetic construct
  source 1..51: mol_type = other DNA
gctcgacgttcctttgcaacagaatggtcatcaaatagatggacagtgaag

SEQ ID NO: 930 - DNA, 46 bp, synthetic construct
  source 1..46: mol_type = other DNA
gctcgacgttcctttgcaacacttggactgctcattgcattcagtg

SEQ ID NO: 931 - DNA, 46 bp, synthetic construct
  source 1..46: mol_type = other DNA
gctcgacgttcctttgcaacattctgttggtgattggtgggatagt

SEQ ID NO: 932 - DNA, 44 bp, synthetic construct
  source 1..44: mol_type = other DNA
gctcgacgttcctttgcaacaccaggcattgcaggactctcctt

SEQ ID NO: 933 - DNA, 47 bp, synthetic construct
  source 1..47: mol_type = other DNA
gctcgacgttcctttgcaacatgtaactctccaactgttgtgttccc

SEQ ID NO: 934 - DNA, 44 bp, synthetic construct
  source 1..44: mol_type = other DNA
gctcgacgttcctttgcaacatcgccaggaacctcagaacagca

SEQ ID NO: 935 - DNA, 49 bp, synthetic construct
  source 1..49: mol_type = other DNA
cgtcggagtgggttcagtctatctagaggcacattaaggtattggcaaa

SEQ ID NO: 936 - DNA, 51 bp, synthetic construct
  source 1..51: mol_type = other DNA
cgtcggagtgggttcagtctacagccaaggatcctgtttgtatctgaatca

SEQ ID NO: 937 - DNA, 49 bp, synthetic construct
  source 1..49: mol_type = other DNA
cgtcggagtgggttcagtctatcctgctgtttttgttagtaccagaagc

SEQ ID NO: 938 - DNA, 47 bp, synthetic construct
  source 1..47: mol_type = other DNA
cgtcggagtgggttcagtctaatccagctttgagagatgagttcagg

SEQ ID NO: 939 - DNA, 50 bp, synthetic construct
  source 1..50: mol_type = other DNA
cgtcggagtgggttcagtctagttccagaagaatgatagtgaggaagagg

SEQ ID NO: 940 - DNA, 48 bp, synthetic construct
  source 1..48: mol_type = other DNA
cgtcggagtgggttcagtctaggaccttggatatctgctatcaaccct

SEQ ID NO: 941 - DNA, 46 bp, synthetic construct
  source 1..46: mol_type = other DNA
cgtcggagtgggttcagtctaaacggatgtctctcgctactgatgg

SEQ ID NO: 942 - DNA, 48 bp, synthetic construct
  source 1..48: mol_type = other DNA
cgtcggagtgggttcagtctacttgctcttcatcctgaaatcttcctg

SEQ ID NO: 943 - DNA, 44 bp, synthetic construct
  source 1..44: mol_type = other DNA
cgtcggagtgggttcagtctaggccatcgatgatggatgaggcc

SEQ ID NO: 944 - DNA, 49 bp, synthetic construct
  source 1..49: mol_type = other DNA
cgtcggagtgggttcagtctatcaataactttattgtgccctagctggg

SEQ ID NO: 945 - DNA, 44 bp, synthetic construct
  source 1..44: mol_type = other DNA
cgtcggagtgggttcagtctacgtggccactttgaacctggctg

SEQ ID NO: 946 - DNA, 44 bp, synthetic construct
  source 1..44: mol_type = other DNA
cgtcggagtgggttcagtctattcaagaggaaactgcaggcacc

SEQ ID NO: 947 - DNA, 46 bp, synthetic construct
  source 1..46: mol_type = other DNA
cgtcggagtgggttcagtctaggcgccattggctgtcaactttagc

SEQ ID NO: 948 - DNA, 78 bp, synthetic construct
  source 1..78: mol_type = other DNA
cgtcaggtgagcatcttacatcagttcctgtttgaccttcttggtggtaacactcccttcatgtaacgtcaggtgagc

SEQ ID NO: 949 - DNA, 78 bp, synthetic construct
  source 1..78: mol_type = other DNA
cgtcaggtgagcatcttacattagtttgccttgacattctcaatggtgtcactgccgttcatgtaacgtcaggtgagc

SEQ ID NO: 950 - DNA, 78 bp, synthetic construct
  source 1..78: mol_type = other DNA
cgtcaggtgagcatcttacataggttgtcctggatctttgccttgacattctcttacttcatgtaacgtcaggtgagc

SEQ ID NO: 951 - DNA, 78 bp, synthetic construct
  source 1..78: mol_type = other DNA
cgtcaggtgagcatcttacattacgttttgcctgtcagggtcttcacaaagatcacgttcatgtaacgtcaggtgagc

SEQ ID NO: 952 - DNA, 78 bp, synthetic construct
  source 1..78: mol_type = other DNA
cgtcaggtgagcatcttacatcaccagggtggactctttctggatgttgtagtcactttcatgtaacgtcaggtgagc

SEQ ID NO: 953 - DNA, 78 bp, synthetic construct
  source 1..78: mol_type = other DNA
cgtcaggtgagcatcttacatccacttcacaaagatctgcatcccacctctgagcgcttcatgtaacgtcaggtgagc

SEQ ID NO: 954 - DNA, 78 bp, synthetic construct
  source 1..78: mol_type = other DNA
cgtcaggtgagcatcttacattacgtcttgcctgtcagggtcttcacaaagatcacgttcatgtaacgtcaggtgagc

SEQ ID NO: 955 - DNA, 47 bp, synthetic construct
  source 1..47: mol_type = other DNA
gtgtgtgccaggatgatcaatcggagggctacaagttaagggtagca

SEQ ID NO: 956 - DNA, 51 bp, synthetic construct
  source 1..51: mol_type = other DNA
gtgtgtgccaggatgatcaattccgggccaccctataataaatgattctca

SEQ ID NO: 957 - DNA, 48 bp, synthetic construct
  source 1..48: mol_type = other DNA
gtgtgtgccaggatgatcaattacatgccgtgttctatcggatacttc

SEQ ID NO: 958 - DNA, 49 bp, synthetic construct
  source 1..49: mol_type = other DNA
gtgtgtgccaggatgatcaatgtgacgaagctcgttatagaaagagtgg

SEQ ID NO: 959 - DNA, 48 bp, synthetic construct
  source 1..48: mol_type = other DNA
gtgtgtgccaggatgatcaatcctcgttgttagcatagagatccttcc

SEQ ID NO: 960 - DNA, 46 bp, synthetic construct
  source 1..46: mol_type = other DNA
gtgtgtgccaggatgatcaatcagcagcacaataccagttgtacgt

SEQ ID NO: 961 - DNA, 46 bp, synthetic construct
  source 1..46: mol_type = other DNA
gtgtgtgccaggatgatcaatggtaccattactccctgatgtctgg

SEQ ID NO: 962 - DNA, 47 bp, synthetic construct
  source 1..47: mol_type = other DNA
gtgtgtgccaggatgatcaatcgacggcagtagtcacgaaggaatag

SEQ ID NO: 963 - DNA, 44 bp, synthetic construct
  source 1..44: mol_type = other DNA
gtgtgtgccaggatgatcaataacgccttagggttcagtggtgc

SEQ ID NO: 964 - DNA, 47 bp, synthetic construct
  source 1..47: mol_type = other DNA
gtgtgtgccaggatgatcaatggtgacagagtacttgcgttctggag

SEQ ID NO: 965 - DNA, 44 bp, synthetic construct
  source 1..44: mol_type = other DNA
gtgtgtgccaggatgatcaatatcgcacgttgtgagtcacacca

SEQ ID NO: 966 - DNA, 46 bp, synthetic construct
  source 1..46: mol_type = other DNA
gtgtgtgccaggatgatcaatgacactccatcccaatgaaagatgg

SEQ ID NO: 967 - DNA, 46 bp, synthetic construct
  source 1..46: mol_type = other DNA
gtgtgtgccaggatgatcaataagatgaggtagtcggtgagatctc

SEQ ID NO: 968 - DNA, 47 bp, synthetic construct
  source 1..47: mol_type = other DNA
gtgtgtgccaggatgatcaattaattcaaagtccagagctacatagc

SEQ ID NO: 969 - DNA, 45 bp, synthetic construct
  source 1..45: mol_type = other DNA
gtgtgtgccaggatgatcaatagtggcagttcgtagctcttctcc

SEQ ID NO: 970 - DNA, 45 bp, synthetic construct
  source 1..45: mol_type = other DNA
gtgtgtgccaggatgatcaatcccgggacttagaagcatttgcgg

SEQ ID NO: 971 - DNA, 47 bp, synthetic construct
  source 1..47: mol_type = other DNA
gtgtgtgccaggatgatcaattacatgctgttataggtggtttcgtg

SEQ ID NO: 972 - DNA, 44 bp, synthetic construct
  source 1..44: mol_type = other DNA
gtgtgtgccaggatgatcaattcgcctgggagcatcatcaccag

SEQ ID NO: 973 - DNA, 48 bp, synthetic construct
  source 1..48: mol_type = other DNA
gtgtgtgccaggatgatcaatgaactccttgatgtcacggacaatctc

SEQ ID NO: 974 - DNA, 44 bp, synthetic construct
  source 1..44: mol_type = other DNA
gtgtgtgccaggatgatcaatcgaggaagagagtctctgggcag

SEQ ID NO: 975 - DNA, 44 bp, synthetic construct
  source 1..44: mol_type = other DNA
gtgtgtgccaggatgatcaatcggtctgtcagcagtgtcggatg

SEQ ID NO: 976 - DNA, 45 bp, synthetic construct
  source 1..45: mol_type = other DNA
gtgtgtgccaggatgatcaatttccgttcgtttccaatggtgatc

SEQ ID NO: 977 - DNA, 44 bp, synthetic construct
  source 1..44: mol_type = other DNA
gtgtgtgccaggatgatcaatagtgcgtcaggatccctctcttg

SEQ ID NO: 978 - DNA, 44 bp, synthetic construct
  source 1..44: mol_type = other DNA
gtgtgtgccaggatgatcaatcagcagaggcatagagggacagc

SEQ ID NO: 979 - DNA, 63 bp, synthetic construct
  source 1..63: mol_type = other DNA
ataggaaatggtggtagtgtttagactgaacccactccgacgatctgtcttcgtcggagtggg

SEQ ID NO: 980 - DNA, 63 bp, synthetic construct
  source 1..63: mol_type = other DNA
cgtcggagtgggttcagtctacccactccgacgaagacagattataggaaatggtggtagtgt

SEQ ID NO: 981 - DNA, 60 bp, synthetic construct
  source 1..60: mol_type = other DNA
gatgtagtagtaagggttattcctaacccgccgagttcactaaggttttgaactcggcgg

SEQ ID NO: 982 - DNA, 60 bp, synthetic construct
  source 1..60: mol_type = other DNA
tgaactcggcgggttaggaatccgccgagttcaaaaccttagtgatgatgtagtagtaag

SEQ ID NO: 983 - DNA, 48 bp, synthetic construct
  source 1..48: mol_type = other DNA
gcacatgctccgtgtagaatatcactgcatgctaaatcggagggtatt

SEQ ID NO: 984 - DNA, 49 bp, synthetic construct
  source 1..49: mol_type = other DNA
gcacatgctccgtgtagaatagtaatcatggtgagttttggtcagatga

SEQ ID NO: 985 - DNA, 49 bp, synthetic construct
  source 1..49: mol_type = other DNA
gcacatgctccgtgtagaatacctaacgctcagttaacttgatccaaag

SEQ ID NO: 986 - DNA, 51 bp, synthetic construct
  source 1..51: mol_type = other DNA
gcacatgctccgtgtagaatagttgtcgtgctgaataataccacttacaac

SEQ ID NO: 987 - DNA, 50 bp, synthetic construct
  source 1..50: mol_type = other DNA
gcacatgctccgtgtagaatacgagcatctctagacttttcttttcgacg

SEQ ID NO: 988 - DNA, 51 bp, synthetic construct
  source 1..51: mol_type = other DNA
gcacatgctccgtgtagaatagtccatctccaaatctaaatcagtgtcctg

SEQ ID NO: 989 - DNA, 51 bp, synthetic construct
  source 1..51: mol_type = other DNA
gcacatgctccgtgtagaataacagtccagttagttcaaactgagttaacc

SEQ ID NO: 990 - DNA, 48 bp, synthetic construct
  source 1..48: mol_type = other DNA
gcacatgctccgtgtagaataaagcaggaaagtgacatagtaggtgca

SEQ ID NO: 991 - DNA, 49 bp, synthetic construct
  source 1..49: mol_type = other DNA
gcacatgctccgtgtagaatatatacagaagctttatcaagatgtgagc

SEQ ID NO: 992 - DNA, 48 bp, synthetic construct
  source 1..48: mol_type = other DNA
gcacatgctccgtgtagaatatcattcaacccagacatatccacctct

SEQ ID NO: 993 - DNA, 50 bp, synthetic construct
  source 1..50: mol_type = other DNA
gcacatgctccgtgtagaataccccttggagaattgctctctaatggtga

SEQ ID NO: 994 - DNA, 51 bp, synthetic construct
  source 1..51: mol_type = other DNA
gcacatgctccgtgtagaataatcggctttcagataaaaacagtccatctg

SEQ ID NO: 995 - DNA, 47 bp, synthetic construct
  source 1..47: mol_type = other DNA
gcacatgctccgtgtagaatagagtcaggtgaactttgtctagtgct

SEQ ID NO: 996 - DNA, 51 bp, synthetic construct
  source 1..51: mol_type = other DNA
gcacatgctccgtgtagaatatacggagcattaacttcacaatcgtaactg

SEQ ID NO: 997 - DNA, 51 bp, synthetic construct
  source 1..51: mol_type = other DNA
gcacatgctccgtgtagaataccgagcttgtatcctctgattcaactttgg

SEQ ID NO: 998 - DNA, 47 bp, synthetic construct
  source 1..47: mol_type = other DNA
gcacatgctccgtgtagaatagacgggcatggtaaaagaaagtccca

SEQ ID NO: 999 - DNA, 49 bp, synthetic construct
  source 1..49: mol_type = other DNA
gcacatgctccgtgtagaatatttggaacgtaactggaaatcatcatcc

SEQ ID NO: 1000 - DNA, 48 bp, synthetic construct
  source 1..48: mol_type = other DNA
gcacatgctccgtgtagaatagtgactgaggttggttactgttggtat

SEQ ID NO: 1001 - DNA, 51 bp, synthetic construct
  source 1..51: mol_type = other DNA
gcacatgctccgtgtagaatacgaccgttccattctgttcactagatatga

SEQ ID NO: 1002 - DNA, 51 bp, synthetic construct
  source 1..51: mol_type = other DNA
gcacatgctccgtgtagaataaaagtctgtctgttctatgactctctttcc

SEQ ID NO: 1003 - DNA, 49 bp, synthetic construct
  source 1..49: mol_type = other DNA
gcacatgctccgtgtagaatataattaatgcaacctcttgattcagtgc

SEQ ID NO: 1004 - DNA, 49 bp, synthetic construct
  source 1..49: mol_type = other DNA
gcacatgctccgtgtagaatatttgttaggagtgtttacgttttcctga

SEQ ID NO: 1005 - DNA, 47 bp, synthetic construct
  source 1..47: mol_type = other DNA
gcacatgctccgtgtagaatatcaggtcgtttcttgaggtacttggg

SEQ ID NO: 1006 - DNA, 54 bp, synthetic construct
  source 1..54: mol_type = other DNA
gcacatgctccgtgtagaataattcccatcaactcagtaattctttcatcacag

SEQ ID NO: 1007 - DNA, 51 bp, synthetic construct; identical to SEQ ID NO: 552
  source 1..51: mol_type = other DNA

SEQ ID NO: 1008 - DNA, 51 bp, synthetic construct; identical to SEQ ID NO: 553
  source 1..51: mol_type = other DNA

SEQ ID NO: 1009 - DNA, 51 bp, synthetic construct; identical to SEQ ID NO: 554
  source 1..51: mol_type = other DNA

SEQ ID NO: 1010 - DNA, 51 bp, synthetic construct; identical to SEQ ID NO: 555
  source 1..51: mol_type = other DNA

SEQ ID NO: 1011 - DNA, 51 bp, synthetic construct; identical to SEQ ID NO: 556
  source 1..51: mol_type = other DNA

SEQ ID NO: 1012 - DNA, 51 bp, synthetic construct; identical to SEQ ID NO: 557
  source 1..51: mol_type = other DNA

SEQ ID NO: 1013 - DNA, 51 bp, synthetic construct; identical to SEQ ID NO: 558
  source 1..51: mol_type = other DNA

SEQ ID NO: 1014 - DNA, 51 bp, synthetic construct; identical to SEQ ID NO: 559
  source 1..51: mol_type = other DNA

SEQ ID NO: 1015 - DNA, 51 bp, synthetic construct; identical to SEQ ID NO: 560
  source 1..51: mol_type = other DNA

SEQ ID NO: 1016 - DNA, 51 bp, synthetic construct; identical to SEQ ID NO: 561
  source 1..51: mol_type = other DNA

SEQ ID NO: 1017 - DNA, 51 bp, synthetic construct; identical to SEQ ID NO: 562
  source 1..51: mol_type = other DNA

SEQ ID NO: 1018 - DNA, 51 bp, synthetic construct; identical to SEQ ID NO: 563
  source 1..51: mol_type = other DNA

SEQ ID NO: 1019 - DNA, 51 bp, synthetic construct; identical to SEQ ID NO: 564
  source 1..51: mol_type = other DNA

SEQ ID NO: 1020 - DNA, 51 bp, synthetic construct; identical to SEQ ID NO: 565
  source 1..51: mol_type = other DNA

SEQ ID NO: 1021 - DNA, 51 bp, synthetic construct; identical to SEQ ID NO: 566
  source 1..51: mol_type = other DNA

SEQ ID NO: 1022 - DNA, 51 bp, synthetic construct; identical to SEQ ID NO: 567
  source 1..51: mol_type = other DNA

SEQ ID NO: 1023 - DNA, 51 bp, synthetic construct; identical to SEQ ID NO: 568
  source 1..51: mol_type = other DNA

SEQ ID NO: 1024 - DNA, 51 bp, synthetic construct; identical to SEQ ID NO: 569
  source 1..51: mol_type = other DNA

SEQ ID NO: 1025 - DNA, 51 bp, synthetic construct
  source 1..51: mol_type = other DNA
gctcgacgttcctttgcaacaagatggaatctgagtagtagatgtctgctt

SEQ ID NO: 1026 - DNA, 51 bp, synthetic construct
  source 1..51: mol_type = other DNA
gctcgacgttcctttgcaacacatcaggattttgtgagatgtcagaatagc

SEQ ID NO: 1027 - DNA, 50 bp, synthetic construct
  source 1..50: mol_type = other DNA
gctcgacgttcctttgcaacacacagaacaggctttatatgctttctcgg

SEQ ID NO: 1028 - DNA, 54 bp, synthetic construct
  source 1..54: mol_type = other DNA
gctcgacgttcctttgcaacaatacccactcgatattttatcttttgtcctagc

SEQ ID NO: 1029 - DNA, 51 bp, synthetic construct
  source 1..51: mol_type = other DNA
gctcgacgttcctttgcaacatcagcatggacaattactgaactctatagc

SEQ ID NO: 1030 - DNA, 53 bp, synthetic construct
  source 1..53: mol_type = other DNA
gctcgacgttcctttgcaacacccgtctgacagtaactataccataggtgaca

SEQ ID NO: 1031 - DNA, 51 bp, synthetic construct
  source 1..51: mol_type = other DNA
gctcgacgttcctttgcaacagttggatacctatggaaaaggcaatttgac

SEQ ID NO: 1032 - DNA, 48 bp, synthetic construct
  source 1..48: mol_type = other DNA
gctcgacgttcctttgcaacatagcggctaaacatttatcacctgtca

SEQ ID NO: 1033 - DNA, 49 bp, synthetic construct
  source 1..49: mol_type = other DNA
gctcgacgttcctttgcaacaaataccatgttattctgtaaagactggc

SEQ ID NO: 1034 - DNA, 49 bp, synthetic construct
  source 1..49: mol_type = other DNA
gctcgacgttcctttgcaacaacgtggtagtaagacatcgtacttgagt

SEQ ID NO: 1035 - DNA, 49 bp, synthetic construct
  source 1..49: mol_type = other DNA
gctcgacgttcctttgcaacaccaggctgactgatgatagtgttttacc

SEQ ID NO: 1036 - DNA, 50 bp, synthetic construct
  source 1..50: mol_type = other DNA
gctcgacgttcctttgcaacaagagcagaaatctgctagtgattgtcgta

SEQ ID NO: 1037 - DNA, 51 bp, synthetic construct
  source 1..51: mol_type = other DNA
gctcgacgttcctttgcaacataatgctgagtagttatgagaaagtgtcac

SEQ ID NO: 1038 - DNA, 52 bp, synthetic construct
  source 1..52: mol_type = other DNA
gctcgacgttcctttgcaacaaacgagattcctctgtaatgtaaagtcaggg

SEQ ID NO: 1039 - DNA, 51 bp, synthetic construct
  source 1..51: mol_type = other DNA
gctcgacgttcctttgcaacaaacgctagaatcttgcccttattaacttgc

SEQ ID NO: 1040 - DNA, 50 bp, synthetic construct
  source 1..50: mol_type = other DNA
gctcgacgttcctttgcaacatagcccagtaacttcttaactccactatg

SEQ ID NO: 1041 - DNA, 50 bp, synthetic construct
  source 1..50: mol_type = other DNA
gctcgacgttcctttgcaacacttgctaagaatgaggaaggtcgtaatgt

SEQ ID NO: 1042 - DNA, 46 bp, synthetic construct
  source 1..46: mol_type = other DNA
gctcgacgttcctttgcaacagtgagctcaaaaggttcatggagag

SEQ ID NO: 1043 - DNA, 48 bp, synthetic construct
  source 1..48: mol_type = other DNA
gctcgacgttcctttgcaacattcaggttttgcctaaatttctgcctg

SEQ ID NO: 1044 - DNA, 47 bp, synthetic construct
  source 1..47: mol_type = other DNA
gctcgacgttcctttgcaacagaagtgtgtttcatgtctctctgtgg

SEQ ID NO: 1045 - DNA, 51 bp, synthetic construct
  source 1..51: mol_type = other DNA
gctcgacgttcctttgcaacagtagggagacaggtagaattttcattgaag

SEQ ID NO: 1046 - DNA, 50 bp, synthetic construct
  source 1..50: mol_type = other DNA
gctcgacgttcctttgcaacatccggtattgctgataatgatggagagct

SEQ ID NO: 1047 - DNA, 49 bp, synthetic construct
  source 1..49: mol_type = other DNA
gctcgacgttcctttgcaacatctagctgtttctgctatttgagagctt

SEQ ID NO: 1048 - DNA, 46 bp, synthetic construct
  source 1..46: mol_type = other DNA
gctcgacgttcctttgcaacagtatgctcatagcacactacaggac

SEQ ID NO: 1049 - DNA, 54 bp, synthetic construct
  source 1..54: mol_type = other DNA
gctggtcggtggattggattttttatagtaagccacatcagctatgtccacgtc

SEQ ID NO: 1050 - DNA, 54 bp, synthetic construct
  source 1..54: mol_type = other DNA
gctggtcggtggattggatttgtcacttgctgtgagtcatttctgctttcatcg

SEQ ID NO: 1051 - DNA, 54 bp, synthetic construct
  source 1..54: mol_type = other DNA
gctggtcggtggattggatttgcggatgactgctaatgacaccaccacaatgac

SEQ ID NO: 1052 - DNA, 54 bp, synthetic construct
  source 1..54: mol_type = other DNA
gctggtcggtggattggatttaaatcatcaacgtagtaaatcagagtctgcccg

SEQ ID NO: 1053 - DNA, 54 bp, synthetic construct
  source 1..54: mol_type = other DNA
gctggtcggtggattggatttacagtaccataggaagtacactggcattcacca

SEQ ID NO: 1054 - DNA, 54 bp, synthetic construct
  source 1..54: mol_type = other DNA
gctggtcggtggattggatttgtcgtccatgctcttagaagaatggaacaggga

SEQ ID NO: 1055 - DNA, 54 bp, synthetic construct
  source 1..54: mol_type = other DNA
gctggtcggtggattggatttcgactgatggtcgtaggggctttctctttcttt

SEQ ID NO: 1056 - DNA, 54 bp, synthetic construct
  source 1..54: mol_type = other DNA
gctggtcggtggattggatttactttcagcttatatcgagatgtgaacgcctct

SEQ ID NO: 1057 - DNA, 54 bp, synthetic construct
  source 1..54: mol_type = other DNA
gctggtcggtggattggattttcccattaagctctctgtggatctcacccatct

SEQ ID NO: 1058 - DNA, 54 bp, synthetic construct
  source 1..54: mol_type = other DNA
gctggtcggtggattggatttgttcttgttgccagcttgtagttgtcacagaca

SEQ ID NO: 1059 - DNA, 54 bp, synthetic construct
  source 1..54: mol_type = other DNA
gctggtcggtggattggatttaagtgagaagagttttgcatcagatcaatggtg

SEQ ID NO: 1060 - DNA, 54 bp, synthetic construct
  source 1..54: mol_type = other DNA
gctggtcggtggattggatttgagagccttctcatattttgctgatttcttcct

SEQ ID NO: 1061 - DNA, 54 bp, synthetic construct
  source 1..54: mol_type = other DNA
cgtcaggtgagcatcttacatacgctgatctgcgtctttgatcatttgtcctcg

SEQ ID NO: 1062 - DNA, 54 bp, synthetic construct
  source 1..54: mol_type = other DNA
cgtcaggtgagcatcttacatagacagaggttttactgagtctggcttttgctg

SEQ ID NO: 1063 - DNA, 54 bp, synthetic construct
  source 1..54: mol_type = other DNA
cgtcaggtgagcatcttacataagccctggcatatgtatacgttttgtcaaggc

SEQ ID NO: 1064 - DNA, 54 bp, synthetic construct
  source 1..54: mol_type = other DNA
cgtcaggtgagcatcttacatctgacgattttcaggtgtttcatcttcacaggc

SEQ ID NO: 1065 - DNA, 54 bp, synthetic construct
  source 1..54: mol_type = other DNA
cgtcaggtgagcatcttacattatctggtgtcatgtgctagtttctcagagtga

SEQ ID NO: 1066 - DNA, 54 bp, synthetic construct
  source 1..54: mol_type = other DNA
cgtcaggtgagcatcttacattcttgtccaccaaaggatacacgtcttctcttc

SEQ ID NO: 1067 - DNA, 54 bp, synthetic construct
  source 1..54: mol_type = other DNA
cgtcaggtgagcatcttacatcgagatatcaacggaactaagaccaggtaggcc

SEQ ID NO: 1068 - DNA, 54 bp, synthetic construct
  source 1..54: mol_type = other DNA
cgtcaggtgagcatcttacataggagattcacagaatgtcgtctgccagtaaca

SEQ ID NO: 1069 - DNA, 54 bp, synthetic construct
  source 1..54: mol_type = other DNA
cgtcaggtgagcatcttacatacacgggcatctttggggttttctcaacaataa

SEQ ID NO: 1070 - DNA, 54 bp, synthetic construct
  source 1..54: mol_type = other DNA
cgtcaggtgagcatcttacataacgttttgacaaccactaatgggccattagca

SEQ ID NO: 1071 - DNA, 54 bp, synthetic construct
  source 1..54: mol_type = other DNA
cgtcaggtgagcatcttacatacagtcatcttctggccctatgggtatgtgtac

SEQ ID NO: 1072 - DNA, 54 bp, synthetic construct
  source 1..54: mol_type = other DNA
cgtcaggtgagcatcttacatccgcccgagatgtagatttcttggctccatcat

SEQ ID NO: 1073 - DNA, 54 bp, synthetic construct
  source 1..54: mol_type = other DNA
cgtcaggtgagcatcttacatcaacctctagacctcaagcatgttcttttccca

SEQ ID NO: 1074 - DNA, 54 bp, synthetic construct
  source 1..54: mol_type = other DNA
cgtcaggtgagcatcttacattcatgtattctgagctgcctctttaaacctgct

SEQ ID NO: 1075 - DNA, 54 bp, synthetic construct
  source 1..54: mol_type = other DNA
cgtcaggtgagcatcttacatttacctggttgtggagatctcaaggacattctt

SEQ ID NO: 1076 - DNA, 54 bp, synthetic construct
  source 1..54: mol_type = other DNA
cgtcaggtgagcatcttacattgttcagactctccagagactcctttctcttcc

SEQ ID NO: 1077 - DNA, 54 bp, synthetic construct
  source 1..54: mol_type = other DNA
cgtcaggtgagcatcttacattgatcaagaggttgaaccccatccttatgtgtc

SEQ ID NO: 1078 - DNA, 54 bp, synthetic construct
  source 1..54: mol_type = other DNA
cgtcaggtgagcatcttacatacatctcgaaatgtcccccttaaatgtcttgct

SEQ ID NO: 1079 - DNA, 54 bp, synthetic construct
  source 1..54: mol_type = other DNA
cgtcaggtgagcatcttacatgtgaatgcttccatctcttgcagacttcctctt

SEQ ID NO: 1080 - DNA, 54 bp, synthetic construct
  source 1..54: mol_type = other DNA
cgtcaggtgagcatcttacatgtgtgtcactgaatccttgttatggcctgatgt

SEQ ID NO: 1081 - DNA, 54 bp, synthetic construct
  source 1..54: mol_type = other DNA
cgtcaggtgagcatcttacatatgagggagagtttgcatggcctgtagtaaatt

SEQ ID NO: 1082 - DNA, 54 bp, synthetic construct
  source 1..54: mol_type = other DNA
cgtcaggtgagcatcttacataggttggttggttcctcctgccagttaaactta

SEQ ID NO: 1083 - DNA, 54 bp, synthetic construct
  source 1..54: mol_type = other DNA
cgtcaggtgagcatcttacatggcaggcctcctcttatgtcctgttgaatttcc

SEQ ID NO: 1084 - DNA, 54 bp, synthetic construct
  source 1..54: mol_type = other DNA
cgtcaggtgagcatcttacattgattttccttaggtgtttgtggctgtctggta

SEQ ID NO: 1085 - DNA, 47 bp, synthetic construct
  source 1..47: mol_type = other DNA
cgtcggagtgggttcagtctaccggcaccaaccagacgagacaccga

SEQ ID NO: 1086 - DNA, 47 bp, synthetic construct
  source 1..47: mol_type = other DNA
cgtcggagtgggttcagtctagacgacggcgacgatccggtcttcat

SEQ ID NO: 1087 - DNA, 47 bp, synthetic construct
  source 1..47: mol_type = other DNA
cgtcggagtgggttcagtctaattcgcgatggtgcagttcttgctcc

SEQ ID NO: 1088 - DNA, 47 bp, synthetic construct
  source 1..47: mol_type = other DNA
cgtcggagtgggttcagtctagacgaaacgacgttccagcgcagcat

SEQ ID NO: 1089 - DNA, 54 bp, synthetic construct
  source 1..54: mol_type = other DNA
gctcgacgttcctttgcaacaacacttagttttgtcgcggttaggatcgaatgg

SEQ ID NO: 1090 - DNA, 54 bp, synthetic construct
  source 1..54: mol_type = other DNA
gctcgacgttcctttgcaacaccaggtgcagtaacggtagtagcagtggtatag

SEQ ID NO: 1091 - DNA, 54 bp, synthetic construct
  source 1..54: mol_type = other DNA
gctcgacgttcctttgcaacaacagaatgtatctcccggacttgcactagtacc

SEQ ID NO: 1092 - DNA, 54 bp, synthetic construct
  source 1..54: mol_type = other DNA
gctcgacgttcctttgcaacatttcccaagaacaacctttatcaccttcacagc

SEQ ID NO: 1093 - DNA, 54 bp, synthetic construct
  source 1..54: mol_type = other DNA
gctcgacgttcctttgcaacaaggatcttaatgtgaaaggtgcacgttgccaat

SEQ ID NO: 1094 - DNA, 54 bp, synthetic construct
  source 1..54: mol_type = other DNA
gctcgacgttcctttgcaacaaccagcggtgacagtagtagtggtagtgtaaga

SEQ ID NO: 1095 - DNA, 54 bp, synthetic construct
  source 1..54: mol_type = other DNA
gctcgacgttcctttgcaacaacctagtggtggttgcgtaagattgagaccaat

SEQ ID NO: 1096 - DNA, 54 bp, synthetic construct
  source 1..54: mol_type = other DNA
gctcgacgttcctttgcaacatcaatggtttggtggttctctgataatcacggt

SEQ ID NO: 1097 - DNA, 54 bp, synthetic construct
  source 1..54: mol_type = other DNA
gctcgacgttcctttgcaacaaggttgatgataactgaatcagtgccacctggt

SEQ ID NO: 1098 - DNA, 54 bp, synthetic construct
  source 1..54: mol_type = other DNA
gctcgacgttcctttgcaacaagatccaaatcaacagaagaaccagttccacct

SEQ ID NO: 1099 - DNA, 54 bp, synthetic construct
  source 1..54: mol_type = other DNA
gctcgacgttcctttgcaacaacgaagtggtaaagtgacagtacccaaagcctt

SEQ ID NO: 1100 - DNA, 54 bp, synthetic construct
  source 1..54: mol_type = other DNA
gctcgacgttcctttgcaacataaaaaacagtatcagtgtttcctggtggtcca

SEQ ID NO: 1101 - DNA, 54 bp, synthetic construct
  source 1..54: mol_type = other DNA
gctcgacgttcctttgcaacataaccaagttggggttcctggtcccttataatt

SEQ ID NO: 1102 - DNA, 54 bp, synthetic construct
  source 1..54: mol_type = other DNA
gctcgacgttcctttgcaacactaaataactgaatcagttccacctggaggagc

SEQ ID NO: 1103 - DNA, 54 bp, synthetic construct
  source 1..54: mol_type = other DNA
gctcgacgttcctttgcaacattctgttagcgaatcccattgtaccagatgtgt

SEQ ID NO: 1104 - DNA, 54 bp, synthetic construct
  source 1..54: mol_type = other DNA
gctcgacgttcctttgcaacacaagcgtaagattgagaccagtactcagtggtt

SEQ ID NO: 1105 - DNA, 54 bp, synthetic construct
  source 1..54: mol_type = other DNA
gctcgacgttcctttgcaacagatgaagaaatgataggcgatgaagcttccacg

SEQ ID NO: 1106 - DNA, 54 bp, synthetic construct
  source 1..54: mol_type = other DNA
gctcgacgttcctttgcaacaatagtgttgtggtggaagtaattgttcctgtcc

SEQ ID NO: 1107 - DNA, 54 bp, synthetic construct
  source 1..54: mol_type = other DNA
gctcgacgttcctttgcaacacctttgcgattgagattggttggttgatgttgt

SEQ ID NO: 1108 - DNA, 54 bp, synthetic construct
  source 1..54: mol_type = other DNA
gctcgacgttcctttgcaacatcttggggattgtaaagtggattctgtggttgt

SEQ ID NO: 1109 - DNA, 54 bp, synthetic construct
  source 1..54: mol_type = other DNA
gctcgacgttcctttgcaacaccctttggcagtggaacttgtacaatgacagtg

SEQ ID NO: 1110 - DNA, 54 bp, synthetic construct
  source 1..54: mol_type = other DNA
gctcgacgttcctttgcaacatcatttggtcaggtaggaagtagtcacaccaac

SEQ ID NO: 1111 - DNA, 54 bp, synthetic construct
  source 1..54: mol_type = other DNA
gctcgacgttcctttgcaacaaagtctaatgataaccgaatcagtgccacctgg

SEQ ID NO: 1112 - DNA, 54 bp, synthetic construct
  source 1..54: mol_type = other DNA
gctcgacgttcctttgcaacaccaggtgcagttacagttgtggttgtagcaaat

SEQ ID NO: 1113 - DNA, 54 bp, synthetic construct
  source 1..54: mol_type = other DNA
gctcgacgttcctttgcaacaaaacatcatagccataggacatctgggaagcaa

SEQ ID NO: 1114 - DNA, 54 bp, synthetic construct
  source 1..54: mol_type = other DNA
gctcgacgttcctttgcaacacgcttcaccacttgatccagaaggaccttgttt

SEQ ID NO: 1115 - DNA, 54 bp, synthetic construct
  source 1..54: mol_type = other DNA
gctcgacgttcctttgcaacagtcgaggaccagcatcacctttaacaccagtat

SEQ ID NO: 1116 - DNA, 54 bp, synthetic construct
  source 1..54: mol_type = other DNA
gctcgacgttcctttgcaacagacttcttgcagtgataggtgatgttctgggag

SEQ ID NO: 1117 - DNA, 54 bp, synthetic construct
  source 1..54: mol_type = other DNA
gctcgacgttcctttgcaacacgtgatacagatcaagcatacctcgggtttcca

SEQ ID NO: 1118 - DNA, 54 bp, synthetic construct
  source 1..54: mol_type = other DNA
gctcgacgttcctttgcaacacagcctcgactcctacatcttctgagtttggtg

SEQ ID NO: 1119 - DNA, 54 bp, synthetic construct
  source 1..54: mol_type = other DNA
gctcgacgttcctttgcaacaagatggtggttttgtattcgatgactgtcttgc

SEQ ID NO: 1120 - DNA, 54 bp, synthetic construct
  source 1..54: mol_type = other DNA
gctcgacgttcctttgcaacagtgcatctttaccaggagaaccatcagcacctt

SEQ ID NO: 1121 - DNA, 54 bp, synthetic construct
  source 1..54: mol_type = other DNA
gctcgacgttcctttgcaacagtgcatcttgagacttctcttgaggtggctgag

SEQ ID NO: 1122 - DNA, 54 bp, synthetic construct
  source 1..54: mol_type = other DNA
gctcgacgttcctttgcaacacgcgatgttctcaatctgctgactcaggctctt

SEQ ID NO: 1123 - DNA, 54 bp, synthetic construct
  source 1..54: mol_type = other DNA
gctcgacgttcctttgcaacacccgttgggacagtccagttcttcattgcattg

SEQ ID NO: 1124 - DNA, 54 bp, synthetic construct
  source 1..54: mol_type = other DNA
gctcgacgttcctttgcaacagtggatgaccctttatgcctctgtcaccttgtt

SEQ ID NO: 1125 - DNA, 54 bp, synthetic construct
  source 1..54: mol_type = other DNA
gctcgacgttcctttgcaacaactcctgtctccatgttgcagtagaccttgatg

SEQ ID NO: 1126 - DNA, 54 bp, synthetic construct
  source 1..54: mol_type = other DNA
gctcgacgttcctttgcaacatggcttaggccattgtgtatgcagctgacttca

SEQ ID NO: 1127 - DNA, 54 bp, synthetic construct
  source 1..54: mol_type = other DNA
gctcgacgttcctttgcaacacgactctccaaaccagacgtgcttcttttcctt

SEQ ID NO: 1128 - DNA, 54 bp, synthetic construct
  source 1..54: mol_type = other DNA
gctcgacgttcctttgcaacacccccaatgtctagtccgaattcctggtctggg

SEQ ID NO: 1129 - DNA, 54 bp, synthetic construct
  source 1..54: mol_type = other DNA
gctcgacgttcctttgcaacacccttctcctttggcaccagtgtctcctttgtt

SEQ ID NO: 1130 - DNA, 54 bp, synthetic construct
  source 1..54: mol_type = other DNA
gctcgacgttcctttgcaacaggcctcttccagtcagagtggcacatcttgagg

SEQ ID NO: 1131 - DNA, 54 bp, synthetic construct
  source 1..54: mol_type = other DNA
gctcgacgttcctttgcaacagtcgaagaccagggaagcctctttctcctctct

SEQ ID NO: 1132 - DNA, 54 bp, synthetic construct
  source 1..54: mol_type = other DNA
gctcgacgttcctttgcaacacgcgtacccttaggtccagggaatcccatcaca

SEQ ID NO: 1133 - DNA, 54 bp, synthetic construct
  source 1..54: mol_type = other DNA
gctcgacgttcctttgcaacaacgagccttggttagggtcgatccagtactctc

SEQ ID NO: 1134 - DNA, 54 bp, synthetic construct
  source 1..54: mol_type = other DNA
gctcgacgttcctttgcaacatcgaggtccatcagcaccaggagatcctttctc

SEQ ID NO: 1135 - DNA, 54 bp, synthetic construct
  source 1..54: mol_type = other DNA
gctcgacgttcctttgcaacactgcttgtacaccacgttcaccagggaaacctc

SEQ ID NO: 1136 - DNA, 54 bp, synthetic construct
  source 1..54: mol_type = other DNA
gctcgacgttcctttgcaacaaggcttaggaccagcaggaccagcatctccttt

SEQ ID NO: 1137 - DNA, 54 bp, synthetic construct
  source 1..54: mol_type = other DNA
cgtcggagtgggttcagtctagagtgatagtaagtgcaaagctcgtcacactga

SEQ ID NO: 1138 - DNA, 54 bp, synthetic construct
  source 1..54: mol_type = other DNA
cgtcggagtgggttcagtctaggacgctggagaacaaagagaaccagattgaac

SEQ ID NO: 1139 - DNA, 54 bp, synthetic construct
  source 1..54: mol_type = other DNA
cgtcggagtgggttcagtctaaggagagaagaaatagacgctctgaatgggctc

SEQ ID NO: 1140 - DNA, 54 bp, synthetic construct
  source 1..54: mol_type = other DNA
cgtcggagtgggttcagtctagtttaatcatcctctggcatagtgaacacgtcc

SEQ ID NO: 1141 - DNA, 54 bp, synthetic construct
  source 1..54: mol_type = other DNA
cgtcggagtgggttcagtctaaacaacaagtaggtcttcccctgacagttgatg

SEQ ID NO: 1142 - DNA, 54 bp, synthetic construct
  source 1..54: mol_type = other DNA
cgtcggagtgggttcagtctaggacaatgccccagacatcttggataagtttgg

SEQ ID NO: 1143 - DNA, 54 bp, synthetic construct
  source 1..54: mol_type = other DNA
cgtcggagtgggttcagtctacggccagaagaggagttcgaaaatgttctccca

SEQ ID NO: 1144 - DNA, 54 bp, synthetic construct
  source 1..54: mol_type = other DNA
cgtcggagtgggttcagtctacgagcatcaacattgtctggtatgccactgaag

SEQ ID NO: 1145 - DNA, 54 bp, synthetic construct
  source 1..54: mol_type = other DNA
cgtcggagtgggttcagtctaagcccgggttctaaggttgactcggtagtattt

SEQ ID NO: 1146 - DNA, 54 bp, synthetic construct
  source 1..54: mol_type = other DNA
cgtcggagtgggttcagtctaacgtgctgaaattcgtactcccagtactgcttc

SEQ ID NO: 1147 - DNA, 54 bp, synthetic construct
  source 1..54: mol_type = other DNA
cgtcggagtgggttcagtctacccctttccactgcacagttcttcctctggaaa

SEQ ID NO: 1148 - DNA, 54 bp, synthetic construct
  source 1..54: mol_type = other DNA
cgtcggagtgggttcagtctaagcgaaggcaaagagggacccattcttgagatc

SEQ ID NO: 1149 - DNA, 54 bp, synthetic construct
  source 1..54: mol_type = other DNA
cgtcggagtgggttcagtctacgcctttcggcttctacgcttagacttctgttt

SEQ ID NO: 1150 - DNA, 54 bp, synthetic construct
  source 1..54: mol_type = other DNA
cgtcggagtgggttcagtctaagtctgccgtctcatctagctcatagcagtact

SEQ ID NO: 1151 - DNA, 54 bp, synthetic construct
  source 1..54: mol_type = other DNA
cgtcggagtgggttcagtctacgtgagtgggataaggagccagtgacgtagatg

SEQ ID NO: 1152 - DNA, 54 bp, synthetic construct
  source 1..54: mol_type = other DNA
cgtcggagtgggttcagtctaaagctcaggatctaggaaggctgtcggctttag

SEQ ID NO: 1153 - DNA, 54 bp, synthetic construct
  source 1..54: mol_type = other DNA
cgtcggagtgggttcagtctaaacgtattgttcttgggctcctccacgtagtca

SEQ ID NO: 1154 - DNA, 54 bp, synthetic construct
  source 1..54: mol_type = other DNA
cgtcggagtgggttcagtctaacgcaccagggctagtatgaaaaagggcctcag

SEQ ID NO: 1155 - DNA, 54 bp, synthetic construct
  source 1..54: mol_type = other DNA
cgtcggagtgggttcagtctaaacaagaagtagaccctttcccggccactgtaa

SEQ ID NO: 1156 - DNA, 54 bp, synthetic construct
  source 1..54: mol_type = other DNA
cgtcggagtgggttcagtctaggcgctgatgaattggggttctctggctccatc

SEQ ID NO: 1157 - DNA, 54 bp, synthetic construct
  source 1..54: mol_type = other DNA
cgtcggagtgggttcagtctactcgatttaggtcaccgggtggagaggtgttct

SEQ ID NO: 1158 - DNA, 54 bp, synthetic construct
  source 1..54: mol_type = other DNA
cgtcggagtgggttcagtctagggttgatcagtggtgtcgggccttaggatctc

SEQ ID NO: 1159 - DNA, 54 bp, synthetic construct
  source 1..54: mol_type = other DNA
cgtcggagtgggttcagtctagggcatcctcaaagcgccagtactgactaccct

SEQ ID NO: 1160 - DNA, 54 bp, synthetic construct
  source 1..54: mol_type = other DNA
cgtcggagtgggttcagtctacggaatactgagcaatggagcgtgggtagggag

SEQ ID NO: 1161 - DNA, 45 bp, synthetic construct
  source 1..45: mol_type = other DNA
tgtggagggattgaaggataacttccccgtggttgagtcaaaaat

SEQ ID NO: 1162 - DNA, 48 bp, synthetic construct
  source 1..48: mol_type = other DNA
tgtggagggattgaaggataaccccagacttggccttccaattgtagg

SEQ ID NO: 1163 - DNA, 44 bp, synthetic construct
  source 1..44: mol_type = other DNA
tgtggagggattgaaggatagagttccagaatgaggttgccagg

SEQ ID NO: 1164 - DNA, 45 bp, synthetic construct
  source 1..45: mol_type = other DNA
tgtggagggattgaaggataagagttccagaatgaggttgccagg

SEQ ID NO: 1165 - DNA, 46 bp, synthetic construct
  source 1..46: mol_type = other DNA
tgtggagggattgaaggataagggttcgccataaatggctaccgtc

SEQ ID NO: 1166 - DNA, 44 bp, synthetic construct
  source 1..44: mol_type = other DNA
tgtggagggattgaaggatatgacatcgacttggagtcgattca

SEQ ID NO: 1167 - DNA, 48 bp, synthetic construct
  source 1..48: mol_type = other DNA
tgtggagggattgaaggatacgttgactactggcaggatcaaccacta

SEQ ID NO: 1168 - DNA, 44 bp, synthetic construct
  source 1..44: mol_type = other DNA
tgtggagggattgaaggataacttccccgtggttgagtcaataa

SEQ ID NO: 1169 - DNA, 45 bp, synthetic construct
  source 1..45: mol_type = other DNA
tgtggagggattgaaggataggattcgccataaatggctaccgtc

SEQ ID NO: 1170 - DNA, 45 bp, synthetic construct
  source 1..45: mol_type = other DNA
tgtggagggattgaaggatagtaacttggagtcgatagtcccaga

SEQ ID NO: 1171 - DNA, 49 bp, synthetic construct
  source 1..49: mol_type = other DNA
tgtggagggattgaaggatatcgatgactactggcaggatcaaccacta

SEQ ID NO: 1172 - DNA, 46 bp, synthetic construct
  source 1..46: mol_type = other DNA
tgtggagggattgaaggataagtacctcccctgaatcgggattccc

SEQ ID NO: 1173 - DNA, 44 bp, synthetic construct; identical to SEQ ID NO: 394
  source 1..44: mol_type = other DNA

SEQ ID NO: 1174 - DNA, 47 bp, synthetic construct; identical to SEQ ID NO: 400
  source 1..47: mol_type = other DNA

SEQ ID NO: 1175 - DNA, 46 bp, synthetic construct; identical to SEQ ID NO: 408
  source 1..46: mol_type = other DNA

SEQ ID NO: 1176 - DNA, 44 bp, synthetic construct; identical to SEQ ID NO: 406
  source 1..44: mol_type = other DNA

SEQ ID NO: 1177 - DNA, 45 bp, synthetic construct; identical to SEQ ID NO: 402
  source 1..45: mol_type = other DNA

SEQ ID NO: 1178 - DNA, 44 bp, synthetic construct; identical to SEQ ID NO: 396
  source 1..44: mol_type = other DNA

SEQ ID NO: 1179 - DNA, 44 bp, synthetic construct; identical to SEQ ID NO: 388
  source 1..44: mol_type = other DNA

SEQ ID NO: 1180 - DNA, 44 bp, synthetic construct; identical to SEQ ID NO: 392
  source 1..44: mol_type = other DNA

SEQ ID NO: 1181 - DNA, 44 bp, synthetic construct; identical to SEQ ID NO: 390
  source 1..44: mol_type = other DNA

SEQ ID NO: 1182 - DNA, 44 bp, synthetic construct; identical to SEQ ID NO: 398
  source 1..44: mol_type = other DNA

SEQ ID NO: 1183 - DNA, 47 bp, synthetic construct; identical to SEQ ID NO: 404
  source 1..47: mol_type = other DNA

SEQ ID NO: 1184 - DNA, 44 bp, synthetic construct; identical to SEQ ID NO: 410
  source 1..44: mol_type = other DNA

SEQ ID NO: 1185 - DNA, 63 bp, synthetic construct
  source 1..63: mol_type = other DNA
ttggaggtgtagggagtaaattgttgcaaaggaacgtcgagctgtaatggtgctcgacgttcc

SEQ ID NO: 1186 - DNA, 63 bp, synthetic construct
  source 1..63: mol_type = other DNA
gctcgacgttcctttgcaacaggaacgtcgagcaccattacatttggaggtgtagggagtaaa

SEQ ID NO: 1187 - DNA, 49 bp, synthetic construct
  source 1..49: mol_type = other DNA
ctggcaccgctaaaccgtattgccaaaccaggcaaagcgccattcgcca

SEQ ID NO: 1188 - DNA, 49 bp, synthetic construct
  source 1..49: mol_type = other DNA
ctggcaccgctaaaccgtattatttgcgaacagcgcacggcgttaaagt

SEQ ID NO: 1189 - DNA, 49 bp, synthetic construct
  source 1..49: mol_type = other DNA
ctggcaccgctaaaccgtattgccttaacgccgcgaatcagcaacggct

SEQ ID NO: 1190 - DNA, 49 bp, synthetic construct
  source 1..49: mol_type = other DNA
ctggcaccgctaaaccgtattgcttaatttcaccgccgaaaggcgcggt

SEQ ID NO: 1191 - DNA, 49 bp, synthetic construct
  source 1..49: mol_type = other DNA
ctggcaccgctaaaccgtattcattcgcttgccaccgcaacatccacat

SEQ ID NO: 1192 - DNA, 49 bp, synthetic construct
  source 1..49: mol_type = other DNA
ctggcaccgctaaaccgtattgggtgccacaaagaaaccgtcacccgca

SEQ ID NO: 1193 - DNA, 49 bp, synthetic construct
  source 1..49: mol_type = other DNA
ctggcaccgctaaaccgtattcgctgcagcagatggcgatggctggttt

SEQ ID NO: 1194 - DNA, 49 bp, synthetic construct
  source 1..49: mol_type = other DNA
ctggcaccgctaaaccgtattacgaacaacgccgcttcggcctggtaat

SEQ ID NO: 1195 - DNA, 49 bp, synthetic construct
  source 1..49: mol_type = other DNA
ctggcaccgctaaaccgtattcgatctgaccatgcggtcgcgtttggtt

SEQ ID NO: 1196 - DNA, 49 bp, synthetic construct
  source 1..49: mol_type = other DNA
ctggcaccgctaaaccgtattgccaaaccgacgtcgcaggcttctgctt

SEQ ID NO: 1197 - DNA, 49 bp, synthetic construct
  source 1..49: mol_type = other DNA
ctggcaccgctaaaccgtattcaaacccatcgcgtgggcatattcgcaa

SEQ ID NO: 1198 - DNA, 49 bp, synthetic construct
  source 1..49: mol_type = other DNA
ctggcaccgctaaaccgtattcagaatgcgggtcgcttcacttacgcca

SEQ ID NO: 1199 - DNA, 49 bp, synthetic construct
  source 1..49: mol_type = other DNA
ctggcaccgctaaaccgtattaactaatcagcaccgcgtcggcaagtgt

SEQ ID NO: 1200 - DNA, 49 bp, synthetic construct
  source 1..49: mol_type = other DNA
ctggcaccgctaaaccgtattctattcggcgctccacagttccggattt

SEQ ID NO: 1201 - DNA, 49 bp, synthetic construct
  source 1..49: mol_type = other DNA
ctggcaccgctaaaccgtattttgtgcttaccttgcgggccaacatcca

SEQ ID NO: 1202 - DNA, 49 bp, synthetic construct
  source 1..49: mol_type = other DNA
ctggcaccgctaaaccgtattcggtccagtaccgcgcggctgaaatcat

SEQ ID NO: 1203 - DNA, 49 bp, synthetic construct
  source 1..49: mol_type = other DNA
ctggcaccgctaaaccgtattcgctcgtgattagcgccgtggcctgatt

SEQ ID NO: 1204 - DNA, 49 bp, synthetic construct
  source 1..49: mol_type = other DNA
ctggcaccgctaaaccgtattgcgacagcgtgtaccacagcggatggtt

SEQ ID NO: 1205 - DNA, 49 bp, synthetic construct
  source 1..49: mol_type = other DNA
ctggcaccgctaaaccgtattagttacagaactggcgatcgttcggcgt

SEQ ID NO: 1206 - DNA, 49 bp, synthetic construct
  source 1..49: mol_type = other DNA
ctggcaccgctaaaccgtatttaacattggcaccatgccgtgggtttca

SEQ ID NO: 1207 - DNA, 49 bp, synthetic construct
  source 1..49: mol_type = other DNA
ctggcaccgctaaaccgtattcggtcttcgctattacgccagctggcga

SEQ ID NO: 1208 - DNA, 49 bp, synthetic construct
  source 1..49: mol_type = other DNA
ctggcaccgctaaaccgtatttagacactcgggtgattacgatcgcgct

SEQ ID NO: 1209 - DNA, 49 bp, synthetic construct
  source 1..49: mol_type = other DNA
ctggcaccgctaaaccgtattaggagataactgccgtcactccagcgca

SEQ ID NO: 1210 - DNA, 49 bp, synthetic construct
  source 1..49: mol_type = other DNA
ctggcaccgctaaaccgtattaaatttgatggaccatttcggcaccgcc

SEQ ID NO: 1211 - DNA, 49 bp, synthetic construct
  source 1..49: mol_type = other DNA
caacgatgcccgtagttgactgccaaaccaggcaaagcgccattcgcca

SEQ ID NO: 1212 - DNA, 49 bp, synthetic construct
  source 1..49: mol_type = other DNA
caacgatgcccgtagttgactatttgcgaacagcgcacggcgttaaagt

SEQ ID NO: 1213 - DNA, 49 bp, synthetic construct
  source 1..49: mol_type = other DNA
caacgatgcccgtagttgactgccttaacgccgcgaatcagcaacggct

SEQ ID NO: 1214 - DNA, 49 bp, synthetic construct
  source 1..49: mol_type = other DNA
caacgatgcccgtagttgactgcttaatttcaccgccgaaaggcgcggt

SEQ ID NO: 1215 - DNA, 49 bp, synthetic construct
  source 1..49: mol_type = other DNA
caacgatgcccgtagttgactcattcgcttgccaccgcaacatccacat

SEQ ID NO: 1216 - DNA, 49 bp, synthetic construct
  source 1..49: mol_type = other DNA
caacgatgcccgtagttgactgggtgccacaaagaaaccgtcacccgca

SEQ ID NO: 1217 - DNA, 49 bp, synthetic construct
  source 1..49: mol_type = other DNA
caacgatgcccgtagttgactcgctgcagcagatggcgatggctggttt

SEQ ID NO: 1218 - DNA, 49 bp, synthetic construct
  source 1..49: mol_type = other DNA
caacgatgcccgtagttgactacgaacaacgccgcttcggcctggtaat

SEQ ID NO: 1219 - DNA, 49 bp, synthetic construct
  source 1..49: mol_type = other DNA
caacgatgcccgtagttgactcgatctgaccatgcggtcgcgtttggtt

SEQ ID NO: 1220 - DNA, 49 bp, synthetic construct
  source 1..49: mol_type = other DNA
caacgatgcccgtagttgactgccaaaccgacgtcgcaggcttctgctt

SEQ ID NO: 1221 - DNA, 49 bp, synthetic construct
  source 1..49: mol_type = other DNA
caacgatgcccgtagttgactcaaacccatcgcgtgggcatattcgcaa

SEQ ID NO: 1222 - DNA, 49 bp, synthetic construct
  source 1..49: mol_type = other DNA
caacgatgcccgtagttgactcagaatgcgggtcgcttcacttacgcca

SEQ ID NO: 1223 - DNA, 49 bp, synthetic construct
  source 1..49: mol_type = other DNA
caacgatgcccgtagttgactaactaatcagcaccgcgtcggcaagtgt

SEQ ID NO: 1224 - DNA, 49 bp, synthetic construct
  source 1..49: mol_type = other DNA
caacgatgcccgtagttgactctattcggcgctccacagttccggattt

SEQ ID NO: 1225 - DNA, 49 bp, synthetic construct
  source 1..49: mol_type = other DNA
caacgatgcccgtagttgactttgtgcttaccttgcgggccaacatcca

SEQ ID NO: 1226 - DNA, 49 bp, synthetic construct
  source 1..49: mol_type = other DNA
caacgatgcccgtagttgactcggtccagtaccgcgcggctgaaatcat

SEQ ID NO: 1227 - DNA, 49 bp, synthetic construct
  source 1..49: mol_type = other DNA
caacgatgcccgtagttgactcgctcgtgattagcgccgtggcctgatt

SEQ ID NO: 1228 - DNA, 49 bp, synthetic construct
  source 1..49: mol_type = other DNA
caacgatgcccgtagttgactgcgacagcgtgtaccacagcggatggtt

SEQ ID NO: 1229 - DNA, 49 bp, synthetic construct
  source 1..49: mol_type = other DNA
caacgatgcccgtagttgactagttacagaactggcgatcgttcggcgt

SEQ ID NO: 1230 - DNA, 49 bp, synthetic construct
  source 1..49: mol_type = other DNA
caacgatgcccgtagttgacttaacattggcaccatgccgtgggtttca

SEQ ID NO: 1231 - DNA, 49 bp, synthetic construct
  source 1..49: mol_type = other DNA
caacgatgcccgtagttgactcggtcttcgctattacgccagctggcga

SEQ ID NO: 1232 - DNA, 49 bp, synthetic construct
  source 1..49: mol_type = other DNA
caacgatgcccgtagttgacttagacactcgggtgattacgatcgcgct

SEQ ID NO: 1233 - DNA, 49 bp, synthetic construct
  source 1..49: mol_type = other DNA
caacgatgcccgtagttgactaggagataactgccgtcactccagcgca

SEQ ID NO: 1234 - DNA, 49 bp, synthetic construct
  source 1..49: mol_type = other DNA
caacgatgcccgtagttgactaaatttgatggaccatttcggcaccgcc

SEQ ID NO: 1235 - DNA, 25 bp, synthetic construct
  source 1..25: mol_type = other DNA
  misc_feature 1: note = Alexa Fluor 488 (NHS Ester)
cccttctactcaattacctcatccc

SEQ ID NO: 1236 - DNA, 25 bp, synthetic construct
  source 1..25: mol_type = other DNA
  misc_feature 1: note = Alexa Fluor 647 (NHS Ester)
caccctcatatctattaccctccca

SEQ ID NO: 1237 - DNA, 20 bp, synthetic construct
  source 1..20: mol_type = other DNA
gatgatgtagtagtaagggt

SEQ ID NO: 1238 - DNA, 25 bp, synthetic construct
  source 1..25: mol_type = other DNA
gggatgaggtaattgagtagaaggg

SEQ ID NO: 1239 - DNA, 25 bp, synthetic construct
  source 1..25: mol_type = other DNA
tgggagggtaatagatatgagggtg

SEQ ID NO: 1240 - DNA, 60 bp, synthetic construct
  source 1..60: mol_type = other DNA
gtaattgagtagaagggtaatacggtttagcggtgccagtttaatgcactggcaccgcta

SEQ ID NO: 1241 - DNA, 60 bp, synthetic construct
  source 1..60: mol_type = other DNA
ctggcaccgctaaaccgtatttagcggtgccagtgcattaaatgggatgaggtaattgag

SEQ ID NO: 1242 - DNA, 60 bp, synthetic construct
  source 1..60: mol_type = other DNA
taatagatatgagggtgtagtcaactacgggcatcgttgttaaggcttcaacgatgcccg

SEQ ID NO: 1243 - DNA, 60 bp, synthetic construct
  source 1..60: mol_type = other DNA
caacgatgcccgtagttgactcgggcatcgttgaagccttaattgggagggtaatagata

SEQ ID NO: 1244 - DNA, 44 bp, synthetic construct
  source 1..44: mol_type = other DNA
tagagttgatagagggagaaagtcttggttttccggatttggga

SEQ ID NO: 1245 - DNA, 47 bp, synthetic construct
  source 1..47: mol_type = other DNA
tagagttgatagagggagaacatgtcaatgagcaaaggtattaagaa

SEQ ID NO: 1246 - DNA, 46 bp, synthetic construct
  source 1..46: mol_type = other DNA
tagagttgatagagggagaacatgtcaatgagcaaaggtattatga

SEQ ID NO: 1247 - DNA, 44 bp, synthetic construct
  source 1..44: mol_type = other DNA
tagagttgatagagggagaacgtcaccccattaagaggctcggt

SEQ ID NO: 1248 - DNA, 45 bp, synthetic construct
  source 1..45: mol_type = other DNA
tagagttgatagagggagaagaaactaacacacacactgattgtc

SEQ ID NO: 1249 - DNA, 44 bp, synthetic construct
  source 1..44: mol_type = other DNA
tagagttgatagagggagaagagccttggttttccggatttcgg

SEQ ID NO: 1250 - DNA, 44 bp, synthetic construct
  source 1..44: mol_type = other DNA
tagagttgatagagggagaaggagccttggttttccggattacg

SEQ ID NO: 1251 - DNA, 44 bp, synthetic construct
  source 1..44: mol_type = other DNA
tagagttgatagagggagaagtaagctcacaatatgtgcataaa

SEQ ID NO: 1252 - DNA, 44 bp, synthetic construct
  source 1..44: mol_type = other DNA
tagagttgatagagggagaagtcaccccattaagaggctccgtg

SEQ ID NO: 1253 - DNA, 44 bp, synthetic construct
  source 1..44: mol_type = other DNA
tagagttgatagagggagaagtgctcagccttggttttccgcta

SEQ ID NO: 1254 - DNA, 47 bp, synthetic construct
  source 1..47: mol_type = other DNA
tagagttgatagagggagaagtgtctcatctctgaaaacttccgacc

SEQ ID NO: 1255 - DNA, 44 bp, synthetic construct
  source 1..44: mol_type = other DNA
tagagttgatagagggagaatgacacacacactgattcagggag

SEQ ID NO: 1256 - DNA, 21 bp, synthetic construct
  source 1..21: mol_type = other DNA
  misc_feature 21: note = 3' amino modifier
cgtcaggtgagcatcttacat

SEQ ID NO: 1257 - DNA, 21 bp, synthetic construct
  source 1..21: mol_type = other DNA
  misc_feature 21: note = 3' amino modifier
gctcgacgttcctttgcaaca